(19) United States
(12) Patent Application Publication
(10) Pub. No.: US 2022/0098578 A1
(43) Pub. Date: Mar. 31, 2022

(54) NEOANTIGENS CREATED BY ABERRANT-INDUCED SPLICING AND USES THEREOF IN ENHANCING IMMUNOTHERAPY

(71) Applicants: BAR ILAN UNIVERSITY, Ramat Gan (IL); YISSUM RESEARCH DEVELOPMENT COMPANY OF THE HEBREW UNIVERSITY OF JERUSALEM LTD., Jerusalem (IL)

(72) Inventors: Erez LEVANON, Petach-Tiqva (IL); Rotem KARNI, Mevaseret Zion (IL); Ariel FEIGLIN, Mazkeret Batya (IL); Adi MOGILEVSKY, Jerusalem (IL); Eli KOPEL, Petach-Tikva (IL); Michal BARAK, Petah-Tikva (IL)

(21) Appl. No.: 17/427,405

(22) PCT Filed: Jan. 30, 2020
(86) PCT No.: PCT/IL2020/050119

§ 371 Date: Jul. 30, 2021

Related U.S. Application Data
(60) Provisional application No. 62/799,377, filed on Jan. 31, 2019.

(51) Int. Cl. C12N 15/11; C12N 15/113; C12N 9/22; A61K 39/00; A61K 31/7105; A61K 38/46; A61K 45/06; A61P 35/00

(52) U.S. Cl. CPC C12N 15/11; C12N 15/113; C12N 9/22; C12N 15/1137; A61K 39/001156; A61K 31/7105; C12N 2800/80; A61K 45/06; A61P 35/00; A61K 39/0011; C12N 2320/33; C12N 2310/20; C12N 2310/11; A61K 38/465

(57) ABSTRACT

The invention relates to methods, compositions, and splicing modulating agents comprising nucleic acid sequences, specifically, antisense oligonucleotides and gene editing compounds. The splicing modulating agents of the invention are used in methods for producing neoantigens in subjects suffering from neoplastic disorders, by the induction of aberrant splicing events. The modulators of the invention are further used by the invention in enhancing immunotherapy.

### FIELD OF THE INVENTION

The invention relates to immunotherapy. More specifically, the invention relates to methods, compositions, agents comprising nucleic acid sequences, oligonucleotides and gene editing compounds for producing neoantigens by the induction of aberrant splicing events, and uses thereof in enhancing immunotherapy.

### BACKGROUND ART

References considered to be relevant as background to the presently disclosed subject matter are listed below:
Ben Hur et al. (2013) Cell Reports 3: 103-115
Anczuków O, et al. (2016) RNA. 22:1285-301;
Shilo A, Siegfried Z, (2014) Mol Cell Oncol. 2014;
Maimon et al. (2014) Cell Reports 7: 501-513;
Mogilevsky, et al. (2018). Nucleic Acids Res. 46:11396-11404;
Rong-Fu Wang and Helen Y Wang (2017) Cell Research 27:11-37;
Levanon et al. (2015) Nucleic Acids Res. 43(10):5130-44;
WO2016/142948;
WO2013/171753;
U.S. Pat. No. 9,717,750;
U.S. Pat. No. 7,807,816;
U.S. Pat. No. 9,506,058;

Acknowledgement of the above references herein is not to be inferred as meaning that these are in any way relevant to the patentability of the presently disclosed subject matter.

### BACKGROUND OF THE INVENTION

In the past few years, new modalities of immunotherapy have opened a new era in the effectiveness of cancer treatment. This novel technology has joined conventional cancer treatments such as surgery, chemotherapy, radiation and targeted biological treatments such as antibodies. Cancers that were completely untreatable (such as metastatic melanoma) are showing dramatic positive responses to immunotherapy. Nevertheless, the huge promise of this treatment is diminished by the fact that only a limited percentage of patients are benefiting from this new approach and completely overcoming the disease. For most patients, current immunotherapy modalities cannot prolong lifespan in a significant way and definitely cannot achieve a “cure”.

The immune system is designed to distinguish between ‘self’—normal cells in the body—and ‘non-self’ or ‘foreign’ components. In order to avoid false and dangerous auto-immune activation, the immune system is under constant tight control, which is mediated by specific proteins that need to be activated or inactivated in a proper manner and timing, termed ‘immune checkpoints’ proteins. Cancer cells have developed elaborate ways to bypass these immune checkpoints and to maintain the immune system in its inactive state of non-recognition of tumor cells, while novel immunotherapeutic treatments aim to re-activate the anti-tumor immune response. One common way by which tumor cells efficiently suppress an anti-tumor immune response is bypassing the immune checkpoint pathway, which negatively regulates the cellular response. Cytotoxic T-lymphocyte protein 4 (CTLA4) and programmed cell death protein 1 (PD-1) have been identified as the main proteins in this pathway. CTLA-4 negatively regulates the T-cell activation by competing with CD28 for CD80/86 ligand binding, while PD-1 binds PD-L1/2, thereby inhibiting the T-cell activation signal. Immunotherapy is a natural and highly specific process that, when properly harnessed, can target tumor cells without affecting normal cells, thereby eliminating many of the unwanted side effects of traditional cancer therapies. Another type of immunotherapy is CAR-T adoptive cell transfer, in which autologous T cells are genetically engineered to produce surface chimeric antigen receptors (CARs), expanded, and infused into the patient, where they recognize and kill cancer cells harboring the surface antigen. Immunotherapy is also a dynamic and flexible process that can adapt to the development of tumors and changes on the tumor cell surface. Immunotherapy encompasses the incredible unique advantage of gaining ‘memory’, and with consequent amplification it prolongs responses and does not require additional drug delivery. Most importantly, there is no conventional resistance to such immunotherapy over time. If the tumor reappears, the patient simply can be treated again.

Hence, immunotherapy presents a new revolutionary direction in cancer treatment, aimed at complete elimination of the cancer and not just prolongation of life span. However, only ˜20% of patients with specific cancers gain long-term survival from immunotherapy. Vast efforts by many groups are being directed toward expanding the percentage of patients who will benefit from this strategy in terms of sustaining a durable response.

Extensive preclinical and clinical studies have been performed aiming to decipher the factors and markers predicting increased response to immunotherapy regimens and types (Morrison et al., 2018, J. Immunother. cancer, 6, 32; Jiang et al., 2018, Nat. Med., 24, 1550-1558; Auslander et al., 2018, Nat. Med., 24, 1545-1549; Miao et al., 2018, Science, 359, 801-806). Mutational burden appears to be one of the most highly diagnostic factors that predict a patient's positive response (Yarchoan et al., 2017, N. Engl. J. Med., 377, 2500-2501). High mutational load in a tumor seems to increase the chance of a having a potent neoantigen that will be displayed on tumor cells and recognized by the immune system as ‘foreign’, hence stimulating immune recognition and attack. There is therefore a crucial unmet need to make immunotherapy relevant to a wider range of cancer types and effective for much higher percentage of cancer patients. The present invention addresses these issues by targeting alteration of cancer RNAs (transcriptome) i.e. modulating the key RNA processing step, splicing, in a way that forces the production of neoantigens in the tumor cells, consequently activating anti-tumor T-cell responses.

Levanon E Y. et al., (2015), reported splicing alterations in hundreds of matched tumor and normal samples of multiple cancer types, identifying highly frequent altered splicing events.

Ben Hur et al. (2013) discloses that different splicing isoforms of the gene Ribosomal S6 kinase 1 (S6K1) have different effect on tumor development. Shilo A (2015) reviews the role of alternative splicing and its regulators in cancer initiation and progression. Similarly, Anczuków 0 (2016), splicing factors alterations detected in human tumors and the resulting changes in splicing. Modulation of splicing is therefore acknowledged by the art as an established approach for cancer treatment.

WO2019/23232449, disclose antibody-drug conjugates comprising splicing modulator, specifically, pladienolide or a pladienolide derivative and antibody that target the antibody-drug conjugates to cancer cells. These modulators bind the SF3b splisosome complex thereby promoting intron retention and/or exon skipping. However, the general splicing modulators used by this publication, cannot direct aberrant splicing events in particular target sites that induce aberrant splicing that results in creation of a neoantigen. Such neoantigen induces a specific immune response against tumor cells that express said neoantigen.

WO2013/171753 and similarly, Maimon et al. (2014), disclose that alternative splicing events may be induced using antisense oligonucleotides. These induced splicing events lead to change in the ratio of different resulting isoforms. However, activation of the immune response using aberrant-induced splicing, is not described or even hinted in these publications. Additional recent publication by some of the inventors, (Mogilevsky (2018)), demonstrated the induction of splicing events to modulate isoform ratio and uses thereof by in tumor inhibition. However, formation of neoantigen that does not exists in the proteome and uses thereof to induce an immune response targeted against tumor cells expressing the newly created neoantigen, is not disclosed by this publication.

Still further, WO2016/142948 discloses oligonucleotides to inhibit overall cellular splicing activity of specific splicing factors. This publication does not suggest the production of neoantigens for activating the immune response.

The use of antisense oligonucleotides for modulating splicing is known in the art. U.S. Pat. Nos. 7,807,816 and 9,506,058, discloses the use of antisense oligonucleotides to induce exon skipping in variety of exons at the dystrophine mRNA, specifically oligonucleotides targeted to induce skipping of exon 51, that results in the creation of an in frame functional dystrophine protein and uses thereof for treating Duchenne muscular dystrophy (DMD).

U.S. Pat. No. 9,717,750, discloses antisense oligonucleotide complementary to intron 7 of a nucleic acid encoding human survival motor neuron (SMN) protein 2 (SMN2) pre-mRNA, and uses thereof in treating spinal muscular atrophy (SMA) that is a genetic neurodegenerative disorder caused by loss of both copies of SMN1 gene. SMN2 contains a mutation at exon 7, which results in inefficient inclusion of exon 7 in SMN2 transcripts, thereby leading to a truncated version, lacking exon 7, which is unstable and inactive. The antisense oligonucleotide described in this publication induce the inclusion of exon 7 in the SMN2 mRNA and thereby, the creation of a functional polypeptides in motorneurons of SMA patients. However, these publications demonstrate the effective use of these antisense oligonucleotides to either exclude or include exons in reconstructing an active form of an in-frame protein product. However, the use of antisense oligonucleotides to induce aberrant splicing that leads to frame shift and creation of novel and immunogenic protein products, that are not expressed naturally, is not disclosed by these publications.

The review of Rong-Fu Wang and Helen Y Wang (2017) describes endogenous mechanisms that lead to the formation of neoantigens that are presented to T cells. This publication however, does not suggest artificial induction of aberrant splicing events.

There is therefore need in the art for in vivo creation of neo-antigens by artificial induction of aberrant splicing for enhancing immunotherapy.

### SUMMARY OF THE INVENTION

In a first aspect, the invention relates to a method for inducing the production of at least one neoantigen to be expressed by at least one target cell of a subject suffering from a neoplastic disorder. In some embodiments, the method comprising the step of contacting the at least one target cell with at least one splicing modulating agent comprising at least one nucleic acid sequence or any vector, vehicle, matrix, nano- or micro-particle or composition comprising the at least one agent. It should be noted that the nucleic acid sequence of this agent targets at least one nucleic acid sequence that participates directly or indirectly in at least one splicing event of at least one target gene. It should be further noted that in some embodiments, introduction of the at least one agent of the invention into the target cell induces at least one aberrant splicing event via said nucleic acid sequence. Such aberrant splicing event results in some embodiments, in the production of at least one neoantigen to be expressed by the target cell.

In yet a further aspect, the invention relates to a method for activating an immune response against at least one target cell, in a subject. In some embodiments, the subject may be a subject suffering from at least one neoplastic disorder. In some embodiments, the method of the invention may comprise the step of administering to the subject at least one splicing modulating agent comprising at least one nucleic acid sequence or any vector, vehicle, matrix, nano- or micro-particle or composition comprising the at least one agent. It should be noted that the nucleic acid sequence of the agent targets at least one target nucleic acid sequence that participates directly or indirectly in at least one splicing event of at least one target gene. It should be further noted that in some embodiments, introduction of the at least one agent of the invention into the target cell induces at least one aberrant splicing event via said nucleic acid sequence. Such aberrant splicing event results in some embodiments, in the production of said at least one neoantigen expressed by the target cell, thereby activating an immune response directed against the target cell in the administered subject.

In yet another aspect, the invention provides a method for treating, inhibiting, preventing, ameliorating or delaying the onset of at least one neoplastic disorder in a subject. In some embodiments, the method comprising the step of administering to the treated subject at least one splicing modulating agent comprising at least one nucleic acid sequence or any vector, vehicle, matrix, nano- or micro-particle or composition comprising the at least one oligonucleotide/s of the invention. It should be noted that the nucleic acid sequence of the agent targets at least one target nucleic acid sequence that participates directly or indirectly in at least one splicing event of at least one target gene. Introduction of the at least one agent of the invention into the target cell induces at least one aberrant splicing event via the nucleic acid sequence. Such aberrant splicing event results in some embodiments, in the production of said at least one neoantigen expressed by the target cell, thereby activating an immune response directed against the target cell in the treated subject. In some embodiments, the method of the invention may further comprise administering prior to, after and/or simultaneously to administration of the splicing modulating agent, at least one polypeptide derived from at least one neoantigen, or any derivative, enantiomer, fusion protein, conjugate, polyvalent dendrimer or any vaccine thereof.

The invention further provides a therapeutic effective amount of at least one splicing modulating agent comprising at least one nucleic acid sequence or of any vector, vehicle, matrix, nano- or micro-particle or composition comprising said at least one agent, for use in a method for treating, inhibiting, preventing, ameliorating or delaying the onset of at least one neoplastic disorder in a subject.

Another aspect of the invention relates to a composition comprising at least one splicing modulating agent comprising at least one nucleic acid sequence targeting at least one target nucleic acid sequence that participates directly or indirectly in at least one splicing event of a target gene, or any vector, vehicle, matrix, nano- or micro-particle comprising the at least one oligonucleotide. In some embodiments, the agent induces at least one aberrant splicing event via the nucleic acid sequence. It should be noted that the aberrant splicing event results in the production of at least one neoantigen expressed by the target cell. In some embodiments, the composition may be particularly applicable for activating an immune response against at least one target cell in a subject suffering from at least one neoplastic disorder.

In yet a further aspect thereof, the invention provides an antisense oligonucleotide targeting at least one target nucleic acid sequence that participates directly or indirectly in at least one splicing event of a target gene in a target cell. In some embodiments, the introduction of the oligonucleotides, specifically, AON of the invention into the target cell induces at least one aberrant splicing event via the target nucleic acid sequence. In more specific embodiments, such aberrant splicing event results in the production of at least one neoantigen expressed by the target cell.

Still further aspect of the invention relates to at least one polypeptide derived from at least one neoantigen, or any derivative, enantiomer, fusion protein, conjugate, polyvalent dendrimer or any vaccine thereof. The neoantigen is produced by at least one aberrant splicing event induced by at least one splicing modulating agent comprising at least one nucleic acid sequence, in a target cell of a subject suffering from a neoplastic disorder. A further aspect of the invention relates to a kit comprising:

First component of the kit of the invention may be at least one splicing modulating agent comprising at least one nucleic acid sequence targeting at least one target nucleic acid sequence that participates directly or indirectly in at least one splicing event of a target gene in a target cell. It should be noted that the introduction of the agent of the invention into a target cell induce at least one aberrant splicing event via the target nucleic acid sequence. Such aberrant splicing event results in the production of at least one neoantigen expressed by the target cell. The kit of the invention may optionally further comprise at least one of: In some embodiments, at least one peptide derived from the neoantigens of the invention. In yet some further embodiments, the kit of the invention may optionally further comprise at least one immuno-modulatory agent. In yet some further embodiments, the immuno-modulatory agent may be at least one immune-checkpoint inhibitor, for example an inhibitor directed against at least one of CTLA-4, PD-1 and PD-L1. A further aspect of the invention relates to a method for identifying a candidate target gene for induction of at least one aberrant splicing event to produce a neoantigen in at least one target cell of a mammalian subject. In more specific embodiments, the method of the invention may comprise the steps of: In a first step (a), selection and/or identification of coding transcripts of the mammalian subject that are characterized by at least one of: (i) said coding transcripts comprise at least three exons; (ii) at least one of the exons is of a length not divisible by three; and (iii) the coding transcripts comprise at least one intron. The next step (b), involves providing at least one predicted mRNA formed or transcribed by at least one aberrant splicing event of at least one of the coding transcripts selected in step (a). in some embodiments, the aberrant splicing event involves a nucleic acid sequence comprised within at least one of: (i) an exon that is of a length not divisible by three; (ii) least one intron located upstream or downstream to said exon; (iii) at least one splicing junction flanking said exon; and (iv) at least one splicing junction within the transcript. It should be noted that in some embodiments the predicted mRNAs formed by said aberrant splicing event encode at least one protein product that differs in at least one amino acid residue from a natural product produced in the mammalian subject. The next step (c) of the method of the invention involves providing at least one predicted peptide translated from the predicted mRNA of step (b). It should be noted that each of the predicted peptides derived from said neoantigen comprise at least one amino acid residue that differ from a natural product produced in said mammalian subject. In yet some further embodiments, such peptides may comprise between 8 to 22 amino acid residues. The next step (d), involves selecting or identifying from the at least one of predicted peptides of (c), peptides that bind MHC class I molecules, and/or MHC class II molecules of said mammalian subject. In the next step (e), identifying from the peptides selected in step (d), peptides that do not naturally occur in said mammalian subject. It should be noted that the identified peptides comprise the neoantigen, or in some embodiments, are part of the neoantigen and are therefore comprised within the neoantigens. In yet some further embodiments, the sequences encoding the peptides are comprised within a gene identified as a candidate target gene.

These and other aspects of the invention will become apparent by the hand of the following drawings.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1. Creation of neo-antigen in tumor cells

Figure illustrates the strategy for creating novel splicing isoforms that are translated into proteins with novel epitopes serving as neoantigens for immune recognition of the tumor cells. Light shaded shapes indicate new segments.

FIG. 2A-2E: TYR expression

FIG. 2A: Gene expression comparison of TCGA melanoma and healthy GTEx skin expression.

FIG. 2B: Expression across GTEx healthy tissues.

FIG. 2C: Gene model colored by expression of each exon in skin (generated using the GTEx portal, dark shading indicates relatively high expression). Targeted exon (182 bp) is marked and is of a length not divisible by 3

FIG. 2D: Expression of TYR across a variety of mouse tissues and cell types.

FIG. 2E: NCBI BLASTp top 20 results show no perfect match for the aberrant TYR peptide within the known human proteome.

FIG. 3A-3E: Inducing novel splice variants using ASOs in B16 Melanoma cells

FIG. 3A: ASO mini screen to identify the best inducer of exon skipping/inclusion. Green ASOs target junctions that block the 3′ or 5′ splice site and usually lead to exon skipping. ASOs may also inhibit binding of unknown splicing factors (SF) by masking exonic or intronic splicing enhancer or silencer sequences. Masking enhancer sequences may lead to exon skipping while blocking silencer sequences may lead to exon inclusion (potential enhancer/silencer sequences are marked in light grey)

FIG. 3B: RT-PCR results of a mini screen with 20 ASOs spanning the boundaries of the target exon.

FIG. 3C: Dose response for the best oligo candidates in TYR.

FIG. 3D: RT-PCR readout using oligo 13 for TYR is shown compared to a randomized oligo preserving base composition (SCRB). The corresponding splice models are depicted.

FIG. 3E: Sanger sequencing displays a novel isoform created by fusion of exons 3 and 5 in TYR. The resulting protein includes 8 new residues (bold letters).

FIG. 4A-4D. Vaccination with immunogenic TYR peptide activates the immune system in C57BL/6 mice

FIGS. 4A-4B. Isolated T cells were seeded in 96-well plates 1·10⁶ cells per well in duplicate and stimulated with different peptides; no stimulation (−), TYR, OVA or anti CD3. Anti CD3 serves as a positive control as it plays a critical role in T cell activation. T cells were stained for CD8 and IFN-γ and analyzed by flow cytometry. The average % of CD8+ cells that are also IFN-γ+ is shown for every group of mice (FIG. 4A). Results for individual mice are shown in FIG. 4B.

FIGS. 4C-4D. Isolated T cells were seeded in 96-well plates 1·10⁶ cells per well in duplicate and stimulated with the different peptides; no stimulation (−), TYR, OVA or CD3. After 72 hours, medium was collected and IFN-γ secretion was measured by ELISA assay. Average IFN-γ concentration for every group of mice is shown in FIG. 4C. Normalized IFN-γ concentrations for individual mice in the TYR immunized group are shown in FIG. 4D.

FIG. 5. TYR splicing modulation with CRISPR/cas9 results in a new isoform of TYR RT-PCR products from B16-F1 cells transduced with either control sgRNA (CRISPR control) or sgRNAs against 3′ or 5′ splice sites of exon 4 of the TYR mouse isoform NM_011661.5 (TYR 3′ss or TYR 5′ss respectively, SEQ ID NOs. 16-19) are shown. A new isoform, matching the length expected by exon 4 exclusion (489 bp−182 bp=307 bp), is evident in TYR 3′ss and TYR 5′ss treated cells but not the control.

FIG. 6A-6C. TYR splicing modulation with CRISPR/cas9 does not affect the cancerous properties of B16-F1 cells

FIG. 6A. A clonogenic assay was performed on B16-F1 cells transduced with either control sgRNA (CRISPR control) or sgRNAs against 3′ or 5′ splice sites of exon 4 of the TYR gene (TYR 3ss or TYR 5ss, respectively). 500 cells per well were seeded in duplicate in 6-well plates with 2 ml of media (DMEM, 10% FBS). After 14 days cells were fixated with 2.5% glutaraldehyde solution for 10 min, stained with 1% methylene blue solution, photographed and counted.

FIG. 6B. Proliferation assay of cells described in (FIG. 6A). 1000 cells per well were seeded in 96-well plates. Every 24 hours, one plate was fixated with 2.5% glutaraldehyde solution for 10 min and stained with 1% methylene blue solution. After incubation with 0.1N HCl for 1 hour, the absorbance (655 nm) of the extracted dye was measured using a plate reader. Error bars, SD from six repeats are shown.

FIG. 6C. Anchorage—independent growth assay performed on cells described in (FIG. 4A). Each well of 6-well plate was coated with 2 ml of bottom agar mixture (DMEM, 10% FBS, 1% agar). After the bottom layer had solidified, 2 ml of top agar mixture (DMEM, 10% FBS, 0.3% agar) containing 30,000 cells per well were seeded in duplicate. After this layer had solidified, 2 ml of media (DMEM, 10% FBS) was added to each well. After 14 days, colonies from 10 different fields per well were counted and the average number of colonies per well was calculated.

FIG. 7A-7D. TYR splicing modulation with CRISPR/cas9 in B16-F1 cells inhibits tumor growth in C57BL/6 mice

FIG. 7A. Average tumor volumes in C57BL/6 mice injected intradermally (200,000 cells/50 μl per mouse) with either B16-F1 CRISPR TYR transduced cells (N=11) or control B16-F1 cells (N=6) at three timepoints. Error bars depict the positive standard deviation. Asterisk indicates a significant difference on the last day of measurement (p=0.03 in a one tailed T-test).

FIG. 7B. Average tumor volumes in NOD-SCID mice injected subcutaneously (1·10⁶ cells/200 μl per mouse) with either B 16-F1 CRISPR TYR transduced cells (N=13) or control B 16-F1 cells (N=12) at three timepoints. Error bars depict positive standard deviation.

FIG. 7C. Tumor volumes on the last day of measurement for C57BL/6 and NOD-SCID mice injected with either B 16-F1 CRISPR TYR transduced cells or control cells. A significant difference was observed for C57BL/6 (p=0.03 in a one tailed T-test) but not NOD-SCID mice (indicated by NS).

FIG. 7D. T Cells isolated from mouse splenocytes were stained for CD8 and IFN-γ and analyzed by flow cytometry. Percent of IFN-γ+ CD8 T-cells are shown for four groups: (1) cells previously exposed to and activated with aberrant TYR peptides (expressed in their tumor)—‘Activated TYR’, (2) cells previously exposed but not activated—‘Naïve TYR’, (3) cells not previously exposed but activated—‘Activated control’ and (4) cells not previously exposed and not activated—‘Naïve control’.

FIG. 8. hnRNPAB splicing modulation with CRISPR/cas9 results in a new isoform of hnRNPAB

RT-PCR products from 4T1 cells transduced with either control sgRNA (CRISPR control) or sgRNAs against 3′ or 5′ splice sites of exon 6 of the hnRNPAB mouse isoform NM_010448.3 (hnRNPAB 3′ss, SEQ ID NOs. 57-58 or hnRNPAB 5′ss respectively) are shown. In addition to two natural hnRNPAB isoforms, a new isoform, matching the length expected by exon 6 exclusion (210 bp−103 bp=107 bp), is evident in hnRNPAB 3′ss but not in hnRNPAB 5′ss or the control, indicating that only CRISPR hnRNPAB 3′ss induced exon skipping.

FIG. 9A-9C. hnRNPAB splicing modulation with CRISPR/cas9 in 4T1 cells inhibits tumor growth in BALB/c mice

FIG. 9A. Average tumor volumes in BALB/c mice injected subcutaneously (500,000 cells/200 μl per mouse) with either 4T1 CRISPR hnRNPAB transduced cells (N=10) or control 4T1 cells (N=10) at six timepoints. Error bars depict the positive standard deviation. Asterisk indicates a significant difference on the two last days of measurement (p=0.0031 and p=0.0039 in a one tailed T-test respectively).

FIG. 9B. Average tumor volumes in NOD-SCID mice injected subcutaneously (500,000 cells/200 μl per mouse) with either 4T1 CRISPR hnRNPAB transduced cells (N=10) or control 4T1 cells (N=10) at three timepoints. Error bars depict positive standard deviation.

FIG. 9C. Tumor volumes on the last day of measurement for BALB/c and NOD-SCID mice injected with either 4T1 CRISPR hnRNPAB transduced cells or control cells. A significant difference was observed for BALB/c (p=0.03 in a one tailed T-test) but not NOD-SCID mice (indicated by NS).

### DETAILED DESCRIPTION OF THE INVENTION

In a first aspect, the invention relates to a method for inducing the production, creation and/or the formation of at least one neoantigen to be expressed by at least one target cell. In some embodiments, such cell is of a subject suffering from a neoplastic disorder.

In some embodiments, the method comprising the step of contacting the at least one target cell with an at least one splicing modulating agent comprising at least one nucleic acid sequence or any vector, vehicle, matrix, nano- or micro-particle or composition comprising the at least one agent. It should be noted that the at least one nucleic acid sequence of the agent, targets at least one target nucleic acid sequence that participates directly or indirectly in at least one splicing event of at least one target gene, or at least one target transcript. It should be further noted that in some embodiments, introduction of the at least one agent of the invention into the target cell induces at least one aberrant splicing event via said target nucleic acid sequence. Such aberrant splicing event results in some embodiments, in the production of said at least one neoantigen to be expressed by the target cell.

The invention thus provides at least one splicing modulating agent comprising at least one nucleic acid sequence that targets at least one target nucleic acid sequence that participates directly or indirectly in at least one splicing event of at least one target gene or at least one transcript thereof. As indicated above, it should be appreciated that embodiments, the splicing modulating agent comprising at least one nucleic acid sequence used by the methods of the invention can also be expressed from a nucleic acid construct administered to the individual or contacted with the target cells employing any suitable mode of administration (i.e., in-vivo gene therapy). Alternatively, the nucleic acid construct is introduced into a suitable cell via an appropriate gene delivery vehicle/method (transfection, transduction, homologous recombination, etc.).

The invention thus provides methods, compositions and splicing modulating agent comprising at least one nucleic acid sequence for creating neoantigens by induction of aberrant splicing. As used herein the term “neoantigen” is an antigen that has at least one alteration that makes it distinct from the corresponding wild-type, parental protein, preferably, by at least one amino acid residue, thereby forming a novel unrecognized antigen. In the context of this invention, this alteration results from an aberrant splicing event and therefore the neoantigen of the methods, compositions and kits of the inventions, may be created as a result of aberrant splicing variants, that do not occur naturally in a mammalian subject, e.g., human or rodents, that undergo the induced aberrant splicing in accordance with the invention. It should be further understood that in some embodiments, the neoantigen produced by the methods of the invention is a protein absent from normal tissues, and moreover, a protein that does not exist in the natural mammalian proteome, particularly, in the human and/or rodent proteomes. In yet some further specific embodiments, a neoantigen is an antigen to which the immune system has not previously been exposed to. It should be noted that the neoantigens produced by the methods of the invention and any peptides derived from such neoantigen of the invention are predicted to elicit an immune response in a subject, specifically, a human subject. In more specific embodiments, the neoantigen or any peptides thereof in accordance with the invention can be specifically recognized by neoantigen-specific T cell receptors in the context of major histocompatibility complexes (MHCs) molecules. In yet some further embodiments, peptides derive from the neoantigens of the invention, particularly any peptide comprising between about eight to about twenty two amino acid residues, specifically, 8, 9, 10, 11, 12, 13, 14, 15, 16, 17, 18, 19, 20, 21, 22 amino acid residues, or more specifically, any 8-mer, 9-mer, 10-mer, 11-mer, 12-mer, 13-mer, 14-mer, 15-mer, 16-mer, 17-mer, 18-mer, 19-mer, 20-mer, 21-mer, 22-mer peptide, specifically any nine-mer (9-mer) peptides derived from such neoantigens of the invention, are predicted (using NetHCpan) according to some embodiments of the invention, to bind with strong affinity to any HLA allele. In some specific and non-limiting embodiments, at least one of the following HLA alleles: HLA-A01:01, HLA-A02:01, HLA-A03:01, HLA-A11:01, HLA-A23:01, HLA-A24:02, HLA-A33:03, HLA-B07:02, HLA-B08:01, HLA-B44:02, HLA-C01:02, HLA-C04:01, HLA-C05:01, HLA-C06:02, HLA-C07:01, HLA-C07:02, HLA-C08:01.

In some embodiments, the splicing modulating agent used by the methods of the invention comprises at least one of the following agents. One option for such agent (a), may be at least one oligonucleotide comprising a nucleic acid sequence complementary to at least part of the target nucleic acid sequence. Another option for such agent (b), is at least one nucleic acid sequence comprising at least one guide RNA (gRNA) that targets at least one protospacer within the target nucleic acid sequence, in the target gene or at least one target transcript thereof. The agent used by the methods of the invention may be any nucleic acid sequence encoding such gRNA. It should be noted that this gRNA guides at least one programmable engineered nuclease (PEN) to the target nucleic acid sequence in said target gene.

Still further, the methods of the invention, as well as the compositions, kits and any of the splicing modulating agents that comprise at least one nucleic acid sequence described herein after modulate and modify splicing events in a target gene, and lead to creation of neoantigens by inducing aberrant splicing. In one aspect, the splicing modulating agent provided herein modulate splicing of a target gene in order to generate aberrant splicing events. Specifically, aberrant splicing event according to the invention relates to exon skipping and/or intron retention. Such modulation includes promoting or inhibiting exon inclusion or exclusion. Still further, in some embodiments, aberrant splicing event result in mRNA transcripts comprised of a different combination of exons. In certain embodiments, aberrant splicing event result in mRNA transcripts with deletions of exons. In certain embodiments, aberrant splicing event result in mRNA transcripts with deletions of portions of exons, or with extensions of exons, or with new exons. In certain embodiments, aberrant splicing event result in mRNA transcripts comprising premature stop codons. In yet some further embodiments, aberrant splicing event may result in intron retention. Further provided herein are splicing modulating agents that comprise at least one nucleic acid sequence (e.g. antisense compounds such as oligonucleotides and gRNAs), that are targeted to cis splicing regulatory elements present in pre-mRNA molecules, including exonic splicing enhancers, exonic splicing silencers, intronic splicing enhancers and intronic splicing silencers. Disruption of cis splicing regulatory elements is thought to alter splice site selection, which may lead to an alteration in the composition of splice products. Alternative splicing or “splicing” as used herein, is the process by which exons of primary transcripts can be spliced into alternative arrangements to produce structurally and functionally different messenger RNA (mRNA) i.e. splicing variants. During splicing, introns are removed and exons are joined together. The splicing process can be regulated by different cis- and trans-acting factors that influence the selection of specific splicing junctions. More specifically, splice junctions are also referred to as splice sites with the junction at the 5′ side of the intron often called the “5′ splice site,” or “splice donor site” and the junction at the 3′ side of the intron called the “3′ splice site” or “splice acceptor site.” In splicing, the 3′ end of an upstream exon is joined to the 5′ end of the downstream exon. Thus the unspliced RNA (or pre-mRNA) has an exon/intron junction at the 5′ end of an intron and an intron/exon junction at the 3′ end of an intron. After the intron is removed, the exons are contiguous at what is sometimes referred to as the exon/exon junction or boundary in the mature mRNA. Point mutations in a gene may weaken or strengthen splice sites, enhancer or silencer elements or lead to their destruction. This in turn causes alteration of splicing events. More specifically, the exons to be retained in the mRNA are determined during the splicing process. The regulation and selection of splice sites are done by trans-acting splicing activator and splicing repressor proteins as well as cis-acting elements within the pre-mRNA itself such as exonic splicing enhancers and exonic splicing silencers.

The typical eukaryotic nuclear intron has consensus sequences defining important regions. Each intron has the sequence GU at its 5′ end. Near the 3′ end there is a branch site. The nucleotide at the branchpoint is always an A; the consensus around this sequence varies somewhat. The branch site is followed by a series of pyrimidines, the polypyrimidine tract then by AG at the 3′ end. Splicing of mRNA is performed by an RNA and protein complex known as the spliceosome, containing snRNPs designated U1, U2, U4, U5, and U6 (U3 is not involved in mRNA splicing). U1 binds to the 5′ GU and U2, with the assistance of the U2AF protein factors, binds to the branchpoint A within the branch site. The complex at this stage is known as the spliceosome A complex. Formation of the A complex is usually the key step in determining the ends of the intron to be spliced out, and defining the ends of the exon to be retained. (The U nomenclature derives from their high uridine content). The U4, U5, U6 complex binds, and U6 replaces the U1 position. U1 and U4 leave. The remaining complex then performs two transesterification reactions. In the first transesterification, 5′ end of the intron is cleaved from the upstream exon and joined to the branch site A by a 2′,5′-phosphodiester linkage. In the second transesterification, the 3′ end of the intron is cleaved from the downstream exon, and the two exons are joined by a phosphodiester bond. The intron is then released in lariat form and degraded.

Splicing is regulated by trans-acting proteins (repressors and activators) and corresponding cis-acting regulatory sites (silencers and enhancers) on the pre-mRNA. However, as part of the complexity of alternative splicing, it is noted that the effects of a splicing factor are frequently position-dependent. That is, a splicing factor that serves as a splicing activator when bound to an intronic enhancer element may serve as a repressor when bound to its splicing element in the context of an exon, and vice versa. The secondary structure of the pre-mRNA transcript also plays a role in regulating splicing, such as by bringing together splicing elements or by masking a sequence that would otherwise serve as a binding element for a splicing factor. Together, these elements form a “splicing code” that governs how splicing will occur under different cellular conditions.

There are two major types of cis-acting RNA sequence elements present in pre-mRNAs and they have corresponding trans-acting RNA-binding proteins. Splicing silencers are sites to which splicing repressor proteins bind, reducing the probability that a nearby site will be used as a splice junction. These can be located in the intron itself (intronic splicing silencers, ISS) or in a neighboring exon (exonic splicing silencers, ESS). They vary in sequence, as well as in the types of proteins that bind to them. The majority of splicing repressors are heterogeneous nuclear ribonucleoproteins (hnRNPs) such as hnRNPA1 and polypyrimidine tract binding protein (PTB). Splicing enhancers are sites to which splicing activator proteins bind, increasing the probability that a nearby site will be used as a splice junction. These also may occur in the intron (intronic splicing enhancers, ISE) or exon (exonic splicing enhancers, ESE). Most of the activator proteins that bind to ISEs and ESEs are members of the SR protein family. Such proteins contain RNA recognition motifs and arginine and serine-rich (RS) domains.

In some embodiments, the aberrant splicing induced by the present invention may involve Exon skipping, where an exon may be spliced out of the primary transcript or retained. More specifically, “Exon skipping” refers generally to the process by which an entire exon, or a portion thereof, is removed from a given pre-processed RNA, and is thereby excluded from being present in the mature RNA, such as the mature mRNA that is translated into a protein. Hence, the portion of the protein that is otherwise encoded by the skipped exon is not present in the expressed form of the protein, typically creating an altered form of the protein. Still further, in some embodiments, alternative donor site, specifically, an alternative 5′ splice junction (donor site), changing the 3′ boundary of the upstream exon, and/or an alternative acceptor site, specifically, an alternative 3′ splice junction (acceptor site), changing the 5′ boundary of the downstream exon, may be utilized in exon skipping.

Additionally, most eukaryotic genes contain several pseudoexons, sequences resembling perfect exons, which are nonetheless ignored by the splicing machinery. Aberrant pseudoexon inclusion due to deep intronic mutations has been uncovered in recent years as a frequent cause of human diseases. When a pseudoexon inclusion leads to premature insertion of a termination codon in the mRNA, the term “nonsense exon” is used, also indicating that the mRNA undergoes rapid degradation by nonsense-mediated decay (NMD) pathways.

An “exon” refers to a defined section of nucleic acid that encodes for a protein, or a nucleic acid sequence that is represented in the mature form of an RNA molecule after either portions of a pre-processed (or precursor) RNA have been removed by splicing. The mature RNA molecule can be a messenger RNA (mRNA) or a functional form of a non-coding RNA, such as rRNA or tRNA. An “intron” refers to a nucleic acid region (within a gene) that is not translated into a protein. An intron is a non-coding section that is transcribed into a precursor mRNA (pre-mRNA), and subsequently removed by splicing during formation of the mature RNA.

It should be understood that the invention further encompasses the option of intron retention, induced by the method of the invention. for example, when the excision of an intron is suspended by the methods of the invention. Thus, in yet some alternative embodiments, in case of induction of intron retention by the aberrant or abnormal splicing event induced by the method of the invention, the least one target nucleic acid sequence that participates directly or indirectly in at least one splicing event may be comprised within at least one splicing junction within the target transcript. “Gene” as used herein, may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA or antisense RNA. Genes are composed of coding and non-coding transcripts, that may utilize the same sequence space on the same and opposite strands often each controlled by their own distinct regulatory regions. In general, “transcript” as used herein may refer to any nucleic acid or its sequence of any gene or gene combination and any variant thereof, in particular mRNA or cDNA sequence variants thereof. “Isoform” is used to relate to a particular variant of a transcript.

The invention involves the use of splicing modulating agent/s, or splicing modulator/s. “Modulator”, as used herein means a compound that leads or causes directly or indirectly to a perturbation of function or activity, specifically, of splicing. In certain embodiments, modulation means an increase, a decrease or alteration in splicing of a specific target. In some specific embodiments, modulators of splicing in accordance with the invention lead to aberrant splicing that in some embodiments results in frame shift and thereby the creation of a neoantigen that is not expressed normally. More specifically, in some embodiments, the splicing modulator agent/s of the invention induce aberrant splicing, as discussed above. In some embodiments, these aberrant splicing events lead to a frameshift, specifically, causing indels (insertions or deletions) of a number of nucleotides in the nucleic acid sequence of the target transcript/s that is not divisible by three which consequently disrupts the triplet reading frame of a nucleic acid sequence. More specifically, due to the triplet nature of gene expression by codons, the insertion or deletion can change the reading frame (the grouping of the codons), resulting in a completely different translation as compared to the original template. A frameshift will in general cause the reading of the codons after the splicing event to code for different amino acids, thereby leading to the creation of a neoantigen.

In some embodiments, the at least one nucleic acid sequence comprised within the splicing modulating agents used by the methods of the invention, target at least one target nucleic acid sequence that participates directly or indirectly in at least one splicing event. In some embodiments, such target nucleic acid sequence comprises at least one of, a splicing junction, a splice donor site, a splice acceptor site, an exonic splicing enhancer, splicing silencer, an intronic splicing enhancer and an intronic splicing silencer of said target gene, as specified above.

In some embodiments, the least one target nucleic acid sequence that participates directly or indirectly in at least one splicing event may be comprised within at least one coding transcript of the target gene. In some embodiments the coding transcript is characterized by at least one of the following parameters: (i) the coding transcript/s comprise at least three exons; (ii) at least one of the exons is of a length not divisible by three; and (iii) the coding transcripts comprise at least one intron. In some specific embodiments, the target nucleic acid sequence that participates directly or indirectly in at least one splicing event is comprised within least one of the at least three exons described above, specifically an exon that is not the first exon in the transcript. In yet some further embodiments, the target nucleic acid sequence that participates directly or indirectly in at least one splicing event, is comprised within an exon that is one of the at least three exons described above, specifically an exon that is not the last exon in the transcript. In yet some further embodiments, the target nucleic acid sequence that participates directly or indirectly in at least one splicing event may be comprised within an exon that is before the last exon in the transcript. In yet some further embodiments, such exon is in a length not divisible by three (3). Still further, in some embodiments, induction of exon skipping by the aberrant splicing event induced by the invention, may involve at least one exon in a length not divisible by three (3), thereby enabling frame shift. More specifically, in case of exclusion or inclusion of a nucleic acid sequence (e.g., an exon) by the aberrant splicing event induced by the splicing modulating agent of the invention, the length of the excluded or included sequence should be a length that cannot be divided by three (3). In such case, the exclusion or inclusion will cause a frame shift in the resulting transcript.

In yet some further embodiments, the target sequence in the target transcript is a target that aberrant splicing mediated by such sequence, leads to a frame shift that creates a neoantigen that does not exists in the human proteome. Still further, in some embodiments, peptides derived from such neoantigen, may be any one of 8-mer, 9-mer, 10-mer, 11-mer, 12-mer, 13-mer, 14-mer, 15-mer, 16-mer, 17-mer, 18-mer, 19-mer, 20-mer, 21-mer, 22-mer peptides, specifically, 8-14-mer peptides, more specifically 9-mer peptides (peptides comprising nine amino acid residues), display high affinity to HLA molecules and are therefore immunogenic. Moreover, such peptides do not exist in a mammalian proteome, specifically, the human proteome.

In some embodiments, the target nucleic acid sequence is comprised within an exon, or within at least one intron located upstream or downstream to the target exon, or within at least one splicing junction flanking the exon. In some embodiments, the target sequence resides within an exon, specifically an exon that is not the first exon in the transcript. In yet some further embodiments, the target sequence is comprised within an exon that is not the last exon in the transcript. In yet some further embodiments, the target sequence may be located in the vicinity of an exon that is not the first or the last exon in the transcript. For example, the target nucleic acid sequence may be located within a 5′ splice junction, that is the intron/exon splice junction located 5′ or upstream to the indicated exon. In other embodiments, the target sequence may be located within the 3′ splice junction, or in other words, in the exon/intron junction located 3′ or downstream to the indicated exon. It should be understood that in certain embodiments the exon as specified herein is not the first or the last exon in the target transcript. Thus, the target sequence for an aberrant splicing event may include any sequence within an exon, or within at least one intron located upstream or downstream to said exon, or within at least one splicing junction flanking said exon. More specifically, the target sequence may include any sequence comprised within a sequence flanking the 5′ end of an exon in a distance from about 1 to about 500 base pairs upstream of the indicated exon, or alternatively or additionally, any sequence comprised within a sequence flanking the 3′ end of an exon in a distance from about 1 to about 500 base pairs downstream of the indicated exon in a preprocessed target transcript. Specifically about 1, 5, 10, 15, 20, 25, 30, 35, 40, 45, 50, 55, 60, 65, 70, 75, 80, 85, 90, 95, 100, 150, 200, 250, 300, 350, 400, 450 or 500 base pairs downstream or upstream of the indicated exon. The term “flanked”, “flanking”, “flank”, as used herein refers to a nucleic acid sequence positioned between two defined regions. For example, the target exon is flanked by an intron/exon splice junction and an exon/intron splice junction, where the intron/exon splice junction is positioned 5′ (or upstream) to the exon and the exon/intron splice junction is positioned 3′ (or downstream) to the exon.

The nucleic acid sequence of the splicing modulating agent of the methods of the invention targets a target nucleic acid. As used herein, “target nucleic acid” means a nucleic acid molecule to which an antisense compound (e.g., oligonucleotide or complementary guide RNAs) hybridizes. As used herein, “targeting” or “targeted to” means the association of an antisense compound to a particular target nucleic acid molecule or a particular region of a target nucleic acid molecule. An antisense compound targets a target nucleic acid if it is sufficiently complementary to the target nucleic acid to allow hybridization under physiological conditions, as will be further detailed herein after.

In some embodiments the splicing modulating agent used by the methods of the invention may comprise at least one oligonucleotide. In more specific embodiments such oligonucleotide is an antisense oligonucleotide (ASO).

The methods, compositions and kits of the invention provide oligonucleotides, for modulating splicing events. As used herein, “oligonucleotide” means a compound comprising a plurality of linked nucleosides. More specifically, single strand or double strand oligomer or polymer of ribonucleic acid molecules, deoxyribonucleic acid molecules and any combinations thereof. In certain embodiments, an oligonucleotide comprises one or more unmodified ribonucleosides (RNA) and/or unmodified deoxyribonucleosides (DNA) and/or one or more modified nucleosides. As used herein, “modified oligonucleotide” means an oligonucleotide comprising at least one modified nucleoside and/or at least one modified internucleoside linkage. More specifically, “Nucleobase” means the heterocyclic base portion of a nucleoside. Nucleobases may be naturally occurring or may be modified. In certain embodiments, a nucleobase may comprise any atom or group of atoms capable of hydrogen bonding to a nucleobase of another nucleic acid. “Nucleotide” means a nucleoside comprising a phosphate linking group. As used herein, nucleosides include nucleotides.

“Modified nucleoside” a nucleoside comprising at least one modification compared to naturally occurring RNA or DNA nucleosides. Such modification may be at the sugar moiety and/or at the nucleobase. “Oligonucleoside” means an oligonucleotide in which none of the internucleoside linkages contains a phosphorus atom. As used herein, oligonucleotides include oligonucleosides. “Modified oligonucleotide” means an oligonucleotide comprising at least one modified nucleoside and/or at least one modified internucleoside linkage.

In some specific embodiments, the oligonucleotides used by the methods of the invention may be antisense oligonucleotides, specifically, AON/s, or ASO (antisense oligo), and Splice-Switching Oligonucleotides (SSOs), that are used herein interchangeably. Provided herein are antisense compounds useful for modulating RNA splicing via antisense mechanisms of action, including antisense mechanisms based on target occupancy. More specifically, an “antisense” is a single strand nucleic acid molecule that is complementary to one of the strands of a target nucleic acid molecule of a specific target gene. Antisense may inhibit or interfere a splicing event by base-pairing to it and physically obstructing the splicing machinery. As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In certain embodiments, complementary nucleobase means a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

As used herein, “complementary” in reference to oligomeric compounds (e.g., linked nucleosides, oligonucleotides, or nucleic acids) means the capacity of such oligomeric compounds or regions thereof to hybridize to another oligomeric compound or region thereof through nucleobase complementarity under stringent conditions. In contrast, “non-complementary” in reference to nucleobases means a pair of nucleobases that do not form hydrogen bonds with one another. Complementary oligomeric compounds need not have nucleobase complementarity at each nucleoside. Rather, some mismatches are tolerated. Complementarity may be “partial,” in which only some of the nucleic acids' bases are matched according to base pairing rules. Or, there may be “complete” or “total” complementarity between the nucleic acids. The degree of complementarity between nucleic acid strands has significant effects on the efficiency and strength of hybridization between nucleic acid strands. In certain embodiments, complementary oligomeric compounds or regions are complementary at 70% of the nucleobases to the target sequence (70% complementary). In certain embodiments, complementary oligomeric compounds or regions are 80% complementary. In certain embodiments, complementary oligomeric compounds or regions are 90% complementary. In certain embodiments, complementary oligomeric compounds or regions are 95% complementary. In certain embodiments, complementary oligomeric compounds or regions are 100% complementary. While perfect complementarity is often desired, some embodiments can include one or more but preferably 6, 5, 4, 3, 2, or 1 mismatches with respect to the target nucleic acid sequence. Variations at any location within the oligomer are included. In certain embodiments, variations in sequence near the termini of an oligomer are generally preferable to variations in the interior, and if present are typically within about 6, 5, 4, 3, 2, or 1 nucleotides of the 5′ and/or 3′ terminus. In yet some further embodiments, the antisense oligomer of the invention may be said to be “directed to” or “targeted against” a target sequence with which it hybridizes. As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases. As used herein, “specifically hybridizes” means the ability of an oligomeric compound to hybridize to one nucleic acid site with greater affinity than it hybridizes to another nucleic acid site. In certain embodiments, an antisense oligonucleotide specifically hybridizes to more than one target site. As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

Still further, “Antisense oligonucleotide” (AON, or ASO/s as used herein interchangeably) means an oligomeric compound, at least a portion of which is at least partially complementary to a target nucleic acid to which it hybridizes, wherein such hybridization results in at least one antisense activity. In some specific embodiments, the ASOs of the invention are Splice-Switching Oligonucleotides (SSOs). In certain embodiments, the present invention provides antisense oligonucleotides of any of a variety of ranges of lengths. In certain embodiments, the present invention provides oligomeric compounds including oligonucleotides of any of a variety of ranges of lengths. In certain embodiments, the invention provides oligomeric compounds or oligonucleotides consisting of X to Y linked nucleosides or nucleotides, where X represents the fewest number of nucleosides in the range and Y represents the largest number of nucleosides in the range. In certain such embodiments, X and Y are each independently selected from 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100 and more nucleosides or nucleotides; provided that X<Y. For example, in certain embodiments, the invention provides antisense compounds or antisense oligonucleotides comprising or consisting of: 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38, 2-39, 2-40, 2-41, 2-42, 2-43, 2-44, 2-45, 2-46, 2-47, 2-48, 2-49, 2-50 and more, specifically, 2-100 and more, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50 and more, specifically, 3-100 and more, 4-9, 4-10, 4-11, 4-12, 4-13, 4-14, 4-15, 4-16, 4-17, 4-18, 4-19, 4-20, 4-21, 4-22, 4-23, 4-24, 4-25, 4-26, 4-27, 4-28, 4-29, 4-30, 4-31, 4-32, 4-33, 4-34, 4-35, 4-36, 4-37, 4-38, 4-39, 4-40, 4-41, 4-42, 4-43, 4-44, 4-45, 4-46, 4-47, 4-48, 4-49, 4-50 and more, specifically, 4-100 and more, 5-9, 5-10, 5-11, 5-12, 5-13, 5-14, 5-15, 5-16, 5-17, 5-18, 5-19, 5-20, 5-21, 5-22, 5-23, 5-24, 5-25, 5-26, 5-27, 5-28, 5-29, 5-30, 5-31, 5-32, 5-33, 5-34, 5-35, 5-36, 5-37, 5-38, 5-39, 5-40, 5-41, 5-42, 5-43, 5-44, 5-45, 5-46, 5-47, 5-48, 5-49, 5-50 and more, specifically, 5-100 and more, 6-9, 6-10, 6-11, 6-12, 6-13, 6-14, 6-15, 6-16, 6-17, 6-18, 6-19, 6-20, 6-21, 6-22, 6-23, 6-24, 6-25, 6-26, 6-27, 6-28, 6-29, 6-30, 6-31, 6-32, 6-33, 6-34, 6-35, 6-36, 6-37, 6-38, 6-39, 6-40, 6-41, 6-42, 6-43, 6-44, 6-45, 6-46, 6-47, 6-48, 6-49, 6-50 and more, specifically, 6-100 and more, 7-9, 7-10, 7-11, 7-12, 7-13, 7-14, 7-15, 7-16, 7-17, 7-18, 7-19, 7-20, 7-21, 7-22, 7-23, 7-24, 7-25, 7-26, 7-27, 7-28, 7-29, 7-30, 7-31, 7-32, 7-33, 7-34, 7-35, 7-36, 7-37, 7-38, 7-39, 7-40, 7-41, 7-42, 7-43, 7-44, 7-45, 7-46, 7-47, 7-48, 7-49, 7-50 and more nucleosides or nucleotides, specifically, 7-100 and more, 8-9, 8-10, 8-11, 8-12, 8-13, 8-14, 8-15, 8-16, 8-17, 8-18, 8-19, 8-20, 8-21, 8-22, 8-23, 8-24, 8-25, 8-26, 8-27, 8-28, 8-29, 8-30, 8-31, 8-32, 8-33, 8-34, 8-35, 8-36, 8-37, 8-38, 8-39, 8-40, 8-41, 8-42, 8-43, 8-44, 8-45, 8-46, 8-47, 8-48, 8-49, 8-50 and more, specifically, 8-100 and more, 9-10, 9-11, 9-12, 9-13, 9-14, 9-15, 9-16, 9-17, 9-18, 9-19, 9-20, 9-21, 9-22, 9-23, 9-24, 9-25, 9-26, 9-27, 9-28, 9-29, 9-30, 9-31, 9-32, 9-33, 9-34, 9-35, 9-36, 9-37, 9-38, 9-39, 9-40, 9-41, 9-42, 9-43, 9-44, 9-45, 9-46, 9-47, 9-48, 9-49, 9-50 and more, specifically, 9-100 and more, 10-11, 10-12, 10-13, 10-14, 10-15, 10-16, 10-17, 10-18, 10-19, 10-20, 10-21, 10-22, 10-23, 10-24, 10-25, 10-26, 10-27, 10-28, 10-29, 10-30, 10-31, 10-32, 10-33, 10-34, 10-35, 10-36, 10-37, 10-38, 10-39, 10-40, 10-41, 10-42, 10-43, 10-44, 10-45, 10-46, 10-47, 10-48, 10-49, 10-50 and more, specifically, 10-100 and more, nucleosides or nucleotides. In some specific embodiments of the invention, oligomers for use in antisense applications generally range in length from about 10 to about 50 subunits, more preferably about 10 to 30 subunits, and typically 15-25 bases. For example, an oligomer of the invention having 15-20 subunits, specifically, 15, 16, 17, 18, 19, 20, or more bases.

In some embodiments the oligonucleotide used by the method of the invention may comprise at least 10 or more contiguous nucleobases complementary to at least part of the at least one nucleic acid sequence that participates directly or indirectly in at least one splicing event. Specifically, at least 10 or more, at least 11 or more, at least 12 or more, at least 13 or more, at least 14 or more, at least 15 or more, at least 16 or more, at least 17 or more, at least 18 or more, at least 19 or more, at least 20 or more, at least 21 or more, at least 22 or more, at least 23 or more, at least 24 or more, at least 25 or more, at least 26 or more, at least 27 or more, at least 28 or more, at least 29 or more, at least 30 or more contiguous nucleobases. In yet some further specific embodiments, the oligonucleotide used by the methods of the invention may comprise at least fifteen contiguous nucleobases complementary to at least part of the at least one nucleic acid sequence that participates directly or indirectly in at least one splicing event. In some embodiments, the oligonucleotides provided and used by the invention may comprise DNA, RNA, any derivatives thereof or any combinations thereof. More specifically, the currently used antisense oligonucleotides are rarely regular RNA or DNA oligonucleotide, as alternative antisense oligonucleotide chemistries have been developed to improve affinity, boost stability in the circulation and in target cells, and enhance cell penetration and nuclear accumulation. The non-bridging oxygen in the phosphate backbone may be replaced with a sulfur atom, generating phosphorothioate (PS) AONs. This modification enhances cellular uptake and improves resistance to nucleases but reduces the affinity of the AON to the target RNA. Addition of a methyl or a methoxyethyl group to the 2′-O atom of the ribose sugar (2′OMe and 2′OMOE, respectively) renders the AON-target RNA hybrid RNase H-resistant and increases the affinity of the AON for the target RNA. Most AONs have both the 2′O and the phosphorothioate (PS) modification (2′OMe-PS and 2′OMOE-PS) since they have a good safety profile and their synthesis is relatively inexpensive. “2′-OMe” or “2′-OCH₃” or “2′-O-methyl” each means a nucleoside comprising a sugar comprising an —OCH₃ group at the 2′ position of the sugar ring.

“MOE” or “2′-MOE” or “2′-OCH₂CH₂OCH₃” or “2′-O-methoxyethyl” each means a nucleoside comprising a sugar comprising a —OCH₂CH₂OCH₃ group at the 2′ position of the sugar ring. In a different available oligonucleotide chemistry, a methylene bridge connects the 2′-O and the 4′-C of the ribose, forcing the nucleotide in the “endo” conformation, in what has been dubbed “locked nucleic acid” (LNA). This modification leads to a very high affinity for the target nucleic acid. In addition to the described negatively charged oligonucleotides (2′OMe-PS, 2′OMOE-PS, and LNA), two more oligonucleotide chemistries may be used in attempts to modulate splicing in accordance with some embodiments of the invention, specifically, peptide nucleic acids (PNAs) and phosphorodiamidate morpholino oligomers (PMOs). Both these types of charge-neutral oligonucleotides are resistant to exo- and endonucleases and RNase H cleavage. PNAs have a 2-aminoethyl glycine backbone linked to nucleobases and show high affinity to both RNA and DNA targets and good sequence specificity. PMOs consist of morpholine rings that are connected through phosphorodiamidate groups. The terms “morpholino oligomer” or “PMO” (phosphoramidate- or phosphorodiamidate morpholino oligomer) refer to an oligonucleotide analog composed of morpholino subunit structures, where (i) the structures are linked together by phosphorus-containing linkages, one to three atoms long, preferably two atoms long, and preferably uncharged or cationic, joining the morpholino nitrogen of one subunit to a 5′ exocyclic carbon of an adjacent subunit, and (ii) each morpholino ring bears a purine or pyrimidine base-pairing moiety effective to bind, by base specific hydrogen bonding, to a base in a polynucleotide. Variations can be made to this linkage as long as they do not interfere with binding or activity. For example, the oxygen attached to phosphorus may be substituted with sulfur (thiophosphorodiamidate). The 5′ oxygen may be substituted with amino or lower alkyl substituted amino. The pendant nitrogen attached to phosphorus may be unsubstituted, monosubstituted, or disubstituted with (optionally substituted) lower alkyl. The purine or pyrimidine base pairing moiety is typically adenine, cytosine, guanine, uracil, thymine or inosine. The synthesis, structures, and binding characteristics of morpholino oligomers are detailed in U.S. Pat. Nos. 5,698,685, 5,217,866, 5,142,047, 5,034,506, 5,166,315, 5,521,063, 5,506,337, 8,076,476, 8,299,206 and 7,943,762 (cationic linkages). It should be understood that the invention further encompasses any oligonucleotide modification known in the art, specifically, any one of LNOs, PMOs, 2′-O-ME, 2′-MOE, 2′-Flour, and the like. In certain embodiments, the antisense oligonucleotides of the present invention are modified by attachment of one or more conjugate groups. In general, conjugate groups modify one or more properties of the attached oligomeric compound including but not limited to, pharmacodynamics, pharmacokinetics, stability, binding, absorption, cellular distribution, cellular uptake, charge and clearance. Conjugate groups are routinely used in the chemical arts and are linked directly or via an optional conjugate linking moiety or conjugate linking group to a parent compound such as an oligomeric compound, such as an oligonucleotide. Conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. Certain conjugate groups have been described previously, for example: cholesterol moiety, cholic acid, a thioether, e.g., hexyl-S-tritylthiol, a thiocholesterol, an aliphatic chain, e.g., do-decan-diol or undecyl residues, a phospholipid, e.g., di-hexadecyl-rac-glycerol or triethyl-ammonium 1,2-di-0-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

Antisense oligonucleotides/AONs include both GAPmers that induce degradation of mRNAs (e.g., e.g., RNase H cleavage) and Splice-Switching Oligonucleotides (SSOs). It should be understood that the invention relate to any SSOs. More specifically, in some embodiments, the ASO of the invention does not include GAPmers. In addition to modulation of the splicing events by ASOs of the invention, one can use a different approach where the endogenous Double-stranded RNA-specific adenosine deaminase (ADAR) RNA editing enzymes are recruited and guided to edit a selected target to change base at key splicing site. This could be achieved by introducing an antisense oligonucleotide that, when bound to its target, assumes a dsRNA configuration similar to that of a typical ADAR editing site and instigates editing. Thus, in some embodiments, the ASOs used and/or provided by the invention may be suitable for guiding and requiting ADAR proteins, thereby leading to aberrant splicing event in the target sequence.

In certain embodiments, the antisense oligonucleotides comprise one or more terminal stabilizing group that enhances properties such as for example nuclease stability. Included in stabilizing groups are cap structures. The terms “cap structure” or “terminal cap moiety,” as used herein, refer to chemical modifications, which can be attached to one or both of the termini of an oligomeric compound. Certain such terminal modifications protect the oligomeric compounds having terminal nucleic acid moieties from exonuclease degradation, and can help in delivery and/or localization within a cell. The cap can be present at the 5′-terminus (5′-cap) or at the 3′-terminus (3′-cap) or can be present on both termini.

Still further, in some embodiments, the antisense compounds of the invention may include an oligonucleotide moiety conjugated to a CPP, preferably an arginine-rich peptide transport moiety effective to enhance transport of the compound into cells. The transport moiety is preferably attached to a terminus of the oligomer. In some embodiments, the cell-penetrating peptide may be an arginine-rich peptide transporter. In other embodiments, the cell-penetrating peptide may be Penetratin or the Tat peptide. These peptides are well known in the art.

Still further, it should be noted that physical methods applied for in vitro and in vivo ASOs delivery are based on making transient penetration in cell membrane by mechanical, electrical, ultrasonic, hydrodynamic, or laser-based energy so that DNA entrance into the targeted cells is facilitated. In yet some alternative embodiments the splicing modulating agent is at least one guide RNA that guides at least one PEN to the at least one target nucleic acid sequence as specified herein. In some embodiments, the PEN comprises at least one clustered regulatory interspaced short palindromic repeat (CRISPR)/CRISPR associated (cas) protein. Thus, according to some embodiments, the splicing modulating agent used by the methods of the invention comprises: first (a), at least one nucleic acid sequence comprising at least one gRNA, or any nucleic acid sequence encoding the gRNA; or any kit, composition, vector or vehicle comprising the gRNA or nucleic acid sequence encoding the gRNA. Optionally, the splicing modulating agent may further comprise (b), at least one CRISPR/cas protein, or any nucleic acid molecule encoding the Cas protein, or any kit, composition, vector or vehicle comprising the CRISPR/cas protein or nucleic acid sequence encoding the CRISPR/cas protein, or any nucleic acid sequence encoding said gRNA; or any kit, composition or vehicle comprising at least one of (a) and (b).

Thus, in some embodiments, the Cas protein and the specific gRNA may be provided either as a protein and gRNA, or alternatively, as nucleic acid sequences encoding these two elements, either in two separate nucleic acid molecules (e.g., two separate constructs), or in one nucleic acid molecule (e.g., a construct encoding both).

The term “programmable engineered nucleases (PEN)” as used herein also known as “molecular DNA scissors”, refers to enzymes either synthetic or natural, used to replace, eliminate or modify target sequences in a highly targeted way. PEN target and cut specific genomic sequences (recognition sequences) such as DNA sequences. The at least one PEN may be derived from natural occurring nucleases or may be an artificial enzyme, all involved in DNA repair of double strand DNA lesions and enabling direct genome editing. In some alternative or additional embodiments the splicing modulating compound according with the present disclosure encompasses also any nucleic acid molecule comprising at least one nucleic acid sequence encoding the PEN or any kit, composition or vehicle comprising the at least one PEN, or any nucleic acid sequence encoding the PEN.

In yet some further specific embodiments, such nucleases may include RNA guided nucleases such as CRISPR-Cas. However, it should be understood that in some alternative embodiments, other nucleases such as ZFN, TALEN, Homing endonuclease, Meganuclease, Mega-TALEN may be used by the methods of the invention for targeting at least one target nucleic acid sequence involved in at least one splicing event, and inducing aberrant splicing of the target transcript.

More specifically, in some embodiments, the at least one PEN may be at least one of a mega nuclease, a zinc finger nuclease (ZFN), a transcription activator-like effector-based nuclease (TALEN), or a clustered regularly interspaced short palindromic repeats (CRISPR/Cas) system.

In some embodiments, the at least one PEN may be a mega nuclease. Mega nucleases are endodeoxyribonucleases characterized by a large recognition site (double-stranded DNA sequences of 12 to 40 base pairs); such that this site generally occurs only once in any given genome. Meganucleases are specific naturally occurring restriction enzymes and include among others, the LAGLIDADG family of homing endonucleases, mostly found in the mitochondria and chloroplasts of eukaryotic unicellular organisms.

In some embodiments, the at least one PEN may be a megaTAL. MegaTALs are fusion proteins that combine homing endonucleases, such as LAGLIDADG family, with the modular DNA binding domains of TALENs.

In some alternative embodiments, the at least one PEN may be a zinc finger nuclease (ZFN). ZFNs are artificial restriction enzymes generated by fusing a zinc finger DNA-binding domain to a DNA-cleavage domain. Zinc finger domains can be engineered to target specific desired DNA sequences, enabling ZFN to target the target sequences within the target transcripts specified by the invention, thereby inducing aberrant splicing events.

In yet some other embodiments, the at least one PEN may be a transcription activator-like effector-based nuclease (TALEN). TALEN are restriction enzymes that can be engineered to cut specific sequences of DNA. TALEN are made by fusing a TAL effector DNA-binding domain to a DNA cleavage domain (a nuclease which cuts DNA strands). In yet some further embodiments, additional technologies that can be used are the combination of engineered base editor proteins. These artificially engineered proteins need, just as the CRISPR system discussed above, antisense oligonucleotides to guide the base editor proteins (e.g., any ADAR protein or any variant thereof, or any apolipoprotein B mRNA editing enzyme, catalytic polypeptide-like (APOBEC) protein and any variant thereof), to the proper location that needs to be edited in order to manipulate the splicing reaction.

As indicated above, in some specific embodiments, the targeting of the target nucleic acid sequence that participate in at least one aberrant splicing event may be mediated by a PEN that may comprise at least one clustered regulatory interspaced short palindromic repeat (CRISPR)/CRISPR associated (cas) protein system. The Clustered Regularly Interspaced Short Palindromic Repeats (CRISPR) system is a bacterial immune system that has been modified for genome engineering.

CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI. Thus, in some embodiments, the Cas protein may be a member of at least one of CRISPR-associated system of Class 1 and Class 2. In some embodiments, the cas protein may be a member of at least one of CRISPR-associated system of any one of type II, type I, type III, type IV, type V and type VI from E. coli, Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly any of the CRISPR systems disclosed herein. The CRISPR-Cas system, targets DNA molecules based on short homologous DNA sequences, called spacers that exist between repeats. These spacers guide CRISPR-associated (Cas) proteins to matching sequences within the target DNA, called proto-spacers, which are subsequently cleaved. The spacers can be rationally designed to form guide RNAs (gRNAs) that target any target DNA sequence. For example, any of the target transcripts disclosed in Table 1, or in the target sequence within or in the vicinity of any of the exons specified by Table 2, as well as exon 4 of the TYR gene transcript and exon 6 of the hnRNPAB transcript, as exemplified by the invention. It should be noted that the splicing modulating agents of the invention comprise in some embodiments at least one gRNA targeted against any of the specific targets specified by the invention, or any nucleic acid sequence encoding such gRNA. In some specific embodiment, the RNA guided DNA binding protein nuclease used by the methods of the invention may be of a CRISPR Class 2 system. In yet some further particular embodiments, such class 2 system may be a CRISPR type II system. The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity responsible for target cleavage.

It should be appreciated that any type II CRISPR-Cas systems may be applicable in the present invention, specifically, any one of type II-A, typeII-B or typeII-C. In more particular embodiments, at least one cas protein of type II CRISPR system used by the methods and systems of the invention may be the cas9 protein, or any fragments, mutants, fusion proteins, variants or derivatives thereof (e.g., Cas9/Cpf1/CTc(1/2/3), SpCas9, SaCas9, engineered Cas9, and any mutants (for example dCas (with no nuclease activity), or any fusion proteins thereof, specifically with any nucleic acid modifying protein, for example, ADAR, as discussed above, dCas9-Fok1, and the like). The CRISPR-associated protein Cas9 is an RNA-guided DNA endonuclease that uses RNA:DNA complementarity to a target site (proto-spacer). After recognition between Cas9 and the target sequence double stranded DNA (dsDNA) cleavage occur, creating the double strand brakes (DSBs).

Still further, CRISPR type II system as used herein requires the inclusion of two essential components: a “guide” RNA (gRNA), that is comprised within the splicing modulating agent of the invention, and a non-specific CRISPR-associated endonuclease (Cas9). Guide RNA (gRNA), as used herein refers to a synthetic fusion of the endogenous tracrRNA with a targeting sequence (also named crRNA), providing both scaffolding/binding ability for Cas9 nuclease and targeting specificity. Also referred to as “single guide RNA” or “sgRNA”. In some embodiments, the gRNA of the invention may comprise between about 15 to about 50 nucleotides, specifically, about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides. More specifically, spacers may comprise between about 20-35 nucleotides.

In yet some further embodiments, where the splicing modulating agent comprises at least one nucleic acid sequence encoding the gRNA, such encoding sequence may be designed to target at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more target protospacers (target sequences recognized by the gRNA, for inducing aberrant splicing) within the target transcript or several target transcripts.

In CRISPR systems based on PAM (protospacer adjacent motif) sequence recognition like CRISPR Type II, the PAM is absolutely necessary for target binding and the exact sequence is dependent upon the species of Cas9 used. Thus, in some embodiments, the target sequences within the target transcript, comprise at least one PAM required for recognition and binding of the CRISPR/Cas protein. In certain embodiments, Cas9 from S. pyogenes may be used in the methods, cells, compositions, and kits of the invention. Nevertheless, it should be appreciated that any known Cas9 may be applicable. Non-limiting examples for Cas9 useful in the present disclosure include but are not limited to Streptococcus pyogenes (SP), also indicated herein as SpCas9, Staphylococcus aureus (SA), also indicated herein as SaCas9, Neisseria meningitidis (NM), also indicated herein as NmCas9, Streptococcus thermophilus (ST), also indicated herein as StCas9 and Treponema denticola (TD), also indicated herein as TdCas9. Still further, it should be appreciated that type V CRISPR/Cas, including Cas12a, Cpf1 (type VI), C2C1 (type V-B), Cas13 (type VI), specifically, C2C2 and CasRx and CasX, as well as any variants or fusion proteins thereof, may be also applicable in the invention.

In more specific embodiments, the gRNA comprised within the splicing modulating agent of the invention, targets the specific target sequence as disclosed by the invention and guides the CRISPR/Cas-protein, specifically, Cas9 to cleave, or perform other modification in the target site. The end result of Cas9-mediated DNA cleavage is a double strand break (DSB) within the target DNA. The resulting DSB may be then repaired by one of two general repair pathways, the efficient but error-prone Non-Homologous End Joining (NHEJ) pathway and the less efficient but high-fidelity Homology Directed Repair (HDR) pathway. In some specific embodiments, the targeted nucleic acid sequences specified above are repaired through the NHEJ pathway, resulting in most cases in alteration of the target sequence (deletions/insertions/non-sense mutations etc.) and thereby leading to aberrant splicing that leads to frameshift and creation of a neoantigen.

As indicated above, the gene editing system used as the splicing modulating agent of the invention may be provided as nucleic acid molecules, specifically in a delivery vector or vehicle. However, it should be appreciated that any of the gene editing systems used, may be also administered as a protein complex, or alternatively, as a ribonucleoprotein complex. More specifically, when gene editing system is used by the invention, such system may be delivered either as nucleic acid sequences encoding the components of this system, e.g., constructs comprising nucleic acid sequences that encode the CRISPR/Cas protein, for example, Cas9 and the specific gRNAs. However, it should be appreciated that the invention further encompasses in some embodiments thereof the option of using Cas9/gRNA Ribonucleoprotein complexes (Cas9 RNPs), that comprise purified Cas9 and purified gRNAs delivered as functional complexes. In some particular embodiments, purified gRNAs can be generated by PCR amplification of annealed gRNA oligos or in vitro transcription of a linearized gRNA containing plasmid. Cas9 (or any variant of Cas9) can be purified from bacteria through the use of bacterial Cas9 expression plasmids. In yet some further embodiments, the Cas9 RNP delivery to target cells may be carried out in some specific and non-limiting embodiments, via lipid-mediated transfection or electroporation. Non-limiting examples for using the CRISPR/Cas9 system in the methods of the invention are provided by Examples 5 to 8, and 11 to 13 for the TYR and hnRPNAB targets.

In more specific embodiments, the splicing modulating agent comprising at least one nucleic acid sequence, specifically, the AON/s, and/or guide RNA sequences of the invention may be incorporated in a vector or construct. In yet some further embodiments, the oligonucleotides, specifically, the AONs provided herein and used by the methods of the invention may be comprised within a nucleic acid vector. In more specific embodiments, such vector may be any one of a viral vector, a non-viral vector and a naked DNA vector.

Vectors, as used herein, are nucleic acid molecules of particular sequence can be incorporated into a vector that is then introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Many vectors, e.g. plasmids, cosmids, minicircles, phage, viruses, etc., useful for transferring nucleic acids into target cells may be applicable in the present invention. The vectors comprising the nucleic acid(s) may be maintained episomally, e.g. as plasmids, minicircle DNAs, viruses such cytomegalovirus, adenovirus, etc., or they may be integrated into the target cell genome, through homologous recombination or random integration, e.g. retrovirus-derived vectors such as AAV, MMLV, HIV-1, ALV, etc.

Vectors may be provided directly to the subject cells. In other words, the cells are contacted with vectors comprising the oligonucleotides of the invention such that the vectors are taken up by the cells. Methods for contacting cells with nucleic acid vectors that are plasmids, such as electroporation, calcium chloride transfection, and lipofection, are well known in the art. DNA can be introduced as naked nucleic acid, as nucleic acid complexed with an agent such as a liposome or poloxamer, or can be delivered by viruses (e.g., adenovirus, AAV).

More specifically, in some embodiments, the vector may be a viral vector. In yet some particular embodiments, such viral vector may be any one of recombinant adeno associated vectors (rAAV), single stranded AAV (ssAAV), self-complementary rAAV (scAAV), Simian vacuolating virus 40 (SV40) vector, Adenovirus vector, helper-dependent Adenoviral vector, retroviral vector and lentiviral vector. As indicated above, in some embodiments, viral vectors may be applicable in the present invention. The term “viral vector” refers to a replication competent or replication-deficient viral particle which are capable of transferring nucleic acid molecules into a host. Still further, in some embodiments, the vector may be a naked DNA vector. More specifically, such vector may be for example, a plasmid, minicircle or linear DNA. Naked DNA alone may facilitate transfer of a gene (2-19 kb) into skin, thymus, cardiac muscle, and especially skeletal muscle and liver cells when directly injected. It enables also long-term expression. Although naked DNA injection is a safe and simple method, its efficiency for gene delivery is quite low. Minicircles are modified plasmid in which a bacterial origin of replication (ori) was removed, and therefore they cannot replicate in bacteria. In yet some further embodiments, the AON/s of then invention may be incorporated in compositions or any micro- or nano-particles, as discussed herein after.

In some embodiments, specifically in case where the splicing modulating agent of the invention comprises AON/s, direct infusion or injection of the AON/s or any formulations thereof, may be also applicable. Still further, absorption via mucosal tissues (e.g., inhalation), may be also applicable for AON/s. The oligonucleotides can be introduced into tissues or host cells by any number of routes, including viral infection, microinjection, or fusion of vesicles. Jet injection may also be used for intramuscular administration. The oligonucleotides can be coated onto gold microparticles, and delivered intradermally by a particle bombardment device, or “gene gun”, where gold microprojectiles are coated with the DNA, then bombarded into skin cells. The oligonucleotides can be directly introduced into the cell (i.e., intracellularly); or introduced extracellularly into a cavity, interstitial space, into the circulation of an organism, introduced orally, etc. Methods for oral introduction include direct mixing of RNA with the food of the organism. Physical methods of introducing nucleic acids include injection directly into the cell or extracellular injection into the organism of an RNA solution.

As indicated above, in yet some specific embodiments, the invention provides methods for inducing the production of at least one neoantigen in at least one target cell. In some specific embodiments, the target cell may be a cell of a subject suffering from at least one neoplastic disorder. By “neoplasia” is meant any disease that is caused by or results in inappropriately high levels of cell division, inappropriately low levels of apoptosis, or both. For example, cancer is an example of a neoplasia. In yet some further embodiments, the target cell of the methods of the invention may be a cell of a subject suffering from at least one neoplastic disorder.

In more specific embodiments, such neoplastic disorder is a cancer. In some specific embodiments, the methods, compositions, kits and any of the splicing modulating agents of the invention that comprise at least one nucleic acid sequence, e.g., any of the AON/s or any of the guide RNAs and any PEN systems of the invention, may be used for treating cancer or any other neoplastic disorders or proliferative disorders. As used herein to describe the present invention, “proliferative disorder”, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods, compositions, kits and AON/s of the invention of the present invention may be applicable for treatment of a patient suffering from any one of non-solid and solid tumors. Malignancy, as contemplated in the present invention may be any one of carcinomas, melanomas, lymphomas, leukemias, myeloma and sarcomas.

Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Melanoma as used herein, is a malignant tumor of melanocytes. Melanocytes are cells that produce the dark pigment, melanin, which is responsible for the color of skin. They predominantly occur in skin, but are also found in other parts of the body, including the bowel and the eye. Melanoma can occur in any part of the body that contains melanocytes.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma. Non limiting examples for lymphoma include Hodgkin's disease, non-Hodgkin's lymphomas and Burkitt's lymphoma.

Further malignancies that may find utility in the present invention can comprise but are not limited to hematological malignancies (including lymphoma, leukemia and myeloproliferative disorders, as described above), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma. The invention may be applicable as well for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma.

Still further, in some embodiments, the term cancer includes but is not limited to, Acute lymphoblastic leukemia; Acute myeloid leukemia; Adrenocortical carcinoma; AIDS-related cancers; AIDS-related lymphoma; Anal cancer; Appendix cancer; Astrocytoma, childhood cerebellar or cerebral; Basal cell carcinoma; Bile duct cancer, extrahepatic; Bladder cancer; Bone cancer, Osteosarcoma/Malignant fibrous histiocytoma; Brainstem glioma; Brain tumor; Brain tumor, cerebellar astrocytoma; Brain tumor, cerebral astrocytoma/malignant glioma; Brain tumor, ependymoma; Brain tumor, medulloblastoma; Brain tumor, supratentorial primitive neuroectodermal tumors; Brain tumor, visual pathway and hypothalamic glioma; Breast cancer; Bronchial adenomas/carcinoids; Burkitt lymphoma; Carcinoid tumor, childhood; Carcinoid tumor, gastrointestinal; Carcinoma of unknown primary; Central nervous system lymphoma, primary; Cerebellar astrocytoma, childhood; Cerebral astrocytoma/Malignant glioma, childhood; Cervical cancer; Childhood cancers; Chronic lymphocytic leukemia; Chronic myelogenous leukemia; Chronic myeloproliferative disorders; Colon Cancer; Cutaneous T-cell lymphoma; Desmoplastic small round cell tumor; Endometrial cancer; Ependymoma; Esophageal cancer; Ewing's sarcoma in the Ewing family of tumors; Extracranial germ cell tumor, Childhood; Extragonadal Germ cell tumor; Extrahepatic bile duct cancer; Eye Cancer, Intraocular melanoma; Eye Cancer, Retinoblastoma; Gallbladder cancer; Gastric (Stomach) cancer; Gastrointestinal Carcinoid Tumor; Gastrointestinal stromal tumor (GIST); Germ cell tumor: extracranial, extragonadal, or ovarian; Gestational trophoblastic tumor; Glioma of the brain stem; Glioma, Childhood Cerebral Astrocytoma; Glioma, Childhood Visual Pathway and Hypothalamic; Gastric carcinoid; Hairy cell leukemia; Head and neck cancer; Heart cancer; Hepatocellular (liver) cancer; Hodgkin lymphoma; Hypopharyngeal cancer; Hypothalamic and visual pathway glioma, childhood; Intraocular Melanoma; Islet Cell Carcinoma (Endocrine Pancreas); Kaposi sarcoma; Kidney cancer (renal cell cancer); Laryngeal Cancer; Leukemias; Leukemia, acute lymphoblastic (also called acute lymphocytic leukemia); Leukemia, acute myeloid (also called acute myelogenous leukemia); Leukemia, chronic lymphocytic (also called chronic lymphocytic leukemia); Leukemia, chronic myelogenous (also called chronic myeloid leukemia); Leukemia, hairy cell; Lip and Oral Cavity Cancer; Liver Cancer (Primary); Lung Cancer, Non-Small Cell; Lung Cancer, Small Cell; Lymphomas; Lymphoma, AIDS-related; Lymphoma, Burkitt; Lymphoma, cutaneous T-Cell; Lymphoma, Hodgkin; Lymphomas, Non-Hodgkin (an old classification of all lymphomas except Hodgkin's); Lymphoma, Primary Central Nervous System; Marcus Whittle, Deadly Disease; Macroglobulinemia, Waldenstrom; Malignant Fibrous Histiocytoma of Bone/Osteosarcoma; Medulloblastoma, Childhood; Melanoma; Melanoma, Intraocular (Eye); Merkel Cell Carcinoma; Mesothelioma, Adult Malignant; Mesothelioma, Childhood; Metastatic Squamous Neck Cancer with Occult Primary; Mouth Cancer; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple (Cancer of the Bone-Marrow); Myeloproliferative Disorders, Chronic; Nasal cavity and paranasal sinus cancer; Nasopharyngeal carcinoma; Neuroblastoma; Non-Hodgkin lymphoma; Non-small cell lung cancer; Oral Cancer; Oropharyngeal cancer; Osteosarcoma/malignant fibrous histiocytoma of bone; Ovarian cancer; Ovarian epithelial cancer (Surface epithelial-stromal tumor); Ovarian germ cell tumor; Ovarian low malignant potential tumor; Pancreatic cancer; Pancreatic cancer, islet cell; Paranasal sinus and nasal cavity cancer; Parathyroid cancer; Penile cancer; Pharyngeal cancer; Pheochromocytoma; Pineal astrocytoma; Pineal germinoma; Pineoblastoma and supratentorial primitive neuroectodermal tumors, childhood; Pituitary adenoma; Plasma cell neoplasia/Multiple myeloma; Pleuropulmonary blastoma; Primary central nervous system lymphoma; Prostate cancer; Rectal cancer; Renal cell carcinoma (kidney cancer); Renal pelvis and ureter, transitional cell cancer; Retinoblastoma; Rhabdomyosarcoma, childhood; Salivary gland cancer; Sarcoma, Ewing family of tumors; Sarcoma, Kaposi; Sarcoma, soft tissue; Sarcoma, uterine; Sezary syndrome; Skin cancer (nonmelanoma); Skin cancer (melanoma); Skin carcinoma, Merkel cell; Small cell lung cancer; Small intestine cancer; Soft tissue sarcoma; Squamous cell carcinoma—see Skin cancer (nonmelanoma); Squamous neck cancer with occult primary, metastatic; Stomach cancer; Supratentorial primitive neuroectodermal tumor, childhood; T-Cell lymphoma, cutaneous (Mycosis Fungoides and Sezary syndrome); Testicular cancer; Throat cancer; Thymoma, childhood; Thymoma and Thymic carcinoma; Thyroid cancer; Thyroid cancer, childhood; Transitional cell cancer of the renal pelvis and ureter; Trophoblastic tumor, gestational; Unknown primary site, carcinoma of, adult; Unknown primary site, cancer of, childhood; Ureter and renal pelvis, transitional cell cancer; Urethral cancer; Uterine cancer, endometrial; Uterine sarcoma; Vaginal cancer; Visual pathway and hypothalamic glioma, childhood; Vulvar cancer; Waldenstrom macroglobulinemia and Wilms tumor (kidney cancer).

As shown by Example 14, particular target genes and transcripts thereof that are applicable in the present invention as targets, were shown to be overexpressed in specific cancers, that are indicated in Tables 1 and 3. Thus, in some particular and non-limiting embodiments, the methods of the invention may be applicable for at least one of the following cancer conditions, specifically, any one of Adrenocortical carcinoma (LAML), Bladder Urothelial Carcinoma (BLCA), Brain Lower Grade Glioma (LGG), Breast invasive carcinoma (BRCA), Cervical squamous cell carcinoma and endocervical adenocarcinoma (CESC), Colon adenocarcinoma (COAD), Esophageal carcinoma (ESCA), Glioblastoma multiforme (GBM), Head and Neck squamous cell carcinoma (HNSC), Kidney Chromophobe (KICH), Kidney renal clear cell carcinoma (KIRC), Kidney renal papillary cell carcinoma (KIRP), Liver hepatocellular carcinoma (LIHC), Lung adenocarcinoma (LUAD), Lung squamous cell carcinoma (LUSC), Lymphoid Neoplasm Diffuse Large B-cell Lymphoma (DLBC), Ovarian serous cystadenocarcinoma (OV), Pancreatic adenocarcinoma (PAAD), Prostate adenocarcinoma (PRAD), Rectum adenocarcinoma (READ), Skin Cutaneous Melanoma (SKCM), Stomach adenocarcinoma (STAD), Testicular Germ Cell Tumors (TGCT), Thymoma (THYM), Thyroid carcinoma (THCA), Uterine Carcinosarcoma (UCS), Uterine Corpus Endometrial Carcinoma (UCEC).

As indicated above, the invention provides methods, compositions, kits and splicing modulating agents comprising at least one nucleic acid sequence (e.g., AON/s and gRNAs) that induce aberrant splicing in a target cell. The cells according to the present disclosure are eukaryotic cells i.e. cells containing a nucleus and other organelles enclosed within membranes, including animal cells, plant cells and fungal cells. Eukaryotic cells refers to any organism having a cell that contains specialized organelles in the cytoplasm, a membrane-bound nucleus enclosing genetic material organized into chromosomes, and an elaborate system of division by mitosis or meiosis. Examples of eukaryotic cells include but are not limited to animal cells, plant cells, fungi and protists. More specifically, animals are multicellular, eukaryotic organisms of the kingdom Animalia (also called Metazoa) and can be divided broadly into vertebrates and invertebrates. Vertebrates have a backbone or spine (vertebral column), and include fish, amphibians, reptiles, birds and mammals. Invertebrates which lack a backbone include mollusks (clams, oysters, octopuses, squid, snails); arthropods (millipedes, centipedes, insects, spiders, scorpions, crabs, lobsters, shrimp); annelids (earthworms, leeches), nematodes (filarial worms, hookworms), flatworms (tapeworms, liver flukes), cnidarians (jellyfish, sea anemones, corals), ctenophores (comb jellies), and sponges. Thus, animal cells as used herein relate to cells derived from any of the animal cells disclosed above, specifically, mammalian cells. In more specific embodiments, the term cell refers to human cell. In yet some further specific embodiment, the target cell is a cancerous cell. Specifically, Cancer cells are cells that divide relentlessly, forming solid tumors or non-solid tumors.

Still further, in some embodiments, the method of the invention induces the production of at least one neoantigen to be expressed by the target cells, using the splicing modulating agents comprising at least one nucleic acid sequence (e.g., AON/s and gRNAs) that lead to aberrant splicing event in a target gene, or any target transcript thereof. In more specific embodiments, such target gene may be a gene differentially expressed in at least one cancer cell and/or at least one cancerous tissue.

The target genes and more specifically, the target transcripts in accordance with the invention may be selected as appropriate targets based on the differential expression of the transcript in a cancerous tissue as compared to the adjacent normal tissue of the subject, or the corresponding tissue of a subject that do not suffer from the same cancer. Differentially expressed as used herein refers to the level of expression of a target gene or of any transcript thereof that is either over expressed or upregulated or alternatively, downregulated or under-expressed, in a cancerous tissue as compared to the adjacent normal tissue or to the same or equivalent tissue in healthy subjects, or at least one subject that do not suffer from the same cancer. The terms “level of expression” or “expression level” are used interchangeably and generally refer to a numerical representation of the amount (quantity), of an amino acid product or polypeptide or protein expressed from a target transcript in a tissue or a sample thereof. “Expression” generally refers to the process by which gene-encoded information is converted into the structures present and operating in the cell. For example, gene expression values may be measured in the mRNA and/or protein level. Therefore, according to the invention “expression” of a gene, or any transcripts thereof may refer to transcription into a polynucleotide and translation into a polypeptide. It should be noted that in some embodiments, the target gene, and specifically, the target transcript targeted by the methods of the invention is differentially overexpressed in cancerous tissue, specifically as compared to the normal and/or healthy counterpart of the tissue in healthy subjects or subjects not suffering from the same cancer. More specifically, overexpressed is meant an expression that is increased, higher, elevated, enhanced, upregulated in about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9% or even 100%, as compared to a an equivalent healthy or non-cancerous tissue. With regards to the above, it is to be understood that, where provided, percentage values such as, for example, 10%, 50%, 100%, 120%, 500%, etc., are interchangeable with “fold change” values, i.e., 2, 3, 4, 5, 6, 7, 8, 9, 10, 15, 20, 25, 30, 40, 50 or more, etc., respectively.

In some specific embodiments, the target genes or at least one transcript thereof disclosed by the invention, specifically those disclosed in Tables 1 and 2, are targets that display upregulation, on in other words, transcripts that display overexpression or enhanced expression of between about 5 to 50 folds, specifically, about 5, 10, 15, 20, 25, 30, 35, 40, 45, 50 folds or more, specifically, 10 folds as compared to the corresponding tissue from healthy subjects or subjects that are not affected by the same cancer. It should be noted that in some embodiments of the invention, the target gene and transcripts, presented herein after in the Examples section, and by Tables 1 and 2, are selected as being overexpressed in cancer tissues. More specifically, these target genes and transcripts display an enhances expression of about 5 to 10 folds as compared to the corresponding normal tissue. In yet some further specific embodiments, the target gene may be any gene over expressed or highly expressed in a cancerous tissue or cell, specifically when compared with the adjacent normal, healthy or non-cancerous tissue. In some embodiments, such gene may encode at least one tumor associated antigen (TAA). Thus, in some embodiments, the targeted specific splicing junctions for the production of neoantigens according to the methods, compositions, kits, splicing modulating agents, specifically, AON/s and gRNAs of the invention, are located in particular genes which are known to specifically expressed, over expressed or differentially expressed in tumor cells. In yet some further embodiments, antigens highly expressed in cancer can stimulate tumor-specific T-cell immune responses. Exemplary tumor antigens include, but are not limited to, RAGE-1, glycoprotein, gp75, MUC1, beta-catenin, PRAME, MUM-1, WT-1, CEA, PR-1 CD45, glypican-3, IGF2B3, Kallikrein4, KIF20A, Lengsin, Meloe, MUC5AC, survivin, CLPP, Cyclin-A1, SSX2, XAGE1b/GAGED2a, TRP-1, Tyrosinase, gp100, MART-1, MAGE-A1, MAGE-A2, MAGE-A3, MAGE-A6, LAGE-1, CAMEL, NY-ESO-1, hTRT and Eph.

TAA may be recognized by CD8+ T cells as well as CD4+ T cells. Non limiting examples of neoantigens recognized by CD8+ T cells may be CSNK1A1 (S>L), GAS7 (H>Y HAUS3 (T>A), PLEKHM2 (H>Y), PPP1R3B (P>H), MATN2 (E>K), CDK2 (E>K), SRPX (P55L), WDR46 (T2271, AHNAK (S4460F), COL18A1 (S126F), ERBB2 (H197Y), TEAD1 (L209F), NSDHL (A290V), GANAB (S184F), TRIP12 (F1544S), TKT (R438W), CDKN2A (E153K), TMEM48 (F169L), AKAP13 (Q285K), SEC24A (P469L), OR8B3 (T190I), EXOC8 (Q656P), MRPS5 (P59L), PABPC1 (R520Q), MLL2 (L>H), ASTN1 (P>L), CDK4 (R>L), GNL3L (R>C), SMARCD3 (H>Y), MAGE-A6 (E>K), MED13 (P>S), PAS5A (Y>F, H>Y), WDR46 (T>L), HELZ2 (D>N), AFMID (A>V), CENPL (P>L), PRDX3 (P>L), FLNA (R>C), KIF16B (L>P), SON (R>C), MTFR2 (D626Y), CHTF18 (L769V), MYADM (R30W), NUP98 (A359D), KRAS (G12D), CASP8 (F67V), TUBGCP2 (P293L), RNF213 (N17025), SKIV2L (R653H), H3F3B (A48T), AP15 (R243Q), RNF10 (E572K), PHLPP1 (G566E) and ZFYVE27 (R6H).

Non limiting examples of neoantigens recognized by CD4+ T cells may be ERBB2IP (E805G), CIRH1A (P333L), GART (V551A), ASAP1 (P941L), RND3 (P49S), LEMD2 (P495L), TNIK (5502F), RPS12 (V104I), ZC3H18 (G269R), GPD2 (E426K), PLEC (E1179K), XPO7 (P274S), AKAP2 (Q418K) and ITGB4 (S10021).

Non-limiting examples of MHC class II-restricted antigens may be Tyrosinase, gp100, MART-1, MAGE-A1, MAGE-A2, MAGE-A3, MAGE-A6, LAGE-1, CAMEL, NY-ESO-1, hTRT and Eph. In some embodiments, the target gene is selected from, and therefore may be any one of the group of genes disclosed by Table 1 that is presented herein after by Example 14, and at least one transcript thereof. This table disclose the target genes of the invention and specifically, the target transcripts that comprise the target nucleic acid sequence that participates directly or indirectly in at least one splicing event of at least one target gene. This table further indicates the particular cancer that overexpresses the target gene and/or at least one transcript thereof. This target nucleic acid sequence is targeted by the splicing modulators of the invention, specifically, the AON/s and gRNAs disclosed by the invention.

In more specific embodiments, the target nucleic acid sequence may be comprised within at least one transcript of any one of the following genes, specifically, MPZL1 (myelin protein zero like 1), AGRN (agrin), WLS (Wnt ligand secretion mediator), KCNAB2 (potassium voltage-gated channel subfamily A regulatory beta subunit 2), SLC2A5 (solute carrier family 2 member 5), SLC9A1 (solute carrier family 9 member A1), NKAIN1 (sodium/potassium transporting ATPase interacting 1), MAP7D1 (MAP7 domain containing 1), RHBDL2 (rhomboid like 2), CYP2J2 (cytochrome P450 family 2 subfamily J member 2), GPSM2 (G protein signaling modulator 2), SORT1 (sortilin 1), CD58 (CD58 molecule), NOTCH2 (notch receptor 2), CTSS (cathepsin S), PMVK (phosphomevalonate kinase), STX6 (syntaxin 6), CAPN8 (calpain 8), CAPN2 (calpain 2), PARP1 (poly(ADP-ribose) polymerase 1), GPR137B (G protein-coupled receptor 137B), COX20 (cytochrome c oxidase assembly factor COX20), RPL22 (ribosomal protein L22), GPR153 (G protein-coupled receptor 153), PDPN (podoplanin), AKR7A2 (aldo-keto reductase family 7 member A2), CDA (cytidine deaminase), GALE (UDP-galactose-4-epimerase), IL22RA1 (interleukin 22 receptor subunit alpha 1), SYTL1 (synaptotagmin like 1), THEMIS2 (thymocyte selection associated family member 2), SMPDL3B (sphingomyelin phosphodiesterase acid like 3B), TINAGL1 (tubulointerstitial nephritis antigen like 1), SPOCD1 (SPOC domain containing 1), MYCL (MYCL proto-oncogene, bHLH transcription factor), PTPRF (protein tyrosine phosphatase receptor type F), ARTN (artemin), MAGOH (mago homolog, exon junction complex subunit), AK4 (adenylate kinase 4), CYR61 (cellular communication network factor 1), GBP1 (guanylate binding protein 1), EPHX4 (epoxide hydrolase 4), GFI1 (growth factor independent 1 transcriptional repressor), CELSR2 (cadherin EGF LAG seven-pass G-type receptor 2), C1orf162 (chromosome 1 open reading frame 162), CTSK (cathepsin K), ILF2 (interleukin enhancer binding factor 2), CKS1B (CDCl₂8 protein kinase regulatory subunit 1B), BCAN (brevican), MRPL24 (mitochondrial ribosomal protein L24), HDGF (heparin binding growth factor), CD1A (CD1a molecule), CD1B (CD1b molecule), CD1E (CD1e molecule), TAGLN2 (transgelin 2), F11R (F11 receptor), TSTD1 (thiosulfate sulfurtransferase like domain containing 1), PFDN2 (prefoldin subunit 2), ELF3 (E74 like ETS transcription factor 3), CHIT1 (chitinase 1), PIGR (polymeric immunoglobulin receptor), MAP4K4 (mitogen-activated protein kinase kinase kinase kinase 4), MBOAT2 (membrane bound O-acyltransferase domain containing 2), ANTXR1 (ANTXR cell adhesion molecule 1), KIAA1211L (KIAA1211 like), FHL2 (four and a half LIM domains 2), CYTIP (cytohesin 1 interacting protein), ACVR1 (activin A receptor type 1), DPP4 (dipeptidyl peptidase 4), WNT10A (Wnt family member 10A), DOCK10 (dedicator of cytokinesis 10), UGT1A10 (UDP glucuronosyltransferase family 1 member A10), UGT1A6 (UDP glucuronosyltransferase family 1 member A6), MLPH (melanophilin), PDIA6 (protein disulfide isomerase family A member 6), SF3B6 (splicing factor 3b subunit 6), ATRAID (all-trans retinoic acid induced differentiation factor), CALM2 (calmodulin 2), ACTR2 (actin related protein 2), PLEK (pleckstrin), ACTG2 (actin gamma 2, smooth muscle), BOLA3 (bolA family member 3), LOXL3 (lysyl oxidase like 3), GNLY (granulysin), CD8A (CD8a molecule), CD8B (CD8b molecule), SULT1C4 (sulfotransferase family 1C member 4), FAP (fibroblast activation protein alpha), CERKL (ceramide kinase like), PPIL3 (peptidylprolyl isomerase like 3), NOP58 (NOP58 ribonucleoprotein), IHH (Indian hedgehog signaling molecule), SP140 (SP140 nuclear body protein), ITM2C (integral membrane protein 2C), PLS1 (plastin 1), FBLN2 (fibulin 2), CDCP1 (CUB domain containing protein 1), ITGB5 (integrin subunit beta 5), CHCHD6 (coiled-coil-helix-coiled-coil-helix domain containing 6), BHLHE40 (basic helix-loop-helix family member e40), MYD88 (MYD88 innate immune signal transduction adaptor), MYRIP (myosin VIIA and Rab interacting protein), GNL3 (G protein nucleolar 3), CMSS1 (cms1 ribosomal small subunit homolog), CD96 (CD96 molecule), PARP9 (poly(ADP-ribose) polymerase family member 9), TMCC1 (transmembrane and coiled-coil domain family 1), CLDN1 (claudin 1), TIMP4 (TIMP metallopeptidase inhibitor 4), RPSA (ribosomal protein SA), CTNNB1 (catenin beta 1), TDGF1 (teratocarcinoma-derived growth factor 1), SEMA3F (semaphorin 3F), RRP9 (ribosomal RNA processing 9, U3 small nucleolar RNA binding protein), FAM107A (family with sequence similarity 107 member A), HGD (homogentisate 1,2-dioxygenase), CD86 (CD86 molecule), PARP15 (poly(ADP-ribose) polymerase family member 15), HMCES (5-hydroxymethylcytosine binding, ES cell specific), RPL22L1 (ribosomal protein L22 like 1), DNAJC19 (DnaJ heat shock protein family (Hsp40) member C19), ECE2 (endothelin converting enzyme 2), MUC20 (mucin 20, cell surface associated), ELOVL6 (ELOVL fatty acid elongase 6), SORBS2 (sorbin and SH3 domain containing 2), WDR1 (WD repeat domain 1), SEL1L3 (SEL1L family member 3), RBM47 (RNA binding motif protein 47), SPON2 (spondin 2), C4orf48 (chromosome 4 open reading frame 48), PPP2R2C (protein phosphatase 2 regulatory subunit Bgamma), HTRA3 (HtrA serine peptidase 3), CPZ (carboxypeptidase Z), SLC34A2 (solute carrier family 34 member 2), SPINK2 (serine peptidase inhibitor Kazal type 2), IGFBP7 (insulin like growth factor binding protein 7), PKD2 (polycystin 2, transient receptor potential cation channel), UGT8 (UDP glycosyltransferase 8), EDNRA (endothelin receptor type A), HMGB2 (high mobility group box 2), CENPU (centromere protein U), PDLIM3 (PDZ and LIM domain 3), ECSCR (endothelial cell surface expressed chemotaxis and apoptosis regulator), SLIT3 (slit guidance ligand 3), SLC45A2 (solute carrier family 45 member 2), UGT3A2 (UDP glycosyltransferase family 3 member A2), OSMR (oncostatin M receptor), CSF1R (colony stimulating factor 1 receptor), GM2A (GM2 ganglioside activator), G3BP1 (G3BP stress granule assembly factor 1), ATP10B (ATPase phospholipid transporting 10B (putative)), WWC1 (WW and C2 domain containing 1), EMB (embigin), ALDH7A1 (aldehyde dehydrogenase 7 family member A1), TCF7 (transcription factor 7), PCDH1 (protocadherin 1), SPINK1 (serine peptidase inhibitor Kazal type 1), SPARC (secreted protein acidic and cysteine rich), DUSP1 (dual specificity phosphatase 1), STC2 (stanniocalcin 2), NOP16 (NOP16 nucleolar protein), PDLIM7 (PDZ and LIM domain 7), RAB44 (RAB44, member RAS oncogene family), SLC22A3 (solute carrier family 22 member 3), IRF4 (interferon regulatory factor 4), ALDH5A1 (aldehyde dehydrogenase 5 family member A1), CCDC167 (coiled-coil domain containing 167), AIM1 (crystallin beta-gamma domain containing 1), RPF2 (ribosome production factor 2 homolog), MOXD1 (monooxygenase DBH like 1), SLC17A4 (solute carrier family 17 member 4), PRSS16 (serine protease 16), SFTA2 (surfactant associated 2), SLC44A4 (solute carrier family 44 member 4), CFB (complement factor B), C4A (complement C4A (Rodgers blood group)), C4B (complement C4B (Chido blood group)), GPSM3 (G protein signaling modulator 3), HLA-DOB (major histocompatibility complex, class II, DO beta), TAP2 (transporter 2, ATP binding cassette subfamily B member), PSMB9 (proteasome 20S subunit beta 9), HLA-DOA (major histocompatibility complex, class II, DO alpha), TREM2 (triggering receptor expressed on myeloid cells 2), C6orf132 (chromosome 6 open reading frame 132), MRPL14 (mitochondrial ribosomal protein L14), HSP90AB1 (heat shock protein 90 alpha family class B member 1), MEP1A (meprin A subunit alpha), TNFRSF21 (TNF receptor superfamily member 21), CTGF (cellular communication network factor 2), TAGAP (T cell activation RhoGTPase activating protein), AOAH (acyloxyacyl hydrolase), PRKAR1B (protein kinase cAMP-dependent type I regulatory subunit beta), ADAP1 (ArfGAP with dual PH domains 1), MICALL2 (MICAL like 2), MAD1L1 (mitotic arrest deficient 1 like 1), AHR (aryl hydrocarbon receptor), CREBS (cAMP responsive element binding protein 5), NUDT1 (nudix hydrolase 1), LFNG (LFNG O-fucosylpeptide 3-beta-N-acetylglucosaminyltransferase), MEOX2 (mesenchyme homeobox 2), SFRP4 (secreted frizzled related protein 4), YAE1D1 (YAE1 maturation factor of ABCE1), MYO1G (myosin IG), RAMP3 (receptor activity modifying protein 3), CNPY4 (canopy FGF signaling regulator 4), MEST (mesoderm specific transcript), AKR1B10 (aldo-keto reductase family 1 member B10), ATP6V0A4 (ATPase H+ transporting V0 subunit a4), CLEC5A (C-type lectin domain containing 5A), PIP (prolactin induced protein), TCAF1 (TRPM8 channel associated factor 1), DNAJC5B (DnaJ heat shock protein family (Hsp40) member C5 beta), MTUS1 (microtubule associated scaffold protein 1), LOXL2 (lysyl oxidase like 2), DOCKS (dedicator of cytokinesis 5), DPYSL2 (dihydropyrimidinase like 2), SULF1 (sulfatase 1), TPD52 (tumor protein D52), FAM83A (family with sequence similarity 83 member A), TATDN1 (TatD DNase domain containing 1), SLA (Src like adaptor), KHDRBS3 (KH RNA binding domain containing, signal transduction associated 3), SMIM19 (small integral membrane protein 19), MRPL15 (mitochondrial ribosomal protein L15), TRAM1 (translocation associated membrane protein 1), CRISPLD1 (cysteine rich secretory protein LCCL domain containing 1), CHMP4C (charged multivesicular body protein 4C), ESRP1 (epithelial splicing regulatory protein 1), NIPAL2 (NIPA like domain containing 2), CTHRC1 (collagen triple helix repeat containing 1), SQLE (squalene epoxidase), FAM189A2 (family with sequence similarity 189 member A2), GDA (guanine deaminase), CORO2A (coronin 2A), NDUFA8 (NADH:ubiquinone oxidoreductase subunit A8), FAM214B (family with sequence similarity 214 member B), TPM2 (tropomyosin 2), CTSL (cathepsin L), CTSV (cathepsin V), GSN (gelsolin), STOM (stomatin), PTGS1 (prostaglandin-endoperoxide synthase 1), ZDHHC12 (zinc finger DHHC-type palmitoyltransferase 12), PRRX2 (paired related homeobox 2), SURF2 (surfeit 2), PHPT1 (phosphohistidine phosphatase 1), PTGDS (prostaglandin D2 synthase), CLIC3 (chloride intracellular channel 3), DHRSX (dehydrogenase/reductase X-linked), SLC9A7 (solute carrier family 9 member A7), POF1B (POF1B actin binding protein), GPC3 (glypican 3), CSF2RA (colony stimulating factor 2 receptor subunit alpha), ARSD (arylsulfatase D), ARSE (arylsulfatase L), GPR143 (G protein-coupled receptor 143), USP11 (ubiquitin specific peptidase 11), SLC38A5 (solute carrier family 38 member 5), PLP2 (proteolipid protein 2), IL2RG (interleukin 2 receptor subunit gamma), CITED1 (Cbp/p300 interacting transactivator with Glu/Asp rich carboxy-terminal domain 1), NOX1 (NADPH oxidase 1), SASH3 (SAM and SH3 domain containing 3), IGSF1 (immunoglobulin superfamily member 1), HMGB3 (high mobility group box 3), CSAG1 (chondrosarcoma associated gene 1), BGN (biglycan), L1CAM (L1 cell adhesion molecule), CCDC3 (coiled-coil domain containing 3), TBATA (thymus, brain and testes associated), PIK3AP1 (phosphoinositide-3-kinase adaptor protein 1), NET1 (solute carrier family 6 member 2), ATP5C1 (ATP synthase F1 subunit gamma), SRGN (serglycin), H2AFY2 (macroH2A.2 histone), PPA1 (inorganic pyrophosphatase 1), UNC5B (unc-5 netrin receptor B), PLAU (plasminogen activator, urokinase), DUSP5 (dual specificity phosphatase 5), VENTX (VENT homeobox), FUOM (fucose mutarotase), LDLRAD3 (low density lipoprotein receptor class A domain containing 3), ANO1 (anoctamin 1), B4GALNT4 (beta-1,4-N-acetyl-galactosaminyltransferase 4), RASSF7 (Ras association domain family member 7), CHID1 (chitinase domain containing 1), MUC5B (mucin 5B, oligomeric mucus/gel-forming), IGF2 (insulin like growth factor 2), SLC22A18 (solute carrier family 22 member 18), DKK3 (dickkopf WNT signaling pathway inhibitor 3), SAA2 (serum amyloid A2), SAA1 (serum amyloid A1), HTATIP2 (HIV-1 Tat interactive protein 2), EHF (ETS homologous factor), CREB3L1 (cAMP responsive element binding protein 3 like 1), UBE2L6 (ubiquitin conjugating enzyme E2 L6), LPXN (leupaxin), PLA2G16 (phospholipase A and acyltransferase 3), FERMT3 (fermitin family member 3), OVOL1 (ovo like transcriptional repressor 1), SLC29A2 (solute carrier family 29 member 2), FOLR1 (folate receptor alpha), MMP10 (matrix metallopeptidase 10), MMP1 (matrix metallopeptidase 1), MMP3 (matrix metallopeptidase 3), MMP12 (matrix metallopeptidase 12), POU2AF1 (POU class 2 homeobox associating factor 1), CD3D (CD3d molecule), TRIM29 (tripartite motif containing 29), VSIG2 (V-set and immunoglobulin domain containing 2), BARX2 (BARX homeobox 2), GUCY2C (guanylate cyclase 2C), B4GALNT3 (beta-1,4-N-acetyl-galactosaminyltransferase 3), PLBD1 (phospholipase B domain containing 1), ARHGDIB (Rho GDP dissociation inhibitor beta), FAM60A (SIN3-HDAC complex associated factor), ERBB3 (erb-b2 receptor tyrosine kinase 3), LRP1 (LDL receptor related protein 1), MSRB3 (methionine sulfoxide reductase B3), ALDH2 (aldehyde dehydrogenase 2 family member), LAGS (lymphocyte activating 3), CLEC12A (C-type lectin domain family 12 member A), GPRC5A (G protein-coupled receptor class C group 5 member A), LDHB (lactate dehydrogenase B), TUBA1A (tubulin alpha 1a), TROAP (trophinin associated protein), KRT6B (keratin 6B), KRT6C (keratin 6C), KRT6A (keratin 6A), KRT8 (keratin 8), SLC39A5 (solute carrier family 39 member 5), DCN (decorin), IKBIP (IKBKB interacting protein), OAS1 (2′-5′-oligoadenylate synthetase 1), OAS3 (2′-5′-oligoadenylate synthetase 3), FLT3 (fms related receptor tyrosine kinase 3), FARP1 (FERM, ARH/RhoGEF and pleckstrin domain protein 1), COL4A1 (collagen type IV alpha 1 chain), COL4A2 (collagen type IV alpha 2 chain), HMGB1 (high mobility group box 1), ALOX5AP (arachidonate 5-lipoxygenase activating protein), TNFSF13B (TNF superfamily member 13b), MNAT1 (MNAT1 component of CDK activating kinase), SLC7A7 (solute carrier family 7 member 7), LRFN5 (leucine rich repeat and fibronectin type III domain containing 5), TMX1 (thioredoxin related transmembrane protein 1), DLGAP5 (DLG associated protein 5), SMOC1 (SPARC related modular calcium binding 1), RIN3 (Ras and Rab interactor 3), VRK1 (VRK serine/threonine kinase 1), PNP (purine nucleoside phosphorylase), MMP14 (matrix metallopeptidase 14), NGDN (neuroguidin), DHRS2 (dehydrogenase/reductase 2), FKBP3 (FKBP prolyl isomerase 3), PYGL (glycogen phosphorylase L), ERH (ERH mRNA splicing and mitosis factor), SERPINA3 (serpin family A member 3), AMN (amnion associated transmembrane protein), SIVA1 (SIVA1 apoptosis inducing factor), PLD4 (phospholipase D family member 4), THSD4 (thrombospondin type 1 domain containing 4), MCTP2 (multiple C2 and transmembrane domain containing 2), THBS1 (thrombospondin 1), FBN1 (fibrillin 1), PKM (pyruvate kinase M1/2), CHSY1 (chondroitin sulfate synthase 1), LPCAT4 (lysophosphatidylcholine acyltransferase 4), PHGR1 (proline, histidine and glycine rich 1), NUSAP1 (nucleolar and spindle associated protein 1), DUOXA2 (dual oxidase maturation factor 2), SQRDL (sulfide quinone oxidoreductase), CA12 (carbonic anhydrase 12), LOXL1 (lysyl oxidase like 1), PML (promyelocytic leukemia), SEMA7A (semaphorin 7A (John Milton Hagen blood group)), CSPG4 (chondroitin sulfate proteoglycan 4), ANPEP (alanyl aminopeptidase, membrane), PLCG2 (phospholipase C gamma 2), SEZ6L2 (seizure related 6 homolog like 2), POLR3K (RNA polymerase III subunit K), SOX8 (SRY-box transcription factor 8), TPSB2 (tryptase beta 2 (gene/pseudogene)), TPSAB1 (tryptase alpha/beta 1), HN1L (Jupiter microtubule associated homolog 2), SYNGR3 (synaptogyrin 3), NTHL1 (nth like DNA glycosylase 1), ECI1 (enoyl-CoA delta isomerase 1), TCEB2 (elongin B), PRSS22 (serine protease 22), PAQR4 (progestin and adipoQ receptor family member 4), SMIM22 (small integral membrane protein 22), NDE1 (nudE neurodevelopment protein 1), ERN2 (endoplasmic reticulum to nucleus signaling 2), IL21R (interleukin 21 receptor), EIF3C (eukaryotic translation initiation factor 3 subunit C), SULT1A4 (sulfotransferase family 1A member 4), DOC2A (double C2 domain alpha), TGFB1I1 (transforming growth factor beta 1 induced transcript 1), MMP2 (matrix metallopeptidase 2), CES1 (carboxylesterase 1), ADGRG1 (adhesion G protein-coupled receptor G1), CMTM3 (CKLF like MARVEL transmembrane domain containing 3), NQO1 (NAD(P)H quinone dehydrogenase 1), CLEC18B (C-type lectin domain family 18 member B), SLC7A5 (solute carrier family 7 member 5), CYBA (cytochrome b-245 alpha chain), CDT1 (chromatin licensing and DNA replication factor 1), SLC22A31 (solute carrier family 22 member 31), SPNS3 (sphingolipid transporter 3 (putative)), ADAP2 (ArfGAP with dual PH domains 2), SLC16A5 (solute carrier family 16 member 5), MYO1C (myosin IC), TM4SF5 (transmembrane 4 L six family member 5), TNFSF13 (TNF superfamily member 13), NAA38 (N-alpha-acetyltransferase 38, NatC auxiliary subunit), PIK3R6 (phosphoinositide-3-kinase regulatory subunit 6), CCL2 (C—C motif chemokine ligand 2), SLFN11 (schlafen family member 11), CCLS (C—C motif chemokine ligand 5), GRB7 (growth factor receptor bound protein 7), KRT16 (keratin 16), KRT17 (keratin 17), JUP (junction plakoglobin), MPO (myeloperoxidase), LIMD2 (LIM domain containing 2), SLC25A19 (solute carrier family 25 member 19), ST6GALNAC2 (ST6 N-acetylgalactosaminide alpha-2,6-sialyltransferase 2), TMC8 (transmembrane channel like 8), BAIAP2 (BAR/IMD domain containing adaptor protein 2), ALYREF (Aly/REF export factor), KLHL14 (kelch like family member 14), MBP (myelin basic protein), NDC80 (NDC80 kinetochore complex component), TTR (transthyretin), CYB5A (cytochrome b5 type A), GNA15 (G protein subunit alpha 15), MUC16 (mucin 16, cell surface associated), DDX39A (DExD-box helicase 39A), KLC3 (kinesin light chain 3), PTPRH (protein tyrosine phosphatase receptor type H), GNA11 (G protein subunit alpha 11), CREB3L3 (cAMP responsive element binding protein 3 like 3), CD70 (CD70 molecule), PRAM1 (PML-RARA regulated adaptor molecule 1), MRPL4 (mitochondrial ribosomal protein L4), AP1M2 (adaptor related protein complex 1 subunit mu 2), SPC24 (SPC24 component of NDC80 kinetochore complex), RAB3D (RAB3D, member RAS oncogene family), ZNF439 (zinc finger protein 439), MAN2B1 (mannosidase alpha class 2B member 1), RNASEH2A (ribonuclease H2 subunit A), FARSA (phenylalanyl-tRNA synthetase subunit alpha), NACC1 (nucleus accumbens associated 1), GIPC1 (GIPC PDZ domain containing family member 1), NOTCH3 (notch receptor 3), TPM4 (tropomyosin 4), PLVAP (plasmalemma vesicle associated protein), LSM4 (LSM4 homolog, U6 small nuclear RNA and mRNA degradation associated), CCNE1 (cyclin E1), GPI (glucose-6-phosphate isomerase), FXYD3 (FXYD domain containing ion transport regulator 3), CD22 (CD22 molecule), TYROBP (transmembrane immune signaling adaptor TYROBP), CAPNS1 (calpain small subunit 1), COX7A1 (cytochrome c oxidase subunit 7A1), LGALS4 (galectin 4), FBXO27 (F-box protein 27), GMFG (glia maturation factor gamma), DLL3 (delta like canonical Notch ligand 3), CYP2S1 (cytochrome P450 family 2 subfamily S member 1), TGFB1 (transforming growth factor beta 1), CNFN (cornifelin), SULT2B1 (sulfotransferase family 2B member 1), FAM83E (family with sequence similarity 83 member E), PLEKHA4 (pleckstrin homology domain containing A4), RRAS (RAS related), PRMT1 (protein arginine methyltransferase 1), IL4I1 (interleukin 4 induced 1), SIGLEC14 (sialic acid binding Ig like lectin 14), TMC4 (transmembrane channel like 4), ISOC2 (isochorismatase domain containing 2), EYA2 (EYA transcriptional coactivator and phosphatase 2), PYGB (glycogen phosphorylase B), E2F1 (E2F transcription factor 1), FAM83D (family with sequence similarity 83 member D), TOMM34 (translocase of outer mitochondrial membrane 34), RASSF2 (Ras association domain family member 2), FERMT1 (fermitin family member 1), OVOL2 (ovo like zinc finger 2), EIF6 (eukaryotic translation initiation factor 6), ADA (adenosine deaminase), PLTP (phospholipid transfer protein), MMP9 (matrix metallopeptidase 9), CD40 (CD40 molecule), RCAN1 (regulator of calcineurin 1), SAMSN1 (SAM domain, SH3 domain and nuclear localization signals 1), BTG3 (BTG anti-proliferation factor 3), SOD1 (superoxide dismutase 1), FAM3B (FAM3 metabolism regulating signaling molecule B), RIPK4 (receptor interacting serine/threonine kinase 4), SLC37A1 (solute carrier family 37 member 1), DNMT3L (DNA methyltransferase 3 like), SLC5A1 (solute carrier family 5 member 1), HMOX1 (heme oxygenase 1), CBY1 (chibby family member 1, beta catenin antagonist), BIK (BCL2 interacting killer), CELSR1 (cadherin EGF LAG seven-pass G-type receptor 1), PRODH (proline dehydrogenase 1), MIF (macrophage migration inhibitory factor), SUSD2 (sushi domain containing 2), SNRPD3 (small nuclear ribonucleoprotein D3 polypeptide), TIMP3 (TIMP metallopeptidase inhibitor 3), APOL4 (apolipoprotein L4), C1QTNF6 (C1q and TNF related 6), KDELR3 (KDEL endoplasmic reticulum protein retention receptor 3), ZC3H7B (zinc finger CCCH-type containing 7B) and WNT7B (Wnt family member 7B). It should be appreciated that each possibility disclosed herein represent a separate embodiment of the invention.

In yet some further embodiments, the target sequence that participates directly or indirectly in at least one splicing event, is comprised within an exon, within at least one intron located upstream or downstream to such exon, or within at least one splicing junction flanking this exon. More specifically, at least one of the 5′ intron/exon junction that is located upstream to the indicated exon, or alternatively, the 3′ exon/intron splice junction located downstream to the indicated exon. It should be appreciated that the methods of the invention may use any combination of splicing modulating agents that are specifically directed against target nucleic acid sequences in at least two of the exons indicated herein. In some embodiments, the invention may use several splicing modulating agents (ASOs or gRNAs), specific against one or more target sequence within any specified transcript. Still further, at least 2, 3, 4, 5, 6, 7, 8, 9, 10, 20, 30, 40, 50, 60, 70, 80, 90, 100 or more different target sequences in a single target transcript. In some specific embodiments, the splicing modulating agents used by the methods of the invention are directed against a target sequence located within an exon, within at least one intron located upstream or downstream to such exon, or within at least one splicing junction flanking at least one of the exons selected from the group of exons disclosed by Table 2, presented herein after by Example 14. More specifically, Table 2 specifies the particular transcripts of the target gene and moreover, the specific coordinates (e.g., start and end nucleotides in the specified chromosome, and the specified strand) for each of the exons in each target transcript that comprise the target sequence, or exons that comprise the target sequence in at least one of the flanking junctions (e.g., intron/exon or exon/intron junction), or within at least one intron located upstream or downstream to the exons specified in in Table 2. In more specific embodiments, the target sequence that participates directly or indirectly in at least one splicing event is comprised within an exon, within at least one intron located upstream or downstream to said exon, or within at least one splicing junction flanking said exon. In yet some further specific embodiments, such exon is any one of, or at least one of the following exons that are indicated herein by the specific coordinates thereof: chr1:167765583-167765749_+, chr1:1054448-1054551_+, chr1:68137780-68137933_−, chr1:6097269-6097357_+, chr1:9037897-9038024_−, chr1:27101203-27101275_−, chr1:31181860-31181941_−, chr1:36179874-36180067_+, chr1:38887963-38888024_−, chr1:59900965-59901103_−, chr1:108924000-108924214_+, chr1:109314261-109314384_−, chr1:116519231-116519267_−, chr1:119937282-119937466_−, chr1:150747777-150747879_−, chr1:154926354-154926483_−, chr1:180984677-180984771_−, chr1:223543108-223543166_−, chr1:223772181-223772239_+, chr1:226361969-226362083_−, chr1:236205126-236205250_+, chr1:244842195-244842258_+, chr1:6192930-6193054_−, chr1:6250440-6250624_−, chr1:13614300-13614405_+, chr1:19306018-19306147_−, chr1:20613842-20613899_+, chr1:23796151-23796265_−, chr1:24123302-24123423_−, chr1:27353283-27353488_+, chr1:27885295-27885451_+, chr1:27955949-27956082_+, chr1:31586710-31586755_+, chr1:31792215-31792401_−, chr1:39900939-39901443_−, chr1:43621097-43621232_+, chr1:43936069-43936231_+, chr1:53228872-53228954_−, chr1:65224752-65224870_+, chr1:85582416-85582624_+, chr1:89054682-89054875_−, chr1:92052510-92052658_+, chr1:92478588-92478753_−, chr1:109273436-109273670_+, chr1:111477651-111477775_+, chr1:111477726-111477775_+, chr1:150799168-150799273_−, chr1:153662705-153662795_−, chr1:154977987-154978114_+, chr1:156658543-156658733_+, chr1:156737646-156737776_−, chr1:156743652-156743878_−, chr1:158257421-158257511_+, chr1:158328921-158329014_−, chr1:158356498-158356591_+, chr1:159919274-159919376_−, chr1:160999043-160999091_−, chr1:161037913-161038075_−, chr1:161102048-161102171_−, chr1:202013829-202014024_+, chr1:203217739-203217865_−, chr1:206931497-206931555_−, chr2:101888796-101888935_+, chr2:8860613-8860764_−, chr2:69124565-69124643_+, chr2:98796120-98796264_−, chr2:105363285-105363471_−, chr2:157418523-157418589_−, chr2:157738440-157738570_−, chr2:161994961-161995034_−, chr2:218889984-218890363_+, chr2:224770211-224770349_−, chr2:233768220-233768439_+, chr2:237552337-237552437_+, chr2:10784934-10785030_−, chr2:24068321-24068459_−, chr2:27216523-27216620_+, chr2:47161723-47161858_−, chr2:65265043-65265175_+, chr2:68394107-68394176_+, chr2:73916584-73916765_+, chr2:74145189-74145303_−, chr2:74533882-74533993_−, chr2:85697506-85697677_+, chr2:86788530-86788560_−, chr2:86844922-86844958_−, chr2:108386192-108386372_+, chr2:162172811-162172884_−, chr2:181539092-181539264_−, chr2:200876919-200877037_−, chr2:202302921-202303057_+, chr2:219057433-219057694_−, chr2:230238213-230238381_+, chr2:230877400-230877550_+, chr3:142711501-142711625_+, chr3:13636445-13636568_+, chr3:45089054-45089141_−, chr3:124764391-124764557_−, chr3:126957416-126957551_+, chr3:4981392-4981515_+, chr3:38140757-38140848_+, chr3:40251881-40251999_+, chr3:52694037-52694103_+, chr3:100176327-100176415_+, chr3:111647543-111647666_+, chr3:122536168-122536342_−, chr3:129654968-129655103_−, chr3:190310169-190310253_−, chr3:12154327-12154451_−, chr3:39411896-39412061_+, chr3:41238016-41238076_+, chr3:46579951-46580060_+, chr3:50186613-50186746_+, chr3:51933708-51933781_−, chr3:58567208-58567364_−, chr3:120633147-120633328_−, chr3:122118048-122118093_+, chr3:122635020-122635194_+, chr3:129301950-129302142_+, chr3:170868013-170868134_−, chr3:180985926-180985996_−, chr3:184257473-184257756_+, chr3:195729648-195729739_+, chr4:110059603-110059754_−, chr4:185589679-185589785_−, chr4:10077304-10077448_−, chr4:25757534-25757606_−, chr4:40432651-40432862_−, chr4:1170402-1170576_−, chr4:2042736-2042824_+, chr4:6329262-6329353_−, chr4:8294087-8294201_+, chr4:8618429-8618528_+, chr4:25674505-25674629_+, chr4:56811685-56811794_−, chr4:57032426-57032552_−, chr4:88074812-88074959_+, chr4:114668085-114668304_+, chr4:147540377-147540485_+, chr4:173332821-173332995_−, chr4:184697647-184697803_−, chr4:185504475-185504586_−, chr5:139449078-139449174_−, chr5:168669783-168669991_−, chr5:33951554-33951677_−, chr5:36037797-36038016_−, chr5:38932463-38932535_+, chr5:150054322-150054430_−, chr5:151266731-151266899_+, chr5:151800760-151800869_+, chr5:160569496-160569683_−, chr5:168467843-168467964_+, chr5:168467840-168467964_+, chr5:50399859-50399913_−, chr5:126546324-126546399_−, chr5:134143592-134143640_+, chr5:141857252-141857471_−, chr5:147828022-147828128_−, chr5:151664087-151664235_−, chr5:172769575-172769794_−, chr5:173323219-173323430_−, chr5:176385221-176385327_−, chr5:177483867-177483982_−, chr5:177490870-177490906_−, chr6:36730673-36730749_+, chr6:160447719-160447818_+, chr6:405018-405130_+, chr6:24532119-24532177_+, chr6:37484810-37484862_−, chr6:106563764-106563926_+, chr6:111024183-111024327_+, chr6:132297787-132297955_−, chr6:25777926-25778016_+, chr6:27254986-27255131_+, chr6:30931707-30931795_−, chr6:31864652-31864736_−, chr6:31951555-31951604_+, chr6:32002123-32002255_+, chr6:32034860-32034992_+, chr6:32191709-32191908_−, chr6:32813440-32813471_−, chr6:32829400-32829536_−, chr6:32858364-32858505_+, chr6:33007080-33007215_−, chr6:41159792-41159882_−, chr6:42104463-42107583_−, chr6:44116541-44116629_−, chr6:44253045-44253378_+, chr6:46835249-46835549_+, chr6:47234670-47234898_−, chr6:131949949-131950160_−, chr6:159038114-159038228_−, chr7:36522039-36522115_−, chr7:551389-551470_−, chr7:899033-899261_−, chr7:1435101-1435147_−, chr7:1898200-1898390_−, chr7:17338986-17340228_+, chr7:28818071-28818179_+, chr7:2249857-2250002_+, chr7:2526836-2526921_+, chr7:15626746-15626918_−, chr7:37909617-37909680_−, chr7:39570506-39570627_+, chr7:44962970-44963124_−, chr7:45177309-45177441_+, chr7:100124514-100124631_+, chr7:130503933-130503996_+, chr7:134538935-134539017_+, chr7:138709624-138709795_−, chr7:141931720-141931826_−, chr7:143139075-143139189_+, chr7:143857100-143857350_−, chr8:66080377-66080548_+, chr8:17646982-17647079_−, chr8:23298836-23298947_−, chr8:25410099-25410202_+, chr8:26653232-26653397_+, chr8:69640808-69640841_+, chr8:80042620-80042668_−, chr8:123194024-123194148_+, chr8:124493833-124493959_−, chr8:133039998-133040130_−, chr8:135645059-135645117_+, chr8:42548656-42548780_+, chr8:54142663-54142786_+, chr8:70583164-70583324_−, chr8:75029387-75029517_+, chr8:81758142-81758295_+, chr8:94678203-94678371_+, chr8:98195942-98196005_−, chr8:103378027-103378243_+, chr8:125020784-125020871_+, chr9:69388158-69388450_+, chr9:72247406-72247433_+, chr9:98126549-98126823_−, chr9:122148112-122148277_−, chr9:35105682-35105857_−, chr9:35684246-35684315_−, chr9:87730381-87730498_+, chr9:97034726-97034843_−, chr9:121331388-121331448_+, chr9:121356053-121356156_−, chr9:122390198-122390345_+, chr9:128721651-128721817_−, chr9:128721651-128721814_−, chr9:129720596-129720774_+, chr9:133360265-133360434_+, chr9:136850013-136850137_+, chr9:136980833-136980855_+, chr9:136994930-136995105_−, chrX:2243023-2243230_−, chrX:46613289-46613394_−, chrX:85282203-85282317_−, chrX:133596440-133596599_−, chrX:1305446-1305527_+, chrX:2908721-2908842_−, chrX:2936742-2936863_−, chrX:9739485-9739719_−, chrX:47247611-47247699_+, chrX:48459536-48459639_−, chrX:49174335-49174425_+, chrX:71108277-71108346_−, chrX:72302810-72302934_−, chrX:100848630-100848754_−, chrX:129792989-129793139_+, chrX:131274083-131274206_−, chrX:150987128-150987302_+, chrX:152728074-152728224_−, chrX:153507047-153507185_+, chrX:153863477-153863549_−, chrY:2243023-2243230_−, chrY:1305446-1305527_+, chr10:12998338-12998512_−, chr10:70772514-70772566_−, chr10:96602280-96602398_−, chr10:5456087-5456273_+, chr10:7802758-7802854_+, chr10:69097084-69097231_+, chr10:70109033-70109207_+, chr10:70204873-70204915_−, chr10:71297909-71298090_+, chr10:73915251-73915399_+, chr10:110506935-110507154_+, chr10:133239676-133239836_+, chr10:133355738-133355811_−, chr11:36227085-36227430_+, chr11:70185590-70185695_+, chr11:380825-380951_+, chr11:562079-562776_+, chr11:870121-870163_−, chr11:1260626-1260728_+, chr11:2133517-2133665_−, chr11:2922436-2922556_+, chr11:11965809-11965965_−, chr11:18245910-18246048_−, chr11:18269195-18269333_+, chr11:20382178-20382239_+, chr11:34658533-34658728_+, chr11:46320264-46320528_+, chr11:57554437-57554623_−, chr11:58528043-58528191_−, chr11:63590100-63590368_−, chr11:64223048-64223189_+, chr11:65794538-65794727_+, chr11:66364225-66364424_−, chr11:72195612-72195747_+, chr11:102772012-102772115_−, chr11:102790703-102790806_−, chr11:102837298-102837401_−, chr11:102864146-102864252_−, chr11:111357445-111357710_−, chr11:118339451-118339494_−, chr11:120115338-120115414_−, chr11:124748390-124748534_−, chr11:129442835-129442919_+, chr12:14614867-14614943_−, chr12:559295-559421_+, chr12:14506162-14506268_−, chr12:14944776-14944839_−, chr12:31287634-31287784_−, chr12:56101061-56101361_+, chr12:57212117-57212261_+, chr12:65453728-65453825_+, chr12:111803859-111803973_+, chr12:6777791-6777921_+, chr12:9982020-9982129_+, chr12:12912084-12912142_+, chr12:21637071-21637194_−, chr12:49186310-49186458_−, chr12:49323845-49324037_+, chr12:52447539-52447573_−, chr12:52469411-52469445_−, chr12:52488069-52488103_−, chr12:52898461-52898519_−, chr12:56237150-56237340_+, chr12:91151654-91151792_−, chr12:98634296-98634413_−, chr12:112917547-112917700_+, chr12:112969608-112969755_+, chr13:28014452-28014557_−, chr13:98446666-98446817_+, chr13:110152334-110152506_−, chr13:110507935-110508221_+, chr13:30462538-30462712_−, chr13:30755944-30756025_+, chr13:108303454-108303604_+, chr14:60879714-60879835_+, chr14:22773933-22774116_−, chr14:41898917-41898960_+, chr14:51249693-51249765_+, chr14:55150799-55150848_−, chr14:70023203-70023447_+, chr14:92684987-92685150_+, chr14:96876030-96876120_+, chr14:20475062-20475252_+, chr14:22845251-22845366_+, chr14:23477503-23477560_+, chr14:23644827-23644882_+, chr14:45118028-45118125_−, chr14:50908271-50908337_−, chr14:69386963-69387083_−, chr14:94622341-94622491_+, chr14:102930406-102930493_+, chr14:104756604-104756760_+, chr14:104932259-104932355_+, chr15:71771064-71771208_+, chr15:94476696-94476793_+, chr15:39594301-39594440_+, chr15:48412569-48412743_−, chr15:72200474-72200655_−, chr15:101235082-101235577_−, chr15:34359589-34359745_−, chr15:40354345-40354352_+, chr15:41377196-41377304_+, chr15:45117091-45117305_+, chr15:45689039-45689217_+, chr15:63327149-63327233_−, chr15:73949459-73949574_+, chr15:74042989-74043139_+, chr15:74411295-74411356_−, chr15:75677703-75677886_−, chr15:89790460-89790541_−, chr16:81956695-81956879_+, chr16:29872187-29872283_−, chr16:51558-51645_−, chr16:983728-983960_+, chr16:1228900-1229063_−, chr16:1241827-1241990_+, chr16:1697812-1697860_+, chr16:1992636-1992778_+, chr16:2040133-2040238_−, chr16:2243046-2243224_−, chr16:2771994-2772102_−, chr16:2853865-2854022_−, chr16:2971515-2971684_+, chr16:4795948-4796033_+, chr16:15696709-15696860_+, chr16:23691129-23691196_−, chr16:27446007-27446088_+, chr16:28735173-28735335_+, chr16:29464160-29464340_+, chr16:30006413-30006509_−, chr16:31476862-31477010_+, chr16:55502779-55502888_+, chr16:55803529-55803601_−, chr16:57661697-57661965_+, chr16:66609883-66610003_+, chr16:69718123-69718253_−, chr16:74409550-74409641_−, chr16:87834414-87834591_−, chr16:88646116-88646197_−, chr16:88807281-88807482_+, chr16:89197298-89197409_−, chr17:4486412-4486583_+, chr17:30956241-30956469_+, chr17:75103970-75104180_+, chr17:1467242-1467341_−, chr17:4782854-4783037_+, chr17:7560350-7560488_+, chr17:7856985-7857198_−, chr17:8804041-8804153_−, chr17:34256222-34256339_+, chr17:35353336-35354059_−, chr17:35878528-35878639_−, chr17:39746109-39746202_+, chr17:41610190-41610236_−, chr17:41620536-41620558_−, chr17:41756175-41756214_−, chr17:58271655-58271892_−, chr17:63698799-63698938_−, chr17:75277353-75277483_−, chr17:76567453-76567552_−, chr17:78139162-78139240_+, chr17:81108475-81108509_+, chr17:81888241-81888418_−, chr18:32677173-32677330_−, chr18:77066298-77066385_−, chr18:2610759-2610861_+, chr18:31595120-31595255_+, chr18:74255741-74255775_−, chr19:3157728-3157881_+, chr19:8851276-8851355_−, chr19:14409037-14409184_−, chr19:45350954-45351017_+, chr19:55182016-55182151_−, chr19:3119206-3119359_+, chr19:4171383-4171479_+, chr19:6590103-6590136_−, chr19:8490338-8490372_−, chr19:10258609-10258685_+, chr19:10574417-10574492_−, chr19:11147818-11147894_−, chr19:11335447-11335571_−, chr19:11866537-11866597_+, chr19:12647233-12647335_−, chr19:12813083-12813206_+, chr19:12924151-12924265_−, chr19:13137478-13137575_+, chr19:14478684-14478765_−, chr19:15162465-15162562_−, chr19:16093684-16093753_+, chr19:17360528-17360609_−, chr19:18309678-18309861_−, chr19:29822446-29822603_+, chr19:34399719-34399785_+, chr19:35123271-35123308_+, chr19:35346151-35346235_+, chr19:35907218-35907264_−, chr19:35907218-35907261_−, chr19:36149578-36149636_+, chr19:36151462-36151546_−, chr19:38801992-38802157_−, chr19:39026870-39027005_−, chr19:39329000-39329073_−, chr19:39507830-39507914_+, chr19:41205958-41206099_+, chr19:41332128-41332281_−, chr19:42387340-42387476_−, chr19:48596739-48596919_+, chr19:48603494-48603911_−, chr19:48838017-48838129_−, chr19:49635734-49635852_−, chr19:49686605-49686726_+, chr19:49890971-49891107_−, chr19:51643537-51643672_−, chr19:54160467-54160545_−, chr19:55454989-55455106_−, chr20:47183291-47183391_+, chr20:25295604-25295670_+, chr20:33677105-33677330_−, chr20:38947876-38948000_+, chr20:44943453-44943579_−, chr20:4786231-4786328_−, chr20:6079436-6079577_−, chr20:18041534-18041723_−, chr20:35279566-35279747_−, chr20:44620299-44620401_−, chr20:45899462-45899538_−, chr20:46014371-46014474_+, chr20:46128330-46128358_+, chr21:34521499-34521658_−, chr21:14498442-14498592_−, chr21:17598617-17598824_−, chr21:31667258-31667375_+, chr21:41348592-41348724_+, chr21:41743882-41744140_−, chr21:42579736-42579800_+, chr21:44249026-44249111_−, chr22:32104786-32104891_+, chr22:35389864-35389963_+, chr22:38671070-38671188_+, chr22:43128496-43128625_+, chr22:46363996-46364251_−, chr22:18913438-18913526_−, chr22:23894772-23894944_+, chr22:24188225-24188327_+, chr22:24567984-24568176_+, chr22:32858017-32858138_+, chr22:36195311-36195437_−, chr22:37185218-37185455_−, chr22:38481212-38481464_+, chr22:41356645-41356808_+ and chr22:45931098-45931369_−. It should be appreciated that each possibility disclosed herein represent a separate embodiment of the invention. It should be understood that this list of exons specifies the particular location of the exon in the genome. More specifically, the format is: chromosome:start-end_strand. Particularly, the specific chromosome (chr), the specific start nucleotide and end nucleotide, as well as the strand [either the positive strand (indicated herein as “+” or the negative strand, indicated herein as “−”)].

It should be appreciated that the nucleic acid sequence of the splicing modulating agent of the invention, specifically, the at least one AON/s and/or gRNA used herein, is complementary to target sequences comprised within any of the targets specified herein, or to any sequence that display at least 75% homology to the indicated targets, more specifically, 80%, 85%, 90%, 95%, 96%, 97%, 98%, 99% and more, homology. More specific embodiments concern complementarity of the splicing targeting agents of the invention to a target sequence that display at least 90% homology to target sequences comprised within any of the targets specified by herein above, and by Table 2.

In more specific embodiments the target gene that comprise the target nucleic acid sequence that participates directly or indirectly in at least one splicing event, is overexpressed in melanoma. In some particular embodiments, the target gene may be the tyrosinase (TYR) gene. Thus, in some embodiments the target gene is the tyrosinase (TYR) gene, and the cancer is melanoma.

Tyrosinase, as used herein, is a protein also known as TYR, Tyr, tyr, ATN, CMM8, OCA1, OCA1A, OCAIA, SHEP3, tyrosinase and Tyrosinase. This protein is an oxidase that is the rate-limiting enzyme for controlling the production of melanin. The enzyme is mainly involved in two distinct reactions of melanin synthesis; firstly, the hydroxylation of a monophenol and secondly, the conversion of an o-diphenol to the corresponding o-quinone. o-Quinone undergoes several reactions to eventually form melanin. Tyrosinase is a copper-containing enzyme present in plant and animal tissues that catalyzes the production of melanin and other pigments from tyrosine by oxidation, as in the blackening of a peeled or sliced potato exposed to air. It is found inside melanosomes which are synthesized in the skin melanocytes. In humans, the tyrosinase enzyme is encoded by the TYR gene.

In yet some further specific embodiments, the protein Tyr referred to herein, in humans refers the uniport protein TYRO_HUMAN, UNIPROT ID P14679 and in mouse refers to TYRO_MOUSE, UNIPROT ID P11344. The human TYR mRNA transcript refers to RefSeq NM_000372.4 as denoted by SEQ ID NO. 10, and/or Ensembl ID ENST00000263321.5 in humans and in mouse refers to Refseq NM_011661.5, as denoted by SEQ ID NO. 11. As indicated above, in yet some further particular and non-limiting embodiments, the TYR gene, may be a specifically relevant target gene in cancer cells of a subject suffering from melanoma.

In some embodiments, for inducing aberrant splicing in the TYR gene, targeting nucleic acid sequences that participate or affect splicing of exon 4 may be used by the method of the invention. In some embodiments, exon 4 of the human TYR gene may comprise the nucleic acid sequence as denoted by SEQ ID NO. 1. In yet some further embodiments, exon 4 of the mouse TYR gene may comprise the nucleic acid sequence as denoted by SEQ ID NO. 8. More specifically, in some particular embodiments, the specific exon targeted in the human TYR transcript is located on chromosome 11, starting at nucleotide 89284773 and ending at nucleotide 89284954 of the positive strand (strand+). In some further embodiments, the mouse TYR transcript is located on chromosome 7, starting at nucleotide 87437937 and ending at nucleotide 87438118 of the negative strand (strand-).

In certain embodiments, the splicing modulating agent used by the methods of the invention may comprise at least one AON. Thus, in some specific embodiments, the nucleic acid sequences that participate or affect splicing of exon 4 is targeted using AONs. In more specific embodiments, the AONs that target the human TYR exon 4 may comprise the nucleic acids sequence as denoted by any one of SEQ ID NOs. 28 to 47, or any variants, homologs and derivatives thereof.

In yet some further embodiments, the mouse TYR gene is targeted by the methods of the invention. More specifically, in some specific embodiments, targeting AONs may comprise the AON designated herein as TYR oligo 9, specifically an oligonucleotide targeting a sequence comprising the nucleic acids sequence as denoted by SEQ ID NO. 6. In more specific embodiment, the oligo 9 AON, may comprise the nucleic acid sequence as denoted by SEQ ID NO. 4 or any variants, homologs or derivatives thereof. In yet some further embodiments, targeting AONs used by the methods of the invention may comprise the AON designated herein as TYR oligo 13, specifically an oligonucleotide targeting a sequence comprising the nucleic acids sequence as denoted by SEQ ID NO. 7. In more specific embodiment, the oligo 13 AON may comprise the nucleic acid sequence as denoted by SEQ ID NO. 5 or any variants, homologs or derivatives thereof.

In yet some further embodiments, the splicing modulating agent used by the methods of the invention may comprise at least one gRNA, specifically, using the CRISPR-Cas system specified above. Thus, in some specific embodiments, the nucleic acid sequences that participate or affect splicing of exon 4 is targeted using at least one gRNA. In more specific embodiments, gRNAs that target the exon 4 of the human TYR transcript may comprise the nucleic acid sequence as denoted by any one of SEQ ID NOs. 20 to 27.

In yet some further embodiments, the gRNAs that target the exon 4 of the mouse TYR transcript may comprise the nucleic acid sequence as denoted by any one of SEQ ID NO. 16 and 17 (targeting the 5′ splice site) and SEQ ID NOs. 18 and 19 (targeting the 3′ splice site).

In more specific embodiments the target gene that comprise the target nucleic acid sequence that participates directly or indirectly in at least one splicing event, is overexpressed in breast cancer. In some particular embodiments, the target gene may be the heterogeneous nuclear ribonucleoprotein A/B (HNRNPAB) gene. Thus, in some embodiments the target gene is the heterogeneous nuclear ribonucleoprotein A/B (HNRNPAB). In some embodiments, the cancer is breast cancer.

As indicated above, in yet some further particular and non-limiting embodiments, the HNRNPAB gene, may be a specifically relevant target gene in cancer cells of a subject suffering from breast cancer. The HNRNPAB gene, as used herein belongs to the subfamily of ubiquitously expressed heterogeneous nuclear ribonucleoproteins (hnRNPs). The hnRNPs are components of the heterogeneous nuclear RNA (hnRNA) complexes, and are associated with pre-mRNAs in the nucleus and appear to influence pre-mRNA processing and other aspects of mRNA metabolism and transport. While all of the hnRNPs are present in the nucleus, some seem to shuttle between the nucleus and the cytoplasm. The hnRNP proteins have distinct nucleic acid binding properties. The protein encoded by the hnRNP gene binds to one of the components of the multiprotein editosome complex, has two repeats of quasi-RRM (RNA recognition motif) domains that bind to RNAs. In yet some further specific embodiments, the human hnRNPAB mRNA transcript refers to RefSeq NM_004499.3 as denoted by SEQ ID NOs. 51 49, and in mouse refers to Refseq NM_010448.3, as denoted by SEQ ID NO. 49.

In some embodiments, for inducing aberrant splicing in the HNRNPAB gene, targeting nucleic acid sequences that participate or affect splicing of exon 6 may be used by the method of the invention. In some embodiments, exon 6 of the human HNRNPAB gene may comprise the nucleic acid sequence as denoted by SEQ ID NO. 50. In yet some further embodiments, exon 6 of the mouse HNRNPAB gene may comprise the nucleic acid sequence as denoted by SEQ ID NO. 48. More specifically, in some particular embodiments, the specific exon targeted in the human hnRNPAB transcript is located on chromosome 5, starting at nucleotide 178209330 and ending at nucleotide 178209447 of the positive strand (strand+). In some further embodiments, the mouse hnRNPAB transcript is located on chromosome 11, starting at nucleotide 51602593 and ending at nucleotide 51602695 of the negative strand (strand-).

In certain embodiments, the splicing modulating agent used by the methods of the invention may comprise at least one AON. Thus, in some specific embodiments, the nucleic acid sequences that participate or affect splicing of exon 6 is targeted using AONs. In more specific embodiments, the AONs that target the human hnRNPAB exon 6 may comprise the nucleic acids sequence as denoted by any one of SEQ ID NOs. 67 to 86, or any variants, homologs and derivatives thereof. In yet some further embodiments, the splicing modulating agent used by the methods of the invention may comprise at least one gRNA, specifically, using the CRISPR-Cas system specified above. Thus, in some specific embodiments, the nucleic acid sequences that participate or affect splicing of exon 6 of hnRNPAB is targeted using at least one gRNA. In more specific embodiments, gRNAs that target the exon 6 of the human hnRNPAB transcript may comprise the nucleic acid sequence as denoted by any one of SEQ ID NOs. 59 to 66.

In yet some further embodiments, the gRNAs that target the exon 6 of the mouse hnRNPAB transcript may comprise the nucleic acid sequence as denoted by any one of SEQ ID NOs. 57 and 58 (targeting the 3′ splice site).

Still further, in some embodiments, the method of the invention induces the production and thereby the expression of the newly created neoantigen by the target cell. In some embodiments, the expression of the neoantigen leads to activation of an immune-response directed against this target cell. The term “immune response” refers herein to any response to an antigen or antigenic determinant by the immune system. Exemplary immune responses include humoral immune responses (e.g. production of antigen-specific antibodies (neutralizing or otherwise) and cell-mediated immune responses (e.g. lymphocyte proliferation). The immune system can be classified into two functional subsystems: the innate and the acquired immune system. The innate immune system is the first line of defense against infections, and most potential pathogens are rapidly neutralized by this system before they can cause, for example, a noticeable infection. The acquired immune system reacts to molecular structures of the intruding organism. There are two types of acquired immune reactions, which include the humoral immune reaction and the cell-mediated immune reaction. In the humoral immune reaction, antibodies secreted by B cells into bodily fluids bind to pathogen-derived antigens, leading to the elimination of the pathogen through a variety of mechanisms, e.g. complement-mediated lysis.

In the cell-mediated immune reaction, T-cells capable of destroying other cells are activated. For example, if proteins associated with a disease are present in a cell, they are fragmented proteolytically to peptides within the cell. Specific cell proteins then attach themselves to the antigen or peptide formed in this manner and transport them to the surface of the cell, where they are presented to the molecular defense mechanisms, in particular T-cells, of the body. Cytotoxic T cells recognize these antigens and kill the cells that harbor the antigens.

Still further, in some embodiments, the presentation of peptides derived from the newly produced or created neoantigen by at least one antigen presenting cell (APC) in the context of at least one of Major histocompatibility class I (MHC Class I) and class II (MHC Class II), results in the activation of an immune-response against the target cell.

More specifically, the molecules that transport and present peptides on the cell surface are referred to as proteins of the major histocompatibility complex (MHC). MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells, and are then presented to naive or cytotoxic T-lymphocytes (CTLs). MHC class II proteins are present on dendritic cells, B-lymphocytes, macrophages and other antigen-presenting cells. They mainly present peptides, which are processed from external antigen sources, i.e. outside of the cells, to T-helper (Th) cells. Most of the peptides bound by the MHC class I proteins originate from cytoplasmic proteins produced in the healthy host cells of an organism itself, and do not normally stimulate an immune reaction. Accordingly, cytotoxic T-lymphocytes that recognize such self-peptide-presenting MHC molecules of class I are deleted in the thymus (central tolerance) or, after their release from the thymus, are deleted or inactivated, i.e. tolerized (peripheral tolerance). MHC molecules are capable of stimulating an immune reaction when they present peptides to non-tolerized T-lymphocytes. Cytotoxic T-lymphocytes have both T-cell receptors (TCR) and CD8 molecules on their surface. T-Cell receptors are capable of recognizing and binding peptides complexed with the molecules of MHC class I. Each cytotoxic T-lymphocyte expresses a unique T-cell receptor which is capable of binding specific MHC/peptide complexes. The peptide antigens attach themselves to the molecules of MHC class I by competitive affinity binding within the endoplasmic reticulum, before they are presented on the cell surface.

MHC proteins have immunoglobulin-like structure. In yet some further embodiments, MHC I occurs as an a chain composed of three domains, α1, α2, and α3. The α1 rests upon a unit of the non-MHC molecule (32 microglobulin (encoded on human chromosome 15). The α3 domain is transmembrane, anchoring the MHC class I molecule to the cell membrane. The peptide being presented is held by the floor of the peptide-binding groove, in the central region of the α1/α2 heterodimer (a molecule composed of two nonidentical subunits). The genetically encoded and expressed sequence of amino acids, the sequence of residues, of the peptide-binding groove's floor determines which particular peptide residues it binds.

MHC class II is formed of two chains, α and β, each having two domains, α1 and α2 and β1 and β2, each chain having a transmembrane domain, α2 and β2, respectively, anchoring the MHC class II molecule to the cell membrane. The peptide-binding groove is formed of the heterodimer of α1 and β1.

The human leukocyte antigen (HLA) system or complex is a gene complex encoding the major histocompatibility complex (MHC) proteins in humans. HLAs corresponding to MHC class I (A, B, and C) present peptides from inside the cell. HLAs corresponding to MHC class II (DP, DM, DOA, DOB, DQ, and DR) present antigens from outside of the cell to T-lymphocytes.

There are three major and three minor MHC class I genes in HLA. The major MHC class I genes are HLA-A, HLA-B and HLA-C. The minor genes are HLA-E, HLA-F and HLA-G. β2-microglobulin binds with major and minor gene subunits to produce a heterodimer.

There are three major and two minor MHC class II proteins encoded by the HLA. The genes of the class II combine to form heterodimeric (4) protein receptors that are typically expressed on the surface of antigen-presenting cells. The major MHC class II genes are HLA-DP, α-chain encoded by HLA-DPA1 locus and β-chain encoded by HLA-DPB1 locus; HLA-DQ, α-chain encoded by HLA-DQA1 locus and β-chain encoded by HLA-DQB1 locus; HLA-DR, α-chain encoded by HLA-DRA locus and 4 β-chains (only 3 possible per person), encoded by HLA-DRB1, DRB3, DRB4, DRB5 loci.

The other MHC class II proteins, DM and DO, are used in the internal processing of antigens, loading the antigenic peptides generated from pathogens onto the HLA molecules of antigen-presenting cell. Tumor antigens are usually recognized by CD8+ T cells via presentation through MHC class I. However, many MHC class II-restricted tumor antigens capable of stimulating CD4+ T helper (Th) cells have been identified. CD4+ T cells recognize peptides bound to MHC class II molecules on the cell surface of Antigen Presenting Cells (APC) or tumor cells through a multistep process, which is distinct from MHC class I endogenous antigen presentation and favors presentation of antigens derived from exogenous proteins. MHC-II α and β molecules form a dimer in endoplasmic reticulum (ER) followed by association with an invariant chain (Ii). Ii chain is a nonpolymorphic type II transmembrane glycoprotein and exists as several isoforms due to alternative splicing and alternative usage of start codons for translation. Association of Ii with MHC-II αβ molecules prevents antigenic peptide binding in the ER. A targeting sequence in the cytoplasmic tail of Ii is responsible for the transport of nonameric (αβIi)3 complexes from the ER to acidic endosomal/lysosomal-like structures called MHC class II compartments (MIIC). Some endogenous antigens can be directly targeted to MIIC or late endosome through lysosomal targeting sequence, while most MHC-II antigens are delivered to MIIC/late endosome through multiple pathways, including phagocytosis, macropinocytosis, endocytosis and autophagy pathways. MHC class II antigen processing and presentation require products of at least five genes (DRα, DRβ, Ii, DMα, and DMβ) in the specialized MIIC. Ii chain is cleaved in acidic endosome or MIIC, but a 20-amino acid peptide of Ii still remains associated with MHC-II molecules, called CLIP (class II-associated invariant chain peptide). DMα and DMβ promote CLIP release from MHC-II, thus making MHC-II molecules ready for antigenic peptide loading. Furthermore, DM molecules can remove low-affinity peptide from MHC-II molecules, and thus only MHC-II/high-affinity antigenic peptide complexes will be presented on the surface of a cell for T cell recognition.

As indicated above, peptides derived from the neoantigens of the invention are presented by at least one antigen presenting cell, and thereby activating an immune response. An antigen-presenting cell (APC) as used herein, is a cell that displays antigen complexed with major histocompatibility complexes (MHCs) on their surfaces. T cells may recognize these complexes using their T cell receptors (TCRs). Almost all cell types can serve as some form of APC. Professional antigen-presenting cells, including macrophages, B cells and dendritic cells, present foreign antigens to helper T cells, while other cell types can present antigens originating inside the cell to cytotoxic T cells. Antigen-presenting cells are vital for effective adaptive immune response, as the functioning of both cytotoxic and helper T cells is dependent on APCs. APC that express MHC class II molecules along with co-stimulatory molecules and pattern recognition receptors are called professional antigen-presenting cells. The non-professional APCs express MHC class I molecules. Professional APCs express both MHC class I and MHC class II molecules and can stimulate CD4+ helper T cells as well as cytotoxic T cells. Thus, in certain embodiments, peptides of the neoantigens created by the methods of the invention are presented by APC, thereby inducing an immune response.

In more specific embodiments of the methods of the invention, the immune-response may be a T cell-dependent immune response.

A “T cell” or “T lymphocyte” as used herein is characterized by the presence of a T-cell receptor (TCR) on the cell surface. It should be noted that T-cells include helper T cells (“effector T cells” or “Th cells”), cytotoxic T cells (“Tc,” “CTL” or “killer T cell”), memory T cells, and regulatory T cells as well as Natural killer T cells, Mucosal associated invariants and Gamma delta T cells.

In yet some further embodiments of the methods of the invention, the T cells may be any one of CD4+ or CD8+ T cells. Still further, peptides derived from the neoantigens created from aberrant splicing induced by the splicing modulating agent of the invention in any of the targets specified in Tables 1 and 2, may be peptides that are capable of binding MHC class I molecules and or MHC class II molecules, and therefore induce an immune response. In some specific embodiments, such peptides may comprise between about 5 to 50 amino acid residues.

In yet a further aspect, the invention relates to a method for activating an immune response against at least one target cell, in a mammalian subject. Specifically, in some embodiments, such subject is suffering from a neoplastic disorder. In some embodiments, the methods comprise the step of administering to the subject at least one splicing modulating agent comprising at least one nucleic acid sequence or of any vector, vehicle, matrix, nano- or micro-particle or composition comprising said at least one splicing modulating agent. The nucleic acid sequence of this splicing modulating agent targets at least one target nucleic acid sequence that participates directly or indirectly in at least one splicing event of at least one target gene or at least one transcript thereof. It should be further noted that in some embodiments, introduction of the at least one splicing modulating agent of the invention into the target cell induces at least one aberrant splicing event via the target nucleic acid sequence. Such aberrant splicing event results in some embodiments, in the production of the at least one neoantigen expressed by the target cell, thereby activating an immune response directed against the target cell in the administered subject.

Thus, according to this aspect, the invention provides a method for activating an immune response against at least one target cell in a mammalian subject, that may further comprise the step of providing at least one splicing modulating agent comprising at least one nucleic acid sequence. Still further, in additional aspects thereof, the invention provides at least one splicing modulating agent comprising at least one nucleic acid sequence for use in a method for activating an immune response against at least one target cell, in a mammalian subject. In more specific embodiments, the method comprises the step of administering to the subject at least one splicing modulating agent comprising at least one nucleic acid sequence or of any vector, vehicle, matrix, nano- or micro-particle or composition comprising said at least one splicing modulating agent. It should be noted that the nucleic acid sequence of this splicing modulating agent targets at least one target nucleic acid sequence that participates directly or indirectly in at least one splicing event of at least one target gene. It should be noted that the splicing modulating agents in accordance with this aspect may be any of the agents disclosed herein before in connection with other aspects of the invention.

In some embodiments, as will be discussed in detail herein after, activation of an immune response against at least one target cell by the method provided by the invention, may be specifically applicable for mammalian subjects suffering from at least one neoplastic disorder. However, it should be appreciated that in some embodiments, the method of the invention may be also applicable for other, non-therapeutical purpose. For example, the mammalian subject may be any mammal used in agriculture or for research purposes. In some specific embodiments, the method of the invention may be used for creating an animal model (e.g., any mammalian model, for example, any rodent model such as a mouse model) for autoimmune disease by triggering an immune response against healthy organs in the rodent, specifically, mouse.

In some embodiments, the splicing modulating agent/s used by the methods of the invention comprise at least one of the following agents. One option for such agent (a), may be at least one oligonucleotide comprising a nucleic acid sequence complementary to at least part of the target nucleic acid sequence. Another option for such agent (b), is at least one nucleic acid sequence comprising at least one gRNA that targets at least one protospacer within the target nucleic acid sequence. The agent used by the methods of the invention may be any nucleic acid sequence encoding such gRNA. It should be noted that this gRNA guides at least one PEN to the target nucleic acid sequence in said target gene.

In some embodiments, the methods of the invention may further comprise an additional step of administering to the subject an effective amount of at least one peptide derived from the neoantigen, or of any derivative, enantiomer, fusion protein, conjugate or polyvalent dendrimer thereof. In some embodiments, any longer peptides that comprise at least one of these peptides, or any modified or variant versions thereof, is also encompassed by the invention, as will be specified herein after in more detail. It should be noted that in some embodiments this peptide/s is administered prior to, after and/or simultaneously to administration of the splicing modulating agent. Still further, peptides derived from the neoantigens created from aberrant splicing induced by the splicing modulating agent of the invention in any of the targets specified in Tables 1 and 2, may be peptides that are capable of binding MHC class I molecules and or MHC class II molecules. In some specific embodiments, such peptides may comprise between about 5 to 50 amino acid residues, specifically, 5, 6, 7, 8, 9, 10, 11, 12, 13, 14, 15, 16, 17, 18, 19, 20, 21, 22, 23, 24, 25, 26, 27, 28, 29, 30, 31, 32, 3, 34, 35, 36, 37, 38, 39, 40, 41, 42, 43, 44, 45, 46, 47, 48, 49, 50 or more amino acid residues. In yet some further specific embodiments, the peptides derived from the neoantigens created by the invention may comprise between about 8 to about 22 amino acid residues. In more specific embodiments, when the aberrant splicing is induced in human target genes, the peptide derived from the neoantigen may bind HLA-I and/or HLA-II molecules. More specifically, peptides comprising between about 8 to about 15 amino acid residues may bind HLA-I molecules, and peptides comprising between about 8 to about 22 amino acid residues may bind HLA-II molecules.

In yet some further embodiments, the splicing modulating agent and the at least one peptide derived from the neoantigen produced (or expected to be produced) by the aberrant splicing induced may be administered either together, simultaneously, or alternatively, administered sequentially in either order. In some embodiments, as also demonstrated by Examples 4, 8, and 13 herein after, the peptide may be administered to the treated subject prior to the administration of the splicing modulating agent/s of the invention to the treated subject. In yet some further embodiments, the peptide may be administered together with the splicing modulating agent of the invention. In some other embodiments, the peptides may be administered after the administration of the splicing modulating agent of the invention. Still further, in some embodiments, the peptides derived from the neoantigen may be administered before, together with and after the administration of the splicing modulating agent of the invention. In other alternative embodiments, the splicing modulating agent of the invention may be administered before, together with and after the administration of the peptides of the invention. This combined administration is also applicable for other aspects of the invention.

In some embodiments, the at least one target nucleic acid sequence that participates directly or indirectly in at least one splicing event comprises at least one of, a splicing junction, a splice donor site, a splice acceptor site, an exonic splicing enhancer, splicing silencer, an intronic splicing enhancer and an intronic splicing silencer of the target gene or at least one transcript thereof.

In yet some further embodiments, the at least one target nucleic acid sequence that participates directly or indirectly in at least one splicing event are comprised within at least one coding transcript characterized by at least one of: (i) the coding transcript/s comprise at least three exons; (ii) the at least one of said exons is of a length not divisible by three; and (iii) the coding transcripts comprise at least one intron.

In yet some further embodiments, the target sequence is a target that aberrant splicing mediated by such sequence leads to a frame shift that creates a neoantigen that does not exist in the human proteome. Still further, in some embodiments, peptides derived from such neoantigen, may be at least one of 8-mer, 9-mer, 10-mer, 11-mer, 12-mer, 13-mer, 14-mer, 15-mer, 16-mer, 17-mer, 18-mer, 19-mer, 20-mer, 21-mer, 22-mer peptides, specifically, 8-14-mer peptides, and in some embodiments, 9-mer peptides (peptides comprising nine amino acid residues), display high affinity to HLA molecules and are therefore immunogenic. Moreover, such peptides do not exist in a mammalian proteome, specifically, the human proteome.

In some specific embodiments, the target nucleic acid sequence that participates directly or indirectly in at least one splicing event may be comprised within least one of the exons described above, specifically, one of the at least three exons, more specifically, an exon that is not the first exon in the transcript. In yet some further embodiments, the target nucleic acid sequence that participates directly or indirectly in at least one splicing event may be comprised within least one of the at least three exons described above, specifically an exon that is not the last exon in the transcript. In yet some further embodiments, the target nucleic acid sequence that participates directly or indirectly in at least one splicing event may be comprised within an exon that is before the last exon in the transcript. In yet some further embodiments, such exon is in a length not divisible by three. Still further, in some embodiments, in case of induction of exon skipping by the aberrant splicing event induced by the method of the invention, the resulting spliced transcript, specifically the mRNA is in a length not divisible by three (3), thereby enabling and forcing in some embodiments, a frame shift.

In some embodiments, the target nucleic acid sequence is comprised within an exon, or within at least one intron located upstream or downstream to the exon, or within at least one splicing junction flanking the exon. For example, the target nucleic acid sequence may be located within a 5′ splice junction, that is the intron/exon splice junction located 5′ or upstream to the indicated exon. In other embodiments, the target sequence may be located within the 3′ splice junction, or in other words, in the exon/intron junction located 3′ or downstream to the indicated exon. It should be understood that in certain embodiments the exon as specified herein is not the first or the last exon in the target transcript. It should be appreciated that all the target specific locations described herein before in connection with other aspects of the invention are also applicable for the present aspect.

In yet some alternative embodiments, in case of induction of intron retention by the aberrant splicing event induced by the method of the invention, the least one nucleic acid sequence that participates directly or indirectly in at least one splicing event may be comprised within at least one splicing junction within the target transcript.

In some embodiments the oligonucleotide used by the method of the invention may comprise at least 10 or more contiguous nucleobases complementary to at least part of said at least one nucleic acid sequence that participates directly or indirectly in at least one splicing event, specifically, at least 10 or more, at least 11 or more, at least 12 or more, at least 13 or more, at least 14 or more, at least 15 or more, at least 16 or more, at least 17 or more, at least 18 or more, at least 19 or more, at least 20 or more, at least 21 or more, at least 22 or more, at least 23 or more, at least 24 or more, at least 25 or more, at least 26 or more, at least 27 or more, at least 28 or more, at least 29 or more, at least 30 or more contiguous nucleobases complementary to the target sequence. In some specific embodiments, the oligonucleotide used by the method of the invention comprise at least 15 or more contiguous nucleobases complementary to the target sequence. In some specific embodiments, the oligonucleotide used by the method of the invention may comprise at least fifteen contiguous nucleobases complementary to at least part of said at least one nucleic acid sequence that participates directly or indirectly in at least one splicing event. It should be understood that any oligonucleotide disclosed in connection with other aspects of the invention is also applicable for the present aspect as well.

Still further, in some embodiments the splicing modulating agent is at least one guide RNA that guides at least one PEN to the target nucleic acid sequence as specified herein. In some embodiments, the PEN comprises at least one CRISPR/cas protein. Thus, according to some embodiments, the splicing modulating agent used by the methods of the invention comprises: first (a), at least one nucleic acid sequence comprising at least one gRNA, or any nucleic acid sequence encoding the gRNA; or any kit, composition, vector or vehicle comprising the gRNA or nucleic acid sequence encoding the gRNA. Optionally, the splicing modulating agent may further comprise (b), at least one CRISPR/cas protein, or any nucleic acid molecule encoding the Cas protein, or any kit, composition, vector or vehicle comprising the CRISPR/cas protein or nucleic acid sequence encoding the CRISPR/cas protein. It should be appreciated that any CRISPR/Cas system disclosed in connection with other aspects of the invention is also applicable for the present aspect as well.

In yet some specific embodiments, the invention provides methods for inducing the production of at least one neoantigen in at least one target cell. In yet some further embodiments, the target cell of the methods of the invention may be a cell of a subject suffering from at least one neoplastic disorder. As indicated above, in some embodiments, the method of the invention is particularly suitable for activating an immune response against at least one target cell, in a mammalian subject suffering from a neoplastic disorder. In more specific embodiments, such neoplastic disorder is a cancer. It should be understood that any of the cancer disorders described herein before in connection with other aspects of the invention is also applicable or the present aspect as well.

In some embodiments, the target gene, and specifically at least one transcript thereof, is selected from, and therefore may be any one of the group of genes disclosed by Table 1 that is presented herein after by Example 14. This table disclose the target genes of the invention and specifically, the target transcripts that comprise the target nucleic acid sequence that participates directly or indirectly in at least one splicing event of at least one target gene. This table further indicates the particular cancers that overexpress each of the target gene/s and/or at least one transcript thereof. This target nucleic acid sequences is targeted by the splicing modulators of the invention, specifically, the AON/s and gRNAs disclosed by the invention. In yet some further embodiments, the splicing modulating agents used by the methods of the invention are directed against a target sequence located within an exon, within at least one intron located upstream or downstream to such exon, or within at least one splicing junction flanking at least one of the exons selected from the group of exons disclosed by Table 2, presented herein after by Example 14. More specifically, Table 2 specifies the particular transcripts of the target gene and moreover, the specific coordinates (e.g., start and end nucleotides in the specified chromosome) for each of the exons in each target transcript that comprise the target sequence that participates directly or indirectly in at least one splicing event, or exons that comprise the target sequence in at least one of the flanking junctions (e.g., intron/exon or exon/intron junction), or within at least one intron located upstream or downstream to the exons specified in in Table 2.

In more specific embodiments, the cancer may be melanoma. Thus, in some embodiments, the invention provides methods for inducing the production of at least one neoantigen to be expressed by at least one target cell of a subject suffering from melanoma. In yet some further specific embodiment, the target cell is a cancerous cell.

Still further, in some embodiments, the method of the invention induces the production of at least one neoantigen to be expressed by at least target cell using the splicing modulating agent comprising at least one nucleic acid sequence, specifically, the AON/s and gRNAs described herein before, that lead to aberrant splicing event in a target gene. In more specific embodiments, such target gene may be a gene differentially expressed in at least one cancer cell and/or at least one cancerous tissue. It should be understood that any of the genes overexpressed in cancer tissues described herein before in connection with other aspects of the invention is also applicable or the present aspect as well.

In some particular embodiments, the target gene may be the tyrosinase (TYR) gene.

In yet some further particular and non-limiting embodiments, the TYR gene, may be a specifically relevant target gene in cancer cells of a subject suffering from melanoma.

In some embodiments, for inducing aberrant splicing in the TYR gene, targeting nucleic acid sequences that participate or affect directly or indirectly the splicing of exon 4 thereof, may be used by the method of the invention.

In certain embodiments, the splicing modulating agent used by the methods of the invention may comprise at least one AON. Thus, in some specific embodiments, the nucleic acid sequences that participate or affect splicing of exon 4 is targeted using AONs. In more specific embodiments, the AONs that target the human TYR exon 4 may comprise the nucleic acids sequence as denoted by any one of SEQ ID NOs. 28 to 47, or any variants, homologs and derivatives thereof.

In yet some further embodiments, the mouse TYR gene is targeted by the methods of the invention.

In some specific embodiments, targeting AONs may comprise the AON designated herein as TYR oligo 9, specifically an oligonucleotide targeting a sequence comprising the nucleic acids sequence as denoted by SEQ ID NO. 6. In more specific embodiment, the oligo 9 AON, may comprise the nucleic acid sequence as denoted by SEQ ID NO. 4 or any variants, homologs or derivatives thereof. In yet some further embodiments, targeting AONs used by the methods of the invention may comprise the AON designated herein as TYR oligo 13, specifically an oligonucleotide targeting a sequence comprising the nucleic acids sequence as denoted by SEQ ID NO. 7. In more specific embodiment, the oligo 13 AON may comprise the nucleic acid sequence as denoted by SEQ ID NO. 5 or any variants, homologs or derivatives thereof.

In yet some further embodiments, the splicing modulating agent/s used by the methods of the invention may comprise at least one gRNA, specifically, using the CRISPR-Cas system specified above. Thus, in some specific embodiments, the nucleic acid sequences that participate or affect splicing of exon 4 is targeted using at least one gRNA. In more specific embodiments, gRNAs that target the exon 4 of the human TYR transcript may comprise the nucleic acid sequence as denoted by any one of SEQ ID NOs. 20, 21, 22, 23, 34, 25, 26, 27. It should be appreciated that each possibility disclosed herein represent a separate embodiment of the invention. In yet some further embodiments, the gRNAs that target the exon 4 of the mouse TYR transcript may comprise the nucleic acid sequence as denoted by any one of SEQ ID NOs. 16 and 17 (targeting the 5′ splice site) and SEQ ID NOs. 18 and 19 (targeting the 3′ splice site).

In more specific embodiments the target gene that comprise the target nucleic acid sequence that participates directly or indirectly in at least one splicing event, is overexpressed in breast cancer.

In some particular embodiments, the target gene may be the heterogeneous nuclear ribonucleoprotein A/B (HNRNPAB) gene. Thus, in some embodiments the target transcript is of the HNRNPAB gene. In yet some further embodiments, the cancer is a breast cancer.

As indicated above, in yet some further particular and non-limiting embodiments, the HNRNPAB gene, may be a specifically relevant target gene in cancer cells of a subject suffering from breast cancer.

In some embodiments, for inducing aberrant splicing in the HNRNPAB gene, targeting nucleic acid sequences that participate or affect splicing of exon 6 may be used by the method of the invention.

In certain embodiments, the splicing modulating agent used by the methods of the invention may comprise at least one AON. Thus, in some specific embodiments, the nucleic acid sequences that participate or affect splicing of exon 6 is targeted using AONs. In more specific embodiments, the AONs that target the human hnRNPAB exon 6 may comprise the nucleic acids sequence as denoted by any one of SEQ ID NOs. 67, 68, 69, 70, 71, 72, 73, 74, 75, 76, 77, 78, 79, 80, 81, 82, 83, 84, 85, 86, or any variants, homologs and derivatives thereof. It should be appreciated that each possibility disclosed herein represent a separate embodiment of the invention.

In yet some further embodiments, the splicing modulating agent/s used by the methods of the invention may comprise at least one gRNA, specifically, using the CRISPR-Cas system specified above. Thus, in some specific embodiments, the nucleic acid sequences that participate or affect splicing of exon 6 of hnRNPAB are targeted using at least one gRNA. In more specific embodiments, gRNAs that target the exon 6 of the human hnRNPAB transcript may comprise the nucleic acid sequence as denoted by any one of SEQ ID NOs. 59, 60, 61, 62, 63, 64, 65, 66. It should be appreciated that each possibility disclosed herein represent a separate embodiment of the invention.

In yet some further embodiments, the gRNAs that target the exon 6 of the mouse hnRNPAB transcript may comprise the nucleic acid sequence as denoted by any one of SEQ ID NOs. 57 and 58 (targeting the 3′ splice site).

Still further, in some embodiments, the methods of the invention induce the production and thereby the expression of the newly created neoantigen by at least one target cell of the administered subject. In some embodiments, the expression of the neoantigen by the target cell, leads to activation of an immune-response directed against this target cell. Still further, in some embodiments, the presentation of peptides of the newly produced or created neoantigen by at least one APC, in the administered subject, in the context of at least one of MHC Class I and MHC Class II, results in the activation of an immune-response against the target cell.

In more specific embodiments of the methods of the invention, the immune-response induced in the administered subject may be a T cell-dependent immune response.

In yet some further embodiments of the methods of the invention, the T cells may be any one of CD4+ or CD8+ T cells.

It should be understood that as discussed above, the invention provides methods for inducing an immune response in a subject suffering from cancer, and therefore may be combined with other therapeutic agents. In some embodiments, such additional therapeutic agents may be combined either with at least one of any of the splicing modulating agents disclosed by the invention or with any combinations of the splicing modulating agents with at least one peptide derived from the neoantigens of the invention, created (or predicted) by the aberrant splicing induced by the methods of the invention. In some further embodiments, the additional therapeutic agents may be combined with at least one of the peptides of the invention. As such, the present invention further provides combined therapy. Thus, in some embodiments, the treated subject may be a subject treated with a therapeutic agent. In yet some further embodiments the subject may be further treated before, after or simultaneously, with at least one additional therapeutic agent, specifically, at least one immuno-modulatory agent.

In some specific embodiments, immuno-modulatory agent may be at least one immune checkpoint inhibitor. More specifically, immune checkpoint molecules are co-stimulatory and co-inhibitory molecules that act in coordination to modulate the immune response of autoreactive T cells. Immune checkpoint molecules, like CTLA-4, TIM-3, PD-1, are negative regulators of immune responses. Additional immune checkpoint targets include the lymphocyte activation gene-3 (LAG-3), T cell immunoglobulin and mucin-domain containing-3 (TIM-3), T cell immunoglobulin and ITIM domain (TIGIT), V-domain Ig suppressor of T cell activation (VISTA), and so on. In some embodiments, a checkpoint inhibitor applicable in the method of the invention may be an antibody targeted against an immune checkpoint molecule selected from the group consisting of human programmed cell death protein 1 (PD-1), PD-L1 and PD-L2, carcinoembryonic antigen-related cell adhesion molecule 1 (CEACAM1), CTLA-4, lymphocyte activation gene 3 (LAG3), CD137, 0X40 (also referred to as CD134), killer cell immunoglobulin-like receptors (KIR), TIGIT, VISTA and any combination thereof. Each possibility represents a separate embodiment of the invention. Still further, in some embodiments, a few examples of antibodies used in the treatment of cancer that may be applicable in the present invention include, but are not limited to monoclonal antibodies such as Ipilimumab (UNII: 6T8C155666, Yervoy), that is a check point inhibitor, specifically, a monoclonal antibody that works to activate the immune system by targeting CTLA-4, Trastuzumab (UNII: P188ANX8CK, formerly ticilimumab, CP-675,206) is a fully human monoclonal antibody against CTLA-4, ibritumomab tiuxetan (UNII: 4Q52C550XK), lambrolizumab (formerly MK-3475, Pembrolizumab, Keytruda® UNII: DPT003T46P), that is a check point inhibitor, specifically, a humanized antibody that targets programmed cell death (PD-1), Nivolumab (Opdivo® UNII: 31Y063LBSN) is an Fab fragment of an antibody that binds the extracellular domain of PD-1, Atezolizumab (trade name Tecentriq) is a fully humanized, engineered monoclonal antibody of IgG1 isotype against the protein programmed cell death-ligand 1 (PD-L1), Avelumab (trade name Bavencio) is a fully human monoclonal antibody that targets PD-L1, Durvalumab (Imfinzi) is a human immunoglobulin G1 kappa (IgG1κ) monoclonal antibody that blocks the interaction of PD-L1 with the PD-1 and CD80 (B7.1) molecules and Tremelimumab (formerly ticilimumab; UNII: QEN1X95CIX) that is a check point inhibitor and ado-trastuzumab emtansine (UNII: SE2KH7T06F). Libtayo (cemiplimab-rwlc) that targets the PD-1 cellular pathway, and any combinations thereof.

In yet some further specific embodiments, such immune-checkpoint inhibitor may be a checkpoint inhibitor directed against at least one of Cytotoxic T-lymphocyte protein 4 (CTLA-4) and programmed cell death protein 1 (PD-1). In some particular embodiments, the splicing modulating agent comprising at least one nucleic acid sequence, specifically, the AON/s and gRNAs disclosed herein may be used in combined therapy with anti-CTLA treatment, specifically, ipilimumab (Yervoy®). In some particular embodiments, the splicing modulating agent comprising at least one nucleic acid sequence, specifically, the AON/s and gRNAs disclosed herein may be used in combined therapy with anti-PD-1 treatment, specifically, pembrolizumab (Keytruda®) or Nivolumab (Opdivo®). In yet some further embodiments, the splicing modulating agent disclosed herein may be used in combined therapy with PD-L1 inhibitor, specifically, atezolizumab.

In yet another aspect, the invention provides a method for treating, inhibiting, preventing, ameliorating or delaying the onset of at least one neoplastic disorder in a subject. In some embodiments, the method comprising the step of administering to the treated subject at least one splicing modulating agent comprising at least one nucleic acid sequence or of any vector, vehicle, matrix, nano- or micro-particle or composition comprising the at least one splicing modulating agent of the invention. It should be noted that the at least one nucleic acid sequence of these agents target at least one target nucleic acid sequence that participates, or is involved with, directly or indirectly in at least one splicing event of at least one target gene. It should be further noted that in some embodiments, introduction of the at least one splicing modulating agent of the invention into the target cell induces at least one aberrant splicing event via the nucleic acid sequence. Such aberrant splicing event results in some embodiments, in the production of the at least one neoantigen expressed by the target cell, thereby activating an immune response directed against the target cell in the treated subject. In some embodiments, the method may further comprise the steps of providing at least one splicing modulating agent comprising at least one nucleic acid sequence.

In some embodiments, the splicing modulating agent used by the methods of the invention comprises at least one of the following agents. One option for such agent (a), may be at least one oligonucleotide comprising a nucleic acid sequence complementary to at least part of the target nucleic acid sequence. Another option for such agent (b), is at least one nucleic acid sequence comprising at least one gRNA that targets at least one protospacer within the target nucleic acid sequence. The agent used by the methods of the invention may be any nucleic acid sequence encoding such gRNA. It should be noted that this gRNA guides at least one PEN to the target nucleic acid sequence in said target gene.

In some embodiments, the therapeutic methods of the invention may further comprise an additional step of administering to the treated subject an effective amount of at least one peptide derived from the neoantigen that is expected to be produced from the aberrant splicing event (e.g., exon skipping and/or intron retention) induced by the splicing modulating agent/s used by the methods of the invention, or of any derivative, enantiomer, fusion protein, conjugate or polyvalent dendrimer of such peptide. It should be noted that in some embodiments, the peptide is administered to the treated subject prior to, after and/or simultaneously to administration of the splicing modulating agent, as discussed herein before.

In some embodiments, the splicing modulating agents used by the methods of the invention, comprise at least one nucleic acid sequence that target, or is directed at, at least one target nucleic acid sequence that participates directly or indirectly in at least one splicing event. In some embodiments, such target nucleic acid sequence comprises at least one of, a splicing junction, a splice donor site, a splice acceptor site, an exonic splicing enhancer, splicing silencer, an intronic splicing enhancer and an intronic splicing silencer of the target gene.

In yet some further embodiments, the at least one target nucleic acid sequence that participates directly or indirectly in at least one splicing event and comprised within at least one coding transcript may be characterized by at least one of: (i) the coding transcript/s may comprise at least three exons; (ii) at least one of these exons is of a length not divisible by three; and (iii) the coding transcripts comprise at least one intron.

In yet some further embodiments, the target sequence is a target that aberrant splicing mediated by such sequence leads to a frame shift that creates a neoantigen that does not exist in the human proteome. Still further, in some embodiments, peptides derived from such neoantigen, specifically 9-mer peptides (peptides comprising nine amino acid residues), display high affinity to HLA molecules and are therefore immunogenic. Moreover, such peptides do not exist in a mammalian proteome, specifically, the human proteome.

In some embodiments, the target nucleic acid sequence is comprised within an exon, or within at least one intron located upstream or downstream to said exon, or within at least one splicing junction flanking said exon. For example, the target nucleic acid sequence may be located within a 5′ splice junction, that is the intron/exon splice junction located 5′ or upstream to the indicated exon. In other embodiments, the target sequence may be located within the 3′ splice junction, or in other words, in the exon/intron junction located 3′ or downstream to the indicated exon. It should be understood that in certain embodiments the exon as specified herein is not the first or the last exon in the target transcript. Thus, the target sequence for an aberrant splicing event may include any sequence within an exon, or within at least one intron located upstream or downstream to said exon, or within at least one splicing junction flanking said exon. More specifically, any target sequence comprised within a sequence flanking the 5′ end of an exon in a distance from about 1 to about 500 base pairs upstream of the indicated exon or alternatively or additionally, any target sequence comprised within a sequence flanking the 3′ end of an exon in a distance from about 1 to about 500 base pairs downstream of the indicated exon in a preprocessed target transcript, specifically about 1, 5, 10, 15, 20, 25, 30, 35, 40, 45, 50, 55, 60, 65, 70, 75, 80, 85, 90, 95, 100, 150, 200, 250, 300, 350, 400, 450 or 500 base pairs downstream or upstream of the indicated exon.

In some specific embodiments, the target nucleic acid sequence that participates directly or indirectly in at least one splicing event may be comprised within least one of the at least three exons described above, specifically an exon that is not the first exon in the transcript. In yet some further embodiments, the target nucleic acid sequence that participates directly or indirectly in at least one splicing event may be comprised within least one of the at least three exons described above, specifically an exon that is not the last exon in the transcript. In yet some further embodiments, the target nucleic acid sequence that participates directly or indirectly in at least one splicing event may be comprised within an exon that is before the last exon in the transcript. In yet some further embodiments, such exon is in a length not divisible by three. Still further, in some embodiments, in case of induction of exon skipping by the aberrant splicing event induced by the method of the invention, the resulting product is in a length not divisible by three (3), thereby enabling frame shift.

In yet some alternative embodiments, in case of induction of intron retention by the aberrant splicing event induced by the methods of the invention, the at least one target nucleic acid sequence that participates directly or indirectly in at least one splicing event may be comprised within at least one splicing junction within the target transcript.

In yet some further embodiments, splicing modulating agent/s applicable for the present aspect of the invention comprises at least one oligonucleotide. Such oligonucleotide is an ASO between about 10 to about 25 or more contiguous nucleobases complementary to at least part of the at least one target nucleic acid sequence that participates directly or indirectly in at least one splicing event, specifically, at least 10, 11, 12, 13, 14, 15, 16, 17, 18, 19, 20, 21, 22, 23, 24 25 nucleobases or more. Specifically, at least 15 contiguous nucleobases complementary to at least part of the at least one target nucleic acid sequence. In more specific embodiments the ASO/s (AON/s) of the invention comprise at least fifteen contiguous nucleobases complementary to at least part of said at least one nucleic acid sequence that participates directly or indirectly in at least one splicing event.

Still further, in some embodiments the splicing modulating agent is at least one guide RNA that guides at least one PEN to the target nucleic acid sequence as specified herein. In some embodiments, the PEN comprises at least one CRISPR/cas protein. Thus, according to some embodiments, the splicing modulating agent used by the methods of the invention comprises: first (a), at least one nucleic acid sequence comprising at least one gRNA, or any nucleic acid sequence encoding the gRNA; or any kit, composition, vector or vehicle comprising the gRNA or nucleic acid sequence encoding the gRNA. Optionally, the splicing modulating agent may further comprise (b), at least one CRISPR/cas protein, or any nucleic acid molecule encoding the Cas protein, or any kit, composition, vector or vehicle comprising the CRISPR/cas protein or nucleic acid sequence encoding the CRISPR/cas protein.

In yet some specific embodiments, the invention provides therapeutic methods that induce the production of at least one neoantigen in at least one target cell of the treated subject. Thus, in some specific embodiments, the treated subject is suffering from at least one neoplastic disorder.

In more specific embodiments, such neoplastic disorder is a cancer. Thus, in some embodiments of the invention, the neoplastic disorder is cancer, and the target cell in the treated subject is a cancerous cell. It should be noted that in some embodiments, the therapeutic methods of the invention are specifically applicable for any of the cancer types disclosed by the invention in connection with other aspects of the invention. In yet some further embodiments, the therapeutic methods of the invention may be specifically applicable for treating at least one of, melanoma, breast cancer, LAML, BLCA, LGG, BRCA, CESC, COAD, ESCA, GBM, HNSC, KICH, KIRC, KIRP, LIHC, LUAD, LUSC, DLBC, OV, PAAD, PRAD, READ, SKCM, STAD, TGCT, THYM, THCA, UCS and UCEC.

Still further, in some embodiments, the therapeutic methods of the invention induce the production of at least one neoantigen by target cells of the treated subject using splicing modulating agent comprising at least one nucleic acid sequence, specifically, the AON/s and gRNAs. These specific modulators lead to aberrant splicing event in a target gene. In more specific embodiments, such target gene or at least one transcript thereof, may be a gene differentially expressed in at least one cancer cell and/or at least one cancerous tissue of the treated subject. In yet some further embodiments, the target gene may be a gene highly expressed in cancer cells, specifically, cancerous cells of the treated subject.

In some embodiments, the target gene targeted by the therapeutic methods of the invention is selected from, and therefore may be any one of the group of genes disclosed by Table 1 that is presented herein after by Example 14. This table disclose the target genes of the invention and specifically, the target transcripts that comprise the target nucleic acid sequence that participates directly or indirectly in at least one splicing event of at least one target gene. This table further indicates the particular cancer that overexpresses the target gene and/or at least one transcript thereof. The target nucleic acid sequences are targeted by the splicing modulators of the invention, specifically, the AON/s and gRNAs disclosed by the invention.

In some specific embodiments, the splicing modulating agents used by the therapeutic methods of the invention are directed against a target sequence located within an exon, within at least one intron located upstream or downstream to such exon, or within at least one splicing junction flanking at least one of the exons selected from the group of exons disclosed by Table 2, presented herein after by Example 14. More specifically, Table 2 specifies the particular transcripts of the target gene and moreover, the specific coordinates (e.g., start and end nucleotides in the specified chromosome) for each of the exons in each target transcript that comprise the target sequence that participates directly or indirectly in at least one splicing event, or exons that comprise the target sequence in at least one of the flanking junctions (e.g., intron/exon or exon/intron junction), or within at least one intron located upstream or downstream to the exons specified in Table 2.

Still further, in some embodiments, the method of the invention induces the production of at least one neoantigen by target cells of the treated subject using splicing modulating agent comprising at least one nucleic acid sequence, specifically, the AON/s and gRNAs that lead to aberrant splicing event in a target gene or transcript in these cells. In more specific embodiments, such target gene may be a gene or at least one transcript thereof differentially expressed in at least one cancer cell and/or at least one cancerous tissue. In more specific embodiments, therapeutic method of the invention may be applicable for melanoma. Thus, in some embodiments, the invention provides therapeutic and prophylactic methods for subjects suffering from melanoma. It should be understood that the therapeutic methods of the invention are applicable for any stage, type or grade of melanoma. In yet some further specific embodiments of the methods of the invention, the target cell of the treated subject is a cancerous cell.

In yet some further embodiments, the target gene may be a gene highly expressed in cancer cells.

In some particular embodiments, the target gene may be the tyrosinase (TYR) gene.

In yet some further particular and non-limiting embodiments, the TYR gene, may be a specifically relevant target gene in cancer cells of a subject suffering from melanoma.

In some embodiments, for inducing aberrant splicing in the TYR gene, targeting nucleic acid sequences that participate or affect splicing of exon 4 may be administered by the method of the invention. In some specific embodiments, exon 4 of the human TYR transcript comprises the nucleic acid sequence as denoted by SEQ ID NO. 1. In yet some further embodiments, exon 4 of the mouse TYR transcript comprises the nucleic acid sequence as denoted by SEQ ID NO. 8.

In certain embodiments, the splicing modulating agent used by the methods of the invention may comprise at least one AON. Thus, in some specific embodiments, the nucleic acid sequences that participate or affect splicing of exon 4 is targeted using AONs. In more specific embodiments, the AONs that target the human TYR exon 4 may comprise the nucleic acids sequence as denoted by any one of SEQ ID NOs. 28, 29, 30, 31, 32, 33, 34, 35, 36, 37, 38, 39, 40, 41, 42, 43, 44, 45, 46, 47, or any variants, homologs and derivatives thereof.

In yet some further embodiments, the mouse TYR gene is targeted by the methods of the invention. More specifically, in some specific embodiments, targeting AONs may comprise the AON designated herein as TYR oligo 9, specifically an oligonucleotide targeting a sequence comprising the nucleic acids sequence as denoted by SEQ ID NO. 6. In more specific embodiment, the oligo 9 AON, may comprise the nucleic acid sequence as denoted by SEQ ID NO. 4. In yet some further embodiments, the targeting AONs administered by the methods of the invention may comprise the AON designated herein as TYR oligo 13, specifically an oligonucleotide targeting a sequence comprising the nucleic acids sequence as denoted by SEQ ID NO. 7. In more specific embodiment, the oligo 13 AON may comprise the nucleic acid sequence as denoted by SEQ ID NO. 5.

In yet some further embodiments, the splicing modulating agent used by the methods of the invention may comprise at least one gRNA, specifically, using the CRISPR-Cas system specified above. Thus, in some specific embodiments, the nucleic acid sequences that participate or affect splicing of exon 4 is targeted using at least one gRNA. In more specific embodiments, gRNAs that target the exon 4 of the human TYR transcript may comprise the nucleic acid sequence as denoted by any one of SEQ ID NOs. 20 to 27.

In yet some further embodiments, the gRNAs that target the exon 4 of the mouse TYR transcript may comprise the nucleic acid sequence as denoted by any one of SEQ ID NOs. 16 and 17 (targeting the 5′ splice site) and SEQ ID NOs. 18 and 19 (targeting the 3′ splice site). CLAIM 43 In more specific embodiments the target gene that comprise the target nucleic acid sequence that participates directly or indirectly in at least one splicing event, is overexpressed in breast cancer. In some particular embodiments, the target gene may be the HNRNPAB gene. Thus, in some embodiments the target gene is HNRNPAB, and the methods of the invention may be applicable for breast cancer. As used herein, the term “breast cancer” refers to a cancer that develops from breast tissue. Development of breast cancer is often associated with a lump in the breast, a change in breast shape, dimpling of the skin, fluid coming from the nipple, or a red scaly patch of skin. Breast cancer classification divides breast cancer into categories according to different schemes, each based on different criteria and serving a different purpose. The major categories are the histopathological type, the grade of the tumor, the stage of the tumor, and the expression of proteins and genes. It should be understood that the therapeutic methods of the invention are applicable for any stage, type or grade of breast cancer. As indicated above, in yet some further particular and non-limiting embodiments, the HNRNPAB gene, may be a specifically relevant target gene in cancer cells of a subject suffering from breast cancer.

In some embodiments, for inducing aberrant splicing in the HNRNPAB gene, targeting nucleic acid sequences that participate or affect splicing of exon 6 may be used by the method of the invention.

In certain embodiments, the splicing modulating agent used by the methods of the invention may comprise at least one AON. Thus, in some specific embodiments, the nucleic acid sequences that participate or affect splicing of exon 6 is targeted using AONs. In more specific embodiments, the AONs that target the human hnRNPAB exon 6 may comprise the nucleic acids sequence as denoted by any one of SEQ ID NOs. 67 to 86, or any variants, homologs and derivatives thereof. In yet some further embodiments, the splicing modulating agent used by the methods of the invention may comprise at least one gRNA, specifically, using the CRISPR-Cas system specified above. Thus, in some specific embodiments, the nucleic acid sequences that participate or affect splicing of exon 6 of hnRNPAB is targeted using at least one gRNA. In more specific embodiments, gRNAs that target the exon 6 of the human hnRNPAB transcript may comprise the nucleic acid sequence as denoted by any one of SEQ ID NOs. 59 to 66. In yet some further embodiments, the gRNAs that target the exon 6 of the mouse hnRNPAB transcript may comprise the nucleic acid sequence as denoted by any one of SEQ ID NOs. 57 and 58 (targeting the 3′ splice site).

Still further, in some embodiments, the therapeutic method of the invention induces the production and thereby the expression of the newly created neoantigen by the target cell in the treated subject.

In some embodiments, the expression of the neoantigen leads to activation of an immune-response directed against this target cell.

In yet some further embodiments, the presentation of peptides of the newly produced or created neoantigen by at least one APC in the treated subject in the context of at least one of (MHC Class I and MHC Class II, results in the activation of an immune-response against the target cell, in the treated subject. In more specific embodiments of the methods of the invention, the immune-response may be a T cell-dependent immune response. In yet some further embodiments of the methods of the invention, the T cells may be any one of CD4+ or CD8+ T cells.

It should be understood that as discussed above, the invention provides therapeutic methods for treating cancer in a subject. It should be however understood that the methods of the invention that result in activation of the immune system in the treated subject may further suggest combined therapy with other therapeutic agents used in cancer. As such, the present invention further provides combined therapy. Thus, in some embodiments, the treated subject may be a subject treated with at least one additional therapeutic agent. In yet some further embodiments the subject may be further treated with at least one immuno-modulatory agent. In some embodiments, such additional therapeutic agents may be combined either with at least one of any of the splicing modulating agents disclosed by the invention or with any combinations of the splicing modulating agents with at least one peptide derived from the neoantigens of the invention, created (or predicted) by the aberrant splicing induced by the methods of the invention. In some further embodiments, the additional therapeutic agents may be combined with at least one of the peptides of the invention. In some specific embodiments immuno-modulatory agent may be at least one immune checkpoint inhibitor.

In yet some further specific embodiments, such immune-checkpoint inhibitor may be a checkpoint inhibitor directed against at least one of human CTLA-4, PD-1, PD-L1 and PD-L2, CEACAM1, LAG3, CD137, 0X40, KIR, TIGIT, VISTA and any combination thereof. Specifically, at least one of CTLA-4 and PD-1.

It should be noted that the methods provided by the therapeutic methods discussed herein involve the administration of at least one splicing modulating agent (and optionally, additional peptides and other therapeutic agents), to the treated subject. Such agents induce in vivo at least one aberrant splicing event in cancerous cells of the administered subject. The aberrant splicing event leads to creation of a neoantigen that triggers an immune response in the treated subject, that is specifically directed against the cancer cells, and optionally other cancer cells in the cancerous tissue, as will be further discussed herein after. It should be however appreciated that in some embodiments thereof, the invention further encompasses the option of ex vivo treatment. More specifically, splicing event may be induced using the modulators of the invention ex vivo or in vitro, in cancer cells obtained from the patient. These cancer cells express the neoantigen and can now be incubated with hematopoietic cells (e.g., NK cells and/or T cells) obtained either from the same subject (autologous source) or from an appropriate donor (allogeneic source). The activated immune cells incubated ex vivo with cancer cells that express the neoantigen are directed against any cell that express this neoantigen and therefore can be introduced back to the patient (adoptive transfer). In yet some further embodiments, the invention further contemplates option of ex vivo engineering NK and/or T cells obtained either from the subject or from an appropriate donor to express a Chimeric antigen receptor (CAR) that specifically recognize and binds the neoantigen created by the aberrant splicing event induced by the invention. Such engineered cells are introduced back to the patient. It should be understood that in some embodiments, the indicated in vivo and ex vivo therapeutic strategies may be combined, specifically, the patient can be treated with the modulating agents of the invention to induce in vivo aberrant splicing, and are also administered with the CAR-engineered T and/or NK cells and/or activated T and/or NK discussed above. These combined approach may be further combined with vaccination using peptides derived from the neoantigens of the invention.

As indicated herein, the invention provides therapeutic methods for treating the specific conditions or diseases disclosed herein before. As used herein, “disease”, “disorder”, “condition” and the like, as they relate to a subject's health, are used interchangeably and have meanings ascribed to each and all of such terms. It should be appreciated that the invention provides therapeutic methods applicable for any of the disorders disclosed above, as well as to any condition or disease associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

In some embodiments, the therapeutic methods described herein may use an effective amount of the splicing modulating agent of the invention (AON/s and gRNAs), particularly for therapeutic purposes. The terms “effective amount” or “sufficient amount” used by the methods of the invention, mean an amount necessary to achieve a selected result. More specifically, the amount of the specific modulating agent that is sufficient to induce aberrant splicing in a target transcript. Moreover, such effective amount is sufficient to induce the production of a neoantigen that induces an immune response against cancer cells of the treated subject expressing the neoantigens produced by the invention. It should be however noted that due to activation of an immune response in the treated subject, in some embodiments inflammation of the tumor tissue may occur and may lead to destruction and elimination of other cancer cells in the cancer tissue that do not express the neoantigen of the invention (the bystander effect).

The “effective treatment amount” is determined by the severity of the disease in conjunction with the preventive or therapeutic objectives, the route of administration and the patient's general condition (age, sex, weight and other considerations known to the attending physician). The AON concentration can range between 5 mg/kg and 50 mg/kg but other concentrations may apply. More specifically, in certain embodiments, the dose for systemic administration is from 0.1 mg/kg to 500 mg/kg. In certain embodiments, the dose for systemic administration is from 0.1 mg/kg to 100 mg/kg, 0.5 mg/kg to 100 mg/kg, 1 mg/kg to 100 mg/kg, 1 mg/kg to 50 mg/kg, 1 mg/kg to 25 mg/kg. In some embodiments, the dose for systemic administration is from 5 mg/kg to 50 mg/kg. In yet some further embodiments, the dose for systemic administration is from 0.1 mg/kg to 25 mg/kg, 0.1 mg/kg to 10 mg/kg, 1 mg/kg to 10 mg/kg or from 1 mg/kg to 5 mg/kg.

The terms “treat, treating, treatment” as used herein and in the claims mean ameliorating one or more clinical indicia of disease activity by administering a pharmaceutical composition of the invention in a patient having a pathologic disorder.

More specifically, the term “treatment”, as used herein refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with cancer disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described below.

The terms “delay”, “delaying the onset”, “retard” and all variations thereof are intended to encompass the slowing of the progress and/or exacerbation of a pathologic disorder or an infectious disease and their symptoms slowing their progress, further exacerbation or development, so as to appear later than in the absence of the treatment according to the invention. Still further, as mentioned above, the term “treatment or prevention” as used herein, refers to the complete range of therapeutically positive effects of administrating to a subject including inhibition, reduction of, alleviation of, and relief from, a cancer and illness, a cancer symptoms or undesired side effects of a cancer. More specifically, treatment or prevention of relapse, or re recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9% or even 100%. With regards to the above, it is to be understood that, where provided, percentage values such as, for example, 10%, 50%, 100%, 120%, 500%, etc., are interchangeable with “fold change” values, i.e., 0.1, 0.5, 1.2, 5, etc., respectively.

The present invention relates to the treatment of subjects or patients, in need thereof. By “patient” or “subject in need” it is meant any organism who may be affected by the above-mentioned conditions, and to whom the therapeutic methods herein described is desired, including humans, domestic and non-domestic mammals such as canine and feline subjects, bovine, simian, equine and murine subjects, rodents, domestic birds, aquaculture, fish and exotic aquarium fish. It should be appreciated that the subject may be also any reptile or zoo animal. More specifically, the methods of the invention are intended for mammals. By “mammalian subject” is meant any mammal for which the proposed therapy is desired, including human, livestock, equine, canine, and feline subjects, most specifically humans.

In yet some further aspect, the invention provides therapeutic effective amount of at least one splicing modulating agent comprising at least one nucleic acid sequence or of any vector, vehicle, matrix, nano- or micro-particle or composition comprising the at least one agent, for use in a method for treating, inhibiting, preventing, ameliorating or delaying the onset of at least one neoplastic disorder in a subject. It should be noted that the at least one nucleic acid sequence of the agent targets at least one target nucleic acid sequence that participates directly or indirectly in at least one splicing event of at least one target gene. The introduction of the at least one agent into the target cell in the treated subject induces at least one aberrant splicing event via the target nucleic acid sequence. This aberrant splicing event results in the production of the at least one neoantigen to be expressed by the target cells in the treated subject. In some optional embodiments, an effective amount of at least one peptide derived from the neoantigen, or any derivative, enantiomer, fusion protein, conjugate or polyvalent dendrimer thereof, is further administered to the treated subject. Such peptide is administered prior to, after and/or simultaneously to administration of the splicing modulating agent/s.

Another aspect of the invention relates to a composition comprising at least one splicing modulating agent comprising at least one nucleic acid sequence or of any vector, vehicle, matrix, nano- or micro-particle thereof. In some embodiments, the nucleic acid sequence of the agent targets at least one target nucleic acid sequence that participates directly or indirectly in at least one splicing event of a target gene. In some embodiments, the splicing modulating agent induces at least one aberrant splicing event via the nucleic acid sequence. It should be noted that the aberrant splicing event results in the production of at least one neoantigen expressed by at least one target cell. In some embodiments, the compositions of the invention may be applicable for activating an immune response against at least one target cell in a subject suffering from at least one neoplastic disorder.

In some embodiments, the splicing modulating agent/s of the of the compositions of the invention comprise at least one of the following agents. One option for such agent (a), may be at least one oligonucleotide comprising a nucleic acid sequence complementary to at least part of the target nucleic acid sequence. Another option for such agent (b), is at least one nucleic acid sequence comprising at least one gRNA that targets at least one protospacer within the target nucleic acid sequence. The agent used by the compositions of the invention may be any nucleic acid sequence encoding such gRNA. It should be noted that this gRNA guides at least one PEN to the target nucleic acid sequence in the target gene.

In some embodiments, the nucleic acid sequence of the splicing modulating agent of the composition of the invention, target at least one target nucleic acid sequence that participates directly or indirectly in at least one splicing event. In some embodiments, such target nucleic acid sequence comprises at least one of, a splicing junction, a splice donor site, a splice acceptor site, an exonic splicing enhancer, splicing silencer, an intronic splicing enhancer and an intronic splicing silencer of said target gene, as discussed above.

In yet some further embodiments, the nucleic acid sequence of the splicing modulating agent of the composition of the invention target at least one nucleic acid sequence that participates directly or indirectly in at least one splicing event. Such target nucleic acid sequences may be comprised within at least one coding transcript of the target gene. Such coding transcript may be characterized by at least one of: (i) the coding transcript/s may comprise at least three exons; (ii) at least one of said exons is of a length not divisible by three, and (iii) the coding transcripts comprise at least one intron.

In yet some further embodiments, the target sequence is a target that aberrant splicing mediated by such sequence leads to a frame shift that creates a neoantigen that does not exist in the human proteome. Still further, in some embodiments, peptides derived from such neoantigen, specifically 9-mer peptides (peptides comprising nine amino acid residues), display high affinity to HLA molecules and are therefore immunogenic. Moreover, such peptides do not exist in a mammalian proteome, specifically, the human proteome.

In some embodiments, the target nucleic acid sequence for the splicing modulating agents of the compositions of the invention is comprised within an exon, or within at least one intron located upstream or downstream to such exon, or within at least one splicing junction flanking the exon. For example, the target nucleic acid sequence may be located within a 5′ splice junction, that is the intron/exon splice junction located 5′ or upstream to the indicated exon. In other embodiments, the target sequence may be located within the 3′ splice junction, or in other words, in the exon/intron junction located 3′ or downstream to the indicated exon. It should be understood that in certain embodiments the exon as specified herein is not the first or the last exon in the target transcript. Still further, the target sequence is located in a sequence that is between about 1 to 500 bases upstream or downstream to the indicated exons, as specified herein before in connection with other aspect of the invention.

In some specific embodiments, the target nucleic acid sequence that participates directly or indirectly in at least one splicing event may be comprised within least one of the at least three exons described above, specifically an exon that is not the first exon in the transcript. In yet some further embodiments, the target nucleic acid sequence that participates directly or indirectly in at least one splicing event may be comprised within least one of the at least three exons described above, specifically an exon that is not the last exon in the transcript. In yet some further embodiments, the target nucleic acid sequence that participates directly or indirectly in at least one splicing event may be comprised within an exon that is before the last exon in the transcript. In yet some further embodiments, such exon is in a length not divisible by three. Still further, in some embodiments, in case of induction of exon skipping by the aberrant splicing event induced by the method of the invention, the resulting product is in a length not divisible by three (3), thereby enabling frame shift.

In yet some alternative embodiments, in case of induction of intron retention by the aberrant splicing event induced by the method of the invention, the at least one nucleic acid sequence that participates directly or indirectly in at least one splicing event may be comprised within at least one splicing junction within the transcript.

In yet some further embodiments, the splicing modulating agent of the compositions of the invention may comprise at least one oligonucleotide. In some specific embodiments of the invention, oligomers for use in antisense applications generally range in length from about 10 to about 50 subunits, more preferably about 10 to 30 subunits, and typically 15-25 bases. For example, an oligomer of the invention having 15-20 subunits, specifically, 15, 16, 17, 18, 19, 20, or more bases. In more specific embodiments, such oligonucleotide is an ASO comprising at least fifteen contiguous nucleobases complementary to at least part of said at least one nucleic acid sequence that participates directly or indirectly in at least one splicing event.

Still further, in some embodiments, the splicing modulating agent of the compositions of the invention is at least one guide RNA that guides at least one PEN to the target nucleic acid sequence as specified herein. In some embodiments, the PEN comprises at least one CRISPR/cas protein. Thus, according to some embodiments, the splicing modulating agent used by the compositions of the invention comprises: first (a), at least one nucleic acid sequence comprising at least one gRNA, or any nucleic acid sequence encoding the gRNA; or any kit, vector or vehicle comprising the gRNA or nucleic acid sequence encoding the gRNA. Optionally, the splicing modulating agent may further comprise (b), at least one CRISPR/cas protein, or any nucleic acid molecule encoding the Cas protein, or any kit, vector or vehicle comprising the CRISPR/cas protein or nucleic acid sequence encoding the CRISPR/cas protein.

In yet some specific embodiments, the invention provides compositions for inducing the production of at least one neoantigen in at least one target cell. In some specific embodiments, the target cell may be a cell of a subject suffering from at least one neoplastic disorder. In more specific embodiments, such neoplastic disorder is a cancer. In yet some further specific embodiment, the target cell is a cancerous cell.

Still further, in some embodiments, the compositions of the invention induce the production of at least one neoantigen to be expressed by at least one target cell using the splicing modulating agents of the invention that comprise at least one nucleic acid sequence, specifically, any of the AON/s and gRNAs discussed herein. The agent/s of the compositions of the invention lead to aberrant splicing event in a target gene or at least one transcript thereof. In more specific embodiments, such target gene may be a gene differentially expressed in at least one cancer cell and/or at least one cancerous tissue. In yet some further embodiments, the target gene may be a gene or at least one transcript thereof overexpressed in cancer tissue. In some embodiments, the target gene and specific transcript thereof is selected from the group of genes disclosed by Table 1. In yet some further embodiments, the target sequence that participates directly or indirectly in at least one splicing event is comprised within an exon, within at least one intron located upstream or downstream to the exon, or within at least one splicing junction flanking this exon. In some embodiments, such exon is any of the exons disclosed by Table 2. In more specific embodiments, the cancer may be melanoma. Thus, in some embodiments, the invention provides compositions for inducing the production of at least one neoantigen to be expressed by at least one target cell of a subject suffering from melanoma. In some particular embodiments, the target gene may be the TYR gene. In yet some further particular and non-limiting embodiments, the TYR gene, may be a specifically relevant target gene in cancer cells of a subject suffering from melanoma, and thus, the invention provides composition applicable for treating melanoma.

In some embodiments, for inducing aberrant splicing in the TYR gene, agents targeting nucleic acid sequences that participate or affect splicing of exon 4 may be used by the compositions of the invention.

In certain embodiments, the splicing modulating agent used in the compositions of the invention may comprise at least one AON. Thus, in some specific embodiments, the nucleic acid sequences that participate or affect splicing of exon 4 is targeted using AONs. In more specific embodiments, the AONs that target the human TYR exon 4 may comprise the nucleic acids sequence as denoted by any one of SEQ ID NOs. 28 to 47, or any variants, homologs and derivatives thereof.

In yet some further embodiments, the mouse TYR gene is targeted by the agents of the compositions of the invention. More specifically, in some specific embodiments, targeting AONs may comprise the AON designated herein as TYR oligo 9, specifically an oligonucleotide targeting a sequence comprising the nucleic acids sequence as denoted by SEQ ID NO. 6. In more specific embodiment, the oligo 9 AON, may comprise the nucleic acid sequence as denoted by SEQ ID NO. 4. In yet some further embodiments, targeting AONs used by the compositions of the invention may comprise the AON designated herein as TYR oligo 13, specifically an oligonucleotide targeting a sequence comprising the nucleic acids sequence as denoted by SEQ ID NO. 7. In more specific embodiment, the oligo 13 AON may comprise the nucleic acid sequence as denoted by SEQ ID NO. 5. In yet some further embodiments, the splicing modulating agent used for the compositions of the invention may comprise at least one gRNA, specifically, using the CRISPR-Cas system specified above. Thus, in some specific embodiments, the nucleic acid sequences that participate or affect splicing of exon 4 is targeted using at least one gRNA. In more specific embodiments, gRNAs that target the exon 4 of the human TYR transcript may comprise the nucleic acid sequence as denoted by any one of SEQ ID NOs. 20 to 27.

In yet some further embodiments, the gRNAs that target the exon 4 of the mouse TYR transcript may comprise the nucleic acid sequence as denoted by any one of SEQ ID NOs. 16 and 17 (targeting the 5′ splice site) and SEQ ID NOs. 18 and 19 (targeting the 3′ splice site).

In more specific embodiments, the target gene that comprise the target nucleic acid sequence that participates directly or indirectly in at least one splicing event, is overexpressed in breast cancer.

In some particular embodiments, the target gene may be the HNRNPAB gene. Thus, in some embodiments the modulating agent of the invention targets the HNRNPAB gene. In some embodiments, such composition is applicable for treating breast cancer.

As indicated above, in yet some further particular and non-limiting embodiments, the HNRNPAB gene, may be a specifically relevant target gene in cancer cells of a subject suffering from breast cancer.

In some embodiments, for inducing aberrant splicing in the HNRNPAB gene, targeting nucleic acid sequences that participate or affect splicing of exon 6 may be used by the method of the invention.

In certain embodiments, the splicing modulating agent used by the compositions of the invention may comprise at least one AON. Thus, in some specific embodiments, the nucleic acid sequences that participate or affect splicing of exon 6 is targeted using AONs. In more specific embodiments, the AONs that target the human hnRNPAB exon 6 may comprise the nucleic acids sequence as denoted by any one of SEQ ID NOs. 67 to 86, or any variants, homologs and derivatives thereof.

In yet some further embodiments, the splicing modulating agent used by the compositions of the invention may comprise at least one gRNA, specifically, using the CRISPR-Cas system specified above. Thus, in some specific embodiments, the nucleic acid sequences that participate or affect splicing of exon 6 of hnRNPAB is targeted using at least one gRNA. In more specific embodiments, gRNAs that target the exon 6 of the human hnRNPAB transcript may comprise the nucleic acid sequence as denoted by any one of SEQ ID NOs. 59 to 66.

In yet some further embodiments, the gRNAs that target the exon 6 of the mouse hnRNPAB transcript may comprise the nucleic acid sequence as denoted by any one of SEQ ID NOs. 57 and 58 (targeting the 3′ splice site).

Still further, in some embodiments, the compositions of the invention induce the production and thereby the expression of the newly created neoantigen by the target cell. In some embodiments, the expression of the neoantigen by the target cell leads to activation of an immune-response directed against this target cell. Thus, in some embodiments of the compositions of the invention, the presentation of peptides of the newly produced or created neoantigen by at least one APC in the context of at least one of MHC Class I and MHC Class II, results in the activation of an immune-response against the target cell. In more specific embodiments of the compositions of the invention, the immune-response may be a T cell-dependent immune response. In yet some further embodiments of the compositions of the invention, the T cells may be any one of CD4+ or CD8+ T cells.

In some embodiments, the composition of the invention may further comprise an effective amount of at least one peptide derived from the neoantigen, or any derivative, enantiomer, fusion protein, conjugate or polyvalent dendrimer thereof.

In yet some further embodiments, the composition of the invention may further comprise at least one additional therapeutic agent. In some specific embodiments, the composition of the invention may further comprise at least one immuno-modulatory agent. In some embodiments, the immuno-modulatory agent comprised as a further therapeutic agent in the compositions of the invention may be at least one immune checkpoint inhibitor.

In yet some further embodiments, such immune-checkpoint inhibitor may be any of the inhibitors disclosed by the invention. In yet some further specific embodiments, the immune-checkpoint inhibitor may be directed against at least one of CTLA-4 and PD-1.

Thus, in some embodiments, the compositions of the invention may comprise the splicing modulating agents of the invention, the at least one peptide derived from the neoantigens of the invention and at least one immune-checkpoint inhibitor, or any combinations thereof. In yet some further embodiments, the compositions of the invention may comprise at least one of the splicing modulating agents of the invention and at least one immune-checkpoint inhibitor. In some other embodiments, the compositions of the invention may comprise at least one of the peptides of the invention and at least one immune-checkpoint inhibitor as disclosed by the invention herein before. Combined therapy using either combined compositions as disclosed herein or combined treatment regimen, will be discussed herein after.

The pharmaceutical compositions of the invention can be administered and dosed by the methods of the invention, in accordance with good medical practice, systemically, for example by parenteral, e.g. intravenous, intraperitoneal or intramuscular injection. In another example, the pharmaceutical composition can be introduced to a site by any suitable route including intravenous, subcutaneous, transcutaneous, topical, intramuscular, intraarticular, subconjunctival, or mucosal, e.g. oral, intranasal, or intraocular administration.

Local administration to the area in need of treatment may be achieved by, for example, by local infusion during surgery, topical application, direct injection into the specific organ or the affected tissue, specifically affected with cancer, etc. Still further, the splicing modulating agent comprising at least one nucleic acid sequence, specifically, the AON/s and gRNAs of the invention or any nanoparticles or compositions thereof, described herein, may be adapted for administration by parenteral, intraperitoneal, transdermal, oral (including buccal or sublingual), rectal, topical (including buccal or sublingual), vaginal, intranasal and any other appropriate routes. Such formulations may be prepared by any method known in the art of pharmacy, for example by bringing into association the active ingredient with the carrier(s) or excipient(s). It should be noted that any of the administration modes discussed herein, may be applicable for any of the methods of the invention as described in further aspects of the invention.

Compositions and formulations for oral administration may include powders or granules, suspensions or solutions in water or non-aqueous media, capsules, sachets, lozenges (including liquid-filled), chews, multi- and nano-particulates, gels, solid solution, liposome, films, ovules, sprays or tablets. Thickeners, flavoring agents, diluents, emulsifiers, dispersing aids or binders may be desirable. Pharmaceutical formulations adapted for rectal administration may be presented as suppositories or enemas. Pharmaceutical formulations adapted for vaginal administration may be presented as pessaries, tampons, creams, gels, pastes, foams or spray formulations.

Pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations. It should be understood that in addition to the ingredients particularly mentioned above, the formulations may also include other agents conventional in the art having regard to the type of formulation in question, for example those suitable for oral administration may include flavoring agents. The compositions of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose or methyl cellulose or a heteropolysaccharide polymer, for example, gelan gum, may be incorporated together with a preservative, such as benzalkonium chloride. Such formulations may also be delivered by iontophoresis. Formulations for ocular and aural administration may be formulated to be immediate and/or modified release. Modified release includes delayed, sustained, pulsed, controlled, targeted, and programmed release. In specific embodiments, the unit dosage formulations are those containing a daily dose or sub-dose, as herein above recited, or an appropriate fraction thereof, of an active ingredient. It should be appreciated that the formulations of the compositions of the invention may be in some embodiments, adapted for use as a nano- or micro-particles. Nanoscale drug delivery systems using liposomes and nanoparticles are emerging technologies for the rational drug delivery, which offers improved pharmacokinetic properties, controlled and sustained release of drugs and, more importantly, lower systemic toxicity. A particularly desired solution allows for externally triggered release of encapsulated compounds. Externally controlled release can be accomplished if drug delivery vehicles, such as liposomes or polyelectrolyte multilayer capsules, incorporate nanoparticle (NP) actuators.

More specifically, Controlled drug delivery systems (DDS) have several advantages compared to the traditional forms of drugs. A drug is transported to the place of action, hence, its influence on vital tissues and undesirable side effects can be minimized. Accumulation of therapeutic compounds in the target site increases and, consequently, the required doses of drugs are lower. This modern form of therapy is especially important when there is a discrepancy between the dose or the concentration of a drug and its therapeutic results or toxic effects. Cell-specific targeting can be accomplished by attaching drugs to specially designed carriers. Various nanostructures, including liposomes, polymers, dendrimers, silicon or carbon materials, and magnetic nanoparticles, have been tested as carriers in drug delivery systems. Polymeric nanoparticles are one technology being developed to enable clinically feasible oral delivery. More specifically, the term “nanostructure” or “nanoparticle” is used herein to denote any microscopic particle smaller than about 100 nm in diameter. In some other embodiments, the carrier is an organized collection of lipids. When referring to the structure forming lipids, specifically, micellar formulations or liposomes comprising at least one of the splicing modulating agents of the invention, and optionally, at least one of the peptides of the invention, and/or optionally, at least one immune-checkpoint inhibitor, it is to be understood to mean any biocompatible lipid that can assemble into an organized collection of lipids (organized structure). In some embodiments, the lipid may be natural, semi-synthetic or fully synthetic lipid, as well as electrically neutral, negatively or positively charged lipid. In some embodiments, the lipid may be a naturally occurring phospholipid. Examples of lipids forming glycerophospholipids include, without being limited thereto, glycerophospholipid. phosphatidylglycerols (PG) including dimyristoyl phosphatidylglycerol (DMPG); phosphatidylcholine (PC), including egg yolk phosphatidylcholine, dimyristoyl phosphatidylcholine (DMPC), 1-palmitoyl-2-oleoylphosphatidylcholine (POPC), hydrogenated soy phosphatidylcholine (HSPC), distearoylphosphatidylcholine (DSPC); phosphatidic acid (PA), phosphatidylinositol (PI), phosphatidylserine (PS). Examples of cationic lipids may include, for example, 1,2-dimyristoyl-3-trimethylammonium propane (DMTAP) 1,2-dioleyloxy-3-(trimethylamino) propane (DOTAP); N-[1-(2,3-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethyl-ammonium bromide (DORIE); N-[1-(2,3-dioleyloxy) propyl]-N,N,N-trimethylammonium chloride (DOTMA); 3β[N—(N′,N′-dimethylaminoethane) carbamoly] cholesterol (DC-Chol); and dimethyl-dioctadecylammonium (DDAB), N-[2-[[2,5-bis [3-aminopropyl)amino]-1-oxopentyl]amino] ethyl]-N,N-dimethyl-2,3-bis[(1-oxo-9-octadecenyl)oxy]-1 propanaminium (DOSPA), and ceramide carbamoyl spermine (CCS), or the neutral lipid dioleoylphosphatidyl ethanolamine (DOPE) derivatized with polylysine to form a cationic lipopolymer.

In some embodiments, the structure forming lipids may be combined with other lipids, such as a sterol. Sterols and in particular cholesterol are known to have an effect on the properties of the lipid's organized structure (lipid assembly), and may be used for stabilization, for affecting surface charge, membrane fluidity. In some embodiments, a sterol, e.g. cholesterol is employed in order to control fluidity of the lipid structure. The greater the ratio sterol:lipids (the structure forming lipids), the more rigid the lipid structure is.

In numerous embodiments, the compositions of the present invention may be administered in a form of combination therapy, i.e. in combination with one or more additional therapeutic agents (specifically, any of the peptides of the invention and/or at least one immune-checkpoint inhibitor). Combination therapy may include administration of a single pharmaceutical dosage formulation comprising at least one composition of the invention and additional therapeutics agent(s); as well as administration of at least one composition of the invention and one or more additional agent(s) in its own separate pharmaceutical dosage formulation. Further, where separate dosage formulations are used, compositions of the invention and one or more additional agents can be administered concurrently or at separately staggered times, i.e. sequentially. Still further, said concurrent or separate administrations may be carried out by the same or different administration routes.

In specific embodiments, compositions of the invention are administered with one or more therapeutic agents specifically relevant to cancer.

More specifically, in the present invention, it is contemplated that the other therapeutic agent may involve the administration or inclusion of at least one additional immuno modulatory agent, for example, at least one checkpoint inhibitor. Alternatively, treatment with the splicing modulating agent comprising at least one nucleic acid sequence, specifically, the AON/s and gRNAs, and optionally, at least one of the peptides of the invention of the invention or any compositions thereof, may precede or follow the other agent treatment by intervals ranging from minutes to weeks. In embodiments where the other therapeutic agent and the compounds are administered separately, one would generally ensure that a significant period of time did not expire between the time of each delivery, such that the other agent and the compounds would still be able to exert an advantageously combined effect. In such instances, it is contemplated that one would administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In yet a further aspect thereof, the invention provides an oligonucleotide targeting at least one target nucleic acid sequence that participates directly or indirectly in at least one splicing event of a target gene in a target cell. In certain embodiments, the oligonucleotide of the invention may comprise an antisense oligonucleotide (AON). In some embodiments, the introduction of the AON of the invention into the target cell induces at least one aberrant splicing event via said nucleic acid sequence. In more specific embodiments, such aberrant splicing event results in the production of at least one neoantigen expressed by at least one target cell.

In yet some further embodiments, the AONs of the invention target at least one target nucleic acid sequence that participates directly or indirectly in at least one splicing event. Thus, the AONs of the invention comprise a sequence that is complementary, at least in part, to the target sequence.

In some embodiments, such target nucleic acid sequence comprises at least one of, a splicing junction, a splice donor site, a splice acceptor site, an exonic splicing enhancer, splicing silencer, an intronic splicing enhancer and an intronic splicing silencer of said target gene.

In yet some further embodiments, the at least one nucleic acid sequence targeted by the AONs of the invention, that participates directly or indirectly in at least one splicing event are comprised within at least one coding transcript may be characterized by at least one of: (i) the coding transcript/s may comprise at least three exons; (ii) at least one of said exons is of a length not divisible by three, and (iii) the coding transcripts comprise at least one intron. In yet some further embodiments, the target sequence is a target that aberrant splicing mediated by such sequence leads to a frame shift that creates a neoantigen that does not exists in the human proteome. Still further, in some embodiments, peptides derived from such neoantigen, specifically 9-mer peptides (peptides comprising nine amino acid residues), display high affinity to HLA molecules and are therefore immunogenic. Moreover, such peptides do not exists in a mammalian proteome, specifically, the human proteome. In some specific embodiments, the target nucleic acid sequence that participates directly or indirectly in at least one splicing event may be comprised within least one of the at least three exons described above, specifically an exon that is not the first exon in the transcript. In yet some further embodiments, the target nucleic acid sequence that participates directly or indirectly in at least one splicing event may be comprised within least one of the at least three exons described above, specifically an exon that is not the last exon in the transcript. In yet some further embodiments, the target nucleic acid sequence that participates directly or indirectly in at least one splicing event may be comprised within an exon that is before the last exon in the transcript. In yet some further embodiments, such exon is in a length not divisible by three. Still further, in some embodiments, in case of induction of exon skipping by the aberrant splicing event induced by the AONs of the invention, may be in a length not divisible by three (3), thereby enabling frame shift.

In yet some alternative embodiments, in case of induction of intron retention by the aberrant splicing event induced by the AONs of the invention, the least one nucleic acid sequence that participates directly or indirectly in at least one splicing event may be comprised within at least one splicing junction within said transcript.

In some embodiments the oligonucleotide (AON/s, ASO/s) of the invention may comprise at least 10 or more contiguous nucleobases complementary to at least part of the at least one nucleic acid sequence that participates directly or indirectly in at least one splicing event, specifically, at least 10 or more, at least 11 or more, at least 12 or more, at least 13 or more, at least 14 or more, at least 15 or more, at least 16 or more, at least 17 or more, at least 18 or more, at least 19 or more, at least 20 or more, at least 21 or more, at least 22 or more, at least 23 or more, at least 24 or more, at least 25 or more, at least 26 or more, at least 27 or more, at least 28 or more, at least 29 or more, at least 30 or more contiguous nucleobases complementary to the target sequence.

In yet some further embodiments, the oligonucleotide (AON) of the invention may comprise at least fifteen contiguous nucleobases complementary to at least part of said at least one nucleic acid sequence that participates directly or indirectly in at least one splicing event. In some specific embodiments, the oligonucleotide of the invention may comprise between 10 to 30 linked nucleosides and having a nucleobase sequence comprising at least 15 contiguous nucleobases complementary to a target region of equal length of the target nucleic acid sequence as described herein. In yet some further embodiments, the oligonucleotide of the invention may be 15 to 20 nucleosides in length. In yet some further embodiments, the oligonucleotide provided by the invention may be a modified oligonucleotide. It should be understood that any oligonucleotide or any modified oligonucleotide described herein before in connection with other aspects of the invention are also encompassed by the present aspect.

In some embodiments the AON/s of the invention may target any one of the target genes selected from the group of genes disclosed by Table 1. In more specific embodiments the AON/s of the invention may target, and therefore complementary, at least in part to a target sequence that participates directly or indirectly in at least one splicing event, is comprised within an exon, within at least one intron located upstream or downstream to the exon, or within at least one splicing junction flanking such exon. In some embodiments, the exon is selected from the group of exons disclosed by Table 2. In some embodiments the AON/s of the invention may target the TYR gene.

In yet some further embodiments, the AONs of the invention may target exon 4 of the TYR gene or any flanking sequences thereof. In some specific embodiments the AONs of the invention that target the human TYR gene may comprise a nucleic acid sequence as denoted by any one of SEQ ID NOs. 28 to 47 or any variants, homologs or derivatives thereof. In yet some further specific embodiments the AONs of the invention may target the mouse TYR gene and may comprise a nucleic acid sequence as denoted by any one of SEQ ID NOs. 4 or 5, or any variants, homologs or derivatives thereof.

In some embodiments the AON/s of the invention may target the HNRNPAB gene. In yet some further embodiments, the AONs of the invention may target exon 6 of the HNRNPAB gene or any flanking sequences thereof. In some specific embodiments the AONs of the invention that target the human HNRNPAB gene may comprise a nucleic acid sequence as denoted by any one of SEQ ID NOs. 67 to 86 or any variants, homologs or derivatives thereof. The invention also encompasses any homologues or variant of the AONs of the invention, specifically, those defined by their nucleic acid sequence according to the invention. The term “homologues” is used to define nucleic acid sequences (oligonucleotide) which maintain a minimal homology to the nucleic acid sequences defined by the invention, e.g. preferably have at least about 65%, more preferably at least about 70%, at least about 75%, even more preferably at least about 80%, at least about 85%, most preferably at least about 90%, at least about 95% overall sequence homology, specifically, with the entire nucleic acid sequence of any of the oligonucleotides as structurally defined above, e.g. of a specified sequence, more specifically, the nucleic acid sequence of the AONs as denoted by any one of SEQ ID NOs. 16-19, 28-47, and 67-86, and any variants and derivatives thereof. The term “derivative” or “variant” is used to define nucleic acid sequences (oligonucleotide), with any insertions, deletions, substitutions and modifications of between about 1 to 10 bases, to the nucleic acid sequences that do not alter the activity of the original AONs (specifically, to induce aberrant splicing event in the target sequence). It should be understood that all AONs modifications disclosed by the invention in connection with other aspects of the invention, are also applicable in the present aspect.

A further aspect of the invention relates to a polypeptide derived from at least one neoantigen, or any derivative, enantiomer, fusion protein, conjugate, polyvalent dendrimer or any vaccine thereof. The neoantigen is produced by at least one aberrant splicing event induced by at least one splicing modulating agent comprising at least one nucleic acid sequence, in a target cell of a subject suffering from a neoplastic disorder. In some embodiments, at least one nucleic acid sequence of the agent targets at least one target nucleic acid sequence that participates directly or indirectly in at least one splicing event of at least one target gene in the target cell. In certain embodiments, the polypeptide is in the length of between about eight to about twenty two amino acid residues.

Still further, peptides derived from the neoantigens created from aberrant splicing induced by the splicing modulating agent of the invention in any of the targets specified in Tables 1 and 2, may be peptides that are capable of binding MHC class I molecules and or MHC class II molecules. In some specific embodiments, such peptides may comprise between about 5 to 50 amino acid residues, specifically, 5, 6, 7, 8, 9, 10, 11, 12, 13, 14, 15, 16, 17, 18, 19, 20, 21, 22, 23, 24, 25, 26, 27, 28, 29, 30, 31, 32, 3, 34, 35, 36, 37, 38, 39, 40, 41, 42, 43, 44, 45, 46, 47, 48, 49, 50 or more amino acid residues. In yet some further specific embodiments, the peptides derived from the neoantigens created by the invention may comprise between about 8 to about 22 amino acid residues. In more specific embodiments, when the aberrant splicing is induced in human target genes, the peptide derived from the neoantigen may bind HLA-I and/or HLA-II molecules. More specifically, peptides comprising between about 8 to about 15 amino acid residues may bind HLA-I molecules, and peptides comprising between about 8 to about 22 amino acid residues may bind HLA-II molecules. Thus, in some embodiments, the polypeptide of the invention, is capable of binding at least one HLA allele.

In some specific embodiments, the polypeptide of the invention, as well as the neoantigen do not exist in the natural human proteome and wherein said polypeptide is capable of binding at least one of the HLA alleles: HLA-A01:01, HLA-A02:01, HLA-A03:01, HLA-A11:01, HLA-A23:01, HLA-A24:02, HLA-A33:03, HLA-B07:02, HLA-B08:01, HLA-B44:02, HLA-C01:02, HLA-C04:01, HLA-C05:01, HLA-C06:02, HLA-C07:01, HLA-C07:02 and HLA-C08:01.

In some embodiments, the peptides of the invention may be derived from a neoantigen created by aberrant splicing event occurring in any of the target genes and/or target transcripts specified in Table 1. In yet some further embodiments, the peptides of the invention may be derived from a neoantigen created by aberrant splicing event occurring in a target sequence located any of the exons, or in the vicinity of the exons specified by Table 2. Still further, in some embodiments the peptides of the invention may be derived from a neoantigen created by aberrant splicing event occurring in the Tyr gene. In more specific embodiments, such splicing event may involve a target sequence within exon 4 of the TYR gene. Still further, in some embodiments, peptides derived from neoantigen produced by an aberrant splicing event in the human TYR gene may comprise the amino acid sequence as denoted by any one of SEQ ID NOs. 2, 3, 14, 89 or any derivative, enantiomer, fusion protein, conjugate, polyvalent dendrimer or any vaccine thereof. In yet some further embodiments, the peptides derived from neoantigen produced by an aberrant splicing event in the mouse TYR gene may comprise the amino acid sequence as denoted by any one of SEQ ID NOs. 12, 13, or any derivative, enantiomer, fusion protein, conjugate, polyvalent dendrimer or any vaccine thereof.

In certain embodiments the peptides of the invention may be derived from a neoantigen created by aberrant splicing event occurring in the HNRNPAB gene. In more specific embodiments, such splicing event may involve a target sequence within exon 6 of the HNRNPAB gene. Still further, in some embodiments, peptides derived from neoantigen produced by an aberrant splicing event in the human HNRNPAB gene may comprise the amino acid sequence as denoted by any one of SEQ ID NO. 6 or any 9-mer peptides thereof, or any derivative, enantiomer, fusion protein, conjugate, polyvalent dendrimer or any vaccine thereof. In yet some further embodiments, the peptides derived from neoantigen produced by an aberrant splicing event in the mouse HNRNPAB gene may comprise the amino acid sequence as denoted by any one of SEQ ID NOs. 52, 53, 55, or any derivative, enantiomer, fusion protein, conjugate, polyvalent dendrimer or any vaccine thereof.

The invention provides polypeptides that are the neoantigents of the invention and as well as peptides derived from the neoantigens produced (or expected to be produced) by the methods and compositions of the invention. Peptide/s derived from the neoantigen of the invention is meant any polypeptide fragment of between about five to fifty (5-50) contiguous amino acid residues of the neoantigen, specifically, any polypeptide that comprises at least five or more contiguous amino acid residues that are identical to those of the neopeptide. Specifically, a polypeptide comprising at least 5, 6, 7, 8, 9, 10 or more contiguous amino acid residues that are identical to those of the neopeptide, more specifically, at least eight or nine amino acid residues that are identical to those of the neopeptide of the invention or any derivative, enantiomer, fusion protein, conjugate, polyvalent dendrimer or any vaccine thereof.

An ‘isolated polypeptide’ is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms. By definition, isolated peptides are also non-naturally occurring, synthetic peptides. Methods for isolating or synthesizing peptides of interest with known amino acid sequences are well known in the art.

It should be noted that the polypeptides according to the invention can be produced either synthetically, or by recombinant DNA technology. Methods for producing polypeptides peptides are well known in the art.

The polypeptide of the invention are therefore considered as proteinaceous material. A “proteinaceous material” is any protein, or fragment thereof, or complex containing one or more proteins formed by any means, such as covalent peptide bonds, disulfide bonds, chemical crosslinks, etc., or non-covalent associations, such as hydrogen bonding, van der Waal's contacts, electrostatic salt bridges, etc.

An ‘amino acid/s’ or an ‘amino acid residue/s’ can be a natural or non-natural amino acid residue/s linked by peptide bonds or bonds different from peptide bonds. The amino acid residues can be in D-configuration or L-configuration (referred to herein as D- or L-enantiomers). An amino acid residue comprises an amino terminal part (NH₂) and a carboxy terminal part (COOH) separated by a central part (R group) comprising a carbon atom, or a chain of carbon atoms, at least one of which comprises at least one side chain or functional group. NH₂ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature are listed in 37 C.F.R. 1.822(b)(2). Examples of non-natural amino acids are also listed in 37 C.F.R. 1.822(b)(4), other non-natural amino acid residues include, but are not limited to, modified amino acid residues, L-amino acid residues, and stereoisomers of D-amino acid residues. Naturally occurring amino acids may be further modified, e.g. hydroxyproline, γ-carboxyglutamate, and 0-phosphoserine.

Thus, the polypeptides of the invention derived from the neoantigens produced by the methods and compositions of the invention may comprise natural or non-natural amino acid residues, or any combination thereof.

Further, amino acids may be amino acid analogs or amino acid mimetics. Amino acid analogs refer to compounds that have the same fundamental chemical structure as naturally occurring amino acids, but modified R groups or modified peptide backbones, e.g. homoserine, norleucine, methionine sulfoxide, methionine methyl sulfonium. Amino acid mimetics refers to chemical compounds that have a structure that is different from the general chemical structure of an amino acid, but that function in a manner similar. Amino acids may be referred to herein by either their commonly known three letter symbols or by the one-letter symbols recommended by the IUPAC-IUB Biochemical Nomenclature Commission.

Further, polypeptides derived from the neoantigens produced by the methods and compositions of the invention may comprise ‘equivalent amino acid residues’. This term refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, equivalent amino acid residues can be regarded as conservative amino acid substitutions.

In the context of the present invention, within the meaning of the term ‘equivalent amino acid substitution’ as applied herein, is meant that in certain embodiments one amino acid may be substituted for another within the groups of amino acids indicated herein below:

(i) Amino acids having polar side chains (Asp, Glu, Lys, Arg, His, Asn, GIn, Ser, Thr, Tyr, and Cys); (ii) Amino acids having non-polar side chains (Gly, Ala, VaI, Leu, lie, Phe, Trp, Pro, and Met); (iii) Amino acids having aliphatic side chains (GIy, Ala VaI, Leu, ile); (iv) Amino acids having cyclic side chains (Phe, Tyr, Trp, His, Pro); (v) Amino acids having aromatic side chains (Phe, Tyr, Trp); (vi) Amino acids having acidic side chains (Asp, Glu); (vii) Amino acids having basic side chains (Lys, Arg, His); (viii) Amino acids having amide side chains (Asn, GIn); (ix) Amino acids having hydroxy side chains (Ser, Thr); (x) Amino acids having sulphur-containing side chains (Cys, Met); (xi) Neutral, weakly hydrophobic amino acids (Pro, Ala, GIy, Ser, Thr); (xii) Hydrophilic, acidic amino acids (GIn, Asn, Glu, Asp), and (xiii) Hydrophobic amino acids (Leu, lie, VaI).

Still further, the polypeptide of the invention of the invention may have secondary modifications, such as phosphorylation, acetylation, glycosylation, sulfhydryl bond formation, cleavage and the likes, as long as said modifications retain the functional properties of the original protein, specifically, the ability to bind at least one HLA allele. In some specific embodiments, the peptides may be capable of binding with strong affinity to at least one of the HLA alleles HLA-A01:01, HLA-A02:01, HLA-A03:01, HLA-A11:01, HLA-A23:01, HLA-A24:02, HLA-A33:03, HLA-B07:02, HLA-B08:01, HLA-B44:02, HLA-C01:02, HLA-C04:01, HLA-C05:01, HLA-C06:02, HLA-C07:01, HLA-C07:02 and HLA-C08:01. More specifically, any modified polypeptide in accordance with the invention should retain the ability to induce an immune response against any tumor cell expressing at least one of the neoantigens of the invention. Secondary modifications are often referred to in terms of relative position to certain amino acid residues. For example, a certain sequence positioned carboxyl-terminal to a reference sequence within a polypeptide is located proximal to the carboxyl terminus of the reference sequence, but is not necessarily at the carboxyl terminus of the complete polypeptide.

It should be understood that in some embodiments, the polypeptides of the invention may be further provided as a polypeptide comprising at least two peptides, optionally connected via at least one linker. The term “linker” in the context of the invention concerns an amino acid sequence of from about 1 to about 10 or more amino acid residues positioned within and/or flanking the polypeptides of the invention. The linker may be positioned at the C-terminus and/or at the N-terminus thereof. The linker is covalently linked or joined to the amino acid residues in its vicinity. For example, a linker in accordance with the invention may be of 1, 2, 3, 4, 5, 6, 7, 8, 9 or 10 or more amino acid residues long. Linkers are often composed of flexible amino acid residues, for example but not limited to glycine and serine so that the adjacent protein domains are free to move relative to one another. The design of a linker that enables proper folding of the various domains of a protein is well known in the art.

The invention further encompasses any derivatives, enantiomers, analogues, variants or homologues of any of the polypeptides disclosed herein. In some specific embodiments, the invention encompasses any derivative of the peptides of the invention, specifically, any peptide derived from the neoantigen of the invention. Non limiting examples for such peptides include, but are not limited to any of the peptides that comprise the amino acid sequence of SEQ ID NOs. 2, 3, 9, 12, 13, 14, 89, 6, 52, 53, 55, 56 and any derivatives thereof. The term “derivative” is used to define amino acid sequences (polypeptide), with any insertions, deletions, substitutions and modifications to the amino acid sequences (polypeptide) that do not alter the activity of the original polypeptides. By the term “derivative” it is also referred to homologues, variants and analogues thereof, as well as covalent modifications of a polypeptides made according to the present invention. Specifically, derivatives include, but are not limited to, polypeptides that differ in one or more amino acids in their overall sequence from the polypeptides defined herein, polypeptides that have deletions, substitutions, inversions or additions. In some embodiments, derivatives refer to polypeptides, which differ from the polypeptides specifically defined in the present invention by insertions of amino acid residues. It should be appreciated that by the terms “insertions” or “deletions”, as used herein it is meant any addition or deletion, respectively, of amino acid residues to the polypeptides used by the invention, of between 1 to 5 amino acid residues or more, and specifically, between 1 to 2 amino acid residues. More particularly, insertions or deletions may be of any one of 1, 2, 3, 4 or 5 amino acids. It should be noted that the insertions or deletions encompassed by the invention may occur in any position of the modified peptide, as well as in any of the N′ or C′ termini thereof. It should be appreciated that in cases the deletion/s or insertion/s are in the N or C-terminus of the peptide, such derivatives may be also referred to as fragments. The polypeptide of the invention may all be positively charged, negatively charged or neutral. In addition, they may be in the form of a dimer, a multimer or in a constrained conformation, which can be attained by internal bridges, short-range cyclizations, extension or other chemical modifications.

The polypeptides of the invention can be coupled (conjugated) through any of their residues to another peptide or agent. For example, the polypeptides of the invention can be coupled through their N-terminus to a lauryl-cysteine (LC) residue and/or through their C-terminus to a cysteine (C) residue.

Further, the polypeptide of the invention may be extended at the N-terminus and/or C-terminus thereof with various identical or different amino acid residues. As an example for such extension, the peptide may be extended at the N-terminus and/or C-terminus thereof with identical or different amino acid residue/s, which may be naturally occurring or synthetic amino acid residue/s. An additional example for such an extension may be provided by peptides extended both at the N-terminus and/or C-terminus thereof with a cysteine residue. Naturally, such an extension may lead to a constrained conformation due to Cys-Cys cyclization resulting from the formation of a disulfide bond. Another example may be the incorporation of an N-terminal lysyl-palmitoyl tail, the lysine serving as linker and the palmitic acid as a hydrophobic anchor. In addition, the peptides may be extended by aromatic amino acid residue/s, which may be naturally occurring or synthetic amino acid residue/s, for example, a specific aromatic amino acid residue may be tryptophan. The peptides may be extended at the N-terminus and/or C-terminus thereof with various identical or different organic moieties, which are not naturally occurring or synthetic amino acids.

For every single peptide sequence defined by the invention and disclosed herein, specifically, peptide comprising the amino acid sequence of any one of SEQ ID NOs. 2, 3, 9, 12, 13, 14, 89, 52, 53, 55, 56, this invention includes the corresponding retro-inverse sequence wherein the direction of the peptide chain has been inverted and wherein all or part of the amino acids belong to the D-series. It should be understood that the present invention includes embodiments wherein one or more of the L-amino acids is replaced with its D isomer.

In yet some further embodiments, the polypeptide of the invention of the invention may comprise at least one amino acid residue in the D-form. It should be noted that every amino acid (except glycine) can occur in two isomeric forms, because of the possibility of forming two different enantiomers (stereoisomers) around the central carbon atom. By convention, these are called L- and D-forms, analogous to left-handed and right-handed configurations.

It should be appreciated that in some embodiments, the enantiomer or any derivatives of the polypeptide of the invention may exhibit at least one of enhanced activity, and superiority. In more specific embodiments, such derivatives and enantiomers may exhibit increased immunogenicity, enhanced stability, and increased resistance to proteolytic degradation.

The invention also encompasses any homologues of the polypeptides specifically defined by their amino acid sequence according to the invention. The term “homologues” is used to define amino acid sequences (polypeptide) which maintain a minimal homology to the amino acid sequences defined by the invention, e.g. preferably have at least about 65%, more preferably at least about 70%, at least about 75%, even more preferably at least about 80%, at least about 85%, most preferably at least about 90%, at least about 95% overall sequence homology with the amino acid sequence of any of the polypeptide as structurally defined above, e.g. of the entire specified sequence, more specifically, an amino acid sequence of the polypeptides as denoted by any one of SEQ ID NOs. 2, 3, 9, 12, 13, 14, 89, 52, 53, 55, 56, and any derivatives, enantiomers and fusion proteins thereof.

In some embodiments, the present invention also encompasses polypeptides which are variants of, or analogues to, the polypeptides specifically defined in the invention by their amino acid sequence. With respect to amino acid sequences, one of skill will recognize that individual substitutions, deletions or additions to peptide, polypeptide, or protein sequence thereby altering, adding or deleting a single amino acid or a small percentage of amino acids in the encoded sequence is a “conservatively modified variant”, where the alteration results in the substitution of an amino acid with a chemically similar amino acid.

Conservative substitution tables providing functionally similar amino acids are well known in the art and disclosed herein before. Such conservatively modified variants are in addition to and do not exclude polymorphic variants, interspecies homologues, and analogous peptides of the invention.

More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved.

Certain commonly encountered amino acids which also provide useful substitutions include, but are not limited to, β-alanine (β-Ala) and other omega-amino acids such as 3-aminopropionic acid, 2,3-diaminopropionic acid (Dpr), 4-aminobutyric acid and so forth; α-aminoisobutyric acid (Aib); ε-aminohexanoic acid (Aha); δ-aminovaleric acid (Ava); N-methylglycine or sarcosine (MeGly); ornithine (Orn); citrulline (Cit); t-butylalanine (t-BuA); t-butylglycine (t-BuG); N-methylisoleucine (Male); phenylglycine (Phg); cyclohexylalanine (Cha); norleucine (NIe); naphthylalanine (NaI); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); β-2-thienylalanine (Thi); methionine sulfoxide (MSO); homoarginine (hArg); N-acetyl lysine (AcLys); 2,4-diaminobutyric acid (Dbu); 2,4-diaminobutyric acid (Dab); p-aminophenylalanine (Phe(pNH.sub.2)); N-methyl valine (MeVaI); homocysteine (hCys), homophenylalanine (hPhe) and homoserine (hSer); hydroxyproline (Hyp), homoproline (hPro), N-methylated amino acids (e.g., N-substituted glycine). Covalent modifications of the peptide are included and may be introduced by reacting targeted amino acid residues of the peptide with an organic derivatizing agent that is capable of reacting with selected side chains or terminal residues. It should be appreciated that at least one of Cysteinyl residues, Histidyl residues, Lysinyl residues, Arginyl residues and modification of tyrosyl residues, as well as carboxyl side groups (aspartyl or glutamyl)

Derivatization with bifunctional agents is useful for cross-linking the peptide to a water-insoluble support matrix or other macromolecular carrier. Still further, commonly used chemical modifications include hydroxylation of proline and lysine, phosphorylation of the hydroxyl groups of seryl or threonyl residues, methylation of the α-amino groups of lysine, arginine, and histidine side chains (Creighton, supra), acetylation of the N-terminal amine, and, in some instances, amidation of the C-terminal carboxyl.

Such chemically modified and derivatized moieties may improve the peptide's solubility, absorption, biological half-life, and the like. These changes may eliminate or attenuate undesirable side effects of the proteins in vivo.

It should be appreciated that the invention further encompass any of the peptides of the invention referred herein, any serogates thereof, any salt, base, ester or amide thereof, any enantiomer, stereoisomer or disterioisomer thereof, or any combination or mixture thereof. The invention particularly encompasses the use of any of the polypeptides of the invention as vaccines, or vaccinating compositions, specifically, the polypeptides derived from the neoantigens of the invention. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts.

Still further, the vaccine/s of the invention may include one or more excipients that enhance the vaccinated patient's immune response (which may include an antibody response, cellular response, or both), thereby increasing the effectiveness of the vaccine. The adjuvant(s) may be a substance that has a direct (e.g., cytokine or Bacille Calmette-Guerin (“BCG”)) or indirect effect (liposomes) on cells of the human patient's immune system. Examples of often suitable adjuvants include oils (e.g., mineral oils), metallic salts (e.g., aluminum hydroxide or aluminum phosphate), bacterial components (e.g., bacterial liposaccharides, Freund's adjuvants, and/or MDP), plant components (e.g., Quil A), and/or one or more substances that have a carrier effect (e.g., bentonite, latex particles, liposomes, and/or Quil A, ISCOM). As noted above, adjuvants also include, for example, CARBIGEN™ and carbopol. It should be recognized that this invention encompasses both vaccines that comprise an adjuvant(s), as well as vaccines that do not comprise any adjuvant. It is contemplated that the vaccine may be freeze-dried (or otherwise reduced in liquid volume) for storage, and then reconstituted in a liquid before or at the time of administration. Such reconstitution may be achieved using, for example, vaccine-grade water.

The following description discloses routs of administration that may be applicable to the any vaccines based on any of the polypeptides of the invention, specifically, any of the 9-mer peptides derived from the neoantigens of the invention, as well as to any of the antisense oligonucleotides of the invention (AON/s, or ASOs) used by any of the methods described herein before, or to any combinations of the peptides and ASOs of the invention.

More specifically, the vaccine or the AON/s disclosed herein can be delivered via a variety of routes. Typical delivery routes include parenteral administration, e.g., intradermal, intramuscular or subcutaneous delivery. Other routes include oral administration, intranasal, intravaginal and mucosal administration (such as intranasal, oral, intratracheal, and ocular).

The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant. The vaccine or the AON/s can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine. The vaccine or the AON/s can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion. The vaccine or the AON/s can be incorporated into liposomes, microspheres or other polymer matrices. Liposomes can consist of phospholipids or other lipids, and can be nontoxic, physiologically acceptable and metabolizable carriers that are relatively simple to make and administer.

Vaccine or the AON/s in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

Mucosal vaccines may be, for example, liquid dosage forms, such as pharmaceutically acceptable emulsions, solutions, suspensions, syrups, and elixirs. Excipients suitable for such vaccines include, for example, inert diluents commonly used in the art, such as, water, saline, dextrose, glycerol, lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol. Excipients also can comprise various wetting, emulsifying, suspending, flavoring (e.g., sweetening), and/or perfuming agents.

Oral mucosal vaccines also may, for example, be tableted or encapsulated for convenient administration. Such capsules or tablets can contain a controlled-release formulation. In the case of capsules, tablets, and pills, the dosage forms also can comprise buffering agents, such as sodium citrate, or magnesium or calcium carbonate or bicarbonate.

Tablets and pills additionally can be prepared with enteric coatings.

It is contemplated that the vaccine may be administered via the human or rodent patient's drinking water and/or food.

“Parenteral administration” that is also contemplated by the invention includes subcutaneous injections, submucosal injections, intravenous injections, intramuscular injections, intrasternal injections, transcutaneous injections, and infusion. Injectable preparations (e.g., sterile injectable aqueous or oleaginous suspensions) can be formulated according to the known art using suitable excipients, such as vehicles, solvents, dispersing, wetting agents, emulsifying agents, and/or suspending agents. These typically include, for example, water, saline, dextrose, glycerol, ethanol, corn oil, cottonseed oil, peanut oil, sesame oil, benzyl alcohol, benzyl alcohol, 1,3-butanediol, Ringer's solution, isotonic sodium chloride solution, bland fixed oils (e.g., synthetic mono- or diglycerides), fatty acids (e.g., oleic acid), dimethyl acetamide, surfactants (e.g., ionic and non-ionic detergents), propylene glycol, and/or polyethylene glycols. Excipients also may include small amounts of other auxiliary substances, such as pH buffering agents. As mentioned above, although indicated specifically for vaccines, it is to be appreciated that the above-administration modes are also applicable for any of the AON/s, or ASOs of the invention, as well as to any combinations thereof with any of the peptide vaccines of the invention.

As indicated above, the present invention further provides combined therapy involving the use of at least two compounds, specifically, the at least one splicing modulating agent comprising at least one nucleic acid sequence, specifically, the AON/s and gRNAs of the invention and at least one additional therapeutic agent, specifically, peptides derived from the neoantigen produced by the invention and/or checkpoint inhibitor that may be administered either together in a pharmaceutical composition, or in separate compositions through different routes, dosages and combinations.

More specifically, the treatment of diseases and conditions with a combination of active ingredients may involve separate administration of each active ingredient. Therefore, a kit providing a convenient modular format of the different constituents of the compounds and related components required for treatment would allow the required flexibility in the above parameters.

Thus, in a further aspect of the invention relates to a kit comprising:

First component of the kit of the invention may be at least one splicing modulating agent comprising at least one nucleic acid sequence or of any vector, vehicle, matrix, nano- or micro-particle or composition comprising said at least one agent. In some embodiments, the nucleic acid sequence of said agent target at least one target nucleic acid sequence that participates directly or indirectly in at least one splicing event of a target gene in a target cell. It should be noted that the introduction of the agent of the invention into a target cell induce at least one aberrant splicing event via the nucleic acid sequence. Such aberrant splicing event results in the production of at least one neoantigen expressed by at least one target cell. The kit of the invention may optionally further comprise as the second component thereof, at least one polypeptide derived from at least one neoantigen, or any derivative, enantiomer, fusion protein, conjugate, polyvalent dendrimer or any vaccine thereof. Such polypeptide is produced by at least one aberrant splicing event induced by at least one splicing modulating agent comprising at least one nucleic acid sequence. In yet some further additional or alternative embodiments, the kit of the invention may optionally further comprise as a further component thereof at least one therapeutic agent, that may be at least one immuno-modulatory agent. In yet some further embodiments, the immuno-modulatory agent may be at least one immune-checkpoint inhibitor. In yet some further specific embodiments, such immune-checkpoint inhibitor may be directed against any of the checkpoint inhibitors disclosed by the invention, specifically, any inhibitor directed against at least one of CTLA-4, PD-1 and PD-L1.

In some embodiments, the splicing modulating agent of the kits of the invention comprises at least one of the following agents. One option for such agent (a), may be at least one oligonucleotide comprising a nucleic acid sequence complementary to at least part of the target nucleic acid sequence. Another option for such agent (b), is at least one nucleic acid sequence comprising at least one gRNA that targets at least one protospacer within the target nucleic acid sequence. The agent used by the methods of the invention may be any nucleic acid sequence encoding such gRNA. It should be noted that this gRNA guides at least one PEN to the target nucleic acid sequence in the target gene.

In some embodiments, the at least one nucleic acid sequence that participates directly or indirectly in at least one splicing event is at least one of, a splicing junction, a splice donor site, a splice acceptor site, an exonic splicing enhancer, splicing silencer, an intronic splicing enhancer and an intronic splicing silencer of the target gene.

In yet some further embodiments, the at least one nucleic acid sequence that participates directly or indirectly in at least one splicing event are comprised within at least one coding transcript may be characterized by at least one of: (i) the coding transcript/s may comprise at least three exons; (ii) at least one of said exons is of a length not divisible by three and (iii) the coding transcripts comprise at least one intron. In yet some further embodiments, the target sequence is a target that aberrant splicing mediated by such sequence leads to a frame shift that creates a neoantigen that does not exist in the human proteome. Still further, in some embodiments, peptides derived from such neoantigen may be at least one of 8-mer, 9-mer, 10-mer, 11-mer, 12-mer, 13-mer, 14-mer, 15-mer, 16-mer, 17-mer, 18-mer, 19-mer, 20-mer, 21-mer, 22-mer peptides, specifically, 8-14-mer peptides, and in some embodiments, 9-mer peptides (peptides comprising nine amino acid residues), display high affinity to HLA molecules and are therefore immunogenic. Moreover, such peptides do not exist in a mammalian proteome, specifically, the human proteome.

In some specific embodiments, the target nucleic acid sequence that participates directly or indirectly in at least one splicing event may be comprised within least one of the at least three exons described above, specifically an exon that is not the first exon in the transcript. In yet some further embodiments, the target nucleic acid sequence that participates directly or indirectly in at least one splicing event may be comprised within least one of the at least three exons described above, specifically an exon that is not the last exon in the transcript. In yet some further embodiments, the target nucleic acid sequence that participates directly or indirectly in at least one splicing event may be comprised within an exon that is before the last exon in the transcript. In yet some further embodiments, such exon is in a length not divisible by three. Still further, in some embodiments, in case of induction of exon skipping by the aberrant splicing event induced by the kit of the invention, may be in a length not divisible by three (3), thereby enabling frame shift. In yet some alternative embodiments, in case of induction of intron retention by the aberrant splicing event induced by the kit of the invention, the least one nucleic acid sequence that participates directly or indirectly in at least one splicing event may be comprised within at least one splicing junction within the transcript.

In yet some further embodiments, the oligonucleotide of the kits of the invention may comprise at least fifteen contiguous nucleobases complementary to at least part of said at least one nucleic acid sequence that participates directly or indirectly in at least one splicing event. Still further, in some embodiments of the kits of the invention, the splicing modulating agent is at least one guide RNA that guides at least one PEN to the target nucleic acid sequence as specified herein. In some embodiments, the PEN comprises at least one CRISPR/cas protein. Thus, according to some embodiments, the splicing modulating agent used by the kits of the invention comprises: first (a), at least one nucleic acid sequence comprising at least one gRNA, or any nucleic acid sequence encoding the gRNA; or any composition, vector or vehicle comprising the gRNA or nucleic acid sequence encoding the gRNA. Optionally, the splicing modulating agent may further comprise (b), at least one CRISPR/cas protein, or any nucleic acid molecule encoding the Cas protein, or any composition, vector or vehicle comprising the CRISPR/cas protein or nucleic acid sequence encoding the CRISPR/cas protein.

In some embodiments the splicing modulating agents of the kits of the invention may target any one of the target genes selected from the group of genes disclosed by Table 1.

In more specific embodiments the splicing modulating agents of the kit invention may target a target sequence that participates directly or indirectly in at least one splicing event is comprised within an exon, within at least one intron located upstream or downstream to said exon, or within at least one splicing junction flanking said exon, wherein said exon is selected from the group of exons disclosed by Table 2.

In some embodiments, the splicing modulatory agent of the invention (e.g., AON/s and gRNAs) may target the TYR gene. In yet some further embodiments, the splicing modulatory agent (e.g., AON/s and gRNAs) of the invention may target exon 4 of the TYR gene or any flanking sequences thereof. In some specific embodiments the splicing modulating agent used in the kit of the invention may be at least one AON that target the human TYR gene. In some embodiments, such AONs may comprise a nucleic acid sequence as denoted by any one of SEQ ID NOs. 28 to 47 or any variants, homologs or derivatives thereof. In yet some further specific embodiments AONs used by the kits of the invention may target the mouse TYR gene and may comprise a nucleic acid sequence as denoted by any one of SEQ ID NOs. 4, 5, 6 or 7, or any variants, homologs or derivatives thereof. In yet some further embodiments, the splicing modulating agent used by the kits of the invention may comprise at least one gRNA, specifically, using the CRISPR-Cas system specified above. In more specific embodiments, gRNAs that target the exon 4 of the human TYR transcript may comprise the nucleic acid sequence as denoted by any one of SEQ ID NOs. 20 to 27, or any variants, homologs or derivatives thereof. In yet some further embodiments, the gRNAs that target the exon 4 of the mouse TYR transcript may comprise the nucleic acid sequence as denoted by any one of SEQ ID NOs. 16 and 17 (targeting the 5′ splice site) and SEQ ID NOs. 18 and 19 (targeting the 3′ splice site), or any variants, homologs or derivatives thereof. In some embodiments the splicing modulating agent/s (e.g., AON/s, gRNA) of the invention may target the HNRNPAB gene. In yet some further embodiments, the splicing modulating agent used by the kits of the invention may comprise at least one AON. Thus, in some specific embodiments, the nucleic acid sequences that participate or affect splicing of exon 6 is targeted using AONs. In more specific embodiments, the AONs that target the human hnRNPAB exon 6 may comprise the nucleic acids sequence as denoted by any one of SEQ ID NOs. 67 to 86, or any variants, homologs and derivatives thereof. In yet some further embodiments, the splicing modulating agent used by the kits of the invention may comprise at least one gRNA, specifically, using the CRISPR-Cas system specified above. Thus, in some specific embodiments, the nucleic acid sequences that participate or affect splicing of exon 6 of hnRNPAB is targeted using at least one gRNA. In more specific embodiments, gRNAs that target the exon 6 of the human hnRNPAB transcript may comprise the nucleic acid sequence as denoted by any one of SEQ ID NOs. 59 to 66, or any variants, homologs or derivatives thereof. In yet some further embodiments, the gRNAs that target the exon 6 of the mouse hnRNPAB transcript may comprise the nucleic acid sequence as denoted by any one of SEQ ID NOs. 57 and 58 (targeting the 3′ splice site), or any variants, homologs or derivatives thereof.

In yet some further aspects thereof, the invention provides methods for identifying a candidate target gene suitable and effective for induction of at least one aberrant splicing event to produce a neoantigen in at least one target cell of a mammalian subject. In some embodiments, the method of the invention may involves as a first step, the use of a computational pipeline to scan all coding transcripts of the mammalian subject (e.g., human and mouse) to identify transcripts that meet the following criteria: The first criteria requires that the transcripts may comprise a minimum of 3 exons. The second criteria requires that at least one of the exons, that is not first or last, is of a length not divisible by three (3). In some specific and non-limiting embodiments, the length of the exon before last, is not divisible by 3. The third an optional criteria requires that the candidate transcripts are significantly overexpressed in at least one human cancer or at least a potential subject. Transcripts meeting these criteria constitute a potential target bank for antisense oligo manipulation.

In the second step of the methods of the invention, new mRNAs and consequent protein sequences are inferred by simulating exclusion (‘skipping’), inclusion of exons with lengths not divisible by 3. Skipping exons of lengths not divisible by 3 is expected to offset protein synthesis machinery and lead to creation of new peptides. Predicted peptides comprising at least one new amino acid (i.e. an amino acid residue that is different than the one in the original protein) before encountering a stop codon are considered new proteins and selected for further stages.

The third step, involves providing at least one predicted peptide translated from the predicted mRNA of the previous step, wherein each of the peptides comprise at least one of the amino acid residue that differ from a natural product produced in the mammalian subject. In more specific and non-limiting embodiments, the amino acid (AA) sequence, including the new peptide sequence plus 7 adjacent AAs from the original protein, are divided into flanking stretches of all possible 8-mers (eight-mer peptides that comprise eight amino acid residues). Similarly, the new sequence plus 8 adjacent AAs from the original protein is divided to create all possible flanking 9-mers (nine-mer peptides that comprise nine amino acid residues); the new sequence plus 9 adjacent AAs from the original protein is divided to create all possible flanking 10-mers; the new sequence plus 10 adjacent AAs from the original protein is divided to create all possible flanking 10-mers; the new sequence plus 11 adjacent AAs from the original protein is divided to create all possible flanking 12-mers; the new sequence plus 12 adjacent AAs from the original protein is divided to create all possible flanking 13-mers; and finally the new sequence plus 13 adjacent AAs from the original protein is divided to create all possible flanking 14-mers (the flanking segments are referred to herein as X-mers). All X-mers are then evaluated with specialized software (e.g. NetMHCpan (Jurtz et al., 2017, J. Immunol., 199, 3360-3368)) for their ability to bind MHC class I molecules. Peptides predicted to bind at least one known human (or mouse) MHC allele are prioritized. Similarly binding to MHC class II molecules can be evaluated for peptides of up to a length of 22 amino acids (22-mers).

In the next step, the X-mers peptides selected by the previous step as potentially binding MHC molecules, are compared with a comprehensive data source of all known human (or mouse) proteins using BLASTp software (Altschul et al., 1990, J. Mol. Biol., 215, 403-410). Only x-mers that do not naturally occur in the mammalian subject, specifically, human or mouse, are further selected.

The next step is an optional step, where additional computational tools directed at evaluating peptide immunogenicity are employed to further prioritize targets e.g. (Calis et al., 2013, PLoS Comput. Biol., 9, e1003266).

Thus, a further aspect of the invention relates to a method for identifying a candidate target gene for induction of at least one aberrant splicing event to produce a neoantigen in at least one target cell of a mammalian subject. In more specific embodiments, the method of the invention may comprise the steps of:

In a first step (a), selection and/or identification of coding transcripts of the mammalian subject that are characterized by at least one of: (i) the coding transcripts comprise at least three exons; (ii) at least one of the exons is of a length not divisible by three; and (iii) the coding transcripts comprise at least one intron. In yet some further embodiments, the coding transcripts comprises at least one target sequence that aberrant splicing mediated by such sequence leads to a frame shift that creates a neoantigen that does not exist in the human proteome. Still further, in some embodiments, peptides derived from such neoantigen, specifically 9-mer peptides (peptides comprising nine amino acid residues), display high affinity to HLA molecules and are therefore immunogenic. Moreover, such peptides do not exist in a mammalian proteome, specifically, the human proteome.

The next step (b), involves providing at least one predicted mRNA formed or transcribed by at least one aberrant splicing event of at least one of the coding transcripts selected in step (a). in some embodiments, the aberrant splicing event involves a nucleic acid sequence comprised within at least one of: (i) an exon that is of a length not divisible by three; (ii) least one intron located upstream or downstream to said exon; (iii) at least one splicing junction flanking said exon; and (iv) at least one splicing junction within the transcript. It should be noted that in some embodiments the predicted mRNAs formed by such aberrant splicing event encode at least one protein product that is the neoantigen of the invention. This neoantigen differs in at least one amino acid residue from a natural product produced in the cell of the mammalian subject.

The next step (c) of the method of the invention involves providing at least one predicted peptide derived from said neoantigen translated from said predicted mRNA of step (b). It should be noted that each of the predicted peptides comprise at least one amino acid residue that differ from a natural product produced in said mammalian subject. In yet some further embodiments, such peptides may comprise between 8 to 22 amino acid residues. More specifically, such peptides may be in the length of any one of 8, 9, 10, 11, 12, 13, 14, 15, 16, 17, 18, 19, 20, 21, 22 amino acids. It must be understood that the neoantigen formed may be of any length not limited to 22 amino acid resides, however, in some embodiments, the predicated in silico peptides derived from such neoantigen for the purpose of further selection, are of length of between about 8 to about 22 residues. Lengths suitable for binding to MHC molecules. In more specific embodiments, when the aberrant splicing is induced in human target genes, the peptide derived from the neoantigen may bind HLA-I and/or HLA-II molecules. More specifically, peptides comprising between about 8 to about 15 amino acid residues may bind HLA-I molecules, and peptides comprising between about 8 to about 22 amino acid residues may bind HLA-II molecules. Still further, in some embodiments the peptides derived from the neoantigen of the invention may comprise nine amino acid residues and are capable of binding, preferably with strong affinity, to any HLA allele. In some specific and non-limiting embodiments, the peptides may be capable of binding with strong affinity to at least one of the HLA alleles: HLA-A01:01, HLA-A02:01, HLA-A03:01, HLA-A11:01, HLA-A23:01, HLA-A24:02, HLA-A33:03, HLA-B07:02, HLA-B08:01, HLA-B44:02, HLA-C01:02, HLA-C04:01, HLA-C05:01, HLA-C06:02, HLA-C07:01, HLA-C07:02 and HLA-C08:01.

The next step (d), involves selecting or identifying from the at least one of the predicted peptides of (c), peptides that bind at least one of MHC class I and MHC class II molecules of said mammalian subject.

In the next step (e), identifying from the peptides selected in step (d), peptides that do not naturally occur in said mammalian subject.

It should be noted that the identified peptides may in some embodiments comprise the neoantigen created by the invention. In yet some further embodiments, the peptides may be comprised within the neoantigens. In yet some further embodiments, the sequences encoding the peptides are comprised within a gene identified as a candidate target gene.

In yet some further embodiments, the target gene or any target transcript thereof may be a gene differentially expressed in cancer tissue or cell, specifically, overexpressed in the cancer tissue as discussed herein before in connection with other aspects of the invention. Still further, the target sequence within the target gene and/or transcript is a target that aberrant splicing mediated by such sequence leads to a frame shift that creates an antigen, that does not exist in the human proteome, and therefore referred to herein as a neoantigen. Still further, in some embodiments, peptides derived from such neoantigen, specifically 9-mer peptides (peptides comprising nine amino acid residues), display high affinity to HLA molecules and are therefore immunogenic. Moreover, such peptides do not exist in a mammalian proteome, specifically, the human proteome.

All scientific and technical terms used herein have meanings commonly used in the art unless otherwise specified. The definitions provided herein are to facilitate understanding of certain terms used frequently herein and are not meant to limit the scope of the present disclosure.

Before specific aspects and embodiments of the invention are described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. More specifically, the terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%. It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

### EXAMPLES

Experimental Procedures

Alignment Analysis (STAR)

Sequence alignment is the comparison DNA, RNA of protein sequences, in order to highlight similarity between them. STAR algorithm is a fast and accurate algorithms, which is crucial for analyzing large transcriptome datasets (Dobin et al., 2013, Bioinformatics, 29, 15-21).

SAM Tools

SAM tools provide various utilities for manipulating alignments in the SAM format, including sorting, merging, indexing and generating alignments in a per-position format (Li et al., 2009, Bioinformatics, 25, 2078-2079).

UCSC Genome Browser

The UCSC browser is used for various imaging purposes that include, profiling, editing candidates and has many tracks such as conservation tracks, lift-over, in-silico PCR and gene sorter (Haeussler et al., 2019, Nucleic Acids Res., 47, D853-D858).

UCSC Table Browser

Provides data on genes and genes prediction (coding, UTR, introns), SNPs annotations etc.

ANNOVAR

It is an efficient tool to annotate functional consequences of genetic variation from high-throughput sequencing data (Wang et al., 2010, Nucleic Acids Res., 38, e164).

Perl Programming

Perl scripts are used for many purposes such as data analysis, text manipulation, format conversions and more. It enables analysis of sequencing output.

R Programming

R provides a wide variety of statistical and graphical techniques that may be used for data analysis.

Java Programming

Java is a useful programing language useful for big data and is used to remove exons from certain genes and predict the resulting new proteins.

Python Programming

Python is used to run many bioinformatics tools. Its modules enables calculations and statistical summary.

Differential Expression Analysis

A basic task in the analysis of count data from RNA-Seq is the detection of differentially expressed genes. The count data are presented as a table which reports, for each sample, the number of reads that have been assigned to a gene. DESeq2 (Love et al., 2014, Genome Biol., 15, 550) and Limma voom (Law et al., 2014, Genome Biol., 15, R29) are algorithms used to quantify and assign significance to the difference in gene expression across samples.

TCGA Database

The Cancer Genome Atlas (TCGA) contains clinical information, genomic characterization data, and high level sequence analysis of the tumor genomes and transcriptomes (Tomczak et al., 2015, Contemp. Oncol. (Poznan, Poland), 19, A68-77).

GTEx

Genotype-Tissue Expression (GTEx) program provides valuable insights into the mechanisms of gene regulation by studying human gene expression and regulation in multiple tissues from health individuals. It can be utilized for studying healthy skin samples in order to compare them with cancer samples (Lonsdale et al., 2013, Nat. Genet., 45, 580-585).

Optitype

It enables precision HLA typing from next-generation sequencing data. This tool can identify HLA for MHC class1 only and is run by python (Szolek et al., 2014, Bioinformatics, 30, 3310-6).

NetMHCpan

A program for HLA binding selection. and enables prediction of peptide-MHC class I binding using artificial neural networks (Lundegaard et al., 2008, Nucleic Acids Res., 36, W509-W512). NetMHCpan is designed to identify MHC-1 binding peptides of a length ranging from eight to 14 amino acids, the inventors primarily used the length of nine as it occurs frequently and can be predicted accurately. However, all other lengths may be included.

Identifying Existing 9Mers Via Exact Matching

Full human proteome sequences were downloaded from UniProtKB database (PMID:30395287) using the following query: “organism: “Homo sapiens (Human) [9606]” AND proteome:up000005640″. Each 9-mer was compared with all human proteome sequences via a python script.

Upregulated Genes/Isoforms in Cancer

Precompiled lists of upregulated genes and isoforms in cancer, achieved by comparing cancer data from the TCGA project with a compendium of normal tissue data from the GTEx project, were downloaded from the GEPA server [PMID:28407145]. The inventors required both the isoform and the full gene be upregulated 10-fold in at least one of the following cancers (see table of cancer abbreviations): “ACC”, “BLCA”, “CESC”, “COAD”, “DLBC”, “ESCA”, “HNSC”, “LAML”, “LGG”, “PRAD” “READ”, “SKCM”, “STAD”, “TGCT”, “THCA”, “THYM”, “UCEC”, “UCS”. Or 5-fold in at least one of the following cancers: “BRCA”, “GBM”, “KICH”, “KIRC”, “KIRP”, “LIHC”, “LUAD”, “LUSC”, “OV”, “PAAD”. The later were chosen because of their high relevance due to existing drug delivery possibilities and high frequencies of occurrence. Abbreviations are listed in Table 3 herein after.

RT-PCR

Total RNA was extracted with TRI Reagent (Sigma), and cDNA was synthesized from 1 μg RNA using MMLV reverse transcriptase and oligo dT in a final volume of 250 according to the manufacturer's instructions (Promega). PCR was conducted on 1 μl of cDNA by KAPA 2G Fast HS ReadyMix PCR kit (KAPA Biosystems). PCR conditions were as described in manufacturer's protocol of ReadyMix with the addition of 5% (v/v) DMSO for 30 or 35 cycles. PCR products were separated on 2% agarose gels or by LabChip GX/GXII Touch (PerkinElmer).

CRISPR/Cas9 Modulation to Induce Aberrant Splicing

sgRNA targeting the splice sites of the target exon is inserted into lenti-CRISPR v2 vector, digested with BsmBI restriction enzyme and the construct is verified by sequencing. Chosen cells are transduced with either control sgRNA, or sgRNAs against 3′ or 5′ splice sites of the target exon. Infected cells are grown under puromycin selection (2 μg/ml) for 72 h. The efficiency of the CRISPR-mediated splicing modulation is determined by RT-PCR: Total RNA is extracted with TRI Reagent and 1 μg of total RNA is used for cDNA synthesis. PCR is conducted on the cDNA using primers from the exon before the targeted exon and from the exon following the target exon and the products are separated on 2% agarose gels.

### Example 1

### Bioinformatics Identification of Novel Antigens Induced by Aberrant-Induced Splicing (AIS)

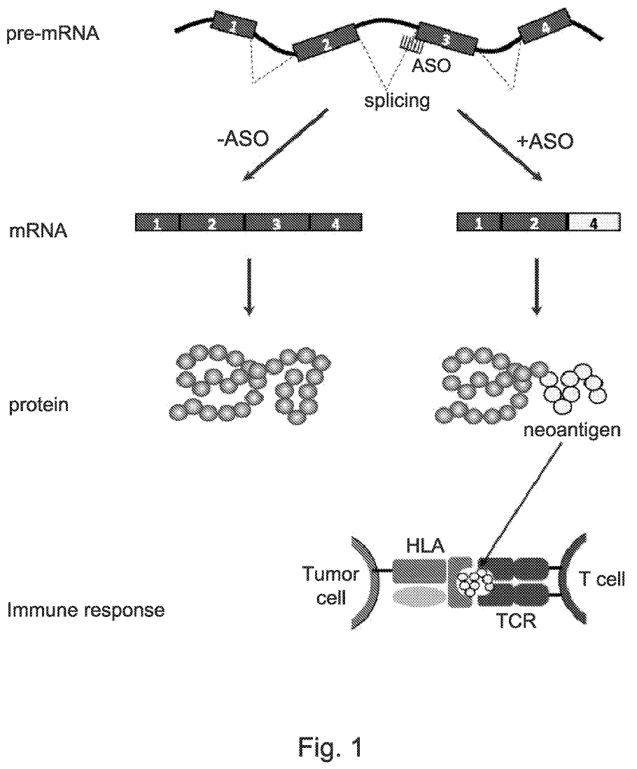

As illustrated by FIG. 1, blockage of specific splice junctions using antisense oligonucleotides (ASOs), creates novel splicing isoforms that are translated into proteins with novel epitopes that may serve as neoantigens for immune recognition of the tumor cells. The display of a new epitope can exert a stronger anti-tumor immune response than the display of only a single point amino acid mutation. Definition of appropriate splice junctions is therefore an initial step. The starting analysis concerns a bioinformatics prediction of all human and mouse genes/transcripts, screening for altered splicing patterns generated hypothetically by exon skipping or insertion. These splicing events enable creation of a novel artificial variant bank, which is not expressed naturally in the body and thus is not immune-tolerant. Selected splicing junctions are those of exons that have a length not divisible by 3 (to induce a shift in the frame of translation). It is preferential but not critical to target exons, that upon exclusion, will not lead to a product that is targeted for degradation by the nonsense mediated RNA decay (NMD) process (e.g. targeting exons that are one before last). For experimental validation it is desired that the splice junctions are shared between human and mouse, but this is not a requirement for a successful drug. Exclusion (or inclusion) of target exons is computationally simulated, resulting with the prediction of aberrant isoforms and new protein products (if any). In order to trigger an immune response, it is required that the new protein should not exist in the natural human (or mouse) proteome. This can be verified via use of NCBI BLASTp or exact sequence matching with the entire human proteome (see Experimental procedures). New predicted proteins are further analyzed for peptides that can potentially bind MHC/HLA-molecules, a key step of immune recognition, using ‘NetMHCpan’ program for HLA binding selection (Lundegaard et al., 2011, J. Immunol. Methods, 374, 26-34). It is possible to identify patient HLA subtype using computational tools such as ‘Optitype’ software (Andreatta and Nielsen, 2016, Bioinformatics, 32, 511-517) from patient sequencing data. Target candidates may be further analyzed with tools such as the Immune Epitope Database (IEDB) to assess their immunogenic potential (Vita et al., 2018, Nucleic Acids Res.). To assure immune recognition as non-tolerized antigens, potential targets may be further filtered based on a collections of thymus-spliced variants e.g. (Danan-Gotthold et al., 2016, Genome Biol., 17, 219).

### Example 2

### Tyrosinase (TYR) is a Potential Target for AIS in Melanoma

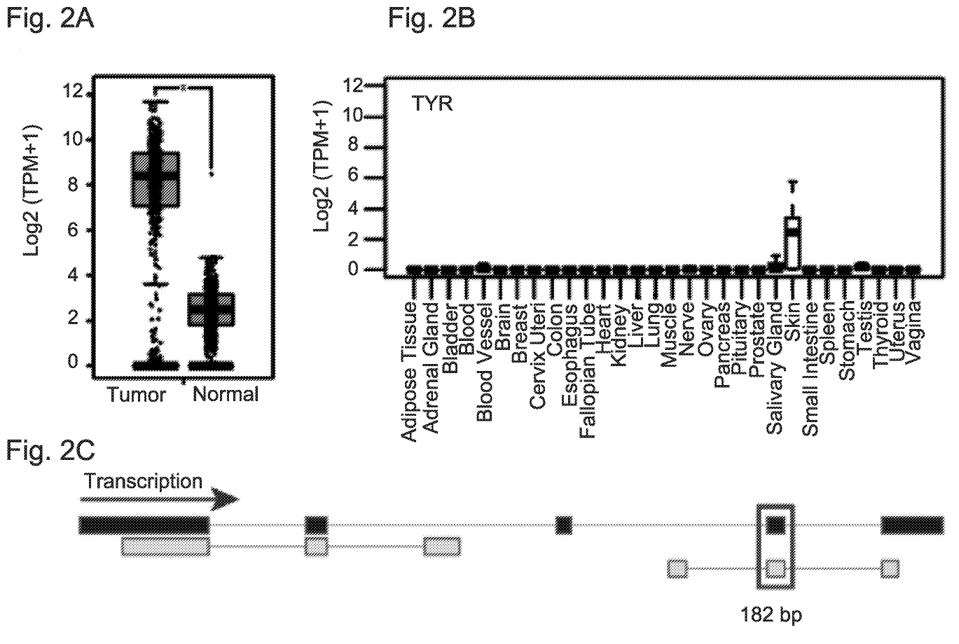

To direct the effect of aberrant splicing to the tumor, it is advantageous to choose target genes which are overexpressed in the tumor compared to matching healthy tissue and also have limited expression across most other healthy tissues. Common targets shared across a large portion of cancer patients, are first priority. However, targets can be tailored personally due to the genomic and transcriptomic repertoire of each patient. Focusing on melanoma, 461 tumor samples from TCGA (The Cancer Genome Atlas Data Portal) were compared with 557 healthy skin samples from GTEx (using differential expression analysis) and 2,477 human genes were identified with significantly elevated expression in the tumor (log 2 fold change >1 and adjusted p-value <0.001). Genes were further filtered to include at least one exon of a length not divisible by 3 and to be conserved in mouse. TYR emerged as suitable candidate (FIG. 2) as its expression in Melanoma is upregulated ˜60 fold compared to normal skin (FDR<0.001, FIG. 2A). Moreover, based on GTEx data, TYR expression across the majority of healthy tissues is limited (FIG. 2B). GTEx data can further be used to identify the gene isoforms expressed in specific tissues—skin in the present case (FIG. 2C). Examining expression levels of TYR across a variety of mouse, healthy and malignant, cell types demonstrated TYR expression only in melanoma (B16), and mouse embryonic fibroblasts (MEF) cells FIG. 2D. The dominantly expressed isoform of Human (and mouse) TYR gene comprises five exons. Excluding human exon 4 (SEQ ID NO. 1), of length 182 bp (not divisible by 3) from transcript NM_000372.4, is predicted to induce a frameshift which would create nine new amino acids (RPRLFSRLH, also denoted by SEQ ID NO. 2) at the C-terminus of the translated protein before encountering a stop codon. (FIG. 2C). FIG. 2E shows protein blast results using the last 17 residues (LLHHAFVDRPRLFSRLH, also denoted by SEQ ID NO. 3) of the new isoform against all human non-redundant sequences (using the NCBI BLASTp). No exact match was found indicating that this isoform does not occur naturally in humans. Analysis of the new protein with NetMHCpan identified two 9-mer peptides (AFVDRPRLF and HAFVDRPRL, referred to as SEQ ID NOs. 14, 89, respectively) predicted to bind multiple HLA molecules and have the potential to induce an immune response. These 9-mers include part of the original and part of the new sequence. Via exact matching with the natural human proteome the inventors verified that these peptides are absent from the natural human proteome (see Experimental procedures). A similar splicing event also occurs in mouse when removing exon 4 from mouse Tyr transcript NM_011661.5 (the transcript is denoted by SEQ ID NO. 11, also 182 bp in length and not divisible by 3). Nine new amino acids are predicted to form in the aberrant protein (RSRLLQKLY, also denoted SEQ ID NO. 9). The new protein includes a 9-mer (HAFVDRSRL, also denoted as SEQ ID NO. 13) predicted by NetMHCpan to bind the H-2-db allele of C57BL/6 mice. This 9-mers includes part of the original and part of the new sequence and does not exist in the natural mouse proteome.

### Example 3

### Inducing Novel Isoforms Using ASOs

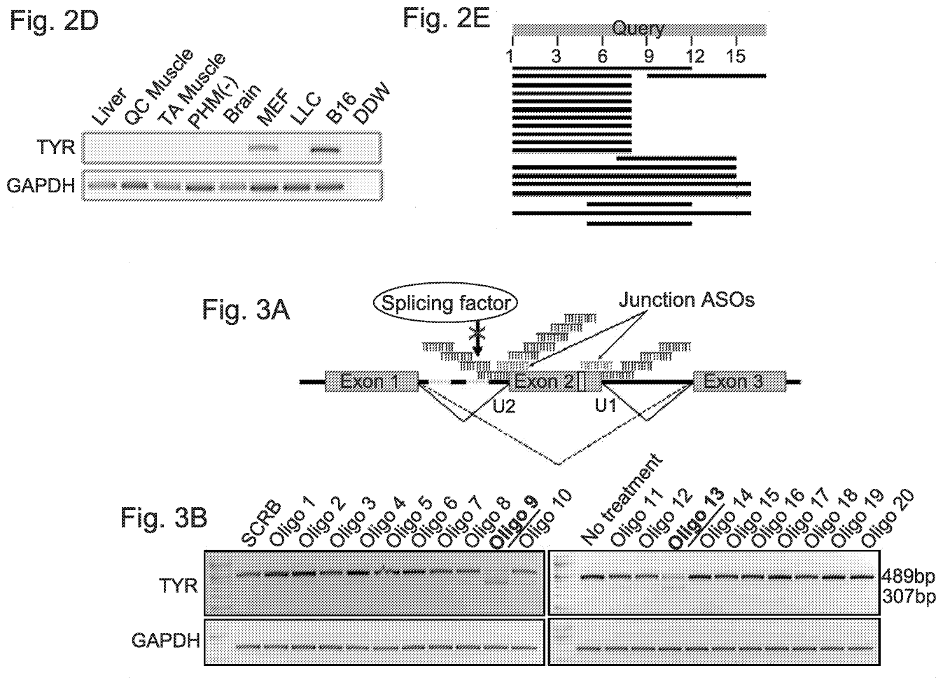

The use of ASOs in order to induce exclusion of exon 4 from TYR and to force synthesis of a novel TYR isoform was next demonstrated. To identify potent ASOs that can efficiently induce exon skipping, a “mini screen” was performed where 20 oligos covering overlapping windows flanking the boundaries of the target exon 4 of the mouse TYR, also denoted by SEQ ID NO. 8, were tested in B16 mouse Melanoma cells (FIGS. 3A-3B). Oligos 9 and 13, as denoted by SEQ ID NO. 4 and SEQ ID NO. 5, respectively, demonstrated efficient skipping of exon 4, as confirmed by RT-PCR (FIG. 3B). The ability to control and optimize abundance of the novel isoform was further demonstrated through changes in oligo dose (FIG. 3C). Finally, Sanger sequencing was used to confirm the existence of the expected novel TYR isoform that comprise the amino acid sequence as denoted by SEQ ID NO. 9 (FIG. 3D-3E), in the C-terminus thereof.

### Example 4

### Vaccination with Immunogenic TYR Peptide Activates the Immune System in C57BL/6 Mice

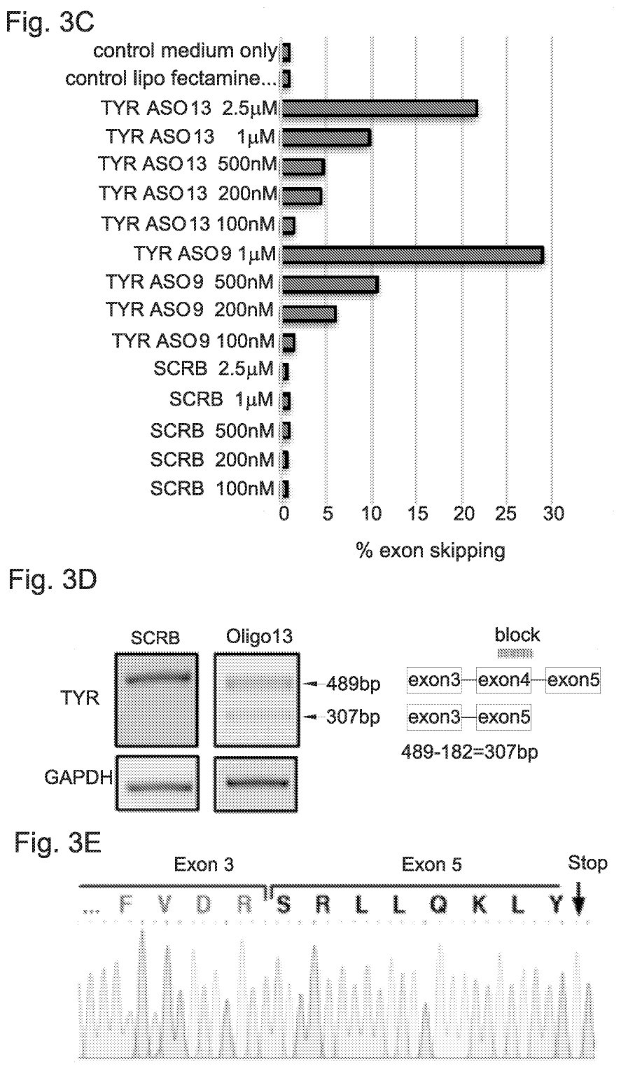

This example demonstrates that the aberrant isoform produced by skipping exon 4 of the TYR gene creates peptides that can activate the immune system in C57BL/6 mice. More specifically, FIG. 4 demonstrates a vaccination experiment where mice were vaccinated and then challenged with peptides from the TYR isoform missing exon 4 and control peptides. C57BL/6 mice were immunized in three groups (10 mice per group) as follows: (1) with adjuvant (10 μg of MPLA and 100 μg of poly(I:C) per mouse)—referred to as “Adj”, (2) with a combination of aberrant TYR peptides (the predicted MHC-I binding 9-mer and 20 C-terminus amino acids of the aberrant TYR isoform, as denoted by SEQ ID NOs. 13 and 12 respectively, 50 μg each) plus the adjuvant—referred to as “TYR” and (3) with Ovalbumin (OVA) peptide (50 μg per mouse) plus the adjuvant—referred to as “OVA”. OVA is an 8mer peptide from the chicken protein Ovalbumin (amino acid sequence: SIINFEKL, ad denoted herein by SEQ ID NO. 15), that serves as an immunogenic positive control. After three immunizations on days 0, 7 and 14, spleens were collected on day 20 and T cells were isolated and tested. FIG. 4A shows the average IFN-γ levels measured for each group of mice. Cells stimulated with CD3 had high levels of IFN-γ, as expected. T cells isolated from mice immunized with OVA and stimulated with OVA produced high levels of IFN-γ while T cells isolated from mice immunized with TYR or with Adj and stimulated with OVA did not. In addition, T cells isolated from mice immunized with TYR and stimulated with TYR displayed higher levels of IFN-γ compared to the other two groups stimulated with TYR but immunized with OVA or Adj. Normalized % of IFN-γ+ cells out of all CD8+ cells is shown for individual mice in the TYR immunized group in FIG. 4B. Six out of 10 mice showed activation of the immune system after stimulation with the TYR peptide compared to stimulation with the OVA peptide and with no stimulation. Using the same experimental design with an ELISA assay shows a similar trend (FIGS. 4C and 4D). Six out of 10 mice showed clear activation of the immune system after stimulation with the TYR peptide compared to the controls. Thus, analysis of the of cells from splenocytes of the vaccinated mice using FACS (FIGS. 4A-4B) and ELISA (FIGS. 4C-4D) indicate successful vaccination and activation of an immune response against the peptides from the aberrant TYR isoform.

### Example 5

### TYR Splicing Modulation with CRISPR/Cas9 Results in a New Isoform of TYR

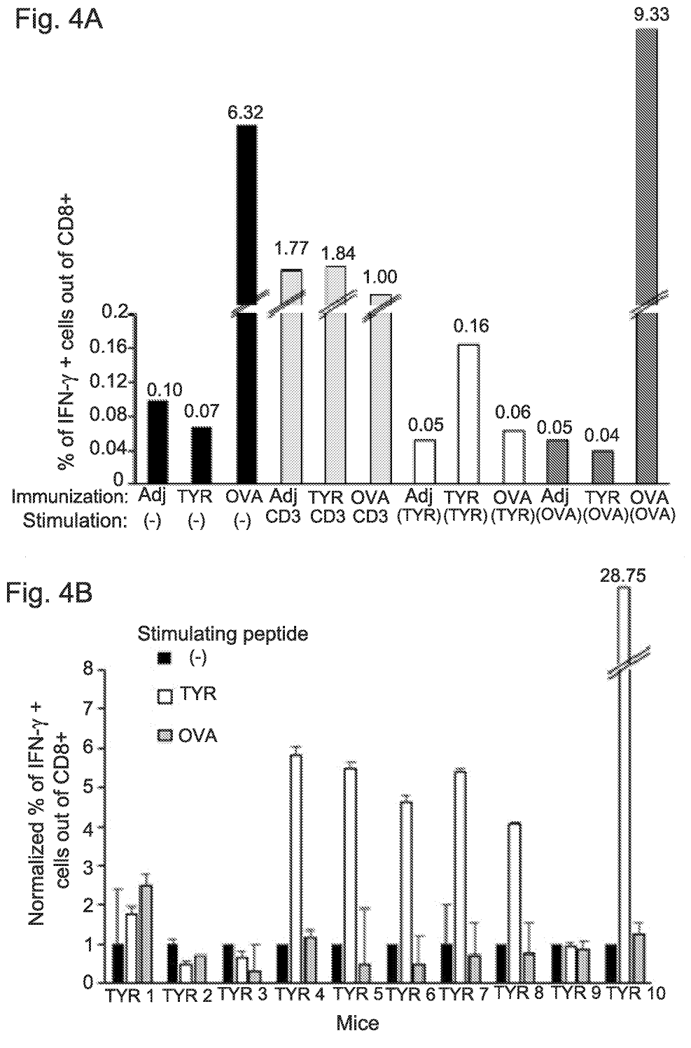

The inventors next used a CRISPR cas9 system targeting the exon-intron junctions of exon 4 in Tyr mouse isoform NM_011661.5 to induce skipping of exon 4 in C57BL/6 mouse cells. RT-PCR performed on B16-F1 mouse melanoma cells transduced with either control sgRNA (CRISPR control) or sgRNAs against 3′ or 5′ splice sites of exon 4 (TYR 3′ss or TYR 5′ss respectively, as denoted by SEQ ID NOs. 16, 17, 18, 19), reveals a new TYR product matching the length expected by exclusion of exon 4, under CRISPR TYR 3′ss and TYR 5′ss treatments but not in the control. This indicates efficient CRISP induced skipping of exon 4 (FIG. 5).

### Example 6

### TYR Splicing Modulation with CRISPR/Cas9 does not Affect the Cancerous Properties of B16-F1 Cells

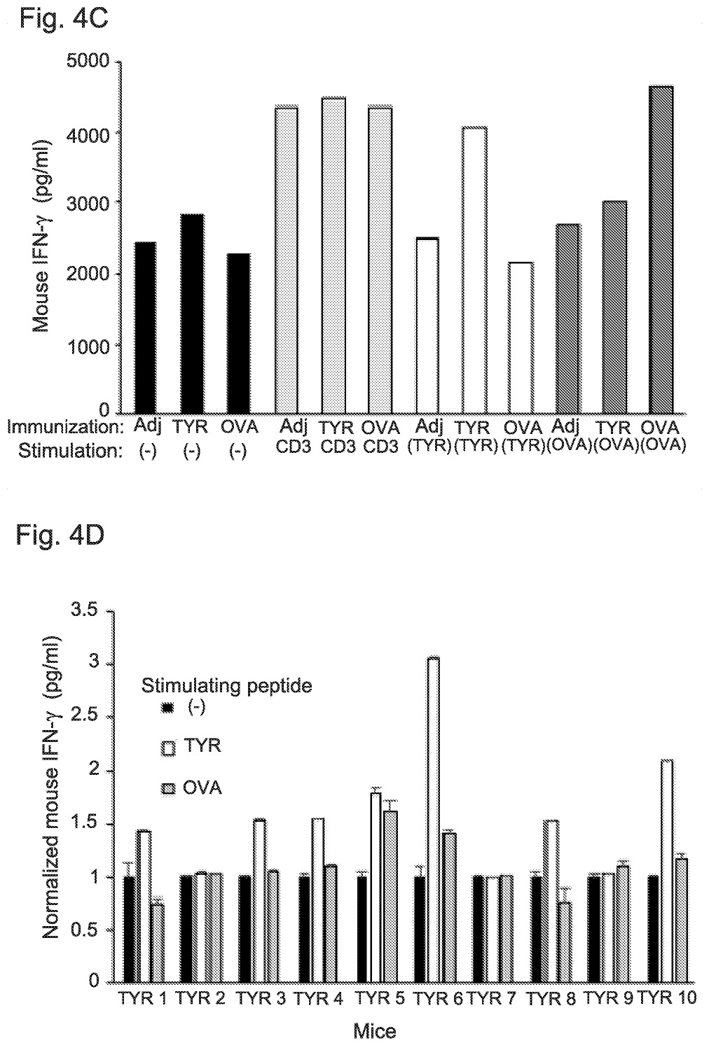

Next, the inventors show that cancer properties of B16-F1 cells under treatment of CRISPR/cas9 that induces skipping of exon 4 are similar to those of WT B16-F1 cells. More specifically, A clonogenic assay was performed on B 16-F1 cells transduced with either control sgRNA (CRISPR control) or sgRNAs against 3′ or 5′ splice sites of exon 4 of the TYR gene (TYR 3ss or TYR 5ss, as denoted by SEQ ID NOs. 16-19, respectively). A clonogenic assay was performed on B16-F1 cells transduced with either control sgRNA (CRISPR control) or sgRNAs against 3′ or 5′ splice sites of exon 4 of the TYR gene (TYR 3ss or TYR 5ss, as denoted by SEQ ID NOs. 16-19, respectively). After 14 days cells were fixated with 2.5% glutaraldehyde solution for 10 min, stained with 1% methylene blue solution, photographed and counted. As shown in FIG. 6A, the number of colonies was similar in all groups. Additionally, a proliferation assay performed on these cells shows similar growth curves for all groups (FIG. 6B). Finally, an anchorage-independent growth assay resulted in a similar number of colonies in all groups (FIG. 6C). Thus, clonogenic, proliferation and anchorage independent growth assays indicate the cancerous properties of the treated B16-F1 cells were significantly altered in the treated cells.

### Example 7

### TYR Splicing Modulation with CRISPR/Cas9 in B16-F1 Cells Inhibits Tumor Growth in C57BL/6 Mice but not in Immune-Deficient (NOD-SCID) Mice

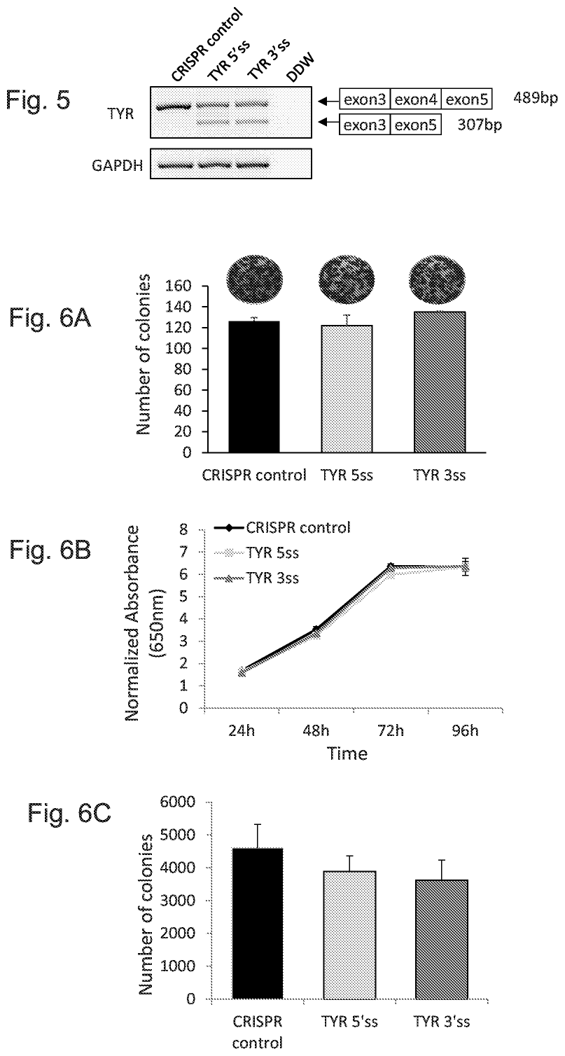

Next the inventors show that CRISPR/cas9 modified B16-F1 cells expressing an aberrant isoform of TYR trigger an immune response that inhibits tumor growth in C57BL/6 mice. B16-F1 cells, transduced with either sgRNAs against the 3′ splice site of exon 4 of the TYR gene (CRISPR TYR gRNAs, as denoted by SEQ ID NOs. 18-19) or with control sgRNAs (CRISPR control), were injected into C57BL/6 (200,000 cells/50 μl per mouse intradermally) and NOD-SCID mice (1·10⁶ cells/200 μl per mouse subcutaneously). FIG. 7 demonstrates that B16-F1 CRISPR TYR cells formed smaller tumors, which developed slower compared to tumors originating from CRISPR control cells (T-test p-value=0.03 on the last day of measurement, FIGS. 7 A and 7C). This difference was not observed in NOD-SCID mice which have T and B cell deficiencies, reduced NK cell function, defective dendritic cells and macrophages—thereby indicating that the effect in the C57BL/6 mice was the result of immune system activity (FIGS. 7B and 7C). To further investigate this immune response, T-cells were isolated from mice splenocytes, and either stimulated with aberrant immunogenic TYR peptides (SEQ ID NOs. 12 and 13) or not stimulated (referred to as ‘activated’ and ‘naïve’ respectively). Cells were then stained for CD8 and IFN-γ and analyzed by flow cytometry. FIG. 7D indicates higher fractions of activated (IFN-γ+) CD8 T-cells for cells from mice that were previously exposed to the aberrant TYR isoform (expressed in their tumors) and then activated with immunogenic TYR peptides (‘Activated Tyr’ group) than cells that were either not previously exposed (‘Activated control’ group) or previously exposed but not activated (Naïve Tyr′ group) or not previously exposed and not activated (‘Naïve control’ group). One unexplained outlier in the Activated control group is noted.

### Example 8

### Immunization of C57BL/6 Mice with Aberrant TYR Peptides Followed by Injection of B16-F1 Melanoma Cells Harboring TYR Splicing Modulation by CRISPR/Cas9 or Control CRISPR/Cas9

C57BL/6 mice have been immunized with either (1) adjuvant (10 μg of MPLA and 100 μg of poly(I:C) per mouse), or (2) a combination of aberrant TYR peptides (as denoted by SEQ ID NOs. 12 and 13), 50 μg each plus the adjuvant—referred to as “TYR”, or (3) Ovalbumin (OVA) peptide (50 μg per mouse) plus the adjuvant, referred to as “OVA” (as denoted by SEQ ID NO. 15). Immunizations are administered once a week for three weeks. Following immunization, each mouse is injected intradermally with 200,000 cells/50 μl of B16-F1 cells, transduced with either sgRNAs against the 3′ splice site of exon 4 of the TYR gene (CRISPR TYR sgRNAs s, as denoted by SEQ ID NOs. 18-19) or with control sgRNAs (CRISPR control)—10 mice in each group. Tumor volumes are measured three times a week until tumors achieve approved maximum size, then the mice are sacrificed, their spleens collected, and T cells isolated. Similar to the experiment described in Example 4, isolated T cells are seeded in 96-well plates 1·10⁶ cells per well in duplicate and stimulated with different peptides; no stimulation (−), TYR, OVA peptides (SEQ ID NOs. 12, 13 and 15) or anti CD3. T cells are stained for CD8 and IFN-γ and analyzed by flow cytometry. In addition, 72 hours after stimulation, medium is collected and IFN-γ secretion is measured by ELISA assay. Identical injections of B16-F1 cells are performed on NOD-SCID mice as a control. Smaller tumors are expected in mice that were immunized with TYR and injected with B16-F1 cells transduced with CRISPR TYR sgRNAs versus controls.

### Example 9

### Evaluation of the Effect of TYR Splicing Modulation by Oligonucleotides Designed for TYR Exon 4 Skipping into C57BL/6 Mice

C57BL/6 mice have been immunized with either (1) an adjuvant (10 μg of MPLA and 100 μg of poly(I:C) per mouse), or (2) a combination of aberrant TYR peptides (50 μg each SEQ ID NOs. 12,13) plus the adjuvant—referred to as “TYR”, or (3) Ovalbumin (OVA) peptide (SEQ ID NO. 15, 50 μg per mouse) plus the adjuvant, referred to as “OVA” (20 mice in each group). Immunizations are administered once a week for three weeks. B16-F1 cells transfected with 2.5 μM of either SCRB (randomized oligo preserving base composition) or TYR oligo 9 or oligo 13 (as denoted by SEQ ID NOs. 4, 5, respectively) that induce skipping of exon 4 of the TYR pre-mRNA) using lipofectamine 2000 (24 h after transfection) are injected intradermally into C57BL/6 mice. Tumor volumes are measured three times a week until tumors achieve approved maximum size, then the mice are sacrificed, their spleens collected, and T cells isolated. The isolated T cells are seeded in 96-well plates, 1·10⁶ cells per well plates in duplicate, and stimulated with the different peptides; no stimulation (−), TYR, OVA peptide or anti CD3. T cells are stained for CD8 and IFN-γ and analyzed by flow cytometry. In addition, 72 hours after stimulation, medium is collected and IFN-γ secretion is measured by ELISA assay. Identical injections of B16-F1 cells are performed on NOD-SCID mice as a control. Smaller tumors are expected in mice that were immunized with TYR and injected with B16-F1 cells transfected with TYR oligo 9 or oligo 13 versus controls.

### Example 10

### HNRNPAB Gene is Overexpressed in Multiple Human Cancers and Aberrant Splicing of its Exon 6 Leads to Formation of Potentially Immunogenic Peptides

The HNRNPAB human gene is overexpressed across multiple cancers. Based on the GEPIA server (PMID:28407145), 16 cancers studied in the TCGA project overexpress hnRNPAB by at least 2-fold (p-value<0.001 using a one-way ANNOVA test). Removing exon 6 which is the exon before last of a length not divisible by 3 (103 bp in mouse, SEQ ID NO. 48 and 118 bp in humans, SEQ ID NO. 50) from transcript NM_010448.3 in mouse (SEQ ID NO. 49) and transcript NM_004499.3 in human (SEQ ID NO. 51) is predicted to induce a frameshift in translation and create new proteins in mouse (SEQ ID NO. 55) and in human (SEQ ID NO. 56). These new proteins include 128 and 31 new amino acids at their C-terminus for mouse and human respectively and contain potentially immunogenic peptides. For example, the new mouse protein segment includes the 9-mer VPNLTWQTF (also denoted as SEQ ID NO. 52) which is predicted to bind the BALB/c mouse MHC allele H-2-Ld at an affinity of 28.5 nM. As hnRNPAB is overexpressed in cancer and has immunogenic potential upon skipping of exon 6 in both human and mouse—it is a suitable target for demonstrating our invention.

### Example 11

### hnRNPAB Splicing Modulation with CRISPR/Cas9 Results in a New Isoform of hnRNPAB

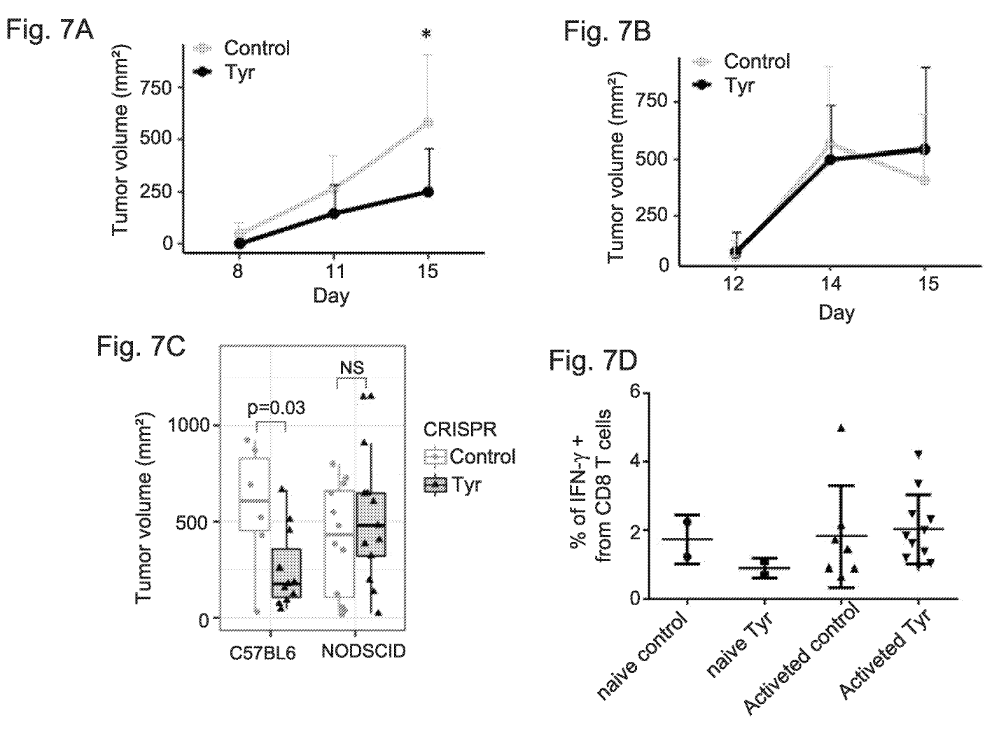

As a preliminary step for in-vivo experimentation, inventors used a CRISPR cas9 system targeting exon-intron junctions (similar to the above) of exon 6 in the mouse hnRNPAB isoform NM_010448.3, to Induce skipping of exon 6 (SEQ ID NO. 48) in 4T1 cells. 4T1 cells were transduced with either control sgRNA (CRISPR control) or sgRNAs against the 3′ and 5′ splice sites of exon 6 (hnRNPAB 3′ss, as denoted by SEQ ID NOs. 57-58 and hnRNPAB 5′ss, as denoted by SEQ ID NOs 87 and 88). RT-PCR products reveal that in addition to the two natural hnRNPAB isoforms, a new product matching the length expected by exclusion of hnRNPAB exon 6 formed only under CRISPR hnRNPAB 3′ss treatment but not under the control and in this case also not under hnRNPAB 5′ss treatment (FIG. 8). Thus, we will use CRISPR hnRNPAB 3′ss for in-vivo experiments.

### Example 12

### hnRNPAB Splicing Modulation with CRISPR/Cas9 in 4T1 Cells Inhibits Tumor Growth in BALB/c Mice but not in Immune-Deficient (NOD-SCID) Mice

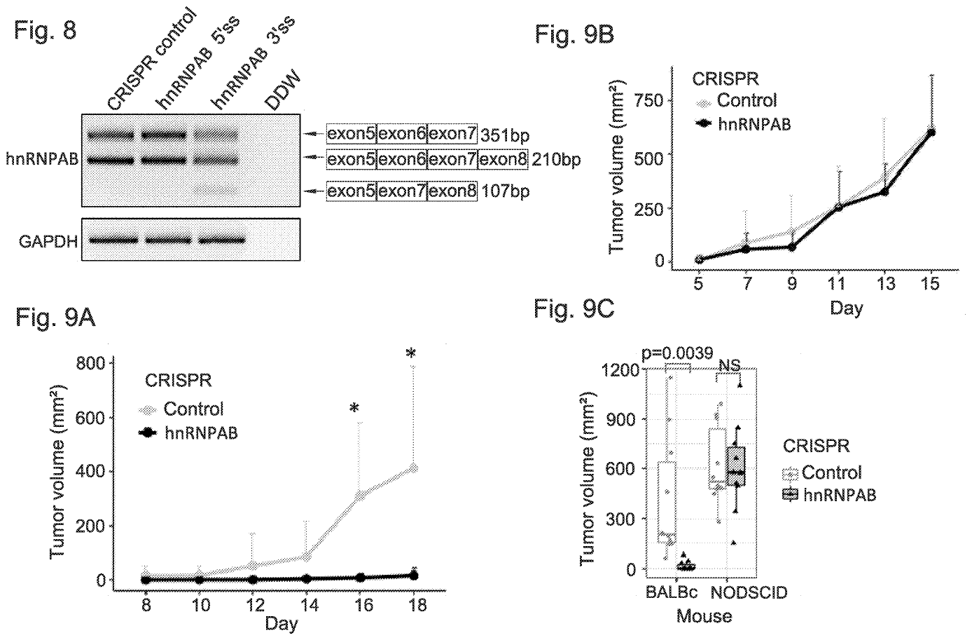

Next the inventors show that CRISPR/cas9 modified 4T1 cells expressing an aberrant isoform of hnRNPAB trigger an immune response that inhibits tumor growth in BALB/c mice. 4T1 cells transduced with sgRNAs against the 3′ splice site of exon 6 of the HNRNPAB gene (CRISPR hnRNPAB, as denoted by SEQ ID NOs 57, 58) or with control sgRNAs (CRISPR control), were injected into BALB/c and NOD-SCID mice (500,000 cells/200 μl per mouse subcutaneously). FIG. 9 demonstrates that 4T1 CRISPR hnRNPAB cells formed smaller tumors (undetectable in some cases), which developed slower compared to tumors originating from CRISPR control cells, (T-test p-value=0.0031 and 0.0039 on the two last days of measurements respectively, FIGS. 9A and 9C). This difference was not observed in NOD-SCID mice that have T and B cell deficiencies, reduced NK cell function, defective dendritic cells and macrophages—thereby indicating that the effect in the BALB/c mice was the result of immune system activity (FIGS. 9B and 9C).

### Example 13

### Immunization of BALB/c Mice with Aberrant hnRNPAB Peptides Followed by Injection of 4T1 Breast Cancer Cells Harboring hnRNPAB Splicing Modulation by CRISPR/Cas9 or Control CRISPR/Cas9

BALB/c mice were immunized with either (1) adjuvant (10 μg of MPLA and 100 μg of poly(I:C) per mouse), or (2) a combination of aberrant hnRNPAB peptides (50 μg each, SEQ ID NOs. 52 and 53, respectively) plus the adjuvant referred to as hnRNPAB, or (3) BALB/c positive control peptide (50 μg per mouse, SEQ ID NO. 54) plus adjuvant—referred to as “positive control”. Immunizations are administered once a week for three weeks. Following immunization each mouse is injected subcutaneously with 500,000 cells/200 μl of 4T1 cells transduced with lentivirus comprising either 3′ splice site sgRNAs against exon 6 of the HNRNPAB gene (CRISPR hnRNPAB, SEQ ID NOs. 57, 58) or control sgRNAs (CRISPR control)—10 mice in each group. Tumors volumes are measured three times a week until tumors achieve approved maximum size, then the mice are sacrificed, their spleens collected, and T cells isolated. The isolated T cells are seeded in 96 plates in duplicate followed by stimulation with the different peptides; no stimulation (−), hnRNPAB, BALB/c positive control peptides or anti CD3. T cells are stained for CD8 and IFN-γ and analyzed by flow cytometry. In addition, 72 hours after stimulation, medium is collected and IFN-γ secretion is measured by ELISA assay. Identical injections of 4T1 cells are performed on NOD-SCID mice as a control. Smaller tumors are expected in mice that were immunized with hnRNPAB and injected with 4T1 cells transduced with CRISPR hnRNPAB sgRNAs versus controls.

### Example 14

### Identification of Target Sequences for Aberrant Splicing in Transcripts Overexpressed in Cancers

To identify therapeutic targets for the induction of aberrant splicing in accordance with the invention, the inventors simulated the removal of the exon before last from all known transcripts in the human genome, provided the exon length was not divisible by three, designed to trigger an offset in the original translation frame. To facilitate use of this method in the treatment of cancer the inventors focused on transcripts that are upregulated in at least one cancer by at least 5-fold compared to normal tissue. This was achieved using information from the GEPIA server which compares TCGA and GTEx data (Tang Z. et al., 2019, Nucleic Acids Res. 47(W1):W556-W560.)—see Experimental procedures. Removal of each exon in these transcripts followed by the consequent formation of an aberrant isoform and its ‘new’ protein product were simulated. Using NetMHCpan software the new protein sequence was analyzed to identify potential strong binding (<=50 nm) 9mers to at least one of the following HLA-alleles, chosen because of their high frequency in various populations: HLA-A01:01, HLA-A02:01, HLA-A03:01, HLA-A11:01, HLA-A23:01, HLA-A24:02, HLA-A33:03, HLA-B07:02, HLA-B08:01, HLA-B44:02, HLA-C01:02, HLA-C04:01, HLA-C05:01, HLA-C06:02, HLA-C07:01, HLA-C07:02, HLA-C08:01.

As the goal is to create immunogenic peptides, the inventors discarded any strong binding 9-mer if it existed in the natural human proteome via exact sequence matching with all human proteome sequences downloaded from UniprotKB (UniProt Consortium, 2013, Nucleic Acids Res. 47(D1):D506-D515.)—see Experimental procedures. Table 2 list 524 exons, that match the criteria above. Genes and transcript ids along with information regarding the predicted strong binding 9-mers are listed as well. More specifically, Table 1 lists the target genes and indicates the transcript and the cancer expressing the specified transcript. The abbreviation for each of the cancers are presented in Table 3.

TABLE 1
Gene names, transcripts and overexpressing cancer

| gene | gene name | transcript | Over-expressing cancers |
|---|---|---|---|
| MPZL1 | myelin protein zero like 1 | NM_001146191.1 | PAAD |
| AGRN | agrin | NM_198576.3 | THYM, OV |
| WLS | Wnt ligand secretion | NM_001002292.3, | GBM |
| | mediator | NM_024911.6 | |
| KCNAB2 | potassium voltage-gated | NM_172130.2 | LAML |
| | channel subfamily A | | |
| | regulatory beta subunit 2 | | |
| SLC2A5 | solute carrier family 2 | NM_003039.2 | KIRC, DLBC, GBM, |
| | member 5 | | LAML |
| SLC9A1 | solute carrier family 9 | NM_003047.4 | PAAD |
| | member A1 | | |
| NKAIN1 | sodium/potassium | NM_024522.2 | BRCA |
| | transporting ATPase | | |
| | interacting 1 | | |
| MAP7D1 | MAP7 domain containing 1 | NM_001286366.1 | PAAD |
| RHBDL2 | rhomboid like 2 | NM_017821.4, | CESC, PAAD |
| | | NM_001304746.1 | |
| CYP2J2 | cytochrome P450 family 2 | NM_000775.3 | KIRC |
| | subfamily J member 2 | | |
| GPSM2 | G protein signaling modulator | NM_001321038.1, | BRCA, OV, RICH |
| | 2 | NM_013296.4 | |
| SORT1 | sortilin 1 | NM_002959.6 | OV |
| CD58 | CD58 molecule | NM_001779.2 | PAAD, GBM |
| NOTCH2 | notch receptor 2 | NM_001200001.1 | LAML |
| CTSS | cathepsin S | NM_001199739.1, | PAAD, LAML, OV, |
| | | NM_004079.4 | TGCT, GBM |
| PMVK | phosphomevalonate kinase | NM_001323011.1, | DLBC, THYM |
| | | NM_006556.3, | |
| | | NM_001323012.1 | |
| STX6 | syntaxin 6 | NM_005819.5 | PAAD |
| CAPN8 | calpain 8 | NM_001143962.1 | PAAD |
| CAPN2 | calpain 2 | NM_001146068.1, | PAAD |
| | | NM_001748.4 | |
| PARP1 | poly(ADP-ribose) polymerase | NM_001618.3 | DLBC, THYM |
| | 1 | | |
| GPR137B | G protein-coupled receptor | NM_003272.3 | PAAD |
| | 137B | | |
| COX20 | cytochrome c oxidase | NM_001312872.1 | PAAD, LUSC, |
| | assembly factor COX20 | | LUAD, DLBC, OV, |
| | | | SKCM |
| RPL22 | ribosomal protein L22 | NM_000983.3 | THYM |
| GPR153 | G protein-coupled receptor | NM_207370.2 | PAAD |
| | 153 | | |
| PDPN | podoplanin | NM_001006625.1, | PAAD, GBM, TGCT |
| | | NM_198389.2 | |
| AKR7A2 | aldo-keto reductase family 7 | NM_003689.3 | THYM |
| | member A2 | | |
| CDA | cytidine deaminase | NM_001785.2 | PAAD |
| GALE | UDP-galactose-4-epimerase | NM_000403.3, | PAAD, THCA |
| | | NM_001127621.1, | |
| | | NM_001008216.1 | |
| IL22RA1 | interleukin 22 receptor | NM_021258.3 | READ, COAD |
| | subunit alpha 1 | | |
| SYTL1 | synaptotagmin like 1 | NM_001193308.1 | CESC, OV |
| THEMIS2 | thymocyte selection | NM_001286115.1. | PAAD |
| | associated family member 2 | NM_001105556.2 | |
| SMPDL3B | sphingomyelin | NM_014474.3 | UCEC, OV |
| | phosphodiesterase acid like | | |
| | 3B | | |
| TINAGL1 | tubulointerstitial nephritis | NM_022164.2 | PAAD |
| | antigen like 1 | | |
| SPOCD1 | SPOC domain containing 1 | NM_001281988.1 | GBM |
| MYCL | MYCL proto-oncogene, | NM_001033082.2 | OV |
| | bHLH transcription factor | | |
| PTPRF | protein tyrosine phosphatase | NM_130440.2 | STAD, OV, ESCA |
| | receptor type F | | |
| ARTN | artemin | NM_001136215.1 | LUSC |
| MAGOH | mago homolog, exon junction | NM_002370.3 | DLBC, THYM |
| | complex subunit | | |
| AK4 | adenylate kinase 4 | NM_013410.3 | PAAD |
| CYR61 | cellular communication | NM_001554.4 | DLBC |
| | network factor 1 | | |
| GBP1 | guanylate binding protein 1 | NM_002053.2 | PAAD, GBM |
| EPHX4 | epoxide hydrolase 4 | NM_173567.4 | OV |
| GFI1 | growth factor independent 1 | NM_001127216.1 | LAML |
| | transcriptional repressor | | |
| CELSR2 | cadherin EGF LAG seven- | NM_001408.2 | OV |
| | pass G-type receptor 2 | | |
| C1orf162 | chromosome 1 open reading | NM_001300835.1. | LAML |
| | frame 162 | NM_174896.3 | |
| C1orf162 | chromosome 1 open reading | NM_001300834.1 | KIRC, GBM, LAML |
| | frame 162 | | |
| CTSK | cathepsin K | NM_000396.3 | DEBC, PAAD |
| ILF2 | interleukin enhancer binding | NM_004515.3 | THYM |
| | factor 2 | | |
| CKS1B | CDC28 protein kinase | NM_001826.2 | PAAD, LUSC, |
| | regulatory subunit 1B | | DEBC, THYM, OV, |
| | | | GBM |
| BCAN | brevican | NM_021948.4 | LGG, GBM |
| MRPL24 | mitochondrial ribosomal | NM_145729.2, | DLBC, THYM |
| | protein L24 | NM_024540.3 | |
| HDGF | heparin binding growth factor | NM_001319188.1, | PAAD, OV |
| | | NM_004494.2, | |
| | | NM_001319187.1, | |
| | | NM_001126051.1 | |
| CD1A | CD1a molecule | NM_001320652.1, | THYM |
| | | NM_001763.2 | |
| CD1B | CD1b molecule | NM_001764.2 | THYM |
| CD1E | CD1e molecule | NM_001185114.1, | THYM |
| | | NM_001042585.2, | |
| | | NM_001042586.2 | |
| TAGLN2 | transgelin 2 | NM_001277223.1, | PAAD, LUSC, |
| | | NM_003564.2 | LUAD, BRCA, |
| | | | READ, STAD, |
| | | | COAD, OV, SKCM, |
| | | | GBM, ESCA |
| F11R | F11 receptor | NM_016946.4 | CESC |
| TSTD1 | thiosulfate sulfurtransferase | NM_001113206.1, | UCEC, CESC, |
| | like domain containing 1 | NM_001113207.1 | THYM |
| PFDN2 | prefoldin subunit 2 | NM_012394.3 | DLBC, THYM, |
| | | | PAAD |
| ELF3 | E74 like ETS transcription | NM_001114309.1, | UCEC, READ, |
| | factor 3 | NM_004433.4 | CESC, OV, COAD |
| CHIT1 | chitinase 1 | NM_001256125.1 | KIRP |
| PIGR | polymeric immunoglobulin | NM_002644.3 | UCEC, PAAD |
| | receptor | | |
| MAP4K4 | mitogen-activated protein | NM_001242559.1 | ESCA |
| | kinase kinase kinase kinase 4 | | |
| MBOAT2 | membrane bound O- | NM_001321266.1, | PAAD |
| | acyltransferase domain | NM_001321267.1, | |
| | containing 2 | NM_138799.3, | |
| | | NM_001321265.1 | |
| ANTXR1 | ANTXR cell adhesion | NM_018153.3 | PAAD |
| | molecule 1 | | |
| KIAA1211L | KIAA1211 like | NM_207362.2 | OV |
| FHL2 | four and a half LIM domains | NM_201555.1, | PAAD, THYM |
| | 2 | NM_001318895.1, | |
| | | NM_001318898.1, | |
| | | NM_001450.3, | |
| | | NM_001039492.2, | |
| | | NM_001318896.1, | |
| | | NM_001318899.1 | |
| CYTIP | cytohesin 1 interacting | NM_004288.4 | LAML |
| | protein | | |
| ACVR1 | activin A receptor type 1 | NM_001111067.2 | PAAD |
| DPP4 | dipeptidyl peptidase 4 | NM_001935.3 | THCA |
| WNT10A | Wnt family member 10A | NM_025216.2 | PAAD, OV |
| DOCK10 | dedicator of cytokinesis 10 | NM_001290263.1 | LAML |
| UGT1A10 | UDP glucuronosyltransferase | NM_019075.2 | PAAD |
| | family 1 member A10 | | |
| UGT1A6 | UDP glucuronosyltransferase | NM_001072.3, | LUSC |
| | family 1 member A6 | NM_205862.1 | |
| MLPH | melanophilin | NM_001281473.1, | PAAD, SKCM |
| | | NM_001042467.2 | |
| PDIA6 | protein disulfide isomerase | NM_005742.3 | DLBC, GBM |
| | family A member 6 | | |
| SF3B6 | splicing factor 3b subunit 6 | NM_016047.3 | DLBC, THYM |
| ATRAID | all-trans retinoic acid induced | NM_016085.4, | GBM |
| | differentiation factor | NM_001170795.1 | |
| CALM2 | calmodulin 2 | NM_001743.5 | PAAD |
| ACTR2 | actin related protein 2 | NM_005722.3 | PAAD |
| PLEK | pleckstrin | NM_002664.2 | GBM, LAML |
| ACTG2 | actin gamma 2, smooth | NM_001615.3 | KIRC, PAAD |
| | muscle | | |
| BOLA3 | bolA family member 3 | NM_001035505.1 | THYM |
| LOXL3 | lysyl oxidase like 3 | NM_001289165.1, | GBM |
| | | NM_001289164.1 | |
| GNLY | granulysin | NM_006433.4 | TGCT |
| CD8A | CD8a molecule | NM_171827.3 | KIRC, THYM |
| CD8B | CD8b molecule | NM_004931.4 | THYM |
| SULT1C4 | sulfotransferase family 1C | NM_006588.3, | KIRP |
| | member 4 | NM_001321770.1 | |
| FAP | fibroblast activation protein | NM_004460.3 | PAAD |
| | alpha | | |
| CERKL | ceramide kinase like | NM_201548.4 | LAML |
| PPIL3 | peptidylprolyl isomerase like | NM_130906.2 | THYM |
| | 3 | | |
| NOP58 | NOP58 ribonucleoprotein | NM_015934.4 | DLBC, THYM |
| IHH | Indian hedgehog signaling | NM_002181.3 | READ, PAAD, |
| | molecule | | COAD |
| SP140 | SP140 nuclear body protein | NM_001005176.2 | DLBC |
| ITM2C | integral membrane protein 2C | NM_001012514.2, | UCS, THYM, |
| | | NM_030926.5, | TGCT, PAAD |
| | | NM_001287241.1, | |
| | | NM_001012516.2 | |
| PLS1 | plastin 1 | NM_001145319.1 | OV, PAAD, READ, |
| | | | COAD |
| FBLN2 | fibulin 2 | NM_001998.2 | PAAD |
| CDCP1 | CUB domain containing | NM_022842.4 | CESC, PAAD, OV |
| | protein 1 | | |
| ITGB5 | integrin subunit beta 5 | NM_002213.4 | PAAD |
| CHCHD6 | coiled-coil-helix-coiled-coil- | NM_001320610.1, | THYM |
| | helix domain containing 6 | NM_032343.2 | |
| BHLHE40 | basic helix-loop-helix family | NM_003670.2 | PAAD |
| | member e40 | | |
| MYD88 | MYD88 innate immune | NM_001172567.1 | PAAD |
| | signal transduction adaptor | | |
| MYRIP | myosin VIIA and Rab | NM_001284426.1 | RICH |
| | interacting protein | | |
| GNL3 | G protein nucleolar 3 | NM_014366.4 | DLBC |
| CMSS1 | cms1 ribosomal small subunit | NM_032359.3 | DLBC, THYM |
| | homolog | | |
| CD96 | CD96 molecule | NM_198196.2 | LAML |
| PARP9 | poly(ADP-ribose) polymerase | NM_001146104.1 | GBM |
| | family member 9 | | |
| TMCC1 | transmembrane and coiled- | NM_001128224.2 | KIRC |
| | coil domain family 1 | | |
| CLDN1 | claudin 1 | NM_021101.4 | LUSC, THCA, |
| | | | READ, CESC, |
| | | | STAD, KIRP, OV, |
| | | | COAD |
| TIMP4 | TIMP metallopeptidase | NM_003256.3 | LGG, GBM |
| | inhibitor 4 | | |
| RPSA | ribosomal protein SA | NM_001304288.1 | PAAD, LUSC, |
| | | | LUAD, DLBC, |
| | | | THYM, LGG, |
| | | | READ, LIHC, TGCT |
| CTNNB1 | catenin beta 1 | NM_001904.3 | PAAD, GBM |
| TDGF1 | teratocarcinoma-derivcd | NM_001174136.1 | READ, THYM, |
| | growth factor 1 | | COAD |
| SEMA3F | semaphorin 3F | NM_001318798.1, | THYM, OV |
| | | NM_004186.4 | |
| RRP9 | ribosomal RNA processing 9, | NM_004704.4 | DLBC |
| | U3 small nucleolar RNA | | |
| | binding protein | | |
| FAM107A | family with sequence | NM_001076778.2 | THYM |
| | similarity 107 member A | | |
| HGD | homogentisate 1,2- | NM_000187.3 | UCEC |
| | dioxygenase | | |
| CD86 | CD86 molecule | NM_175862.4 | GBM |
| PARP15 | poly(ADP-ribose) polymerase | NM_001308320.1 | LAML |
| | family member 15 | | |
| HMCES | 5-hydroxymethylcytosine | NM_001006109.1 | GBM |
| | binding, ES cell specific | | |
| RPL22L1 | ribosomal protein L22 like 1 | NM_001320451.1 | DLBC, THYM |
| DNAJC19 | DnaJ heat shock protein | NM_145261.3, | THYM, GBM, OV, |
| | family (Hsp40) member C19 | NM_001190233.1 | LUSC |
| ECE2 | endothelin converting enzyme | NM_032331.3 | OV, LUSC |
| | 2 | | |
| MUC20 | mucin 20, cell surface | NM_001282506.1 | OV, RICH |
| | associated | | |
| ELOVL6 | ELOVL fatty acid elongase 6 | NM_001130721.1, | TGCT, GBM |
| | | NM_024090.2 | |
| SORBS2 | sorbin and SH3 domain | NM_001145672.1 | THYM |
| | containing 2 | | |
| WDR1 | WD repeat domain 1 | NM_017491.3, | PAAD, GBM, OV |
| | | NM_005112.4 | |
| SEL1L3 | SEL1L family member 3 | NM_001297592.1, | READ, OV, ESCA |
| | | NM_015187.4 | |
| RBM47 | RNA binding motif protein | NM_019027.3 | OV |
| | 47 | | |
| SPON2 | spondin 2 | NM_012445.3 | PAAD, KIRP |
| C4orf48 | chromosome 4 open reading | NM_001168243.1 | UCEC, OV |
| | frame 48 | | |
| PPP2R2C | protein phosphatase 2 | NM_181876.2 | LUSC |
| | regulatory subunit B gamma | | |
| HTRA3 | HtrA serine peptidase 3 | NM_001297559.1 | PAAD |
| CPZ | carboxypeptidase Z | NM_003652.3, | PAAD, THYM |
| | | NM_001014447.2 | |
| SLC34A2 | solute carrier family 34 | NM_006424.2, | UCEC, THCA, |
| | member 2 | NM_001177998.1 | KIRP, OV |
| SPINK2 | serine peptidase inhibitor | NM_021114.3 | LAML |
| | Kazal type 2 | | |
| IGFBP7 | insulin like growth factor | NM_001553.2 | THYM, PAAD, |
| | binding protein 7 | | GBM, LAML |
| PKD2 | polycystin 2, transient | NM_000297.3 | LAML |
| | receptor potential cation | | |
| | channel | | |
| UGT8 | UDP glycosyltransferase 8 | NM_001322113.1, | READ |
| | | NM_001322112.1, | |
| | | NM_001128174.2, | |
| | | NM_001322114.1 | |
| EDNRA | endothelin receptor type A | NM_001957.3 | PAAD |
| HMGB2 | high mobility group box 2 | NM_001130689.1, | PAAD, LUSC, |
| | | NM_002129.3, | LUAD, DLBC, |
| | | NM_001130688.1 | BRCA, THYM, |
| | | | LGG, CESC, ACC, |
| | | | READ, UCS, |
| | | | COAD, LIHC, |
| | | | TGCT, GBM |
| CENPU | centromere protein U | NM_024629.3 | CESC, GBM, LUSC |
| PDLIM3 | PDZ and LIM domain 3 | NM_001114107.4 | PAAD, GBM |
| ECSCR | endothelial cell surface | NM_001077693.3 | GBM |
| | expressed chemotaxis and | | |
| | apoptosis regulator | | |
| SLIT3 | slit guidance ligand 3 | NM_001271946.1 | THYM |
| SLC45A2 | solute carrier family 45 | NM_001012509.3 | SKCM |
| | member 2 | | |
| UGT3A2 | UDP glycosyltransferase | NM_174914.3 | THYM |
| | family 3 member A2 | | |
| OSMR | oncostatin M receptor | NM_001323505.1, | GBM |
| | | NM_003999.2, | |
| | | NM_001323506.1 | |
| CSF1R | colony stimulating factor 1 | NM_005211.3, | LGG, PAAD, GBM, |
| | receptor | NM_001288705.1 | LAML |
| GM2A | GM2 ganglioside activator | NM_001167607.1 | PAAD |
| G3BP1 | G3BP stress granule | NM_005754.2, | DLBC, THYM |
| | assembly factor 1 | NM_198395.1 | |
| ATP10B | ATPase phospholipid | NM_025153.2 | READ, COAD |
| | transporting 10B (putative) | | |
| WWC1 | WW and C2 domain | NM_001161662.1 | OV |
| | containing 1 | | |
| WWC1 | WW and C2 domain | NM_001161661.1 | OV |
| | containing 1 | | |
| EMB | embigin | NM_198449.2 | PAAD, LAML |
| ALDH7A1 | aldehyde dehydrogenase 7 | NM_001202404.1 | THYM |
| | family member A1 | | |
| TCF7 | transcription factor 7 | NM_213648.3, | THYM |
| | | NM_201632.3 | |
| PCDH1 | protocadherin 1 | NM_032420.3 | OV |
| SPINK1 | serine peptidase inhibitor | NM_003122.4 | LIHC, LUAD, |
| | Kazal type 1 | | READ, COAD |
| SPARC | secreted protein acidic and | NM_003118.3, | PAAD, DLBC, |
| | cysteine rich | NM_001309443.1 | THYM, LGG, GBM |
| DUSP1 | dual specificity phosphatase 1 | NM_004417.3 | LAML |
| STC2 | stanniocalcin 2 | NM_003714.2 | KIRC |
| NOP16 | NOP16 nucleolar protein | NM_001291305.2, | DLBC |
| | | NM_001256540.3 | |
| PDLIM7 | PDZ and LIM domain 7 | NM_005451.4 | PAAD, GBM |
| PDLIM7 | PDZ and LIM domain 7 | NM_213636.2 | PAAD |
| RAB44 | RAB44, member RAS | NM_001257357.1 | LAML |
| | oncogene family | | |
| SLC22A3 | solute carrier family 22 | NM_021977.3 | PAAD |
| | member 3 | | |
| IRF4 | interferon regulatory factor 4 | NM_001195286.1, | DLBC |
| | | NM_002460.3 | |
| ALDH5A1 | aldehyde dehydrogenase 5 | NM_001080.3 | THYM |
| | family member A1 | | |
| CCDC167 | coiled-coil domain containing | NM_138493.2 | THYM, DLBC, |
| | 167 | | GBM, OV |
| AIM1 | crystallin beta-gamma | NM_001624.3 | LAML |
| | domain containing 1 | | |
| RPF2 | ribosome production factor 2 | NM_032194.2, | DLBC |
| | homolog | NM_001289111.1 | |
| MOXD1 | monooxygenase DBH like 1 | NM_015529.3 | PAAD |
| SLC17A4 | solute carrier family 17 | NM_005495.2 | KIRC |
| | member 4 | | |
| PRSS16 | serine protease 16 | NM_005865.3 | THYM |
| SFTA2 | surfactant associated 2 | NM_205854.2 | PAAD |
| SLC44A4 | solute carrier family 44 | NM_001178044.1, | READ, PAAD, |
| | member 4 | NM_025257.2 | BRCA, OV |
| CFB | complement factor B | NM_001710.5 | THYM, PAAD, |
| | | | BRCA, OV |
| C4A | complement C4A (Rodgers | NM_007293.2 | KIRC, THYM, GBM |
| | blood group) | | |
| C4B | complement C4B (Chido | NM_001002029.3 | LGG, KIRC, THYM, |
| | blood group) | | GBM |
| GPSM3 | G protein signaling modulator | NM_001276501.1 | PAAD |
| | 3 | | |
| HLA-DOB | major histocompatibility | NM_002120.3 | DLBC |
| | complex, class II, DO beta | | |
| TAP2 | transporter 2, ATP binding | NM_001290043.1 | PAAD |
| | cassette subfamily B member | | |
| PSMB9 | proteasome 20S subunit beta | NM_002800.4 | PAAD, GBM |
| | 9 | | |
| HLA-DOA | major histocompatibility | NM_002119.3 | DLBC, GBM, PAAD |
| | complex, class II, DO alpha | | |
| TREM2 | triggering receptor expressed | NM_001271821.1 | GBM |
| | on myeloid cells 2 | | |
| C6orf132 | chromosome 6 open reading | NM_001164446.2 | OV |
| | frame 132 | | |
| MRPL14 | mitochondrial ribosomal | NM_001318770.1, | DLBC, THYM, |
| | protein L14 | NM_001318769.1 | PAAD |
| HSP90AB1 | heat shock protein 90 alpha | NM_001271971.1, | DLBC |
| | family class B member 1 | NM_007355.3, | |
| | | NM_001271972.1 | |
| MEP1A | meprin A subunit alpha | NM_005588.2 | READ |
| TNFRSF21 | TNF receptor superfamily | NM_014452.4 | PAAD, THYM |
| | member 21 | | |
| CTGF | cellular communication | NM_001901.2 | THYM, DLBC, |
| | network factor 2 | | GBM, PAAD |
| TAGAP | T cell activation RhoGTPase | NM_001278733.1, | LAML |
| | activating protein | NM_054114.4 | |
| AOAH | acyloxyacyl hydrolase | NM_001637.3 | LAML |
| PRKAR1B | protein kinase cAMP- | NM_001164759.1, | THYM |
| | dependent type I regulatory | NM_001164762.1, | |
| | subunit beta | NM_001164760.1 | |
| ADAP1 | ArfGAP with dual PH | NM_001284311.1 | PAAD |
| | domains 1 | | |
| MICALL2 | MICAL like 2 | NM_182924.3 | PAAD |
| MAD1L1 | mitotic arrest deficient 1 like | NM_001304525.1 | SKCM |
| | 1 | | |
| AHR | aryl hydrocarbon receptor | NM_001621.4 | PAAD, LAML |
| CREB5 | cAMP responsive element | NM_182898.3 | KIRP |
| | binding protein 5 | | |
| NUDT1 | nudix hydrolase 1 | NM_198950.1, | PAAD, LUSC, |
| | | NM_002452.3, | THYM, DLBC, |
| | | NM_198948.1 | CESC, GBM |
| LFNG | LFNG O-fucosylpeptide 3- | NM_001040167.1 | GBM |
| | beta-N- | | |
| | acetylglucosaminyltransferase | | |
| MEOX2 | mesenchyme homeobox 2 | NM_005924.4 | GBM |
| SFRP4 | secreted frizzled related | NM_003014.3 | STAD, PAAD, GBM |
| | protein 4 | | |
| YAE1D1 | YAE1 maturation factor of | NM_020192.4 | THYM |
| | ABCE1 | | |
| MYO1G | myosin IG | NM_033054.2 | LAML |
| RAMP3 | receptor activity modifying | NM_005856.2 | DLBC |
| | protein 3 | | |
| CNPY4 | canopy FGF signaling | NM_152755.1 | PAAD |
| | regulator 4 | | |
| MEST | mesoderm specific transcript | NM_001253900.1, | UCEC, LUSC, |
| | | NM_002402.3, | LUAD, READ, UCS, |
| | | NM_001253901.1, | CESC, COAD, OV, |
| | | NM_177524.2, | GBM |
| | | NM_177525.2 | |
| AKR1B10 | aldo-keto reductase family 1 | NM_020299.4 | LIHC, PAAD, LUSC |
| | member B10 | | |
| ATP6V0A4 | ATPase H+ transporting VO | NM_130841.2 | RICH |
| | subunit a4 | | |
| CLEC5A | C-type lectin domain | NM_013252.2 | GBM |
| | containing 5A | | |
| PIP | prolactin induced protein | NM_002652.2 | RICH |
| TCAF1 | TRPM8 channel associated | NM_001206941.1, | THYM |
| | factor 1 | NM_014719.2 | |
| DNAJC5B | DnaJ heat shock protein | NM_033105.4 | DLBC |
| | family (Hsp40) member C5 | | |
| | beta | | |
| MTUS1 | microtubule associated | NM_001166393.1 | THYM |
| | scaffold protein 1 | | |
| LOXL2 | lysyl oxidase like 2 | NM_002318.2 | PAAD, RIRC, |
| | | | DLBC, TGCT, GBM |
| DOCR5 | dedicator of cytokinesis 5 | NM_024940.7 | LAML |
| DPYSL2 | dihydropyrimidinase like 2 | NM_001197293.2, | RIRC, PAAD |
| | | NM_001386.5 | |
| SULF1 | sulfatase 1 | NM_001128206.1, | PAAD |
| | | NM_001128204.1, | |
| | | NM_015170.2, | |
| | | NM_001128205.1 | |
| TPD52 | tumor protein D52 | NM_001025253.2 | OV, LUSC |
| FAM83A | family with sequence | NM_032899.6 | CESC, LUAD, |
| | similarity 83 member A | | LUSC |
| TATDN1 | TatD DNase domain | NM_032026.3 | DLBC, THYM |
| | containing 1 | | |
| SLA | Src like adaptor | NM_001045556.2 | PAAD, GBM, |
| | | | LAML |
| KHDRBS3 | KH RNA binding domain | NM_006558.2 | THYM |
| | containing, signal | | |
| | transduction associated 3 | | |
| SMIM19 | small integral membrane | NM_138436.3 | THYM |
| | protein 19 | | |
| MRPL15 | mitochondrial ribosomal | NM_014175.3 | DLBC |
| | protein L15 | | |
| TRAM1 | translocation associated | NM_001317804.1, | GBM |
| | membrane protein 1 | NM_014294.5 | |
| CRISPLD1 | cysteine rich secretory protein | NM_001286778.1, | GBM |
| | LCCL domain containing 1 | NM_031461.5 | |
| CHMP4C | charged multivesicular body | NM_152284.3 | OV, READ, COAD |
| | protein 4C | | |
| ESRP1 | epithelial splicing regulatory | NM_001122827.1 | OV |
| | protein 1 | | |
| NIPAL2 | NIPA like domain containing | NM_001321636.1 | LAML |
| | 2 | | |
| CTHRC1 | collagen triple helix repeat | NM_001256099.1, | HNSC, PAAD, |
| | containing 1 | NM_138455.3 | LUSC, KIRC, |
| | | | DLBC, LUAD, UCS, |
| | | | STAD, OV, ESCA |
| SQLE | squalene epoxidase | NM_003129.3 | PAAD |
| FAM189A2 | family with sequence | NM_001127608.1 | KICH |
| | similarity 189 member A2 | | |
| GDA | guanine deaminase | NM_004293.4 | PAAD |
| CORO2A | coronin 2A | NM_052820.3 | PAAD, OV |
| NDUFA8 | NADH:ubiquinone | NM_014222.2 | DLBC, THYM |
| | oxidoreductase subunit A8 | | |
| FAM214B | family with sequence | NM_025182.3 | PAAD |
| | similarity 214 member B | | |
| TPM2 | tropomyosin 2 | NM_001301226.1 | THYM, DLBC, |
| | | | GBM, PAAD |
| CTSL | cathepsin L | NM_001257973.1, | DLBC |
| | | NM_001257971.1, | |
| | | NM_001912.4, | |
| | | NM_001257972.1, | |
| | | NM_145918.2 | |
| CTSV | cathepsin V | NM_001333.3 | UCEC, LUSC, |
| | | | THYM, READ, |
| | | | CESC, OV |
| GSN | gelsolin | NM_001127666.1, | PAAD, THYM |
| | | NM_001258029.1, | |
| | | NM_001127667.1 | |
| STOM | stomatin | NM_198194.2 | STAD, OV, TGCT, |
| | | | GBM, ESCA |
| PTGS1 | prostaglandin-endoperoxide | NM_080591.2, | GBM, OV |
| | synthase 1 | NM_000962.3 | |
| ZDHHC12 | zinc finger DHHC-type | NM_032799.4, | OV |
| | palmitoyltransferase 12 | NM_001318016.1, | |
| | | NM_001318015.1 | |
| ZDHHC12 | zinc finger DHHC-type | NM_001318023.1 | OV |
| | palmitoyltransferase 12 | | |
| PRRX2 | paired related homeobox 2 | NM_016307.3 | PAAD, OV |
| SURF2 | surfeit 2 | NM_017503.4, | THYM |
| | | NM_001278928.1 | |
| PHPT1 | phosphohistidine phosphatase | NM_014172.5. | DLBC, THYM |
| | 1 | NM_001287342.1 | |
| PTGDS | prostaglandin D2 synthase | NM_000954.5 | DLBC, THYM, OV |
| CLIC3 | chloride intracellular channel | NM_004669.2 | PAAD, OV |
| | 3 | | |
| DHRSX | dehydrogenase/reductase X- | NM_145177.2 | GBM |
| | linked | | |
| SLC9A7 | solute carrier family 9 | NM_001257291.1 | LAML |
| | member A7 | | |
| POF1B | POF1B actin binding protein | NM_024921.3 | READ, COAD |
| GPC3 | glypican 3 | NM_001164617.1, | LIHC |
| | | NM_001164619.1, | |
| | | NM_004484.3, | |
| | | NM_001164618.1 | |
| CSF2RA | colony stimulating factor 2 | NM_006140.4, | GBM |
| | receptor subunit alpha | NM_172245.2 | |
| ARSD | arylsulfatase D | NM_001669.3 | LAML |
| ARSE | arylsulfatase L | NM_000047.2 | READ |
| GPR143 | G protein-coupled receptor | NM_000273.2 | SKCM |
| | 143 | | |
| USP11 | ubiquitin specific peptidase | NM_004651.3 | THYM |
| | 11 | | |
| SLC38A5 | solute carrier family 38 | NM_033518.3 | READ |
| | member 5 | | |
| PLP2 | proteolipid protein 2 | NM_002668.2 | GBM |
| IL2RG | interleukin 2 receptor subunit | NM_000206.2 | PAAD, READ, |
| | gamma | | STAD, OV, TGCT |
| CITED1 | Cbp/p300 interacting | NM_001144885.1 | THCA |
| | trans activator with Glu/Asp | | |
| | rich carboxy-terminal domain | | |
| | 1 | | |
| NOX1 | NADPH oxidase 1 | NM_007052.4. | READ, COAD |
| | | NM_013955.2 | |
| SASH3 | SAM and SH3 domain | NM_018990.3 | GBM, LAML |
| | containing 3 | | |
| IGSF1 | immunoglobulin superfamily | NM_001170962.1. | THCA |
| | member 1 | NM_001555.4 | |
| HMGB3 | high mobility group box 3 | NM_005342.3. | UCEC, LUSC, |
| | | NM_001301231.1. | LUAD, BRCA, UCS, |
| | | NM_001301228.1. | OV |
| | | NM_001301229.1 | |
| CSAG1 | chondrosarcoma associated | NM_001102576.2 | SKCM |
| | gene 1 | | |
| BGN | biglycan | NM_001711.5 | THYM, DLBC, |
| | | | GBM, PAAD |
| L1CAM | L1 cell adhesion molecule | NM_001143963.2 | OV |
| CCDC3 | coiled-coil domain containing | NM_031455.3 | THYM |
| | 3 | | |
| TBATA | thymus, brain and testes | NM_001318243.1, | THYM |
| | associated | NM_001318242.1, | |
| | | NM_152710.3 | |
| PIK3AP1 | phosphoinositide-3-kinase | NM_152309.2 | GBM, LAML |
| | adaptor protein 1 | | |
| NET1 | solute carrier family 6 | NM_005863.4 | PAAD, THYM |
| | member 2 | | |
| ATP5C1 | ATP synthase F1 subunit | NM_005174.3 | PAAD. THYM |
| | gamma | | |
| SRGN | serglycin | NM_002727.3, | PAAD, GBM |
| | | NM_001321053.1 | |
| H2AFY2 | macroH2A.2 histone | NM_018649.2 | THYM |
| PPA1 | inorganic pyrophosphatase 1 | NM_021129.3 | DLBC, THYM |
| UNC5B | unc-5 netrin receptor B | NM_001244889.1 | PAAD |
| PLAU | plasminogen activator, | NM_002658.4 | PAAD, DLBC, OV, |
| | urokinase | | TGCT, GBM |
| DUSP5 | dual specificity phosphatase 5 | NM_004419.3 | PAAD |
| VENTX | VENT homeobox | NM_014468.3 | TGCT |
| FUOM | fucose mutarotase | NM_001098483.2 | DLBC |
| LDLRAD3 | low density lipoprotein | NM_174902.3 | GBM |
| | receptor class A domain | | |
| | containing 3 | | |
| ANO1 | anoctamin 1 | NM_018043.5 | PAAD |
| B4GALNT4 | beta-1,4-N-acetyl- | NM_178537.4 | LUSC |
| | galactosaminyltransferase 4 | | |
| RASSF7 | Ras association domain | NM_001143993.1 | OV |
| | family member 7 | | |
| CHID1 | chitinase domain containing 1 | NM_001142674.1, | THYM |
| | | NM_001142677.1 | |
| MUC5B | mucin 5B, oligomeric | NM_002458.2 | LUAD, PAAD |
| | mucus/gel-forming | | |
| IGF2 | insulin like growth factor 2 | NM_001291861.2, | ACC, PAAD |
| | | NM_000612.5 | |
| SLC22A18 | solute carrier family 22 | NM_183233.2, | PAAD |
| | member 18 | NM_001315502.1 | |
| DKK3 | dickkopf WNT signaling | NM_013253.4 | PAAD |
| | pathway inhibitor 3 | | |
| SAA2 | serum amyloid A2 | NM_030754.4 | THYM |
| SAA1 | serum amyloid A1 | NM_199161.4 | THYM, PAAD, |
| | | | GBM |
| HTATIP2 | HIV-1 Tat interactive protein | NM_001098520.1, | PAAD |
| | 2 | NM_006410.4 | |
| EHF | ETS homologous factor | NM_012153.5 | UCEC, READ, |
| | | | CESC, OV, COAD |
| CREB3L1 | cAMP responsive element | NM_052854.3 | ESCA |
| | binding protein 3 like 1 | | |
| UBE2L6 | ubiquitin conjugating enzyme | NM_004223.4 | PAAD |
| | E2 L6 | | |
| LPXN | leupaxin | NM_004811.2 | LAML |
| PLA2G16 | phospholipase A and | NM_007069.3, | PAAD |
| | acyltransferase 3 | NM_001128203.1 | |
| FERMT3 | fermitin family member 3 | NM_031471.5 | PAAD, GBM |
| OVOL1 | ovo like transcriptional | NM_004561.3 | READ, CESC, |
| | repressor 1 | | LUSC |
| SLC29A2 | solute carrier family 29 | NM_001532.2 | OV |
| | member 2 | | |
| FOLR1 | folate receptor alpha | NM_016729.2, | UCEC, UCS, PAAD, |
| | | NM_016725.2, | OV |
| | | NM_000802.3, | |
| | | NM_016724.2 | |
| MMP10 | matrix metallopeptidase 10 | NM_002425.2 | HNSC, LUSC |
| MMP1 | matrix metallopeptidase 1 | NM_001145938.1, | HNSC, PAAD, |
| | | NM_002421.3 | LUSC, LUAD, |
| | | | READ, COAD, |
| | | | ESCA |
| MMP3 | matrix metallopeptidase 3 | NM_002422.4 | READ, HNSC, |
| | | | ESCA |
| MMP12 | matrix metallopeptidase 12 | NM_002426.5 | HNSC, PAAD, |
| | | | LUSC, LUAD, |
| | | | READ, STAD, |
| | | | COAD, ESCA |
| POU2AF1 | POU class 2 homeobox | NM_006235.2 | DLBC |
| | associating factor 1 | | |
| CD3D | CD3d molecule | NM_001040651.1 | THYM |
| TRIM29 | tripartite motif containing 29 | NM_012101.3 | PAAD, LUSC, |
| | | | THYM, CESC, OV |
| VSIG2 | V-set and immunoglobulin | NM_014312.3 | PAAD |
| | domain containing 2 | | |
| BARX2 | BARX homeobox 2 | NM_003658.4 | KIRC |
| GUCY2C | guanylate cyclase 2C | NM_004963.3 | READ, COAD |
| B4GALNT3 | beta-1,4-N-acetyl- | NM_173593.3 | PAAD |
| | galactosaminyltransferase 3 | | |
| PLBD1 | phospholipase B domain | NM_024829.5 | PAAD, TGCT |
| | containing 1 | | |
| ARHGDIB | Rho GDP dissociation | NM_001175.6, | PAAD, GBM |
| | inhibitor beta | NM_001321421.1 | |
| FAM60A | SIN3-HDAC complex | NM_021238.2, | GBM |
| | associated factor | NM_001135812.1, | |
| | | NM_001135811.1 | |
| ERBB3 | erb-b2 receptor tyrosine | NM_001982.3 | OV |
| | kinase 3 | | |
| LRP1 | LDL receptor related protein | NM_002332.2 | PAAD |
| | 1 | | |
| MSRB3 | methionine sulfoxide | NM_198080.3, | LAML |
| | reductase B3 | NM_001193461.1 | |
| ALDH2 | aldehyde dehydrogenase 2 | NM_001204889.1 | PAAD |
| | family member | | |
| LAG3 | lymphocyte activating 3 | NM_002286.5 | DLBC |
| CLEC12A | C-type lectin domain family | NM_001300730.1 | LAML |
| | 12 member A | | |
| GPRC5A | G protein-coupled receptor | NM_003979.3 | READ, PAAD, |
| | class C group 5 member A | | BRCA, TGCT |
| LDHB | lactate dehydrogenase B | NM_001315537.1, | DLBC, THYM |
| | | NM_002300.7 | |
| TUBA1A | tubulin alpha 1a | NM_006009.3, | PAAD, GBM |
| | | NM_001270400.1 | |
| TROAP | trophinin associated protein | NM_001100620.1 | LUSC |
| KRT6B | keratin 6B | NM_005555.3 | CESC, PAAD, |
| | | | LUSC |
| KRT6C | keratin 6C | NM_173086.4 | LUSC |
| KRT6A | keratin 6A | NM_005554.3 | CESC, PAAD, |
| | | | LUSC |
| KRT8 | keratin 8 | NM_002273.3 | UCEC, PAAD, |
| | | | THYM, BRCA, |
| | | | READ, CESC, UCS, |
| | | | OV, COAD, TGCT, |
| | | | ESCA |
| SLC39A5 | solute carrier family 39 | NM_001135195.1 | READ |
| | member 5 | | |
| DCN | decorin | NM_133503.3 | DLBC, THYM, |
| | | | PAAD |
| IKBIP | IKBKB interacting protein | NM_153687.3 | GBM |
| OAS1 | 2′-5′-oligoadenylate | NM_016816.3 | PAAD, GBM |
| | synthetase 1 | | |
| OAS3 | 2′-5′-oligoadenylate | NM_006187.3 | PAAD, OV |
| | synthetase 3 | | |
| FLT3 | fms related receptor tyrosine | NM_004119.2 | LAML |
| | kinase 3 | | |
| FARP1 | FERM, ARH/RhoGEF and | NM_001286839.1 | THYM |
| | pleckstrin domain protein 1 | | |
| COL4A1 | collagen type IV alpha 1 | NM_001845.5 | PAAD, DLBC, |
| | chain | | THYM, LIHC, GBM |
| COL4A2 | collagen type IV alpha 2 | NM_001846.2 | THYM, DLBC, |
| | chain | | GBM. PAAD |
| HMGB1 | high mobility group box 1 | NM_001313892.1 | THYM |
| ALOX5AP | arachidonate 5-lipoxygenase | NM_001629.3 | PAAD, GBM, |
| | activating protein | | LAML |
| TNFSF13B | TNF superfamily member | NM_006573.4 | LAML |
| | 13b | | |
| MNAT1 | MNAT1 component of CDK | NM_001177963.1 | THYM |
| | activating kinase | | |
| SLC7A7 | solute carrier family 7 | NM_001126106.2 | PAAD |
| | member 7 | | |
| LRFN5 | leucine rich repeat and | NM_152447.3 | RICH |
| | fibronectin type III domain | | |
| | containing 5 | | |
| TMX1 | thioredoxin related | NM_030755.4 | GBM |
| | transmembrane protein 1 | | |
| DLGAP5 | DLG associated protein 5 | NM_014750.4 | LUSC, DLBC, |
| | | | BRCA, CESC, UCS, |
| | | | OV, GBM |
| SMOC1 | SPARC related modular | NM_001034852.2, | LGG, RICH |
| | calcium binding 1 | NM_022137.5 | |
| RIN3 | Ras and Rab interactor 3 | NM_001319987.1 | LAML |
| VRR1 | VRR serine/threonine kinase | NM_003384.2 | DLBC |
| | 1 | | |
| PNP | purine nucleoside | NM_000270.3 | PAAD, GBM, OV |
| | phosphorylase | | |
| MMP14 | matrix metallopeptidase 14 | NM_004995.3 | THYM, DLBC, |
| | | | GBM, PAAD |
| NGDN | neuroguidin | NM_001042635.1 | THYM |
| DHRS2 | dehydrogenase/reductase 2 | NM_005794.3 | RICH |
| FKBP3 | FRBP prolyl isomerase 3 | NM_002013.3 | DLBC, THYM |
| PYGL | glycogen phosphorylase L | NM_001163940.1, | GBM |
| | | NM_002863.4 | |
| ERH | ERH mRNA splicing and | NM_004450.2 | DLBC, THYM |
| | mitosis factor | | |
| SERPINA3 | serpin family A member 3 | NM_001085.4 | LGG, GBM |
| AMN | amnion associated | NM_030943.3 | PAAD |
| | transmembrane protein | | |
| SIVA1 | SIVA1 apoptosis inducing | NM_006427.3, | THYM, DLBC |
| | factor | NM_021709.2 | |
| PLD4 | phospholipase D family | NM_001308174.1 | GBM |
| | member 4 | | |
| THSD4 | thrombospondin type 1 | NM_001286429.1, | OV |
| | domain containing 4 | NM_024817.2 | |
| MCTP2 | multiple C2 and | NM_018349.3 | LAML |
| | transmembrane domain | | |
| | containing 2 | | |
| THBS1 | thrombospondin 1 | NM_003246.3 | PAAD |
| FBN1 | fibrillin 1 | NM_000138.4 | PAAD |
| PRM | pyruvate kinase M1/2 | NM_002654.5, | PAAD, OV, DLBC |
| | | NM_001206797.2 | |
| CHSY1 | chondroitin sulfate synthase 1 | NM_014918.4 | PAAD |
| LPCAT4 | lysophosphatidylcholine | NM_153613.2 | PAAD |
| | acyltransferase 4 | | |
| PHGR1 | proline, histidine and glycine | NM_001145643.1 | PAAD |
| | rich 1 | | |
| NUSAP1 | nucleolar and spindle | NM_018454.7, | UCEC, PAAD, |
| | associated protein 1 | NM_001301136.1, | LUSC, DLBC, |
| | | NM_001243143.1 | BRCA, THYM, |
| | | | CESC, UCS, GBM |
| DUOXA2 | dual oxidase maturation | NM_207581.3 | READ, PAAD |
| | factor 2 | | |
| SQRDL | sulfide quinone | NM_001271213.1 | GBM |
| | oxidoreductase | | |
| CA12 | carbonic anhydrase 12 | NM_001218.4, | GBM, LUSC |
| | | NM_206925.2 | |
| LOXL1 | lysyl oxidase like 1 | NM_005576.3 | PAAD |
| PML | promyelocytic leukemia | NM_033238.2 | PAAD |
| SEMA7A | semaphorin 7A (John Milton | NM_001146030.2 | PAAD |
| | Hagen blood group) | | |
| CSPG4 | chondroitin sulfate | NM_001897.4 | KIRC, SKCM |
| | proteoglycan 4 | | |
| ANPEP | alanyl aminopeptidase, | NM_001150.2 | LAML |
| | membrane | | |
| PLCG2 | phospholipase C gamma 2 | NM_002661.4 | KICH |
| SEZ6L2 | seizure related 6 homolog like | NM_012410.3, | LUAD, KIRP, OV |
| | 2 | NM_001114099.2, | |
| | | NM_001243332.1, | |
| | | NM_201575.3, | |
| | | NM_001114100.2 | |
| POLR3K | RNA polymerase III subunit | NM_016310.4 | DLBC |
| | K | | |
| SOX8 | SRY-box transcription factor | NM_014587.4 | LGG |
| | 8 | | |
| TPSB2 | tryptase beta 2 | NM_024164.5 | KIRC, PAAD |
| | (gene/pseudogene) | | |
| TPSAB1 | tryptase alpha/beta 1 | NM_003294.3 | LAML |
| HN1L | Jupiter microtubule | NM_144570.2 | THYM |
| | associated homolog 2 | | |
| SYNGR3 | synaptogyrin 3 | NM_004209.5 | KICH |
| NTHL1 | nth like DNA glycosylase 1 | NM_001318194.1, | DLBC, THYM |
| | | NM_002528.6 | |
| ECI1 | enoyl-CoA delta isomerase 1 | NM_001178029.1 | THYM |
| TCEB2 | elongin B | NM_207013.2 | PAAD |
| PRSS22 | serine protease 22 | NM_022119.3 | PAAD, READ, |
| | | | CESC, OV, COAD |
| PAQR4 | progestin and adipoQ | NM_001324118.1 | OV |
| | receptor family member 4 | | |
| SMIM22 | small integral membrane | NM_001253793.1 | UCEC, PAAD, |
| | protein 22 | | LUAD, BRCA, |
| | | | READ, CESC, UCS, |
| | | | COAD, OV, ESCA |
| NDE1 | nudE neurodevelopment | NM_017668.2 | PAAD |
| | protein 1 | | |
| ERN2 | endoplasmic reticulum to | NM_001308220.1, | PAAD |
| | nucleus signaling 2 | NM_033266.3 | |
| IL21R | interleukin 21 receptor | NM_181078.2 | DLBC |
| EIF3C | eukaryotic translation | NM_001286478.1 | DLBC, THYM |
| | initiation factor 3 subunit C | | |
| SULT1A4 | sulfotransferase family 1A | NM_001017390.2 | PAAD |
| | member 4 | | |
| DOC2A | double C2 domain alpha | NM_003586.2, | KIRC |
| | | NM_001282062.1 | |
| TGFB1I1 | transforming growth factor | NM_001164719.1, | PAAD, GBM |
| | beta 1 induced transcript 1 | NM_001042454.2 | |
| MMP2 | matrix metallopeptidase 2 | NM_001302510.1, | PAAD, BRCA, |
| | | NM_001302508.1, | STAD, GBM, ESCA |
| | | NM_001302509.1, | |
| | | NM_004530.5 | |
| CES1 | carboxylesterase 1 | NM_001025195.1 | PAAD |
| ADGRG1 | adhesion G protein-coupled | NM_001145771.2, | LUSC, LUAD, |
| | receptor G1 | NM_001145774.2, | BRCA, READ, |
| | | NM_001145770.2, | CESC, COAD, OV, |
| | | NM_001290142.1, | GBM |
| | | NM_005682.6, | |
| | | NM_001290144.1 | |
| CMTM3 | CKLF like MARVEL | NM_181553.2 | PAAD, GBM |
| | transmembrane domain | | |
| | containing 3 | | |
| NQO1 | NAD(P)H quinone | NM_001286137.1 | PAAD, LUSC |
| | dehydrogenase 1 | | |
| CLEC18B | C-type lectin domain family | NM_001011880.2 | GBM |
| | 18 member B | | |
| SLC7A5 | solute carrier family 7 | NM_003486.5 | LUSC |
| | member 5 | | |
| CYBA | cytochrome b-245 alpha | NM_000101.3 | PAAD, GBM. OV, |
| | chain | | TGCT |
| CDT1 | chromatin licensing and DNA | NM_030928.3 | LUSC, DLBC. |
| | replication factor 1 | | THYM. CESC. OV |
| SLC22A31 | solute carrier family 22 | NM_001242757.1 | THCA |
| | member 31 | | |
| SPNS3 | sphingolipid transporter 3 | NM_001320449.1, | LAML |
| | (putative) | NM_182538.4 | |
| ADAP2 | ArfGAP with dual PH | NM_018404.2 | GBM |
| | domains 2 | | |
| SLC16A5 | solute carrier family 16 | NM_004695.3 | PAAD |
| | member 5 | | |
| MYO1C | myosin IC | NM_033375.4 | DLBC, THYM |
| TM4SF5 | transmembrane 4 L six family | NM_003963.2 | READ, PAAD, |
| | member 5 | | COAD |
| TNFSF13 | TNF superfamily member 13 | NM_003808.3 | LAML |
| NAA38 | N-alpha-acetyltransferase 38, | NM_001320924.1 | DLBC, THYM, OV |
| | NatC auxiliary subunit | | |
| PIK3R6 | phosphoinositide-3-kinase | NM_001010855.3, | LAML |
| | regulatory subunit 6 | NM_001290211.1 | |
| CCL2 | C-C motif chemokine ligand | NM_002982.3 | THYM, DLBC, |
| | 2 | | GBM, OV |
| SLFN11 | schlafen family member 11 | NM_001104589.1, | LAML |
| | | NM_152270.3, | |
| | | NM_001104588.1, | |
| | | NM_001104587.1, | |
| | | NM_001104590.1 | |
| CCL5 | C-C motif chemokine ligand | NM_002985.2 | PAAD, LAML, |
| | 5 | | TGCT, KIRC, OV, |
| | | | SKCM |
| GRB7 | growth factor receptor bound | NM_001030002.2, | CESC, OV |
| | protein 7 | NM_005310.3 | |
| KRT16 | keratin 16 | NM_005557.3 | CESC, PAAD, |
| | | | LUSC |
| KRT17 | keratin 17 | NM_000422.2 | UCEC, PAAD, |
| | | | LUSC, THYM, |
| | | | CESC, UCS, STAD, |
| | | | OV |
| JUP | junction plakoglobin | NM_002230.2 | PAAD, LUSC, |
| | | | THYM, OV, TGCT |
| MPO | myeloperoxidase | NM_000250.1 | LAML |
| LIMD2 | LIM domain containing 2 | NM_030576.3 | LAML |
| SLC25A19 | solute carrier family 25 | NM_001126122.1 | DLBC |
| | member 19 | | |
| ST6GALNAC2 | ST6 N-acetylgalactosaminide | NM_006456.2 | OV |
| | alpha-2,6-sialyltransferase 2 | | |
| TMC8 | transmembrane channel like 8 | NM_152468.4 | LAML |
| BAIAP2 | BAR/IMD domain containing | NM_001144888.1, | THYM, OV |
| | adaptor protein 2 | NM_017450.2 | |
| ALYREF | Aly/REF export factor | NM_005782.3 | DLBC, THYM, |
| | | | PAAD |
| KLHL14 | kelch like family member 14 | NM_020805.2 | OV |
| MBP | myelin basic protein | NM_001025100.1 | LAML |
| NDC80 | NDC80 kinetochore complex | NM_006101.2 | LUSC, DLBC, |
| | component | | BRCA, THYM, OV, |
| | | | GBM |
| TTR | transthyretin | NM_000371.3 | KICH |
| CYB5A | cytochrome b5 type A | NM_001190807.2 | THYM |
| GNA15 | G protein subunit alpha 15 | NM_002068.3 | CESC, PAAD, |
| | | | LAML |
| MUC16 | mucin 16, cell surface | NM_024690.2 | OV |
| | associated | | |
| DDX39A | DExD-box helicase 39A | NM_005804.3 | DLBC, GBM, PAAD |
| KLC3 | kinesin light chain 3 | NM_177417.2 | CESC, LUSC |
| PTPRH | protein tyrosine phosphatase | NM_002842.4 | PAAD |
| | receptor type H | | |
| GNA11 | G protein subunit alpha 11 | NM_002067.4 | THYM |
| CREB3L3 | cAMP responsive element | NM_032607.2 | KIRC |
| | binding protein 3 like 3 | | |
| CD70 | CD70 molecule | NM_001252.4 | KIRC, DLBC, KIRP |
| PRAM1 | PML-RARA regulated | NM_032152.4 | LAML |
| | adaptor molecule 1 | | |
| MRPL4 | mitochondrial ribosomal | NM_146387.1 | DLBC |
| | protein L4 | | |
| AP1M2 | adaptor related protein | NM_001300887.1, | UCEC, READ, |
| | complex 1 subunit mu 2 | NM_005498.4 | CESC, UCS, COAD, |
| | | | OV |
| SPC24 | SPC24 component of NDC80 | NM_182513.3, | THYM, GBM, OV |
| | kinetochore complex | NM_001317033.1 | |
| RAB3D | RAB3D, member RAS | NM_004283.3 | LAML |
| | oncogene family | | |
| ZNF439 | zinc finger protein 439 | NM_152262.2 | LAML |
| MAN2B1 | mannosidase alpha class 2B | NM_000528.3 | GBM |
| | member 1 | | |
| RNASEH2A | ribonuclease H2 subunit A | NM_006397.2 | THYM, DLBC, |
| | | | GBM |
| FARSA | phenylalanyl-tRNA | NM_004461.2 | DLBC |
| | synthetase subunit alpha | | |
| NACC1 | nucleus accumbens associated | NM_052876.3 | OV |
| | 1 | | |
| GIPC1 | GIPC PDZ domain | NM_202468.2, | PAAD, THYM, OV |
| | containing family member 1 | NM_202494.2, | |
| | | NM_005716.3 | |
| NOTCH3 | notch receptor 3 | NM_000435.2 | PAAD, THYM |
| TPM4 | tropomyosin 4 | NM_003290.2 | PAAD |
| PLVAP | plasmalemma vesicle | NM_031310.2 | LIHC, THYM, GBM |
| | associated protein | | |
| LSM4 | LSM4 homolog, U6 small | NM_001252129.1 | THYM |
| | nuclear RNA and mRNA | | |
| | degradation associated | | |
| CCNE1 | cyclin E1 | NM_001322262.1, | OV |
| | | NM_001322261.1, | |
| | | NM_001322259.1, | |
| | | NM_001238.3 | |
| GPI | glucose-6-phosphate | NM_001184722.1 | PAAD |
| | isomerase | | |
| FXYD3 | FXYD domain containing ion | NM_001136007.1, | PAAD, LUSC, |
| | transport regulator 3 | NM_001136011.1, | READ, CESC, |
| | | NM_005971.3, | COAD, OV |
| | | NM_001136012.1 | |
| CD22 | CD22 molecule | NM_001185101.1, | DLBC |
| | | NM_001771.3 | |
| TYROBP | transmembrane immune | NM_001173514.1, | PAAD, LAML, |
| | signaling adaptor TYROBP | NM_003332.3 | KIRC, STAD, OV, |
| | | | TGCT, GBM |
| TYROBP | transmembrane immune | NM_001173515.1, | PAAD, LAML, |
| | signaling adaptor TYROBP | NM_198125.2 | KIRC, OV, GBM |
| CAPNS1 | calpain small subunit 1 | NM_001302633.1 | PAAD, THYM |
| COX7A1 | cytochrome c oxidase subunit | NM_001864.3 | THYM |
| | 7A1 | | |
| LGALS4 | galectin 4 | NM_006149.3 | LIHC, STAD, |
| | | | PAAD, READ |
| FBXO27 | F-box protein 27 | NM_178820.4 | LUSC |
| GMFG | glia maturation factor gamma | NM_004877.3 | PAAD, GBM |
| DLL3 | delta like canonical Notch | NM_203486.2 | LGG |
| | ligand 3 | | |
| CYP2S1 | cytochrome P450 family 2 | NM_030622.7 | PAAD, LUSC, |
| | subfamily S member 1 | | READ, OV, COAD, |
| | | | TGCT |
| TGFB1 | transforming growth factor | NM_000660.5 | PAAD, GBM |
| | beta 1 | | |
| CNFN | comifelin | NM_032488.3 | OV, LUSC |
| SULT2B1 | sulfotransferase family 2B | NM_177973.1 | READ, CESC, |
| | member 1 | | PAAD, COAD |
| FAM83E | family with sequence | NM_017708.3 | PAAD |
| | similarity 83 member E | | |
| PLEKHA4 | pleckstrin homology domain | NM_020904.2 | GBM |
| | containing A4 | | |
| RRAS | RAS related | NM_006270.4 | PAAD |
| PRMT1 | protein arginine | NM_001536.5, | DLBC, THYM |
| | methyltransferase 1 | NM_198318.4 | |
| IL4I1 | interleukin 4 induced 1 | NM_152899.1 | UCEC, DLBC, OV |
| SIGLEC14 | sialic acid binding Ig like | NM_001098612.1 | LAML |
| | lectin 14 | | |
| TMC4 | transmembrane channel like 4 | NM_144686.3 | READ, UCEC, OV |
| ISOC2 | isochorismatase domain | NM_024710.2, | DLBC |
| | containing 2 | NM_00U36201.1 | |
| EYA2 | EYA transcriptional | NM_005244.4 | OV |
| | coactivator and phosphatase 2 | | |
| PYGB | glycogen phosphorylase B | NM_002862.3 | PAAD |
| E2F1 | E2F transcription factor 1 | NM_005225.2 | LIHC, CESC, DLBC |
| FAM83D | family with sequence | NM_030919.2 | LUSC, BRCA, |
| | similarity 83 member D | | CESC, UCS, OV |
| TOMM34 | translocase of outer | NM_006809.4 | THYM |
| | mitochondrial membrane 34 | | |
| RASSF2 | Ras association domain | NM_014737.2, | LAML |
| | family member 2 | NM_170774.1 | |
| FERMT1 | fermitin family member 1 | NM_017671.4 | PAAD, LUSC, |
| | | | READ, STAD, |
| | | | COAD |
| OVOL2 | ovo like zinc finger 2 | NM_021220.3 | OV |
| EIF6 | eukaryotic translation | NM_001267810.1, | PAAD |
| | initiation factor 6 | NM_181468.2 | |
| ADA | adenosine deaminase | NM_001322050.1, | DLBC, THYM |
| | | NM_000022.3 | |
| PLTP | phospholipid transfer protein | NM_001242921.1, | THYM, PAAD, |
| | | NM_182676.2, | GBM, DLBC |
| | | NM_006227.3, | |
| | | NM_001242920.1 | |
| MMP9 | matrix metallopeptidase 9 | NM_004994.2 | HNSC, PAAD, |
| | | | TGCT, BRCA, |
| | | | READ, CESC, UCS, |
| | | | STAD, COAD, OV, |
| | | | SKCM, GBM, ESCA |
| CD40 | CD40 molecule | NM_001322422.1, | DLBC, THYM |
| | | NM_001322421.1, | |
| | | NM_001250.5 | |
| RCAN1 | regulator of calcineurin 1 | NM_203418.2, | THYM, GBM |
| | | NM_001285389.2 | |
| SAMSN1 | SAM domain, SH3 domain | NM_022136.4 | GBM |
| | and nuclear localization | | |
| | signals 1 | | |
| BTG3 | BTG anti-proliferation factor | NM_006806.4 | THYM. GBM |
| | 3 | | |
| SOD1 | superoxide dismutase 1 | NM_000454.4 | DLBC, THYM, |
| | | | PAAD |
| FAM3B | FAM3 metabolism regulating | NM_058186.3 | OV |
| | signaling molecule B | | |
| RIPK4 | receptor interacting | NM_020639.2 | UCEC, CESC. OV |
| | serine/threonine kinase 4 | | |
| SLC37A1 | solute carrier family 37 | NM_018964.3 | PAAD |
| | member 1 | | |
| DNMT3L | DNA methyltransferase 3 like | NM_175867.2 | TGCT |
| SLC5A1 | solute earner family 5 | NM_000343.3 | READ, PAAD, |
| | member 1 | | COAD |
| HMOX1 | heme oxygenase 1 | NM_002133.2 | PAAD, GBM |
| CBY1 | chibby family member 1, beta | NM_015373.3 | THYM |
| | catenin antagonist | | |
| BIK | BCL2 interacting killer | NM_001197.4 | UCEC, PAAD, |
| | | | LUSC, DLBC, |
| | | | READ, CESC, OV |
| CELSR1 | cadherin EGF LAG seven- | NM_014246.1 | OV |
| | pass G-type receptor 1 | | |
| PRODH | proline dehydrogenase 1 | NM_001195226.1 | TGCT |
| MIF | macrophage migration | NM_002415.1 | DLBC |
| | inhibitory factor | | |
| SUSD2 | sushi domain containing 2 | NM_019601.3 | TGCT, OV |
| SNRPD3 | small nuclear | NM_004175.4, | THYM |
| | ribonucleoprotein D3 | NM_001278656.1 | |
| | polypeptide | | |
| TIMP3 | TIMP metallopeptidase | NM_000362.4 | DLBC, THYM, |
| | inhibitor 3 | | PAAD |
| APOL4 | apolipoprotein L4 | NM_030643.4 | GBM |
| C1QTNF6 | C1q and TNF related 6 | NM_031910.3 | PAAD, GBM |
| KDELR3 | KDEL endoplasmic reticulum | NM_006855.3 | PAAD, OV |
| | protein retention receptor 3 | | |
| ZC3H7B | zinc finger CCCH-type | NM_017590.5 | THYM |
| | containing 7B | | |
| WNT7B | Wnt family member 7B | NM_058238.2 | CESC, PAAD, |
| | | | LUSC |

TABLE 2
Target transcripts and exons

| gene | transcript | chr | start | end | strand |
|---|---|---|---|---|---|
| MPZL1 | NM_001146191.1 | chr1 | 167765583 | 167765749 | + |
| AGRN | NM_198576.3 | chr1 | 1054448 | 1054551 | + |
| WLS | NM_001002292.3, NM_024911.6 | chr1 | 68137780 | 68137933 | − |
| KCNAB2 | NM_172130.2 | chr1 | 6097269 | 6097357 | + |
| SLC2A5 | NM_003039.2 | chr1 | 9037897 | 9038024 | − |
| SLC9A1 | NM_003047.4 | chr1 | 27101203 | 27101275 | − |
| NKAIN1 | NM_024522.2 | chr1 | 31181860 | 31181941 | − |
| MAP7D1 | NM_001286366.1 | chr1 | 36179874 | 36180067 | + |
| RHBDL2 | NM_017821.4, NM_001304746.1 | chr1 | 38887963 | 38888024 | − |
| CYP2J2 | NM_000775.3 | chr1 | 59900965 | 59901103 | − |
| GPSM2 | NM_001321038.1, NM_013296.4 | chr1 | 108924000 | 108924214 | + |
| SORT1 | NM_002959.6 | chr1 | 109314261 | 109314384 | − |
| CD58 | NM_001779.2 | chr1 | 116519231 | 116519267 | − |
| NOTCH2 | NM_001200001.1 | chr1 | 119937282 | 119937466 | − |
| CTSS | NM_001199739.1, NM_004079.4 | chr1 | 150747777 | 150747879 | − |
| PMVK | NM_001323011.1, NM_006556.3, | chr1 | 154926354 | 154926483 | − |
| | NM_001323012.1 | | | | |
| STX6 | NM_005819.5 | chr1 | 180984677 | 180984771 | − |
| CAPN8 | NM_001143962.1 | chr1 | 223543108 | 223543166 | − |
| CAPN2 | NM_001146068.1, NM_001748.4 | chr1 | 223772181 | 223772239 | + |
| PARP1 | NM_001618.3 | chr1 | 226361969 | 226362083 | − |
| GPR137B | NM_003272.3 | chr1 | 236205126 | 236205250 | + |
| COX20 | NM_001312872.1 | chr1 | 244842195 | 244842258 | + |
| RPL22 | NM_000983.3 | chr1 | 6192930 | 6193054 | − |
| GPR153 | NM_207370.2 | chr1 | 6250440 | 6250624 | − |
| PDPN | NM_001006625.1, NM_198389.2 | chr1 | 13614300 | 13614405 | + |
| AKR7A2 | NM_003689.3 | chr1 | 19306018 | 19306147 | − |
| CDA | NM_001785.2 | chr1 | 20613842 | 20613899 | + |
| GALE | NM_000403.3, NM_001127621.1, | chr1 | 23796151 | 23796265 | − |
| | NM_001008216.1 | | | | |
| IL22RA1 | NM_021258.3 | chr1 | 24123302 | 24123423 | − |
| SYTL1 | NM_001193308.1 | chr1 | 27353283 | 27353488 | + |
| THEMIS2 | NM_001286115.1, | chr1 | 27885295 | 27885451 | + |
| | NM_001105556.2 | | | | |
| SMPDL3B | NM_014474.3 | chr1 | 27955949 | 27956082 | + |
| TINAGL1 | NM_022164.2 | chr1 | 31586710 | 31586755 | + |
| SPOCD1 | NM_001281988.1 | chr1 | 31792215 | 31792401 | − |
| MYCL | NM_001033082.2 | chr1 | 39900939 | 39901443 | − |
| PTPRF | NM_130440.2 | chr1 | 43621097 | 43621232 | + |
| ARTN | NM_001136215.1 | chr1 | 43936069 | 43936231 | + |
| MAGOH | NM_002370.3 | chr1 | 53228872 | 53228954 | − |
| AK4 | NM_013410.3 | chr1 | 65224752 | 65224870 | + |
| CYR61 | NM_001554.4 | chr1 | 85582416 | 85582624 | + |
| GBP1 | NM_002053.2 | chr1 | 89054682 | 89054875 | − |
| EPHX4 | NM_173567.4 | chr1 | 92052510 | 92052658 | + |
| GFI1 | NM_001127216.1 | chr1 | 92478588 | 92478753 | − |
| CELSR2 | NM_001408.2 | chr1 | 109273436 | 109273670 | + |
| C1orf162 | NM_001300835.1, NM_174896.3 | chr1 | 111477651 | 111477775 | + |
| C1orf162 | NM_001300834.1 | chr1 | 111477726 | 111477775 | + |
| CTSK | NM_000396.3 | chr1 | 150799168 | 150799273 | − |
| ILF2 | NM_004515.3 | chr1 | 153662705 | 153662795 | − |
| CKS1B | NM_001826.2 | chr1 | 154977987 | 154978114 | + |
| BCAN | NM_021948.4 | chr1 | 156658543 | 156658733 | + |
| MRPL24 | NM_145729.2, NM_024540.3 | chr1 | 156737646 | 156737776 | − |
| HDGF | NM_001319188.1, NM_004494.2, | chr1 | 156743652 | 156743878 | |
| | NM_001319187.1, | | | | |
| | NM_001126051.1 | | | | |
| CD1A | NM_001320652.1, NM_001763.2 | chr1 | 158257421 | 158257511 | + |
| CD1B | NM_001764.2 | chr1 | 158328921 | 158329014 | − |
| CD1E | NM_001185114.1, | chr1 | 158356498 | 158356591 | + |
| | NM_001042585.2, | | | | |
| | NM_001042586.2 | | | | |
| TAGLN2 | NM_001277223.1, NM_003564.2 | chr1 | 159919274 | 159919376 | − |
| F11R | NM_016946.4 | chr1 | 160999043 | 160999091 | − |
| TSTD1 | NM_001113206.1, | chr1 | 161037913 | 161038075 | − |
| | NM_001113207.1 | | | | |
| PFDN2 | NM_012394.3 | chr1 | 161102048 | 161102171 | − |
| ELF3 | NM_001114309.1, NM_004433.4 | chr1 | 202013829 | 202014024 | + |
| CHIT1 | NM_001256125.1 | chr1 | 203217739 | 203217865 | − |
| PIGR | NM_002644.3 | chr1 | 206931497 | 206931555 | − |
| MAP4K4 | NM_001242559.1 | chr2 | 101888796 | 101888935 | + |
| MBOAT2 | NM_001321266.1, | chr2 | 8860613 | 8860764 | |
| | NM_001321267.1, NM_138799.3, | | | | |
| | NM_001321265.1 | | | | |
| ANTXR1 | NM_018153.3 | chr2 | 69124565 | 69124643 | + |
| KIAA1211L | NM_207362.2 | chr2 | 98796120 | 98796264 | − |
| FHL2 | NM_201555.1, NM_001318895.1, | chr2 | 105363285 | 105363471 | − |
| | NM_001318898.1, NM_001450.3, | | | | |
| | NM_001039492.2, | | | | |
| | NM_001318896.1, | | | | |
| | NM_001318899.1 | | | | |
| CYTIP | NM_004288.4 | chr2 | 157418523 | 157418589 | − |
| ACVR1 | NM_001111067.2 | chr2 | 157738440 | 157738570 | − |
| DPP4 | NM_001935.3 | chr2 | 161994961 | 161995034 | − |
| WNT10A | NM_025216.2 | chr2 | 218889984 | 218890363 | + |
| DOCK10 | NM_001290263.1 | chr2 | 224770211 | 224770349 | − |
| UGT1A10, | NM_001072.3, NM_205862.1, | chr2 | 233768220 | 233768439 | + |
| UGT1A6 | NM_019075.2 | | | | |
| MLPH | NM_001281473.1, | chr2 | 237552337 | 237552437 | + |
| | NM_001042467.2 | | | | |
| PDIA6 | NM_005742.3 | chr2 | 10784934 | 10785030 | − |
| SF3B6 | NM_016047.3 | chr2 | 24068321 | 24068459 | − |
| ATRAID | NM_016085.4, NM_001170795.1 | chr2 | 27216523 | 27216620 | + |
| CALM2 | NM_001743.5 | chr2 | 47161723 | 47161858 | − |
| ACTR2 | NM_005722.3 | chr2 | 65265043 | 65265175 | + |
| PLEK | NM_002664.2 | chr2 | 68394107 | 68394176 | + |
| ACTG2 | NM_001615.3 | chr2 | 73916584 | 73916765 | + |
| BOLA3 | NM_001035505.1 | chr2 | 74145189 | 74145303 | − |
| LOXL3 | NM_001289165.1, | chr2 | 74533882 | 74533993 | − |
| | NM_001289164.1 | | | | |
| GNLY | NM_006433.4 | chr2 | 85697506 | 85697677 | + |
| CD8A | NM_171827.3 | chr2 | 86788530 | 86788560 | − |
| CD8B | NM_004931.4 | chr2 | 86844922 | 86844958 | − |
| SULT1C4 | NM_006588.3, NM_001321770.1 | chr2 | 108386192 | 108386372 | + |
| FAP | NM_004460.3 | chr2 | 162172811 | 162172884 | − |
| CERKL | NM_201548.4 | chr2 | 181539092 | 181539264 | − |
| PPIL3 | NM_130906.2 | chr2 | 200876919 | 200877037 | − |
| NOP58 | NM_015934.4 | chr2 | 202302921 | 202303057 | + |
| IHH | NM_002181.3 | chr2 | 219057433 | 219057694 | − |
| SP140 | NM_001005176.2 | chr2 | 230238213 | 230238381 | + |
| ITM2C | NM_001012514.2, NM_030926.5, | chr2 | 230877400 | 230877550 | + |
| | NM_001287241.1, | | | | |
| | NM_001012516.2 | | | | |
| PLS1 | NM_001145319.1 | chr3 | 142711501 | 142711625 | + |
| FBLN2 | NM_001998.2 | chr3 | 13636445 | 13636568 | + |
| CDCP1 | NM_022842.4 | chr3 | 45089054 | 45089141 | − |
| ITGB5 | NM_002213.4 | chr3 | 124764391 | 124764557 | − |
| CHCHD6 | NM_001320610.1, NM_032343.2 | chr3 | 126957416 | 126957551 | + |
| BHLHE40 | NM_003670.2 | chr3 | 4981392 | 4981515 | + |
| MYD88 | NM_001172567.1 | chr3 | 38140757 | 38140848 | + |
| MYRIP | NM_001284426.1 | chr3 | 40251881 | 40251999 | + |
| GNL3 | NM_014366.4 | chr3 | 52694037 | 52694103 | + |
| CMSS1 | NM_032359.3 | chr3 | 100176327 | 100176415 | + |
| CD96 | NM_198196.2 | chr3 | 111647543 | 111647666 | + |
| PARP9 | NM_001146104.1 | chr3 | 122536168 | 122536342 | − |
| TMCC1 | NM_001128224.2 | chr3 | 129654968 | 129655103 | − |
| CLDN1 | NM_021101.4 | chr3 | 190310169 | 190310253 | − |
| TIMP4 | NM_003256.3 | chr3 | 12154327 | 12154451 | − |
| RPSA | NM_001304288.1 | chr3 | 39411896 | 39412061 | + |
| CTNNB1 | NM_001904.3 | chr3 | 41238016 | 41238076 | + |
| TDGF1 | NM_001174136.1 | chr3 | 46579951 | 46580060 | + |
| SEMA3F | NM_001318798.1, NM_004186.4 | chr3 | 50186613 | 50186746 | + |
| RRP9 | NM_004704.4 | chr3 | 51933708 | 51933781 | − |
| FAM107A | NM_001076778.2 | chr3 | 58567208 | 58567364 | − |
| HGD | NM_000187.3 | chr3 | 120633147 | 120633328 | − |
| CD86 | NM_175862.4 | chr3 | 122118048 | 122118093 | + |
| PARP15 | NM_001308320.1 | chr3 | 122635020 | 122635194 | + |
| HMCES | NM_001006109.1 | chr3 | 129301950 | 129302142 | + |
| RPL22L1 | NM_001320451.1 | chr3 | 170868013 | 170868134 | − |
| DNAJC19 | NM_145261.3, NM_001190233.1 | chr3 | 180985926 | 180985996 | − |
| ECE2 | NM_032331.3 | chr3 | 184257473 | 184257756 | + |
| MUC20 | NM_001282506.1 | chr3 | 195729648 | 195729739 | + |
| ELOVL6 | NM_001130721.1, NM_024090.2 | chr4 | 110059603 | 110059754 | − |
| SORBS2 | NM_001145672.1 | chr4 | 185589679 | 185589785 | − |
| WDR1 | NM_017491.3, NM_005112.4 | chr4 | 10077304 | 10077448 | − |
| SEL1L3 | NM_001297592.1, NM_015187.4 | chr4 | 25757534 | 25757606 | − |
| RBM47 | NM_019027.3 | chr4 | 40432651 | 40432862 | − |
| SPON2 | NM_012445.3 | chr4 | 1170402 | 1170576 | − |
| C4orf48 | NM_001168243.1 | chr4 | 2042736 | 2042824 | + |
| PPP2R2C | NM_181876.2 | chr4 | 6329262 | 6329353 | − |
| HTRA3 | NM_001297559.1 | chr4 | 8294087 | 8294201 | + |
| CPZ | NM_003652.3, NM_001014447.2 | chr4 | 8618429 | 8618528 | + |
| SLC34A2 | NM_006424.2, NM_001177998.1 | chr4 | 25674505 | 25674629 | + |
| SPINK2 | NM_021114.3 | chr4 | 56811685 | 56811794 | − |
| IGFBP7 | NM_001553.2 | chr4 | 57032426 | 57032552 | − |
| PKD2 | NM_000297.3 | chr4 | 88074812 | 88074959 | + |
| UGT8 | NM_001322113.1, | chr4 | 114668085 | 114668304 | + |
| | NM_001322112.1, | | | | |
| | NM_001128174.2, | | | | |
| | NM_001322114.1 | | | | |
| EDNRA | NM_001957.3 | chr4 | 147540377 | 147540485 | + |
| HMGB2 | NM_001130689.1, NM_002129.3, | chr4 | 173332821 | 173332995 | − |
| | NM_001130688.1 | | | | |
| CENPU | NM_024629.3 | chr4 | 184697647 | 184697803 | − |
| PDLIM3 | NM_001114107.4 | chr4 | 185504475 | 185504586 | − |
| ECSCR | NM_001077693.3 | chr5 | 139449078 | 139449174 | − |
| SLIT3 | NM_001271946.1 | chr5 | 168669783 | 168669991 | − |
| SLC45A2 | NM_001012509.3 | chr5 | 33951554 | 33951677 | − |
| UGT3A2 | NM_174914.3 | chr5 | 36037797 | 36038016 | − |
| OSMR | NM_001323505.1, NM_003999.2, | chr5 | 38932463 | 38932535 | + |
| | NM_001323506.1 | | | | |
| CSF1R | NM_005211.3, NM_001288705.1 | chr5 | 150054322 | 150054430 | − |
| GM2A | NM_001167607.1 | chr5 | 151266731 | 151266899 | + |
| G3BP1 | NM_005754.2, NM_198395.1 | chr5 | 151800760 | 151800869 | + |
| ATP10B | NM_025153.2 | chr5 | 160569496 | 160569683 | − |
| WWC1 | NM_001161662.1 | chr5 | 168467843 | 168467964 | + |
| WWC1 | NM_001161661.1 | chr5 | 168467840 | 168467964 | + |
| EMB | NM_198449.2 | chr5 | 50399859 | 50399913 | − |
| ALDH7A1 | NM_001202404.1 | chr5 | 126546324 | 126546399 | − |
| TCF7 | NM_213648.3, NM_201632.3 | chr5 | 134143592 | 134143640 | + |
| PCDH1 | NM_032420.3 | chr5 | 141857252 | 141857471 | − |
| SPINK1 | NM_003122.4 | chr5 | 147828022 | 147828128 | − |
| SPARC | NM_003118.3, NM_001309443.1 | chr5 | 151664087 | 151664235 | − |
| DUSP1 | NM_004417.3 | chr5 | 172769575 | 172769794 | − |
| STC2 | NM_003714.2 | chr5 | 173323219 | 173323430 | − |
| NOP16 | NM_001291305.2, | chr5 | 176385221 | 176385327 | − |
| | NM_001256540.3 | | | | |
| PDLIM7 | NM_005451.4 | chr5 | 177483867 | 177483982 | − |
| PDLIM7 | NM_213636.2 | chr5 | 177490870 | 177490906 | − |
| RAB44 | NM_001257357.1 | chr6 | 36730673 | 36730749 | + |
| SLC22A3 | NM_021977.3 | chr6 | 160447719 | 160447818 | + |
| IRF4 | NM_001195286.1, NM_002460.3 | chr6 | 405018 | 405130 | + |
| ALDH5A1 | NM_001080.3 | chr6 | 24532119 | 24532177 | + |
| CCDC167 | NM_138493.2 | chr6 | 37484810 | 37484862 | − |
| AIM1 | NM_001624.3 | chr6 | 106563764 | 106563926 | + |
| RPF2 | NM_032194.2, NM_001289111.1 | chr6 | 111024183 | 111024327 | + |
| MOXD1 | NM_015529.3 | chr6 | 132297787 | 132297955 | − |
| SLC17A4 | NM_005495.2 | chr6 | 25777926 | 25778016 | + |
| PRSS16 | NM_005865.3 | chr6 | 27254986 | 27255131 | + |
| SFTA2 | NM_205854.2 | chr6 | 30931707 | 30931795 | − |
| SLC44A4 | NM_001178044.1, NM_025257.2 | chr6 | 31864652 | 31864736 | − |
| CFB | NM_001710.5 | chr6 | 31951555 | 31951604 | + |
| C4A | NM_007293.2 | chr6 | 32002123 | 32002255 | + |
| C4B | NM_001002029.3 | chr6 | 32034860 | 32034992 | + |
| GPSM3 | NM_001276501.1 | chr6 | 32191709 | 32191908 | − |
| HLA-DOB | NM_002120.3 | chr6 | 32813440 | 32813471 | − |
| TAP2 | NM_001290043.1 | chr6 | 32829400 | 32829536 | − |
| PSMB9 | NM_002800.4 | chr6 | 32858364 | 32858505 | + |
| HLA-DOA | NM_002119.3 | chr6 | 33007080 | 33007215 | − |
| TREM2 | NM_001271821.1 | chr6 | 41159792 | 41159882 | − |
| C6orf132 | NM_001164446.2 | chr6 | 42104463 | 42107583 | − |
| MRPL14 | NM_001318770.1, | chr6 | 44116541 | 44116629 | − |
| | NM_001318769.1 | | | | |
| HSP90AB1 | NM_001271971.1, NM_007355.3, | chr6 | 44253045 | 44253378 | + |
| | NM_001271972.1 | | | | |
| MEP1A | NM_005588.2 | chr6 | 46835249 | 46835549 | + |
| TNFRSF21 | NM_014452.4 | chr6 | 47234670 | 47234898 | − |
| CTGF | NM_001901.2 | chr6 | 131949949 | 131950160 | − |
| TAGAP | NM_001278733.1, NM_054114.4 | chr6 | 159038114 | 159038228 | − |
| AOAH | NM_001637.3 | chr7 | 36522039 | 36522115 | − |
| PRKAR1B | NM_001164759.1, | chr7 | 551389 | 551470 | − |
| | NM_001164762.1, | | | | |
| | NM_001164760.1 | | | | |
| ADAP1 | NM_001284311.1 | chr7 | 899033 | 899261 | − |
| MICALL2 | NM_182924.3 | chr7 | 1435101 | 1435147 | − |
| MAD1L1 | NM_001304525.1 | chr7 | 1898200 | 1898390 | − |
| AHR | NM_001621.4 | chr7 | 17338986 | 17340228 | + |
| CREB5 | NM_182898.3 | chr7 | 28818071 | 28818179 | + |
| NUDT1 | NM_198950.1, NM_002452.3, | chr7 | 2249857 | 2250002 | + |
| | NM_198948.1 | | | | |
| LFNG | NM_001040167.1 | chr7 | 2526836 | 2526921 | + |
| MEOX2 | NM_005924.4 | chr7 | 15626746 | 15626918 | − |
| SFRP4 | NM_003014.3 | chr7 | 37909617 | 37909680 | − |
| YAE1D1 | NM_020192.4 | chr7 | 39570506 | 39570627 | + |
| MYO1G | NM_033054.2 | chr7 | 44962970 | 44963124 | − |
| RAMP3 | NM_005856.2 | chr7 | 45177309 | 45177441 | + |
| CNPY4 | NM_152755.1 | chr7 | 100124514 | 100124631 | + |
| MEST | NM_001253900.1, NM_002402.3, | chr7 | 130503933 | 130503996 | + |
| | NM_001253901.1, NM_177524.2, | | | | |
| | NM_177525.2 | | | | |
| AKR1B10 | NM_020299.4 | chr7 | 134538935 | 134539017 | + |
| ATP6V0A4 | NM_130841.2 | chr7 | 138709624 | 138709795 | − |
| CLEC5A | NM_013252.2 | chr7 | 141931720 | 141931826 | − |
| PIP | NM_002652.2 | chr7 | 143139075 | 143139189 | + |
| TCAF1 | NM_001206941.1, NM_014719.2 | chr7 | 143857100 | 143857350 | − |
| DNAJC5B | NM_033105.4 | chr8 | 66080377 | 66080548 | + |
| MTUS1 | NM_001166393.1 | chr8 | 17646982 | 17647079 | − |
| LOXL2 | NM_002318.2 | chr8 | 23298836 | 23298947 | − |
| DOCK5 | NM_024940.7 | chr8 | 25410099 | 25410202 | + |
| DPYSL2 | NM_001197293.2, NM_001386.5 | chr8 | 26653232 | 26653397 | + |
| SULF1 | NM_001128206.1, | chr8 | 69640808 | 69640841 | + |
| | NM_001128204.1, NM_015170.2, | | | | |
| | NM_001128205.1 | | | | |
| TPD52 | NM_001025253.2 | chr8 | 80042620 | 80042668 | − |
| FAM83A | NM_032899.6 | chr8 | 123194024 | 123194148 | + |
| TATDN1 | NM_032026.3 | chr8 | 124493833 | 124493959 | − |
| SLA | NM_001045556.2 | chr8 | 133039998 | 133040130 | − |
| KHDRBS3 | NM_006558.2 | chr8 | 135645059 | 135645117 | + |
| SMIM19 | NM_138436.3 | chr8 | 42548656 | 42548780 | + |
| MRPL15 | NM_014175.3 | chr8 | 54142663 | 54142786 | + |
| TRAM1 | NM_001317804.1, NM_014294.5 | chr8 | 70583164 | 70583324 | − |
| CRISPLD1 | NM_001286778.1, NM_031461.5 | chr8 | 75029387 | 75029517 | + |
| CHMP4C | NM_152284.3 | chr8 | 81758142 | 81758295 | + |
| ESRP1 | NM_001122827.1 | chr8 | 94678203 | 94678371 | + |
| NIPAL2 | NM_001321636.1 | chr8 | 98195942 | 98196005 | − |
| CTHRC1 | NM_001256099.1, NM_138455.3 | chr8 | 103378027 | 103378243 | + |
| SQLE | NM_003129.3 | chr8 | 125020784 | 125020871 | + |
| FAM189A2 | NM_001127608.1 | chr9 | 69388158 | 69388450 | + |
| GDA | NM_004293.4 | chr9 | 72247406 | 72247433 | + |
| CORO2A | NM_052820.3 | chr9 | 98126549 | 98126823 | − |
| NDUFA8 | NM_014222.2 | chr9 | 122148112 | 122148277 | − |
| FAM214B | NM_025182.3 | chr9 | 35105682 | 35105857 | − |
| TPM2 | NM_001301226.1 | chr9 | 35684246 | 35684315 | − |
| CTSL | NM_001257973.1, | chr9 | 87730381 | 87730498 | + |
| | NM_001257971.1, NM_001912.4, | | | | |
| | NM_001257972.1, NM_145918.2 | | | | |
| CTSV | NM_001333.3 | chr9 | 97034726 | 97034843 | − |
| GSN | NM_001127666.1, | chr9 | 121331388 | 121331448 | + |
| | NM_001258029.1, | | | | |
| | NM_001127667.1 | | | | |
| STOM | NM_198194.2 | chr9 | 121356053 | 121356156 | − |
| PTGS1 | NM_080591.2, NM_000962.3 | chr9 | 122390198 | 122390345 | + |
| ZDHHC12 | NM_032799.4, NM_001318016.1, | chr9 | 128721651 | 128721817 | − |
| | NM_001318015.1 | | | | |
| ZDHHC12 | NM_001318023.1 | chr9 | 128721651 | 128721814 | − |
| PRRX2 | NM_016307.3 | chr9 | 129720596 | 129720774 | + |
| SURF2 | NM_017503.4, NM_001278928.1 | chr9 | 133360265 | 133360434 | + |
| PHPT1 | NM_014172.5, NM_001287342.1 | chr9 | 136850013 | 136850137 | + |
| PTGDS | NM_000954.5 | chr9 | 136980833 | 136980855 | + |
| CLIC3 | NM_004669.2 | chr9 | 136994930 | 136995105 | − |
| DHRSX | NM_145177.2 | chrX | 2243023 | 2243230 | − |
| SLC9A7 | NM_001257291.1 | chrX | 46613289 | 46613394 | − |
| POF1B | NM_024921.3 | chrX | 85282203 | 85282317 | − |
| GPC3 | NM_001164617.1, | chrX | 133596440 | 133596599 | − |
| | NM_001164619.1, NM_004484.3, | | | | |
| | NM_001164618.1 | | | | |
| CSF2RA | NM_006140.4, NM_172245.2 | chrX | 1305446 | 1305527 | + |
| ARSD | NM_001669.3 | chrX | 2908721 | 2908842 | − |
| ARSE | NM_000047.2 | chrX | 2936742 | 2936863 | − |
| GPR143 | NM_000273.2 | chrX | 9739485 | 9739719 | − |
| USP11 | NM_004651.3 | chrX | 47247611 | 47247699 | + |
| SLC38A5 | NM_033518.3 | chrX | 48459536 | 48459639 | − |
| PLP2 | NM_002668.2 | chrX | 49174335 | 49174425 | + |
| IL2RG | NM_000206.2 | chrX | 71108277 | 71108346 | − |
| CITED1 | NM_001144885.1 | chrX | 72302810 | 72302934 | − |
| NOX1 | NM_007052.4, NM_013955.2 | chrX | 100848630 | 100848754 | − |
| SASH3 | NM_018990.3 | chrX | 129792989 | 129793139 | + |
| IGSF1 | NM_001170962.1, NM_001555.4 | chrX | 131274083 | 131274206 | − |
| HMGB3 | NM_005342.3, NM_001301231.1, | chrX | 150987128 | 150987302 | + |
| | NM_001301228.1, | | | | |
| | NM_001301229.1 | | | | |
| CSAG1 | NM_001102576.2 | chrX | 152728074 | 152728224 | − |
| BGN | NM_001711.5 | chrX | 153507047 | 153507185 | + |
| L1CAM | NM_001143963.2 | chrX | 153863477 | 153863549 | − |
| DHRSX | NM_145177.2 | chrY | 2243023 | 2243230 | − |
| CSF2RA | NM_006140.4, NM_172245.2 | chrY | 1305446 | 1305527 | + |
| CCDC3 | NM_031455.3 | chr10 | 12998338 | 12998512 | − |
| TBATA | NM_001318243.1, | chr10 | 70772514 | 70772566 | − |
| | NM_001318242.1, NM_152710.3 | | | | |
| PIK3AP1 | NM_152309.2 | chr10 | 96602280 | 96602398 | − |
| NET1 | NM_005863.4 | chr10 | 5456087 | 5456273 | + |
| ATP5C1 | NM_005174.3 | chr10 | 7802758 | 7802854 | + |
| SRGN | NM_002727.3, NM_001321053.1 | chr10 | 69097084 | 69097231 | + |
| H2AFY2 | NM_018649.2 | chr10 | 70109033 | 70109207 | + |
| PPA1 | NM_021129.3 | chr10 | 70204873 | 70204915 | − |
| UNC5B | NM_001244889.1 | chr10 | 71297909 | 71298090 | + |
| PLAU | NM_002658.4 | chr10 | 73915251 | 73915399 | + |
| DUSP5 | NM_004419.3 | chr10 | 110506935 | 110507154 | + |
| VENTX | NM_014468.3 | chr10 | 133239676 | 133239836 | + |
| FUOM | NM_001098483.2 | chr10 | 133355738 | 133355811 | − |
| LDLRAD3 | NM_174902.3 | chr11 | 36227085 | 36227430 | + |
| ANO1 | NM_018043.5 | chr11 | 70185590 | 70185695 | + |
| B4GALNT4 | NM_178537.4 | chr11 | 380825 | 380951 | + |
| RASSF7 | NM_001143993.1 | chr11 | 562079 | 562776 | + |
| CHID1 | NM_001142674.1, | chr11 | 870121 | 870163 | − |
| | NM_001142677.1 | | | | |
| MUC5B | NM_002458.2 | chr11 | 1260626 | 1260728 | + |
| IGF2 | NM_001291861.2, NM_000612.5 | chr11 | 2133517 | 2133665 | − |
| SEC22A18 | NM_183233.2, NM_001315502.1 | chr11 | 2922436 | 2922556 | + |
| DKK3 | NM_013253.4 | chr11 | 11965809 | 11965965 | − |
| SAA2 | NM_030754.4 | chr11 | 18245910 | 18246048 | − |
| SAA1 | NM_199161.4 | chr11 | 18269195 | 18269333 | + |
| HTATIP2 | NM_001098520.1, NM_006410.4 | chr11 | 20382178 | 20382239 | + |
| EHF | NM_012153.5 | chr11 | 34658533 | 34658728 | + |
| CREB3L1 | NM_052854.3 | chr11 | 46320264 | 46320528 | + |
| UBE2L6 | NM_004223.4 | chr11 | 57554437 | 57554623 | − |
| LPXN | NM_004811.2 | chr11 | 58528043 | 58528191 | − |
| PLA2G16 | NM_007069.3, NM_001128203.1 | chr11 | 63590100 | 63590368 | − |
| FERMT3 | NM_031471.5 | chr11 | 64223048 | 64223189 | + |
| OVOL1 | NM_004561.3 | chr11 | 65794538 | 65794727 | + |
| SLC29A2 | NM_001532.2 | chr11 | 66364225 | 66364424 | − |
| FOLR1 | NM_016729.2, NM_016725.2, | chr11 | 72195612 | 72195747 | + |
| | NM_000802.3, NM_016724.2 | | | | |
| MMP10 | NM_002425.2 | chr11 | 102772012 | 102772115 | − |
| MMP1 | NM_001145938.1, NM_002421.3 | chr11 | 102790703 | 102790806 | − |
| MMP3 | NM_002422.4 | chr11 | 102837298 | 102837401 | − |
| MMP12 | NM_002426.5 | chr11 | 102864146 | 102864252 | − |
| POU2AF1 | NM_006235.2 | chr11 | 111357445 | 111357710 | − |
| CD3D | NM_001040651.1 | chr11 | 118339451 | 118339494 | − |
| TRIM29 | NM_012101.3 | chr11 | 120115338 | 120115414 | − |
| VSIG2 | NM_014312.3 | chr11 | 124748390 | 124748534 | − |
| BARX2 | NM_003658.4 | chr11 | 129442835 | 129442919 | + |
| GUCY2C | NM_004963.3 | chr12 | 14614867 | 14614943 | − |
| B4GALNT3 | NM_173593.3 | chr12 | 559295 | 559421 | + |
| PLBD1 | NM_024829.5 | chr12 | 14506162 | 14506268 | − |
| ARHGDIB | NM_001175.6, NM_001321421.1 | chr12 | 14944776 | 14944839 | − |
| FAM60A | NM_021238.2, NM_001135812.1, | chr12 | 31287634 | 31287784 | − |
| | NM_001135811.1 | | | | |
| ERBB3 | NM_001982.3 | chr12 | 56101061 | 56101361 | + |
| LRP1 | NM_002332.2 | chr12 | 57212117 | 57212261 | + |
| MSRB3 | NM_198080.3, NM_001193461.1 | chr12 | 65453728 | 65453825 | + |
| ALDH2 | NM_001204889.1 | chr12 | 111803859 | 111803973 | + |
| LAG3 | NM_002286.5 | chr12 | 6777791 | 6777921 | + |
| CLEC12A | NM_001300730.1 | chr12 | 9982020 | 9982129 | + |
| GPRC5A | NM_003979.3 | chr12 | 12912084 | 12912142 | + |
| LDHB | NM_001315537.1, NM_002300.7 | chr12 | 21637071 | 21637194 | − |
| TUBA1A | NM_006009.3, NM_001270400.1 | chr12 | 49186310 | 49186458 | − |
| TROAP | NM_001100620.1 | chr12 | 49323845 | 49324037 | + |
| KRT6B | NM_005555.3 | chr12 | 52447539 | 52447573 | − |
| KRT6C | NM_173086.4 | chr12 | 52469411 | 52469445 | − |
| KRT6A | NM_005554.3 | chr12 | 52488069 | 52488103 | − |
| KRT8 | NM_002273.3 | chr12 | 52898461 | 52898519 | − |
| SLC39A5 | NM_001135195.1 | chr12 | 56237150 | 56237340 | + |
| DCN | NM_133503.3 | chr12 | 91151654 | 91151792 | − |
| IKBIP | NM_153687.3 | chr12 | 98634296 | 98634413 | − |
| OAS1 | NM_016816.3 | chr12 | 112917547 | 112917700 | + |
| OAS3 | NM_006187.3 | chr12 | 112969608 | 112969755 | + |
| FLT3 | NM_004119.2 | chr13 | 28014452 | 28014557 | − |
| FARP1 | NM_001286839.1 | chr13 | 98446666 | 98446817 | + |
| COL4A1 | NM_001845.5 | chr13 | 110152334 | 110152506 | − |
| COL4A2 | NM_001846.2 | chr13 | 110507935 | 110508221 | + |
| HMGB1 | NM_001313892.1 | chr13 | 30462538 | 30462712 | − |
| ALOX5AP | NM_001629.3 | chr13 | 30755944 | 30756025 | + |
| TNFSF13B | NM_006573.4 | chr13 | 108303454 | 108303604 | + |
| MNAT1 | NM_001177963.1 | chr14 | 60879714 | 60879835 | + |
| SLC7A7 | NM_001126106.2 | chr14 | 22773933 | 22774116 | − |
| LRFN5 | NM_152447.3 | chr14 | 41898917 | 41898960 | + |
| TMX1 | NM_030755.4 | chr14 | 51249693 | 51249765 | + |
| DLGAP5 | NM_014750.4 | chr14 | 55150799 | 55150848 | − |
| SMOC1 | NM_001034852.2, NM_022137.5 | chr14 | 70023203 | 70023447 | + |
| RIN3 | NM_001319987.1 | chr14 | 92684987 | 92685150 | + |
| VRK1 | NM_003384.2 | chr14 | 96876030 | 96876120 | + |
| PNP | NM_000270.3 | chr14 | 20475062 | 20475252 | + |
| MMP14 | NM_004995.3 | chr14 | 22845251 | 22845366 | + |
| NGDN | NM_001042635.1 | chr14 | 23477503 | 23477560 | + |
| DHRS2 | NM_005794.3 | chr14 | 23644827 | 23644882 | + |
| FKBP3 | NM_002013.3 | chr14 | 45118028 | 45118125 | − |
| PYGL | NM_001163940.1, NM_002863.4 | chr14 | 50908271 | 50908337 | − |
| ERH | NM_004450.2 | chr14 | 69386963 | 69387083 | − |
| SERPINA3 | NM_001085.4 | chr14 | 94622341 | 94622491 | + |
| AMN | NM_030943.3 | chr14 | 102930406 | 102930493 | + |
| SIVA1 | NM_006427.3, NM_021709.2 | chr14 | 104756604 | 104756760 | + |
| PLD4 | NM_001308174.1 | chr14 | 104932259 | 104932355 | + |
| THSD4 | NM_001286429.1, NM_024817.2 | chr15 | 71771064 | 71771208 | + |
| MCTP2 | NM_018349.3 | chr15 | 94476696 | 94476793 | + |
| THBS1 | NM_003246.3 | chr15 | 39594301 | 39594440 | + |
| FBN1 | NM_000138.4 | chr15 | 48412569 | 48412743 | − |
| PKM | NM_002654.5, NM_001206797.2 | chr15 | 72200474 | 72200655 | − |
| CHSY1 | NM_014918.4 | chr15 | 101235082 | 101235577 | − |
| LPCAT4 | NM_153613.2 | chr15 | 34359589 | 34359745 | − |
| PHGR1 | NM_001145643.1 | chr15 | 40354345 | 40354352 | + |
| NUSAP1 | NM_018454.7, NM_001301136.1, | chr15 | 41377196 | 41377304 | + |
| | NM_001243143.1 | | | | |
| DUOXA2 | NM_207581.3 | chr15 | 45117091 | 45117305 | + |
| SQRDL | NM_001271213.1 | chr15 | 45689039 | 45689217 | + |
| CA12 | NM_001218.4, NM_206925.2 | chr15 | 63327149 | 63327233 | − |
| LOXL1 | NM_005576.3 | chr15 | 73949459 | 73949574 | + |
| PML | NM_033238.2 | chr15 | 74042989 | 74043139 | + |
| SEMA7A | NM_001146030.2 | chr15 | 74411295 | 74411356 | − |
| CSPG4 | NM_001897.4 | chr15 | 75677703 | 75677886 | − |
| ANPEP | NM_001150.2 | chr15 | 89790460 | 89790541 | − |
| PLCG2 | NM_002661.4 | chr16 | 81956695 | 81956879 | + |
| SEZ6L2 | NM_012410.3, NM_001114099.2, | chr16 | 29872187 | 29872283 | − |
| | NM_001243332.1, NM_201575.3, | | | | |
| | NM_001114100.2 | | | | |
| POLR3K | NM_016310.4 | chr16 | 51558 | 51645 | − |
| SOX8 | NM_014587.4 | chr16 | 983728 | 983960 | + |
| TPSB2 | NM_024164.5 | chr16 | 1228900 | 1229063 | − |
| TPS AB 1 | NM_003294.3 | chr16 | 1241827 | 1241990 | + |
| HN1L | NM_144570.2 | chr16 | 1697812 | 1697860 | + |
| SYNGR3 | NM_004209.5 | chr16 | 1992636 | 1992778 | + |
| NTHL1 | NM_001318194.1, NM_002528.6 | chr16 | 2040133 | 2040238 | − |
| ECU | NM_001178029.1 | chr16 | 2243046 | 2243224 | − |
| TCEB2 | NM_207013.2 | chr16 | 2771994 | 2772102 | − |
| PRSS22 | NM_022119.3 | chr16 | 2853865 | 2854022 | − |
| PAQR4 | NM_001324118.1 | chr16 | 2971515 | 2971684 | + |
| SMIM22 | NM_001253793.1 | chr16 | 4795948 | 4796033 | + |
| NDE1 | NM_017668.2 | chr16 | 15696709 | 15696860 | + |
| ERN2 | NM_001308220.1, NM_033266.3 | chr16 | 23691129 | 23691196 | − |
| IL21R | NM_181078.2 | chr16 | 27446007 | 27446088 | + |
| EIF3C | NM_001286478.1 | chr16 | 28735173 | 28735335 | + |
| SULT1A4 | NM_001017390.2 | chr16 | 29464160 | 29464340 | + |
| DOC2A | NM_003586.2, NM_001282062.1 | chr16 | 30006413 | 30006509 | − |
| TGFB1I1 | NM_001164719.1, | chr16 | 31476862 | 31477010 | + |
| | NM_001042454.2 | | | | |
| MMP2 | NM_001302510.1, | chr16 | 55502779 | 55502888 | + |
| | NM_001302508.1, | | | | |
| | NM_001302509.1, NM_004530.5 | | | | |
| CES1 | NM_001025195.1 | chr16 | 55803529 | 55803601 | − |
| ADGRG1 | NM_001145771.2, | chr16 | 57661697 | 57661965 | + |
| | NM_001145774.2, | | | | |
| | NM_001145770.2, | | | | |
| | NM_001290142.1, NM_005682.6, | | | | |
| | NM_001290144.1 | | | | |
| CMTM3 | NM_181553.2 | chr16 | 66609883 | 66610003 | + |
| NQO1 | NM_001286137.1 | chr16 | 69718123 | 69718253 | − |
| CLEC18B | NM_001011880.2 | chr16 | 74409550 | 74409641 | − |
| SLC7A5 | NM_003486.5 | chr16 | 87834414 | 87834591 | − |
| CYBA | NM_000101.3 | chr16 | 88646116 | 88646197 | − |
| CDT1 | NM_030928.3 | chr16 | 88807281 | 88807482 | + |
| SLC22A31 | NM_001242757.1 | chr16 | 89197298 | 89197409 | − |
| SPNS3 | NM_001320449.1, NM_182538.4 | chr17 | 4486412 | 4486583 | + |
| ADAP2 | NM_018404.2 | chr17 | 30956241 | 30956469 | + |
| SLC16A5 | NM_004695.3 | chr17 | 75103970 | 75104180 | + |
| MYO1C | NM_033375.4 | chr17 | 1467242 | 1467341 | − |
| TM4SF5 | NM_003963.2 | chr17 | 4782854 | 4783037 | + |
| TNFSF13 | NM_003808.3 | chr17 | 7560350 | 7560488 | + |
| NAA38 | NM_001320924.1 | chr17 | 7856985 | 7857198 | − |
| PIK3R6 | NM_001010855.3, | chr17 | 8804041 | 8804153 | − |
| | NM_001290211.1 | | | | |
| CCL2 | NM_002982.3 | chr17 | 34256222 | 34256339 | + |
| SLFN11 | NM_001104589.1, NM_152270.3, | chr17 | 35353336 | 35354059 | − |
| | NM_001104588.1, | | | | |
| | NM_001104587.1, | | | | |
| | NM_001104590.1 | | | | |
| CCL5 | NM_002985.2 | chr17 | 35878528 | 35878639 | − |
| GRB7 | NM_001030002.2, NM_005310.3 | chr17 | 39746109 | 39746202 | + |
| KRT16 | NM_005557.3 | chr17 | 41610190 | 41610236 | − |
| KRT17 | NM_000422.2 | chr17 | 41620536 | 41620558 | − |
| JUP | NM_002230.2 | chr17 | 41756175 | 41756214 | − |
| MPO | NM_000250.1 | chr17 | 58271655 | 58271892 | − |
| LIMD2 | NM_030576.3 | chr17 | 63698799 | 63698938 | − |
| SLC25A19 | NM_001126122.1 | chr17 | 75277353 | 75277483 | − |
| ST6GALNAC2 | NM_006456.2 | chr17 | 76567453 | 76567552 | − |
| TMC8 | NM_152468.4 | chr17 | 78139162 | 78139240 | + |
| BAIAP2 | NM_001144888.1, NM_017450.2 | chr17 | 81108475 | 81108509 | + |
| ALYREF | NM_005782.3 | chr17 | 81888241 | 81888418 | − |
| KLHL14 | NM_020805.2 | chr18 | 32677173 | 32677330 | − |
| MBP | NM_001025100.1 | chr18 | 77066298 | 77066385 | − |
| NDC80 | NM_006101.2 | chr18 | 2610759 | 2610861 | + |
| TTR | NM_000371.3 | chr18 | 31595120 | 31595255 | + |
| CYB5A | NM_001190807.2 | chr18 | 74255741 | 74255775 | − |
| GNA15 | NM_002068.3 | chr19 | 3157728 | 3157881 | + |
| MUC16 | NM_024690.2 | chr19 | 8851276 | 8851355 | − |
| DDX39A | NM_005804.3 | chr19 | 14409037 | 14409184 | − |
| KLC3 | NM_177417.2 | chr19 | 45350954 | 45351017 | + |
| PTPRH | NM_002842.4 | chr19 | 55182016 | 55182151 | − |
| GNA11 | NM_002067.4 | chr19 | 3119206 | 3119359 | + |
| CREB3L3 | NM_032607.2 | chr19 | 4171383 | 4171479 | + |
| CD70 | NM_001252.4 | chr19 | 6590103 | 6590136 | − |
| PRAM1 | NM_032152.4 | chr19 | 8490338 | 8490372 | − |
| MRPL4 | NM_146387.1 | chr19 | 10258609 | 10258685 | + |
| AP1M2 | NM_001300887.1, NM_005498.4 | chr19 | 10574417 | 10574492 | − |
| SPC24 | NM_182513.3, NM_001317033.1 | chr19 | 11147818 | 11147894 | − |
| RAB3D | NM_004283.3 | chr19 | 11335447 | 11335571 | − |
| ZNF439 | NM_152262.2 | chr19 | 11866537 | 11866597 | + |
| MAN2B1 | NM_000528.3 | chr19 | 12647233 | 12647335 | − |
| RNASEH2A | NM_006397.2 | chr19 | 12813083 | 12813206 | + |
| FARSA | NM_004461.2 | chr19 | 12924151 | 12924265 | − |
| NACC1 | NM_052876.3 | chr19 | 13137478 | 13137575 | + |
| GIPC1 | NM_202468.2, NM_202494.2, | chr19 | 14478684 | 14478765 | − |
| | NM_005716.3 | | | | |
| NOTCH3 | NM_000435.2 | chr19 | 15162465 | 15162562 | − |
| TPM4 | NM_003290.2 | chr19 | 16093684 | 16093753 | + |
| PLVAP | NM_031310.2 | chr19 | 17360528 | 17360609 | − |
| LSM4 | NM_001252129.1 | chr19 | 18309678 | 18309861 | − |
| CCNE1 | NM_001322262.1, | chr19 | 29822446 | 29822603 | + |
| | NM_001322261.1, | | | | |
| | NM_001322259.1, NM_001238.3 | | | | |
| GPI | NM_001184722.1 | chr19 | 34399719 | 34399785 | + |
| FXYD3 | NM_001136007.1, | chiT9 | 35123271 | 35123308 | + |
| | NM_001136011.1, NM_005971.3, | | | | |
| | NM_001136012.1 | | | | |
| CD22 | NM_001185101.1, NM_001771.3 | chr19 | 35346151 | 35346235 | + |
| TYROBP | NM_001173514.1, NM_003332.3 | chr19 | 35907218 | 35907264 | − |
| TYROBP | NM_001173515.1, NM_198125.2 | chr19 | 35907218 | 35907261 | − |
| CAPNS1 | NM_001302633.1 | chr19 | 36149578 | 36149636 | + |
| COX7A1 | NM_001864.3 | chr19 | 36151462 | 36151546 | − |
| LGALS4 | NM_006149.3 | chr19 | 38801992 | 38802157 | − |
| FBXO27 | NM_178820.4 | chr19 | 39026870 | 39027005 | − |
| GMFG | NM_004877.3 | chr19 | 39329000 | 39329073 | − |
| DLL3 | NM_203486.2 | chr19 | 39507830 | 39507914 | + |
| CYP2S1 | NM_030622.7 | chr19 | 41205958 | 41206099 | + |
| TGFB1 | NM_000660.5 | chr19 | 41332128 | 41332281 | − |
| CNFN | NM_032488.3 | chr19 | 42387340 | 42387476 | − |
| SUET2B1 | NM_177973.1 | chr19 | 48596739 | 48596919 | + |
| FAM83E | NM_017708.3 | chr19 | 48603494 | 48603911 | − |
| PLEKHA4 | NM_020904.2 | chr19 | 48838017 | 48838129 | − |
| RRAS | NM_006270.4 | chr19 | 49635734 | 49635852 | − |
| PRMT1 | NM_001536.5, NM_198318.4 | chr19 | 49686605 | 49686726 | + |
| IL4I1 | NM_152899.1 | chr19 | 49890971 | 49891107 | − |
| SIGLEC14 | NM_001098612.1 | chr19 | 51643537 | 51643672 | − |
| TMC4 | NM_144686.3 | chr19 | 54160467 | 54160545 | − |
| ISOC2 | NM_024710.2, NM_001136201.1 | chr19 | 55454989 | 55455106 | − |
| EYA2 | NM_005244.4 | chr20 | 47183291 | 47183391 | + |
| PYGB | NM_002862.3 | chr20 | 25295604 | 25295670 | + |
| E2F1 | NM_005225.2 | chr20 | 33677105 | 33677330 | − |
| FAM83D | NM_030919.2 | chr20 | 38947876 | 38948000 | + |
| TOMM34 | NM_006809.4 | chr20 | 44943453 | 44943579 | − |
| RASSF2 | NM_014737.2, NM_170774.1 | chr20 | 4786231 | 4786328 | − |
| FERMT1 | NM_017671.4 | chr20 | 6079436 | 6079577 | − |
| OVOL2 | NM_021220.3 | chr20 | 18041534 | 18041723 | − |
| EIF6 | NM_001267810.1, NM_181468.2 | chr20 | 35279566 | 35279747 | − |
| ADA | NM_001322050.1, NM_000022.3 | chr20 | 44620299 | 44620401 | − |
| PLTP | NM_001242921.1, NM_182676.2, | chr20 | 45899462 | 45899538 | − |
| | NM_006227.3, NM_001242920.1 | | | | |
| MMP9 | NM_004994.2 | chr20 | 46014371 | 46014474 | + |
| CD40 | NM_001322422.1, | chr20 | 46128330 | 46128358 | + |
| | NM_001322421.1, NM_001250.5 | | | | |
| RCAN1 | NM_203418.2, NM_001285389.2 | chr21 | 34521499 | 34521658 | − |
| SAMSN1 | NM_022136.4 | chr21 | 14498442 | 14498592 | − |
| BTG3 | NM_006806.4 | chr21 | 17598617 | 17598824 | − |
| SOD1 | NM_000454.4 | chr21 | 31667258 | 31667375 | + |
| FAM3B | NM_058186.3 | chr21 | 41348592 | 41348724 | + |
| RIPK4 | NM_020639.2 | chr21 | 41743882 | 41744140 | − |
| SLC37A1 | NM_018964.3 | chr21 | 42579736 | 42579800 | + |
| DNMT3L | NM_175867.2 | chr21 | 44249026 | 44249111 | − |
| SLC5A1 | NM_000343.3 | chr22 | 32104786 | 32104891 | + |
| HMOX1 | NM_002133.2 | chr22 | 35389864 | 35389963 | + |
| CBY1 | NM_015373.3 | chr22 | 38671070 | 38671188 | + |
| BIK | NM_001197.4 | chr22 | 43128496 | 43128625 | + |
| CELSR1 | NM_014246.1 | chr22 | 46363996 | 46364251 | − |
| PRODH | NM_001195226.1 | chr22 | 18913438 | 18913526 | − |
| MIF | NM_002415.1 | chr22 | 23894772 | 23894944 | + |
| SUSD2 | NM_019601.3 | chr22 | 24188225 | 24188327 | + |
| SNRPD3 | NM_004175.4, NM_001278656.1 | chr22 | 24567984 | 24568176 | + |
| TIMP3 | NM_000362.4 | chr22 | 32858017 | 32858138 | + |
| APOL4 | NM_030643.4 | chr22 | 36195311 | 36195437 | − |
| C1QTNF6 | NM_031910.3 | chr22 | 37185218 | 37185455 | − |
| KDELR3 | NM_006855.3 | chr22 | 38481212 | 38481464 | + |
| ZC3H7B | NM_017590.5 | chr22 | 41356645 | 41356808 | + |
| WNT7B | NM_058238.2 | chr22 | 45931098 | 45931369 | − |

Table 2 lists the coordinates, matched to the HG38 genome build, of exons that:

1. Are of length not divisible by 3 and

2. Are the exon before last in at least one gene transcript, and

3. Are part of genes and/or transcripts that are significantly overexpressed in at least one cancer (based on comparison between TCGA and GTEx expression levels) and

4. When excluded (skipped) from at least one transcript will lead to creation of an unnatural aberrant isoform that does not exist in the natural human proteome and that this isoform contains at least one 9-mer that does not exist in the natural human proteome that is predicted to bind with strong affinity to at least one of the HLA alleles listed above.

Table 3 listing the cancer abbreviations used in the description and in Table 1 (as determined by the TCGA consortium):

TABLE 3
Cancers and abbreviations

| Abbreviation | Cancer Name |
|---|---|
| LAML | Acute Myeloid Leukemia |
| ACC | Adrenocortical carcinoma |
| BLCA | Bladder Urothelial Carcinoma |
| LGG | Brain Lower Grade Glioma |
| BRCA | Breast invasive carcinoma |
| CESC | Cervical squamous cell carcinoma and endocervical |
| | adenocarcinoma |
| COAD | Colon adenocarcinoma |
| ESCA | Esophageal carcinoma |
| GBM | Glioblastoma multiforme |
| HNSC | Head and Neck squamous cell carcinoma |
| KICH | Kidney Chromophobe |
| KIRC | Kidney renal clear cell carcinoma |
| KIRP | Kidney renal papillary cell carcinoma |
| LIHC | Liver hepatocellular carcinoma |
| LUAD | Lung adenocarcinoma |
| LUSC | Lung squamous cell carcinoma |
| DLBC | Lymphoid Neoplasm Diffuse Large B-cell Lymphoma |
| OV | Ovarian serous cystadenocarcinoma |
| PAAD | Pancreatic adenocarcinoma |
| PRAD | Prostate adenocarcinoma |
| READ | Rectum adenocarcinoma |
| SKCM | Skin Cutaneous Melanoma |
| STAD | Stomach adenocarcinoma |
| TGCT | Testicular Germ Cell Tumors |
| THYM | Thymoma |
| THCA | Thyroid carcinoma |
| UCS | Uterine Carcinosarcoma |
| UCEC | Uterine Corpus Endometrial Carcinoma |

1. A method for inducing the production of at least one neoantigen to be expressed by at least one target cell of a subject suffering from a neoplastic disorder, the method comprising the step of contacting said at least one target cell with at least one splicing modulating agent comprising at least one nucleic acid sequence or of any vector, vehicle, matrix, nano- or micro-particle or composition comprising said at least one agent;
wherein said at least one nucleic acid sequence of said agent targets at least one target nucleic acid sequence that participates directly or indirectly in at least one splicing event of at least one target gene, wherein the introduction of said at least one agent into said target cell induces at least one aberrant splicing event via said target nucleic acid sequence;
and wherein said aberrant splicing event results in the production of said at least one neoantigen to be expressed by said target cell.

2. The method according to claim 1, wherein said splicing modulating agent comprises at least one of:
(a) at least one oligonucleotide comprising a nucleic acid sequence complementary to at least part of said target nucleic acid sequence; and
(b) at least one nucleic acid sequence comprising at least one guide RNA (gRNA) that targets at least one protospacer within said target nucleic acid sequence, or any nucleic acid sequence encoding said gRNA, said gRNA guides at least one programmable engineered nuclease (PEN) to said target nucleic acid sequence in said target gene.

3. The method according to claim 1, wherein said at least one target nucleic acid sequence that participates directly or indirectly in at least one splicing event comprises at least one of, a splicing junction, a splice donor site, a splice acceptor site, an exonic splicing enhancer, splicing silencer, an intronic splicing enhancer and an intronic splicing silencer of said target gene.

4. The method according to claim 1, wherein said at least one target nucleic acid sequence that participates directly or indirectly in at least one splicing event are comprised within at least one coding transcript characterized by at least one of:
(i) said coding transcript/s comprise at least three exons;
(ii) at least one of said exons is of a length not divisible by three; and
(iii) the coding transcripts comprise at least one intron.

5. The method according to claim 1, wherein said target nucleic acid sequence is comprised within an exon, or within at least one intron located upstream or downstream to said exon, or within at least one splicing junction flanking said exon, wherein said exon is not the first or the last exon in said transcript.

6. The method according to claim 1, wherein at least one of:
(I) said splicing modulating agent comprises at least one oligonucleotide, said oligonucleotide is an antisense oligonucleotide (ASO) comprising at least fifteen contiguous nucleobases complementary to at least part of said at least one nucleic acid sequence that participates directly or indirectly in at least one splicing event;
(II) said PEN comprises at least one clustered regulatory interspaced short palindromic repeat (CRISPR)/CRISPR associated (cas) protein, and wherein said splicing modulating agent comprises:
(a) at least one nucleic acid sequence comprising at least one gRNA, or any nucleic acid sequence encoding said gRNA; or any kit, composition, vector or vehicle comprising said gRNA or nucleic acid sequence encoding said gRNA; and optionally
(b) at least one CRISPR/cas protein, or any nucleic acid molecule encoding said Cas protein, or any kit, composition, vector or vehicle comprising said CRISPR/cas protein or nucleic acid sequence encoding said CRISPR/cas protein.

7. (canceled)

8. The method according to claim 1, wherein at least one of:
(a) said neoplastic disorder is cancer, and wherein said target cell is a cancerous cell;
(b) said target gene is a gene differentially expressed in at least one of at least one cancer cell and at least one cancerous tissue; and
(c) said target gene is selected from the group of genes disclosed by Table 1;
(d) wherein said target sequence that participates directly or indirectly in at least one splicing event is comprised within an exon, within at least one intron located upstream or downstream to said exon, or within at least one splicing junction flanking said exon, wherein said exon is selected from the group of exons disclosed by Table 2.

9-11. (canceled)

12. The method according to claim 1, wherein:
(a) said target gene is the tyrosinase (TYR) gene, optionally, said cancer is melanoma; or
(b) said target gene is the heterogeneous nuclear ribonucleoprotein A/B (HNRNPAB), optionally, said cancer is breast cancer.

13. (canceled)

14. The method according to claim 1, wherein expression of said neoantigen by said target cell, leads to activation of an immune-response directed against said target cell.

15. (canceled)

16. The method according to claim 1, wherein said contacting step is performed in a mammalian subject suffering from a neoplastic disorder and wherein the method results in activating an immune response against at least one target cell in said subject, the method comprising the step of: administering to said subject at least one splicing modulating agent comprising at least one nucleic acid sequence, or of any vector, vehicle, matrix, nano- or micro-particle or composition comprising said at least one agent, wherein said at least one nucleic acid sequence of said agent targets at least one target nucleic acid sequence that participates directly or indirectly in at least one splicing event of a target gene;
wherein introduction of said agent into said target cell in said subject induces at least one aberrant splicing event via said target nucleic acid sequence;
and wherein said aberrant splicing event results in the production of at least one neoantigen, thereby activating an immune response directed against said target cell in said subject, optionally, wherein said neoplastic disorder is cancer, and wherein said target cell is a cancerous cell.

17. (canceled)

18. The method according to claim 16, wherein at least one of:
(a) the method further comprising the step of administering to said subject an effective amount of at least one peptide derived from said neoantigen, or any derivative, enantiomer, fusion protein, conjugate or polyvalent dendrimer thereof, wherein said peptide is administered prior to, after and/or simultaneously to administration of said splicing modulating agent; and
(b) said subject is further treated with at least one therapeutic agent, optionally, said therapeutic agent is an immuno-modulatory agent being at least one immune checkpoint inhibitor.

19-30. (canceled)

31. A method for treating, inhibiting, preventing, ameliorating or delaying the onset of at least one neoplastic disorder in a subject, the method comprising the step of:
administering to said subject at least one splicing modulating agent comprising at least one nucleic acid sequence, or of any vector, vehicle, matrix, nano- or micro-particle or composition comprising said at least one agent, wherein said at least one nucleic acid sequence of said agent targets at least one target nucleic acid sequence that participates directly or indirectly in at least one splicing event of a target gene; wherein introduction of said agent into said target cell in said subject induces at least one aberrant splicing event via said target nucleic acid sequence; and wherein said aberrant splicing event results in the production of at least one neoantigen expressed by said target cell, thereby activating an immune response directed against said target cell in said subject.

32. The method according to claim 31, wherein said splicing modulating agent comprises at least one of:
(a) at least one oligonucleotide comprising a nucleic acid sequence complementary to at least part of said target nucleic acid sequence; and
(b) at least one nucleic acid sequence comprising at least one gRNA that targets at least one protospacer within said target nucleic acid sequence, or any nucleic acid sequence encoding said gRNA, said gRNA guides at least one PEN to said target nucleic acid sequence in said target gene.

33. The method according to claim 31, wherein at least one of:
(a) said method further comprising the step of administering to said subject an effective amount of at least one peptide derived from said neoantigen, or any derivative, enantiomer, fusion protein, conjugate or polyvalent dendrimer thereof, wherein said peptide is administered prior to, after and/or simultaneously to administration of said splicing modulating agent; and
(b) said subject is further treated with at least one immuno-modulatory agent, optionally, said immuno-modulatory agent is at least one immune checkpoint inhibitor.

34. The method according to claim 31, wherein at least one of:
(I) said at least one target nucleic acid sequence that participates directly or indirectly in at least one splicing event comprises at least one of, a splicing junction, a splice donor site, a splice acceptor site, an exonic splicing enhancer, splicing silencer, an intronic splicing enhancer and an intronic splicing silencer of said target gene;
(II) said at least one nucleic acid sequence that participates directly or indirectly in at least one splicing event are comprised within at least one coding transcript characterized by at least one of:
(i) said coding transcript/s comprise at least three exons;
(ii) at least one of said exons is of a length not divisible by three; and
(iii) the coding transcripts comprise at least one intron; and
(III) said target nucleic acid sequence is comprised within an exon, or within at least one intron located upstream or downstream to said exon, or within at least one splicing junction flanking said exon, wherein said exon is not the first or the last exon in said transcript.

35-36. (canceled)

37. The method according to claim 31, wherein at least one of:
(I) said splicing modulating agent comprises at least one oligonucleotide, said oligonucleotide is an ASO comprising at least fifteen contiguous nucleobases complementary to at least part of said at least one nucleic acid sequence that participates directly or indirectly in at least one splicing event; and
(II) said PEN comprises at least one CRISPR/cas protein, and wherein said splicing modulating agent comprises:
(a) at least one nucleic acid sequence comprising at least one gRNA, or any nucleic acid sequence encoding said gRNA; or any kit, composition, vector or vehicle comprising said gRNA or nucleic acid sequence encoding said gRNA; and optionally
(b) at least one CRISPR/cas protein, or any nucleic acid molecule encoding said Cas protein, or any kit, composition, vector or vehicle comprising said CRISPR/cas protein or nucleic acid sequence encoding said CRISPR/cas protein.

38. (canceled)

39. The method according to claim 31, wherein at least one of:
(a) said neoplastic disorder is cancer, and wherein said target cell is a cancerous cell; and
(b) said target gene is selected from the group of genes disclosed by Table 1, optionally, said target sequence that participates directly or indirectly in at least one splicing event is comprised within an exon, within at least one intron located upstream or downstream to said exon, or within at least one splicing junction flanking said exon, wherein said exon is selected from the group of exons disclosed by Table 2.

40-41. (canceled)

42. The method according to claim 31, wherein:
(a) said target gene is the TYR gene, and optionally, said cancer is melanoma; or
(b) said target gene is HNRNPAB, and optionally, said cancer is breast cancer.

43. (canceled)

44. The method according to claim 31, wherein expression of said neoantigen by said target cell, leads to activation of an immune-response directed against said target cell.

45-47. (canceled)

48. A composition or kit comprising of at least one splicing modulating agent comprising at least one nucleic acid sequence or of any vector, vehicle, matrix, nano- or micro-particle thereof, wherein said nucleic acid sequence of said agent targets at least one target nucleic acid sequence that participates directly or indirectly in at least one splicing event of a target gene, said agent induces at least one aberrant splicing event via said nucleic acid sequence, said aberrant splicing event results in the production of at least one neoantigen expressed by at least one target cell of a subject suffering from a neoplastic disorder, optionally, wherein said splicing modulating agent comprises at least one of:
(a) at least one oligonucleotide comprising a nucleic acid sequence complementary to at least part of said target nucleic acid sequence; and
(b) at least one nucleic acid sequence comprising at least one gRNA that targets at least one protospacer within said target nucleic acid sequence, or any nucleic acid sequence encoding said gRNA, said gRNA guides at least one PEN to said target nucleic acid sequence in said target gene.

49-65. (canceled)


SEQUENCE LISTING (89 sequences)

SEQ ID NO: 1 - DNA, 182 bp, Homo sapiens
  misc_feature: TYR exon 4, of length 182bp from transcript NM_000372.4_3 (HG38)
tatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcag

SEQ ID NO: 2 - protein, 9 aa, Artificial Sequence
  New amino acids at the C-terminus of aberrant TYR isoform (derived from human)
RPRLFSRLH

SEQ ID NO: 3 - protein, 17 aa, Artificial Sequence
  Last 17 residues of the new aberrant TYR isoform (derived from human)
LLHHAFVDRPRLFSRLH

SEQ ID NO: 4 - RNA, 20 nt, Artificial Sequence
  TYR oligo 9 (designed for mouse)
gagggcggugccuucgcagc

SEQ ID NO: 5 - RNA, 20 nt, Artificial Sequence
  TYR oligo 13, (designed for mouse)
ucuuggagguagcuguaguc

SEQ ID NO: 6 - DNA, 20 bp, Artificial Sequence
  Oligo 9 target sequence in mouse TYR
gctgcgaaggcaccgccctc

SEQ ID NO: 7 - DNA, 20 bp, Artificial Sequence
  Oligos 13 target sequence in mouse TYR
gactacagctacctccaaga

SEQ ID NO: 8 - DNA, 182 bp, Mus musculus
  misc_feature: TYR exon 4, of length 182bp from transcript NM_001317397.1_2
tatttttgaacaatggctgcgaaggcaccgccctcttttggaagtttacccagaagccaatgcacctatcggccataacagagactcttacatggttcctttcataccgctctatagaaatggtgatttcttcataacatccaaggatctgggatatgactacagctacctccaagagtcag

SEQ ID NO: 9 - protein, 9 aa, Artificial Sequence
  New amino acids at the C-terminus of the aberrant TYR isoform (derived from mouse)
RSRLLQKLY

SEQ ID NO: 10 - DNA, 2065 bp, Homo sapiens
  misc_feature: TYR transcript NM_000372.4 (mRNA including UTRs) (HG38)
atcactgtagtagtagctggaaagagaaatctgtgactccaattagccagttcctgcagaccttgtgaggactagaggaagaatgctcctggctgttttgtactgcctgctgtggagtttccagacctccgctggccatttccctagagcctgtgtctcctctaagaacctgatggagaaggaatgctgtccaccgtggagcggggacaggagtccctgtggccagctttcaggcagaggttcctgtcagaatatccttctgtccaatgcaccacttgggcctcaatttcccttcacaggggtggatgaccgggagtcgtggccttccgtcttttataataggacctgccagtgctctggcaacttcatgggattcaactgtggaaactgcaagtttggcttttggggaccaaactgcacagagagacgactcttggtgagaagaaacatcttcgatttgagtgccccagagaaggacaaattttttgcctacctcactttagcaaagcataccatcagctcagactatgtcatccccatagggacctatggccaaatgaaaaatggatcaacacccatgtttaacgacatcaatatttatgacctctttgtctggatgcattattatgtgtcaatggatgcactgcttgggggatctgaaatctggagagacattgattttgcccatgaagcaccagcttttctgccttggcatagactcttcttgttgcggtgggaacaagaaatccagaagctgacaggagatgaaaacttcactattccatattgggactggcgggatgcagaaaagtgtgacatttgcacagatgagtacatgggaggtcagcaccccacaaatcctaacttactcagcccagcatcattcttctcctcttggcagattgtctgtagccgattggaggagtacaacagccatcagtctttatgcaatggaacgcccgagggacctttacggcgtaatcctggaaaccatgacaaatccagaaccccaaggctcccctcttcagctgatgtagaattttgcctgagtttgacccaatatgaatctggttccatggataaagctgccaatttcagctttagaaatacactggaaggatttgctagtccacttactgggatagcggatgcctctcaaagcagcatgcacaatgccttgcacatctatatgaatggaacaatgtcccaggtacagggatctgccaacgatcctatcttccttcttcaccatgcatttgttgacagtatttttgagcagtggctccgaaggcaccgtcctcttcaagaagtttatccagaagccaatgcacccattggacataaccgggaatcctacatggttccttttataccactgtacagaaatggtgatttctttatttcatccaaagatctgggctatgactatagctatctacaagattcagacccagactcttttcaagactacattaagtcctatttggaacaagcgagtcggatctggtcatggctccttggggcggcgatggtaggggccgtcctcactgccctgctggcagggcttgtgagcttgctgtgtcgtcacaagagaaagcagcttcctgaagaaaagcagccactcctcatggagaaagaggattaccacagcttgtatcagagccatttataaaaggcttaggcaatagagtagggccaaaaagcctgacctcactctaactcaaagtaatgtccaggttcccagagaatatctgctggtatttttctgtaaagaccatttgcaaaattgtaacctaatacaaagtgtagccttcttccaactcaggtagaacacacctgtctttgtcttgctgttttcactcagcccttttaacattttcccctaagcccatatgtctaaggaaaggatgctatttggtaatgaggaactgttatttgtatgtgaattaaagtgctcttattttaaaaaattgaaataattttgatttttgccttctgattatttaaagatctatatatgttttattggccccttctttattttaataaaacagtgagaaatcta

SEQ ID NO: 11 - DNA, 6042 bp, Mus musculus
  misc_feature: TYR transcript NM_011661.5 (mRNA including UTRs)
aagcttagtgtaaaacaggctgagagtatttgatgtaagaaggggagtggttatataggtcttagccaaaacatgtgatagtcactccaggggttgctggaaaagaagtctgtgacactcattaacctattggtgcagattttgtatgatctaaaggagaaaatgttcttggctgttttgtattgccttctgtggagtttccagatctctgatggccattttcctcgagcctgtgcctcctctaagaacttgttggcaaaagaatgctgcccaccatggatgggtgatgggagtccctgcggccagctttcaggcagaggttcctgccaggatatccttctgtccagtgcaccatctggacctcagttccccttcaaaggggtggatgaccgtgagtcctggccctctgtgttttataataggacctgccagtgctcaggcaacttcatgggtttcaactgcggaaactgtaagtttggatttgggggcccaaattgtacagagaagcgagtcttgattagaagaaacatttttgatttgagtgtctccgaaaagaataagttcttttcttacctcactttagcaaaacatactatcagctcagtctatgtcatccccacaggcacctatggccaaatgaacaatgggtcaacacccatgtttaatgatatcaacatctacgacctctttgtatggatgcattactatgtgtcaagggacacactgcttgggggctctgaaatatggagggacattgattttgcccatgaagcaccagggtttctgccttggcacagacttttcttgttattgtgggaacaagaaattcgagaactaactggggatgagaacttcactgttccatactgggattggagagatgcagaaaactgtgacatttgcacagatgagtacttgggaggtcgtcaccctgaaaatcctaacttactcagcccagcatccttcttctcctcctggcagatcatttgtagcagatcagaagagtataatagccatcaggttttatgcgatggaacacctgagggaccactattacgtaatcctggaaaccatgacaaagccaaaacccccaggctcccatcttcagcagatgtggaattttgtctgagtttgacccagtatgaatctggatcaatggatagaactgccaatttcagctttagaaacacactggaaggatttgccagtccactcacagggatagcagatccttctcaaagtagcatgcacaatgccttacatatctttatgaatggaacaatgtcccaagtacagggatcggccaacgatcccatttttcttcttcaccatgcttttgtggacagtatttttgaacaatggctgcgaaggcaccgccctcttttggaagtttacccagaagccaatgcacctatcggccataacagagactcttacatggttcctttcataccgctctatagaaatggtgatttcttcataacatccaaggatctgggatatgactacagctacctccaagagtcagatccaggcttttacagaaattatattgagccttacttggaacaagccagtcgtatctggccatggcttcttggggcagcactggtgggagctgttattgctgcagctctctctgggcttagcagtaggctatgccttcagaagaagaagaagaagaagcaaccccaggaggaaaggcagccactcctcatggacaaagacgactaccacagcttgctgtatcagagccatctgtgaacatcctaggaaacagagtgggactgaaaggttttacctcactcgacctatttgttggtgtttctacaaatttaaactagtataaaacatagaccatagctgtttggctttttttcagacccatgttttttcctaagtcctagtttctaagaaatgactgggatttgctaaaatatatatatatatataaataataacttactaatagctaaataaaatttcctcttacaactaattgagctggtttttatgaatgtgtcttaattatttaaacttgaggcacatttttgttttccttacttcattgtgaatttccaagaaaaatattctctctctctctctctctcgtgtgtttgtgtgtatgtgtgtgttaactgattcaaacaattttgaaaatcttggattgatagaaatgattcattaatttatgaaattatttcattaatgattaggaaagacgaataattactaaattagtaacagaggagaacatctgccagcttttattaattgtcatttaagttaccttatctaccttctgtgactggtggaaaaatatcaggcaagagatgggaatgctctgcctaataggatagtggctcctggaaggagtgggttattactagagattattacctgaagtttaccatagccagaaaattaatcaaaacagatgactcagtaacatctgaagcttcaagtcggcttgactgcaatctgaaatcatcaagcccaagagccaaaggaatgggaacagcgatgggaaactatctgaatcagattctagtgtgatagtgtcaggggcacatgggtcatctttgagaccttcacacctgttgagtcaccaaaatttgctgtgaatgtaaatttttactgtaaattaattttttcttttctttttaaaaagatttatttattattatacataagtacactgtagctgtcttcagacacaccagaagagggtgtcagatctcattacagatggttgtgagccaccatgtggttgctgggatttgaactcaggacctctggaagaacagtcagtgctcttacccgctgagccatctcgccagcccagtaaatttttactttagtgaaagtaaaatttaagttttagtttttagtttagtaaaattttaggaagcaaatttttagttttctaaactaatttttttttctagtactggacatcaacccagtgccttgtatatgcaatgcaagcattttcttgtactctgctacctagcatgtatatataaatctacccaacaaatgttcattacagctgacaagggtctttataaactcagtgtttccctttatcacaatacaattccctcctttgccacttcatgtcatcatagaatattgtttttttctctagcggttcaaggtatgtatttgtatagcagtcacacctttgataaaagttaccatctctttgattatatattctcattatggtaacaaaattatattatgactatttcaatatatctgaaagtttcattaaattctcattaactttgtatatttcagtcttgcttattgtgaagcttttataaattgcttcacttttttctgaaattgtcctgttgctacatcattctgttaagaaataaataagtggcaatattttccccctttcttcttcctctaggcttttaaaattaattttagtattcatatatttttatcaaatcaaacatgcaattctctatctgtagagattaacctgtgatcgatacaagtcattttaactagtgtccttatgaggctgacagcaagcataaatcattgatgttcagcatttatataagctttcatttatgagtagagtttgtcagtttatacaatagaatataaattataaacaagctattaataccacaaaagccttaacattatttagctgttgcaaactacatatgaacaactttagtaattttcatgttctatttaagttttatatgcttcaaatttattatttggggcctcttattgtttttttatatggcagcattctaggatggagtgtgaagagatttcttagcctcaatccccatctgatgccctatgggcagtgatggcttctgtttgagggacactcagttttctattcgagtcctggtagagttatcatgttctggtagaaggattcaagcccagtagtataggtacaccatgccttataatgactgagctattacatttaaaaaagaataaaagcatgaagctgaggaggcagtgaggtgggggtggatctgcggagagtcacatggagaagtggagggcagatttacattatatcaaattttcaaaaaataataaaatatttataaaatgtaattgaacgaacaatatgtgggttggaatcaaaactcatgtagaactttcttcagtcactagatctgattgattgtgaattgattattagccatttaaactagtagactcgagacttaccttaattatttagttgccaaatttgggatcacaattgaatatgtctcctcaaaaaatatcttaaaaattcatagtaatcactttcactttccttttgcctattttaaaatatatgcattctctttaattaaacagctctgcatcctgtttgtcccataggtaaataaaatgttgagtgtgaatatttagtgaaatatagttcttaataaatttgctttactaaaattatttttactttcttagtcttttttttttttagaaaattaaaagtaaatgtatagaagtaaaaagtttgttttctaaatcagtgttctatatgttgcataaactggttcctactgctggattttgttggctttccagtctcagagcagtgtctggattacaggtagatattgctgtactaggatgaaagtagactgttttaatgagagatactttcaatttcacaggaaaggtgatttctattatgttaggccaataatcctaactttaatggcatttcataatgggtgctgagttaactgtctgatgctgaaattatactaaaaataatgtggtgatcatggaacctcaaaataaaacaaaataaactaaaaatttatcaccttttaaaaatctagtattatcttttcattaactagaaaacaaatgttgaaatgtttaaaaagaaatatttagcaatatacacaataattgattaatttcagtttaatttttaaaatattttattctttcttaatgacaactactgtatgatctggctcataagtagtagcggtagcagcttttgcagagcatataccccctaaaatctgtgtcaatatactatttgagttccctgaacatctgtttattacagtgctactcacaacaactaggttgtggagacaccccagatgtctatccacagacaaatggatagagaaaccatggcatgtatacatggtggaggtttgttcagccataaagaagaattaaattatgtcatttgcaagaaaactaatgtatttataattcatgatgatgaatgcaataggtcacactaaaaatcacgtttttctatattatatatacatataagtgtatacatgcacattttcaggaggaactatttgttttttttttaacgcattctagaatgtagcaggaaagacaatattgtgggtagtttggggttgcatcagtataagcaaagaaaaagttatacaagtatgaaaattttataatgagtacattataaaatttgtaatgtgtacaataaatatataccaatatgctgtataataaatatataccaatgattaaagaaaaaatccaagtagttatgtattttacttataaagacaaaatgaaatcagcattaacttatttcacagatatttcctagaatttgggggtataaagatgattaagacacataaaccatatgctacattgaaaaatgcaagtggggtaataggaaagtagataactacttataaaattatgatggaaataatcattctagagtaatattctaaaacacaatttaaaaagtaaggtcataaaatagaaaaataattacccatctcaattctaagacataattttttagattagattaatctcttctttaacaattataatagaaccttttcatcatttatatactggatgcatgtctaagcactactctatataatttctgcttgtcatatgaacactgagtatggttatattactttattttaatgatgaagaagcttgaataacaaagatattttaagaagtcaaaaaccctattaaattgtatgtatcacaattttt

SEQ ID NO: 12 - protein, 20 aa, Artificial Sequence
  20 amino acids at the C-terminus of the aberrant TYR isoform (derived from mouse)
PIFLLHHAFVDRSRLLQKLY

SEQ ID NO: 13 - protein, 9 aa, Artificial Sequence
  Synthetic immunogenic 9mer in Tyr mouse (derived from mouse) used for vaccination
HAFVDRSRL

SEQ ID NO: 14 - protein, 9 aa, Homo sapiens
  MISC_FEATURE: Synthetic immunogenic 9mer derived from human Tyr
AFVDRPRLF

SEQ ID NO: 15 - protein, 8 aa, Gallus gallus
  MISC_FEATURE: OVA - 8 amino acids from the ovalbumin protein
SIINFEKL

SEQ ID NO: 16 - DNA, 25 bp, Artificial Sequence
  CRISPR  mTYR 5′ guide 1-1 (mouse target)
caccgattgttcaaaaatactacag

SEQ ID NO: 17 - DNA, 25 bp, Artificial Sequence
  CRISPR mTYR 5′ guide 1-2 (mouse target)
aaacctgtagtatttttgaacaatc

SEQ ID NO: 18 - DNA, 25 bp, Artificial Sequence
  CRISPR mTYR 3′ guide 1-1 (mouse target)
caccgtacagctacctccaagagtc

SEQ ID NO: 19 - DNA, 25 bp, Artificial Sequence
  CRISPR mTYR 3′ guide 1-2 (mouse target)
aaacgactcttggaggtagctgtac

SEQ ID NO: 20 - DNA, 25 bp, Artificial Sequence
  hTYR 5′ guide 1-1, (human target)
caccgtcctctgcagtatttttgag

SEQ ID NO: 21 - DNA, 25 bp, Artificial Sequence
  hTYR 5′ guide 1-2 (human target)
aaacctcaaaaatactgcagaggac

SEQ ID NO: 22 - DNA, 25 bp, Artificial Sequence
  hTYR 5′ guide 2-1 (human target)
caccgactgctcaaaaatactgcag

SEQ ID NO: 23 - DNA, 25 bp, Artificial Sequence
  hTYR 5′ guide 2-2 (human target)
aaacctgcagtatttttgagcagtc

SEQ ID NO: 24 - DNA, 25 bp, Artificial Sequence
  hTYR 3′ guide 3-1, (human target)
caccgtatagctatctacaagattc

SEQ ID NO: 25 - DNA, 25 bp, Artificial Sequence
  hTYR 3′ guide 3-2, (human target)
aaacgaatcttgtagatagctatac

SEQ ID NO: 26 - DNA, 25 bp, Artificial Sequence
  hTYR 3′ guide 4-1, (human target)
caccgctatctacaagattcaggta

SEQ ID NO: 27 - DNA, 25 bp, Artificial Sequence
  hTYR 3′ guide 4-2, (human target)
aaactacctgaatcttgtagatagc

SEQ ID NO: 28 - RNA, 20 nt, Artificial Sequence
  hTYR oligo 1 (designed for human)
cuuauuuuacuuuaaaaauu

SEQ ID NO: 29 - RNA, 20 nt, Artificial Sequence
  hTYR oligo 2 (designed for human)
uucaaauguuucuuuuauac

SEQ ID NO: 30 - RNA, 20 nt, Artificial Sequence
  hTYR oligo 3 (designed for human)
acaauauguuucuuagucug

SEQ ID NO: 31 - RNA, 20 nt, Artificial Sequence
  hTYR oligo 4 (designed for human)
aauaaccuuuuccucugcag

SEQ ID NO: 32 - RNA, 20 nt, Artificial Sequence
  hTYR oligo 5 (designed for human)
ccuuuuccucugcaguauuu

SEQ ID NO: 33 - RNA, 20 nt, Artificial Sequence
  hTYR oligo 6 (designed for human)
uccucugcaguauuuuugag

SEQ ID NO: 34 - RNA, 20 nt, Artificial Sequence
  hTYR oligo 7 (designed for human)
ugcaguauuuuugagcagug

SEQ ID NO: 35 - RNA, 20 nt, Artificial Sequence
  hTYR oligo 8 (designed for human)
uugagcaguggcuccgaagg

SEQ ID NO: 36 - RNA, 20 nt, Artificial Sequence
  hTYR oligo 9 (designed for human)
gcuccgaaggcaccguccuc

SEQ ID NO: 37 - RNA, 20 nt, Artificial Sequence
  hTYR oligo 10 (designed for human)
caccguccucuucaagaagu

SEQ ID NO: 38 - RNA, 20 nt, Artificial Sequence
  hTYR oligo 11 (designed for human)
cauccaaagaucugggcuau

SEQ ID NO: 39 - RNA, 20 nt, Artificial Sequence
  hTYR oligo 12 (designed for human)
ucugggcuaugacuauagcu

SEQ ID NO: 40 - RNA, 20 nt, Artificial Sequence
  hTYR oligo 13 (designed for human)
gacuauagcuaucuacaaga

SEQ ID NO: 41 - RNA, 20 nt, Artificial Sequence
  hTYR oligo 14 (designed for human)
aucuacaagauucagguaaa

SEQ ID NO: 42 - RNA, 20 nt, Artificial Sequence
  hTYR oligo 15 (designed for human)
caagauucagguaaaguuua

SEQ ID NO: 43 - RNA, 20 nt, Artificial Sequence
  hTYR oligo 16 (designed for human)
uucagguaaaguuuacuuuc

SEQ ID NO: 44 - RNA, 20 nt, Artificial Sequence
  hTYR oligo 17 (designed for human)
guaaaguuuacuuucuuuca

SEQ ID NO: 45 - RNA, 20 nt, Artificial Sequence
  hTYR oligo 18 (designed for human)
gaggaauugcugaaucuagu

SEQ ID NO: 46 - RNA, 20 nt, Artificial Sequence
  hTYR oligo 19 (designed for human)
guuaccaauuuauuuugaga

SEQ ID NO: 47 - RNA, 20 nt, Artificial Sequence
  hTYR oligo 20 (designed for human)
uaacacaaaacuuuaugcuu

SEQ ID NO: 48 - DNA, 103 bp, Mus musculus
  misc_feature: Hnrnpab exon 6 in transcript NM_010448.3, mm10_dna range=chr1151602593-51602695
tgtgaaatcaaggttgcccagcccaaagaggtgtatcagcaacagcagtatggctctgggggcagaggaaatcgcaatcgagggaaccgaggcagtggtggag

SEQ ID NO: 49 - DNA, 2404 bp, Mus musculus
  misc_feature: Hnrnpab transcript, mm10_ncbiRefSeq_NM_010448.3 range=chr1151600100-51606881
ggcattataaagagctccacgagttggcattgtcaggcggtggcaccgcgcgggacggagttaaggtcttctttatcggcagtgtacggcggcggccgagcggagcgatagtcagtggctgcggacgagccctcgggggtgcgagcgtgaccgcggccaagcccgcgcgtcgccttctgcggcgagtagggcctcgcggcgtcatcggcggcgagtagccgctgcggcctggccgaccatgtcggacgcggctgaggagcagcccatggagacgacgggcgccaccgagaacggacacgaggccgcccccgaaggcgaggccccggtcgagcccagcgcggctgccgcggcccccgcggcctcggccgggtctgggggcgggaccacgaccgcaccgagcgggaaccagaacggcgcggagggcgaccagatcaacgccagcaagaacgaggaggacgcgggaaaaatgttcgttggtggtctgagctgggataccagcaaaaaagacttaaaggattattttactaagtttggagaggttgttgactgtacaataaaaatggatcccaacactggacgatcaagagggtttgggtttattctcttcaaagattcttctagtgtggaaaaggtcttagatcagaaggagcacaggctggatggtcgtgtcattgaccctaaaaaggctatggctatgaagaaggaccctgtgaagaaaatctttgtgggaggtctaaaccctgaagccacagaggaaaagatcagagaatacttcggccagtttggggagattgaggccattgagcttccaatagatcccaagttgaacaaaagacggggttttgtttttattacatttaaagaggaagatcctgtgaagaaagttctggagaagaaatttcatactgtcagtggaagcaagtgtgaaatcaaggttgcccagcccaaagaggtgtatcagcaacagcagtatggctctgggggcagaggaaatcgcaatcgagggaaccgaggcagtggtggaggtcagggtagtacaaattacggaaagagccagcgacgtggtggtcatcagaataactacaagccatactgagaggcagcaggagcggccgagtgacgaccgcacacgctttgtttggacgcggagtgaacacaattatgtaccaaatttaacttggcaaacttttatggcctgtcccatgtgcatcttatttaaaatttcccccagaaatcactctcctgtttactattccagagctctagttgtaggcagcgtgtggcgtctcagaggccagagcggcactagggggctgattttattaccaggttaccctgttctagatcaggagggtctatttcctgttgccgctctgcagcctggacctgtggaccctggttgtaaagagtaaaactgtatcttaggaaaccagtgtcacctttttcaccttttaattttatattatttgtgtcatacatttccagtaatggaagtgttaattttactgtactttttggtaccttttgggaatctaatgtattgtaaggtattttacacgtgtcctgattttgccacgacctggatattgaagctatccaagcttttgaaataaaaatgaaaaacaaaacccccaagcctgggtaaatgtgggatgctctgtgagaccttgcccagtgttggagatgccaggtctcctgtagcaaagcagaggtcctttagttatcccatctgatctttttagctgcccttttgtgcccattgtctccatagcagagaccatcttgccatgtattatatatattccatgttcttagtttcagtcaagtacaagttgaaatgttggggatctgctgatggaggtggggccaagtctggcttacctggggggtgcttgatcctcagggtttcacaacttcttactttttcttccatgtctggaaaatgatcacagatcgtttctttttccccccaagacagggtttctctgtagccctggttaggttatcctggaactctgtagaccaggctggccttgaactcagaaatccacctggctctgcctcccaagtgctgggattaaaggcgtgcgccaccacgcctggcctaccctcagtttctaagatgcttgggatagtttagagattggcttatgtctcatcatttccctagcacagatggtccctttgtaagcccagtcaccactcactgttctgtacacatagaaccctctaagaaatgtatcaattccaaaacgcagtaggggtggctaacaagcaggaataggaagggatccttggtagcttgttttttgtctgttgggatctgagctttgtataacaaccactttggtgtatagtcatttctagttcggtagttttcttaaataaattgagggagaccttg

SEQ ID NO: 50 - DNA, 118 bp, Homo sapiens
  misc_feature: HNRNPAB exon 6 in transcript, hg38_ncbiRefSeq_NM_004499.3_5 range=chr5178209330-178209447
tgtgagatcaaggtggcccagcccaaagaagtctatcagcagcagcagtatggctctgggggccgtggaaaccgcaaccgagggaaccgaggcagcggaggtggtggtggaggtggag

SEQ ID NO: 51 - DNA, 1675 bp, Homo sapiens
  misc_feature: HNRNPAB transcript, hg38_ncbiRefSeq_NM_004499.3 chr5178204507-178211183
aaagggcgccacgagtcggcattgtcaggcggcggcaccgcgcgggacggagcttggctgttggtcggtgggttcccgtgcggcggcggccaaggaggaggagacacagttggagcagctccgtgggctgactggggcgaggcctcagcagcgcgagcttgagtgcggccgagcctgcggcgccttcccctgcgggtggggacgagcgggccccgcggcgtcatcggcggcgaggagccgccgcgcctcggcctagcatgtcggaagcgggcgaggagcagcccatggagacgacgggcgccaccgagaacggacatgaggccgtccccgaaggcgagtcgccggccggggctggcacgggcgccgcggcgggggctggaggcgcgaccgcggcgcccccgagcgggaatcagaacggcgccgagggcgaccagatcaacgccagcaagaacgaggaggacgcgggaaaaatgttcgttggtggcctgagctgggatactagcaaaaaagatttaaaagactattttactaaatttggagaggtcgttgactgtacaataaaaatggatcccaacactggacggtcaagagggtttgggtttatcctgttcaaagatgcagccagtgtggagaaggtcctagaccagaaggagcacaggctggatggccgtgtcattgaccctaaaaaggccatggctatgaagaaggacccggtgaagaaaatcttcgttgggggtctgaatcctgaagccactgaggaaaagatcagggagtactttggcgagtttggggagattgaggccattgaattgccaatggatccaaagttgaacaaaagacgaggttttgtgtttatcacctttaaagaagaagaacccgtgaagaaggttctggagaaaaagttccatactgtcagtggaagcaagtgtgagatcaaggtggcccagcccaaagaagtctatcagcagcagcagtatggctctgggggccgtggaaaccgcaaccgagggaaccgaggcagcggaggtggtggtggaggtggaggtcagggtagtacaaactacggcaagagccagcgacgtggtggccatcagaataactacaagccatactgaggcggcagcaggagcgaccaactgatcgcacacatgctttgtttggatatggagtgaacacaattatgtaccaaatttaacttggcaaactttctattgcctgtcccatgtgcatcttatttaaaatttcccccatggaaatcactctcctgttgactatttccagagctctaggtgtttaggcagcgtgtggtgtctgagaggccatagcgccatcatgggctgatttttattaccaggtcccccagaagcaggtgggaggctctgcttcctgctgccgctctgcagcctggacctgtggaccctggttgtaaagagtaaattgtatcttaggaaaccagtgtcacctttttttcaccttttaattttatattatttgcgtcatacatttcctgtaacggaagtgttaattttactgtactttttggtaccttttgggaatctaatgtattgtaaggtattttacacgtgtcctgattttgccacaacctggatattgaagctatccaagcttttgaaataaaatttaaaaacccccaagcctgggtgagtgtgggata

SEQ ID NO: 52 - protein, 9 aa, Artificial Sequence
  Immunogenic 9mer 1 of aberrant Hnrnpab isoform (derived from mouse)
VPNLTWQTF

SEQ ID NO: 53 - protein, 9 aa, Artificial Sequence
  Immunogenic 9mer 2 of aberrant Hnrnpab isoform (derived from mouse)
VYYSRALVV

SEQ ID NO: 54 - protein, 10 aa, Artificial Sequence
  Immunogenic peptide in Balb/c mice (Viral)
RGPGRAFVTI

SEQ ID NO: 55 - protein, 356 aa, Artificial Sequence
  New protein excluding exon 6 from Hnrnpab transcript NM_010448.3, (derived from mouse)
MSDAAEEQPMETTGATENGHEAAPEGEAPVEPSAAAAAPAASAGSGGGTTTAPSGNQNGAEGDQINASKNEEDAGKMFVGGLSWDTSKKDLKDYFTKFGEVVDCTIKMDPNTGRSRGFGFILFKDSSSVEKVLDQKEHRLDGRVIDPKKAMAMKKDPVKKIFVGGLNPEATEEKIREYFGQFGEIEAIELPIDPKLNKRRGFVFITFKEEDPVKKVLEKKFHTVSGSKVRVVQITERASDVVVIRITTSHTERQQERPSDDRTRFVWTRSEHNYVPNLTWQTFMACPMCILFKISPRNHSPVYYSRALVVGSVWRLRGQSGTRGLILLPGYPVLDQEGLFPVAALQPGPVDPGCKE

SEQ ID NO: 56 - protein, 254 aa, Artificial Sequence
  New protein excluding exon 6 from Hnrnpab transcript NM_004499.3, (derived from HUNAN)
MSEAGEEQPMETTGATENGHEAVPEGESPAGAGTGAAAGAGGATAAPPSGNQNGAEGDQINASKNEEDAGKMFVGGLSWDTSKKDLKDYFTKFGEVVDCTIKMDPNTGRSRGFGFILFKDAASVEKVLDQKEHRLDGRVIDPKKAMAMKKDPVKKIFVGGLNPEATEEKIREYFGEFGEIEAIELPMDPKLNKRRGFVFITFKEEEPVKKVLEKKFHTVSGSKVRVVQTTARASDVVAIRITTSHTEAAAGATN

SEQ ID NO: 57 - DNA, 25 bp, Artificial Sequence
  CRISPR mHnrnpab 3′ guide 1-1 (mouse target)
caccggggaaccgaggcagtggtgg

SEQ ID NO: 58 - DNA, 25 bp, Artificial Sequence
  CRISPR mHnrnpab 3′ guide 1-2 (mouse target)
aaacccaccactgcctcggttcccc

SEQ ID NO: 59 - DNA, 25 bp, Artificial Sequence
  hHnrnpab 5′ guide 1-1, (human target)
caccgtgacttttagtgtgagatca

SEQ ID NO: 60 - DNA, 25 bp, Artificial Sequence
  hHnrnpab 5′ guide 1-2, (human target)
aaactgatctcacactaaaagtcac

SEQ ID NO: 61 - DNA, 25 bp, Artificial Sequence
  hHnrnpab 5′ guide 2-1, (human target)
caccggtcctcttgacttttagtgt

SEQ ID NO: 62 - DNA, 25 bp, Artificial Sequence
  hHnrnpab 5′ guide 2-2, (human target)
aaacacactaaaagtcaagaggacc

SEQ ID NO: 63 - DNA, 25 bp, Artificial Sequence
  hHnrnpab 3′ guide 3-1, (human target)
caccgggaggtggtggtggaggtgg

SEQ ID NO: 64 - DNA, 25 bp, Artificial Sequence
  hHnrnpab 3′ guide 3-2, (human target)
aaacccacctccaccaccacctccc

SEQ ID NO: 65 - DNA, 25 bp, Artificial Sequence
  hHnrnpab 3′ guide 4-1, (human target)
caccggtggtggaggtggaggtgag

SEQ ID NO: 66 - DNA, 25 bp, Artificial Sequence
  hHnrnpab 3′ guide 4-2, (human target)
aaacctcacctccacctccaccacc

SEQ ID NO: 67 - RNA, 20 nt, Artificial Sequence
  hHNRNPAB oligo 1 (designed for human)
uuugucgcagugaagguguu

SEQ ID NO: 68 - RNA, 20 nt, Artificial Sequence
  hHNRNPAB oligo 2 (designed for human)
ugggcagucacugcccugag

SEQ ID NO: 69 - RNA, 20 nt, Artificial Sequence
  hHNRNPAB oligo 3 (designed for human)
uuuguccuacuggccugacc

SEQ ID NO: 70 - RNA, 20 nt, Artificial Sequence
  hHNRNPAB oligo 4 (designed for human)
acuguccucuugacuuuuag

SEQ ID NO: 71 - RNA, 20 nt, Artificial Sequence
  hHNRNPAB oligo 5 (designed for human)
ccucuugacuuuuaguguga

SEQ ID NO: 72 - RNA, 20 nt, Artificial Sequence
  hHNRNPAB oligo 6 (designed for human)
ugacuuuuagugugagauca

SEQ ID NO: 73 - RNA, 20 nt, Artificial Sequence
  hHNRNPAB oligo 7 (designed for human)
uuuagugugagaucaaggug

SEQ ID NO: 74 - RNA, 20 nt, Artificial Sequence
  hHNRNPAB oligo 8 (designed for human)
gaucaagguggcccagccca

SEQ ID NO: 75 - RNA, 20 nt, Artificial Sequence
  hHNRNPAB oligo 9 (designed for human)
gcccagcccaaagaagucua

SEQ ID NO: 76 - RNA, 20 nt, Artificial Sequence
  hHNRNPAB oligo 10 (designed for human)
aagaagucuaucagcagcag

SEQ ID NO: 77 - RNA, 20 nt, Artificial Sequence
  hHNRNPAB oligo 11 (designed for human)
gcaaccgagggaaccgaggc

SEQ ID NO: 78 - RNA, 20 nt, Artificial Sequence
  hHNRNPAB oligo 12 (designed for human)
gaaccgaggcagcggaggug

SEQ ID NO: 79 - RNA, 20 nt, Artificial Sequence
  hHNRNPAB oligo 13 (designed for human)
agcggagguggugguggagg

SEQ ID NO: 80 - RNA, 20 nt, Artificial Sequence
  hHNRNPAB oligo 14 (designed for human)
gugguggagguggaggugag

SEQ ID NO: 81 - RNA, 20 nt, Artificial Sequence
  hHNRNPAB oligo 15 (designed for human)
ggagguggaggugaguggaa

SEQ ID NO: 82 - RNA, 20 nt, Artificial Sequence
  hHNRNPAB oligo 16 (designed for human)
uggaggugaguggaacgugg

SEQ ID NO: 83 - RNA, 20 nt, Artificial Sequence
  hHNRNPAB oligo 17 (designed for human)
gugaguggaacguggaugaa

SEQ ID NO: 84 - RNA, 20 nt, Artificial Sequence
  hHNRNPAB oligo 18 (designed for human)
gauagguccuguuucccugc

SEQ ID NO: 85 - RNA, 20 nt, Artificial Sequence
  hHNRNPAB oligo 19 (designed for human)
cuacauggagccuuccaagc

SEQ ID NO: 86 - RNA, 20 nt, Artificial Sequence
  hHNRNPAB oligo 20 (designed for human)
cugucuuggggcucccucug

SEQ ID NO: 87 - DNA, 20 bp, Artificial Sequence
  CRISPR mHnrnpab 5′ guide 1-1 (mouse target)
tggcttttagtgtgaaatca

SEQ ID NO: 88 - DNA, 20 bp, Artificial Sequence
  CRISPR mHnrnpab 5′ guide 1-2  (mouse target)
tgatttcacactaaaagcca

SEQ ID NO: 89 - protein, 9 aa, Homo sapiens
  MISC_FEATURE: Synthetic immunogenic 9mer derived from human Tyr
HAFVDRPRL